(19) United States
(12) Patent Application Publication
(10) Pub. No.: US 2025/0154584 A1
(43) Pub. Date: May 15, 2025

(54) METHODS FOR IDENTIFYING POLYPEPTIDES

(71) Applicant: Encodia, Inc., San Diego, CA (US)

(72) Inventors: Jason C. Klima, San Diego, CA (US); Mark Alec Kowarsky, Redwood City, CA (US); Aaron Wise, San Diego, CA (US); Devin Sullivan, San Diego, CA (US)

(21) Appl. No.: 18/951,277

(22) Filed: Nov. 18, 2024

Related U.S. Application Data
(63) Continuation of application No. PCT/US2024/055265, filed on Nov. 8, 2024.
(60) Provisional application No. 63/597,668, filed on Nov. 9, 2023.

(51) Int. Cl. C12Q 1/6874; G01N 33/68; G16B 30/00; G16B 40/20

(52) U.S. Cl. CPC C12Q 1/6874; G01N 33/6803; G16B 30/00; G16B 40/20

(57) ABSTRACT

The present disclosure relates to methods and systems for identifying polypeptides using nucleic acid data obtained from a polypeptide sequencing device that encode amino acid sequence information into barcoded nucleic acid sequences that are later analyzed in a nucleic acid sequencer. Nucleic acid data generated as an output of the nucleic acid sequencer are processed to retrieve information about the analyzed polypeptides. The process includes generating a plurality of binder identifier strings for the generated nucleic acid data, and providing the plurality of binder identifier strings as input to a computer model configured to infer amino acid sequences of polypeptides from binder identifier strings. The disclosure finds utility at least in a variety of methods and related systems for high-throughput polypeptide analysis and sequencing.

### CROSS-REFERENCE TO RELATED APPLICATIONS

The present application is a continuation of International Application No. PCT/US2024/055265, filed Nov. 8, 2024, which claims priority to U.S. Provisional Application No. 63/597,668, filed Nov. 9, 2023, the disclosures of which are herein incorporated by reference in their entirety.

### REFERENCE TO AN ELECTRONIC SEQUENCE LISTING

The contents of the electronic sequence listing (776532005201SEQLIST.xml; Size: 35,043 bytes; and Date of Creation: Nov. 12, 2024) is herein incorporated by reference in its entirety.

### TECHNICAL FIELD

The present disclosure generally relates to biotechnology, in particular to methods for identifying polypeptides using nucleic acid sequence data obtained from a polypeptide sequencing device that encodes amino acid sequence information as barcoded nucleic acid sequences. The disclosed methods find utility in a variety of high-throughput polypeptide analysis and sequencing applications.

### BACKGROUND

High-throughput sequencing of polypeptide analytes in biological samples remains a challenge. Several approaches to high-throughput polypeptide sequencing have been published, including U.S. Pat. No. 9,435,810 B2, WO2010/065531A1, US 2019/0145982 A1, US 2020/0348308 A1, US 20200209255 A1, U.S. Ser. No. 11/549,942 B2, US 20180299460 A1, US 20210079557A1, and US 2023/0136966 A1, which utilize sequential amino acid or epitope recognition by a limited set of degenerate binding agents as a critical step during a polypeptide sequencing assay. In addition, reagents for cleaving components of polypeptides recognized by binding agents have been separately adopted or developed (see, e.g., U.S. Ser. No. 11/427,814 B2). However, intrinsic difficulty in discriminating structurally similar individual amino acid residues within polypeptides creates a high level of errors, making decoding of output data from polypeptide sequencing assays a challenging task, compromising wide adoption of high-throughput polypeptide sequencing methods. There remains a need in the art for improved techniques relating to decoding methods for high-throughput polypeptide sequencing data.

The present disclosure describes algorithms for polypeptide sequencing data processing that fulfill the described and other needs. These and other embodiments of the invention will be apparent upon reference to the following detailed description. To this end, various references are set forth herein which describe in more detail certain background information, procedures, compounds and/or compositions, and are each hereby incorporated by reference in their entireties.

### BRIEF SUMMARY

Disclosed herein are bioinformatics tools for analyzing biological macromolecules, including peptides, polypeptides, and polypeptides, that complement recently described methods for polypeptide analysis that employ a nucleic acid-based polypeptide encoding technique. The disclosed bioinformatics methods enable high-throughput processing of nucleic acid-encoded polypeptide data to identify and quantify thousands of peptides and/or polypeptides present in a biological sample.

Recently, methods for high-throughput polypeptide characterization have been published, e.g., US 2019/0145982 A1, U.S. Ser. No. 11/513,126 B2, and US 2023/0136966 A1, which utilize use of nucleic acid-barcoded binding agents recognizing particular components of an immobilized polypeptide in a cyclic manner and encoding binding history of each polypeptide after each binding event in a nucleic acid recording tag, thus generating an extended recording tag. Accordingly, upon recognition of a portion of a polypeptide by a nucleic acid-barcoded binding agent, a nucleic acid signal about the binding event is created and recorded in the recording tag associated with the polypeptide, which could later lead to probabilistic identification of the recognized portion. The recognized portion (e.g., terminal amino acid residue) is cleaved off, creating opportunity for recognition of a new component (e.g., newly formed terminal amino acid residue) of the immobilized polypeptide. This process can be repeated until enough information regarding polypeptide components is encoded (e.g., 2-10 binding/encoding cycles may be enough for polypeptide identification), see also FIG. 2. After encoding of information regarding binding agents that are bound to a particular immobilized polypeptide in the extended recording tag associated with the polypeptide, the extended recording tags can be analyzed in parallel by, for example, next-generation sequencing (NGS). and information regarding components of the polypeptides is elucidated by decoding the information regarding binding agents that are bound to these polypeptides during each binding cycle.

Provided herein is a computer-implemented method for analyzing a plurality of polypeptides encoded in a plurality of nucleic acid sequences by an encoding assay, the method comprising: (a) receiving, at one or more processors, the plurality of nucleic acid sequences generated from the encoding assay, wherein each of the plurality of nucleic acid sequences comprises a series of encoder barcode sequences, and wherein each encoder barcode sequence of a given series of encoder barcode sequences corresponds to a binder, from a set of binders, that binds to one or more components of a polypeptide of the plurality of polypeptides; (b) generating a binder identifier string for each nucleic acid sequence of the plurality of nucleic acid sequences based on a corresponding series of encoder barcode sequences, thereby generating a plurality of binder identifier strings corresponding to the plurality of nucleic acid sequences; (c) inferring, using the one or more processors, an amino acid sequence of a polypeptide of the plurality of polypeptides from binder identifiers of a binder identifier string of the plurality of binder identifier strings based on (i) binding profiles of the binders from the set of binders that correspond to the binder identifiers of the binder identifier string, and (ii) calculated probability scores of an association between one or more binder identifiers of the binder identifier string and one or more amino acid sequences of polypeptides of the plurality of polypeptides; and (d) based on the calculated probability scores and the inferred amino acid sequences, outputting data related to at least a partial identity and/or quantity for at least one polypeptide of the plurality of polypeptides.

Provided herein is a computer-implemented method for analyzing a plurality of polypeptides encoded in a plurality of nucleic acid sequences by an encoding assay, the method comprising: a) receiving, at one or more processors, the plurality of nucleic acid sequences generated from the encoding assay, wherein each of the plurality of nucleic acid sequences comprises a series of encoder barcode sequences, and wherein each barcode sequence of the series of encoder barcode sequences corresponds to a binder that binds to a component of a polypeptide of the plurality of polypeptides in the encoding assay; b) assigning, using the one or more processors, a binder identifier to each of the series of encoder barcode sequences in each of the plurality of nucleic acid sequences to generate a plurality of binder identifier strings for the plurality of nucleic acid sequences; c) providing, using the one or more processors, the plurality of binder identifier strings as input to a trained machine learning model, wherein the trained machine learning model is configured to infer amino acid sequences of polypeptides of the plurality of polypeptides from binder identifier strings based on binding profiles of binders that correspond to binder identifiers assigned in a given binder identifier string, and output data related to at least a partial identity and/or quantity for at least one polypeptide of the plurality of polypeptides.

Provided herein is also a computer-implemented method for analyzing a plurality of polypeptides encoded in a plurality of nucleic acid sequences by an encoding assay, the method comprising: a) receiving, at one or more processors, the plurality of nucleic acid sequences generated from the encoding assay, wherein each of the plurality of nucleic acid sequences comprises a series of encoder barcode sequences, and wherein each barcode sequence of the series of encoder barcode sequences corresponds to a binder that binds to a component of a polypeptide of the plurality of polypeptides in the encoding assay; b) assigning, using the one or more processors, a binder identifier to each of the series of encoder barcode sequences in each of the plurality of nucleic acid sequences to generate a plurality of binder identifier strings for the plurality of nucleic acid sequences; c) for each polypeptide and/or polypeptide fragment of the plurality of polypeptides, generating in silico a set of simulated binder identifier strings based on pre-determined parameters of the encoding assay, wherein the pre-determined parameters comprise: i) probabilities of assigning to at least one component of a given polypeptide or polypeptide fragment one or more binder identifiers either correctly or incorrectly based on binding profiles of binders used in the encoding assay; and ii) optionally, for at least one component of a given polypeptide or polypeptide fragment, a probability of successfully cleaving the at least one component after a binder binds to the at least one component in the encoding assay, thereby determining probabilities for each simulated binder identifier string generated from a given polypeptide or polypeptide fragment; d) for each binder identifier string of the plurality of binder identifier strings generated in (b), calculate probabilities that a given binder identifier string is generated from one or more polypeptides or fragments thereof based on probabilities for simulated binder identifier strings determined in (c), thereby determining at least a partial identity and/or quantity for at least one polypeptide of the plurality of polypeptides.

Provided herein is also a system, comprising: one or more processors; and a memory communicatively coupled to the one or more processors and configured to store instructions that, when executed by the one or more processors, cause the system to perform the above-mentioned computer-implemented methods.

Provided herein is also a non-transitory computer-readable storage medium storing one or more programs, the one or more programs comprising instructions, which when executed by one or more processors of a system, cause the system to perform the above-mentioned computer-implemented methods.

The summary is not intended to be used to limit the scope of the claimed subject matter. Other features, details, utilities, and advantages of the claimed subject matter will be apparent from the detailed description including those embodiments disclosed in the accompanying drawings and in the appended claims.

### BRIEF DESCRIPTION OF THE DRAWINGS

Non-limiting embodiments of the present invention will be described by way of example with reference to the accompanying figures, which are schematic and are not intended to be drawn to scale. For purposes of illustration, not every component is labeled in every figure, nor is every component of each embodiment of the invention shown where illustration is not necessary to allow those of ordinary skill in the art to understand the invention.

FIG. 1. Exemplary flow diagram for a method of analyzing polypeptides showing key steps of the sample preparation and analysis.

FIG. 2. Exemplary schematic illustration of an assay for performing reverse translation (also known as encoding assay) to encode polypeptide (e.g., peptide or polypeptide) sequences as barcoded nucleic acid sequences.

FIG. 3A. Exemplary flow diagram for a method of identifying polypeptides based on an analysis of nucleic acid sequence data generated from a polypeptide encoding process that utilizes partially selective binders and that may exhibit a high error rate for calling individual amino acid residues.

FIG. 3B. Exemplary flow diagram for a method of identifying polypeptides based on an analysis of nucleic acid sequence data generated from a polypeptide encoding process such as those depicted in FIG. 2.

FIGS. 4A-D. Exemplary flow diagrams for a method of identifying polypeptides based on an analysis of nucleic acid sequence data generated from a polypeptide encoding process that utilizes partially selective binders and that may exhibit a high error rate for calling individual amino acid residues. FIG. 4A shows exemplary steps of one embodiment of the method that comprises generating in silico a set of simulated binder identifier strings based on pre-determined parameters of the encoding assay (see Example 8 for more detail), whereas FIG. 4C shows a general scheme of this embodiment. FIG. 4B shows exemplary steps of a second embodiment of the method that comprises generating a plurality of peptidic reads and a probability score for each peptidic read based on likelihood that a given peptidic read is produced from a given binder identifier string (see Example 9 for more detail), whereas FIG. 4D shows a general scheme of the second embodiment.

FIG. 5. Exemplary schematic illustration of a computing device in accordance with one or more examples of this disclosure.

FIG. 6. Exemplary schematic illustration of the structure of a nucleic acid recording tag used to encode peptide and/or polypeptide sequence information.

FIGS. 7A-B. Exemplary schematic illustrations of an assay for performing reverse translation to encode peptide and/or polypeptide sequence information as barcoded nucleic acid sequences. FIG. 7A: assay utilizing only double-stranded ligation. FIG. 7B: assay utilizing both ligation on one strand of a double-stranded region and extension.

FIG. 8. Exemplary barcode-level alignments of nucleic acid sequence reads. A model nucleic acid sequence (SEQ ID NO: 12) is analyzed.

FIG. 9. Exemplary output of the computer model that generates at least one binder identifier string for each analyzed nucleic acid sequence read as described in FIG. 8. Indicated nucleic acid sequence reads comprise the following sequences: SEQ ID NO: 13-SEQ ID NO: 17.

FIG. 10. Exemplary schematic illustration of a proteome analysis system according to one implementation as described herein.

FIG. 11. Exemplary schematic illustration of potential errors that may occur when encoding polypeptide sequence information as barcoded nucleic acid sequences.

FIG. 12. Exemplary false discovery rate (FDR) data as a function of polypeptide concentration in a sample analyzed using the disclosed assay and bioinformatics techniques.

FIG. 13. Exemplary schematic illustration of the difference between read-level analysis and bulk-level analysis.

FIG. 14. Exemplary schematic illustration of the use of an Expectation-Maximization (EM) algorithm to refine predictions of polypeptide abundances in a sample output by a trained machine learning model.

FIG. 15. Exemplary simulated false discovery rate (FDR) data as a function of the relative abundance for polypeptides in a sample analyzed using the disclosed assay and bioinformatics techniques.

FIG. 16A-FIG. 16G illustrate exemplary heatmap data showing encoding efficiency for seven engineered binders having sequences set forth in SEQ ID NO:1-SEQ ID NO:7, respectively, which are used together in the multiplex encoding assay on immobilized set of at least 289 peptides as a set of binders. The more intense white color of a cell representing a particular P1-P2 combination indicates higher encoding efficiency.

FIG. 17 shows exemplary heatmap data showing encoding efficiency for individual binder having the sequence set forth in SEQ ID NO:7 that was used in the multiplex encoding assay at different concentrations (15 nM, 31 nM, 63 nM, 125 nM, 250 nM and 500 nM). The more intense white color of a cell representing a particular P1-P2 combination indicates higher encoding efficiency.

FIG. 18A-FIG. 18B show theoretical modeling and experimental data demonstrating dependence of the encoding yield of an individual binder from the binder concentration in the encoding assay. FIG. 18A shows exemplary modeling of an encoding yield of an individual binder depending on the binder concentration [A]. FIG. 18B shows exemplary experimental data for concentration-dependent encoding yields of two individual engineered binders.

FIG. 19A-FIG. 19B show exemplary method for estimation of NTF efficiency for an N-terminal modifier agent reacting with NTAA residue of peptides. FIG. 19A shows exemplary scheme that utilizes biotinylated N-terminal modifier agent and detection of successful NTAA installation by binding with streptavidin and encoding of the binding information. FIG. 19B shows exemplary experimental heatmap data for the encoding efficiency which depend on particular P1-P2 residues of immobilized peptides.

FIG. 20 shows exemplary calculation of modified NTAA cleavage efficiencies (NTC efficiencies) for immobilized set of peptides using experimental heatmap data and a particular Cleavase enzyme acting on the array of peptides having different P1-P2 residues.

FIG. 21A-FIG. 21B illustrate exemplary simulation process of generating probability distributions for simulated binder identifier strings as described in Example 8. A1A2A3 . . . represents sequence of analyzed polypeptide. FIG. 21A shows potential outcomes for cleavage of a terminal amino acid residue after the first and the second encoding cycle. FIG. 21B shows outcomes for assignment of a binder identifier for a given terminal amino acid residue within the encoding cycle. For A1 residue, BBI, BI1 and BI2 are assigned (based on simulated binding of binding agents 1 and 2). For A2 residue, BBI, BI3 and BI4 are assigned (based on simulated binding of binding agents 3 and 4).

FIG. 22A-FIG. 22B show exemplary calculation of probability scores for peptidic reads generated for two particular binder identifier strings using a model system.

FIG. 23A-FIG. 23B show another example of calculating probabilities of peptidic reads based on a given binder identifier string “LVNDVZDF” (SEQ ID NO: 18). Generated peptidic reads contain amino acid sequences as set forth in SEQ ID NO: 19-SEQ ID NO: 27.

FIG. 24 illustrates exemplary generation of a large number of custom polypeptide-polynucleotide conjugates immobilized on a solid support that may be used for large-scale encoding assays with data used for training of machine learning models (see Example 10).

### DETAILED DESCRIPTION

Highly parallel characterization and recognition of polypeptides remains a challenge. In proteomics, one goal is to identify and quantitate numerous polypeptides in a sample, which is a formidable task to accomplish in a high-throughput way. Assays such as immunoassays and mass spectrometry-based methods have been used but are limited at both the sample and analyte level, with limited sensitivity and dynamic range, and with cross-reactivity and background signals. Multiplexing the readout of a collection of affinity agents to a collection of cognate polypeptides, for example, using affinity agents with detectable labels, remains challenging.

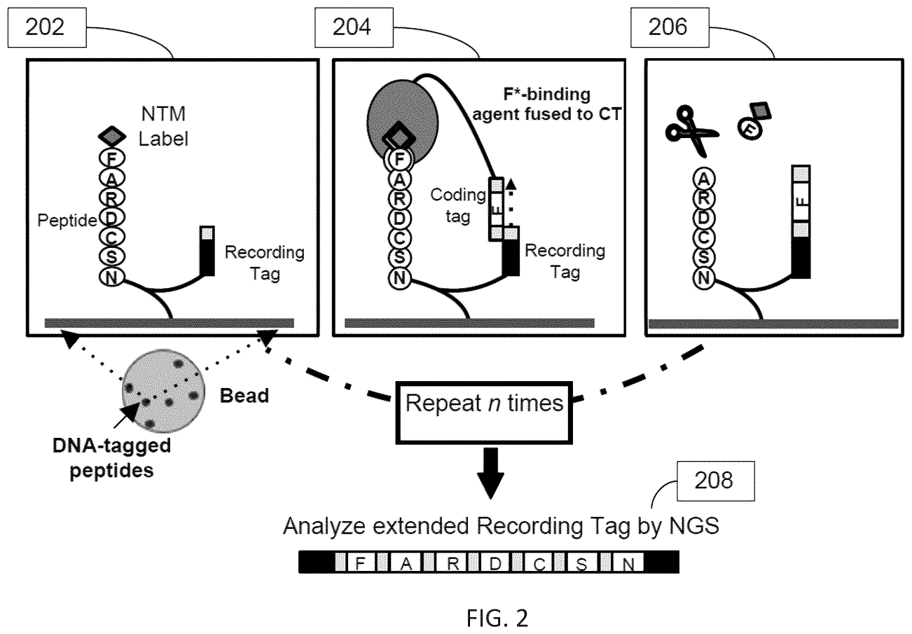

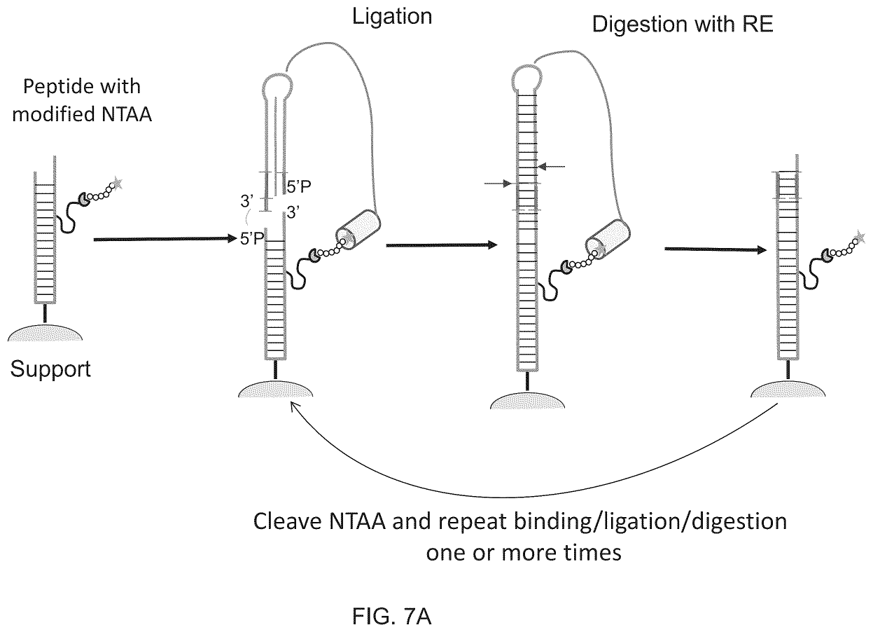

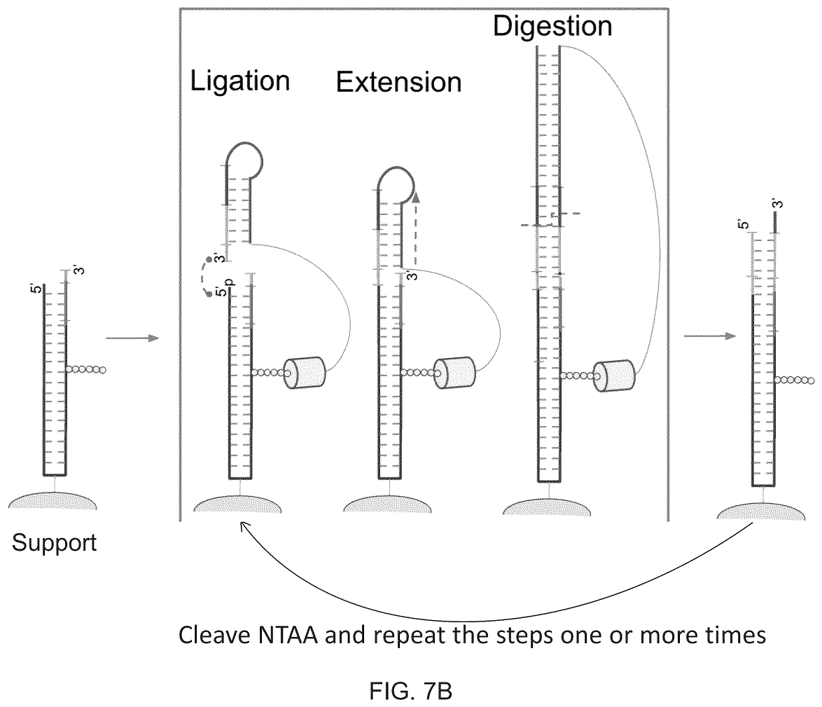

Recently, a new format of high-throughput polypeptide sequencing assay (called a NGPS (next generation peptide sequencing) assay, ProteoCode® assay or an encoding assay) was reported in the published US patent applications US 2019/0145982 A1 and US 2023/0136966 A1, see also FIG. 2 and FIG. 7A-FIG. 7B. During an exemplary assay, immobilized polypeptides each associated with a nucleic acid recording tag are contacted with a set of binding agents (or binders) each capable of binding to a different set of modified N-terminal amino acid (NTAA) residues of polypeptides, wherein each binding agent comprises a nucleic acid coding tag with identifying information regarding the binding agent. During each binding cycle, the coding tag and the recording tag are placed in a sufficient proximity for interaction, and the information regarding the binding agent that bound to the polypeptide in the current cycle is transferred between the coding tag and the recording tag, e.g., from the coding tag to the recording tag, thus generating an extended recording tag associated with the polypeptide. After completion of the transfer, the modified NTAA residues of polypeptides are cleaved with a specific reagent (see, e.g., U.S. Ser. No. 11/427,814 B2 or US 2022/0227889 A1), thereby generating new NTAA residues of polypeptides, which can be assayed in a similar manner in the next binding cycle with a new set of binding agents.

After at least one successive binding cycle, a nucleic acid encoded library representative of the binding history of each polypeptide is generated, wherein information about each binding event is encoded in the extended recording tags. Following analysis of the extended recording tags (usually by a nucleic acid sequencing method), information about the binding agents (binders) bound to the polypeptides at each cycle can be decoded, providing information regarding components (e.g., NTAA residues) of the polypeptides to which the binding agents were bound. Thus, the disclosed variants of ProteoCode® assay (which are collectively referred to as “encoding assay” throughout this disclosure) represent an unconventional way of characterizing, identifying or quantifying a large number of polypeptides in parallel, and provides means for sequential identification of individual amino acid residues of polypeptides with a certain probability.

In some embodiments, the Encoding assay generates a nucleic acid encoded library representation of the binding history of each polypeptide of the plurality of target polypeptides (i.e., polypeptide analytes). This nucleic acid encoded library can be amplified, and analyzed using high-throughput next generation digital sequencing methods, enabling millions to billions of molecules to be analyzed per run. The creation of a nucleic acid encoded library of binding information is useful in another way in that it enables enrichment, subtraction, and normalization by DNA-based techniques that make use of hybridization. These DNA-based methods are easily and rapidly scalable and customizable, and more cost-effective than those available for direct manipulation of other types of macromolecule libraries, such as polypeptide libraries. Thus, nucleic acid encoded libraries of binding information can be processed prior to sequencing by one or more techniques to enrich and/or subtract and/or normalize the representation of sequences. This enables information of maximum interest to be extracted much more efficiently, rapidly and cost-effectively from very large libraries whose individual members may initially vary in abundance over many orders of magnitude.

In some embodiments, analysis of composition of extended recording tags (e.g., by determining incorporated nucleic acid moieties by using nanopore sequencing, or by full sequencing of the extended recording tags) is used to obtain information regarding one or more binders that were specifically bound to the analyzed polypeptide, as well as the order in which these binders were bound. Binding profiles and the binding order of the binders provide identifying information regarding specific components (e.g., modified NTAA residues) of the analyzed polypeptide to which the binders were bound, as well as regarding overall sequence of the analyzed polypeptide. As a result of the encoding cycles described above, structural information of immobilized polypeptides is encoded as nucleic acid sequences. Accordingly, identities of immobilized polypeptides can be decoded from sequences of corresponding associated recording tags by calculating probabilities of occurrence of specific types of amino acid residues in corresponding places in amino acid sequences of the immobilized polypeptides. Binders in the described approach do not need to be strictly selective and may recognize, for example, structure/functional classes of NTAA residues, such as negatively charged residues, positively charged residues, small hydrophobic residues, aromatic residues, and so on, or recognize other NTAA residue types. This is the case because several binders can be used simultaneously for decoding a single NTAA residue.

In preferred embodiments of the Encoding assay, after several cycles of binding/transferring, each immobilized polypeptide is back-translated into a series of unique nucleic acid barcodes on the corresponding recording tag associated with the immobilized polypeptide. During the analysis step, sequence of the extended recording tag can be analyzed to extract the above-mentioned nucleic acid barcodes that correspond to each encoding cycle. Then, to associate the extracted nucleic acid barcodes with corresponding amino acid residues, an artificial intelligence (AI) model can be applied to calculate probabilities of occurrence of specific types of amino acid residues in corresponding places in amino acid sequence of the analyzed peptide. In some embodiments, the AI model can be pre-trained using multiple known polypeptide sequences, which were used to generate nucleic acid encoding data on associated recording tags. Modeling encoding of multiple known polypeptide using known set of binders allows for training the AI model to faithfully predict amino acid residues based on provided encoder barcode sequences.

Existing challenges for high-throughput polypeptide identification with the Encoding assay include absence of binders with strong selectivity towards individual terminal amino acid residues of polypeptide analytes, which results in high error rate for individual amino acid call during the analysis. Most binders have “diffusive” specificity (may interact with several structurally similar NTAAs with certain probability). In addition, most binders possess “P1P2 specificity”, meaning that their affinity towards particular terminal amino acid residue (i.e., P1 residue) depends on penultimate terminal amino acid of the polypeptide (i.e., P2 residue), so binders tend to bind better polypeptides having certain combinations of P1 and P2 residues, further complicating the analysis and making information about the encoded NTAA ambiguous. Other sources of errors in Encoding data include i) an absence of signal in a particular binding cycle due to failed encoding event (failure to transfer identifying information onto extended RT); and ii) incorrect signal generated due to non-specific binding or off-target binding. Non-specific binding occurs when a binder binds non-specifically to a support or to other components of the system. Off-target binding occurs when a binder binds to a non-cognate component of a polypeptide (e.g., binds to an NTAA which it is not supposed to bind). Such non-specific binding or off-target binding may occasionally generate a misleading encoding signal. All of the discussed factors create a significant variability in the Encoding performance and form a basis for an error in identification of particular residue and identity of a polypeptide in general.

Due to the potential for error introduced by the Encoding assay, computer models (e.g., comprising the use of AI- or machine learning-based algorithms running on one or more processors) can be used to identify potential sources of error, predict the likelihood of generating multiple binder identifier “signature” reads from a known polypeptide, and then calculate the probability of specific peptide identification based on the existing binder identifier string data. Due to the inherent complexity of the assay (as explained above), multiple proteomic “signature” reads (e.g., dozens, hundreds or even thousands of proteomic “signature” reads; also referred to herein as “peptidic reads”) might be generated as encoding signals from a single polypeptide, and the computer models discussed herein enable one to match the generated proteomic “signature” reads back to the single polypeptide.

### Exemplary Bioinformatic Analysis Workflow:

As discussed above, the output of the Encoding assay for analysis of a plurality of polypeptides comprises a library of nucleic acid sequences, wherein each nucleic acid sequence of the library corresponds to an extended recording tag associated with a polypeptide derived from one of analyzed polypeptides. Accordingly, each nucleic acid sequence of the library comprises a series of encoder barcode sequences, wherein each of the series of encoder barcode sequences is indicative of a binder that binds to the analyzed polypeptide derived from one of analyzed polypeptides. In some embodiments, each nucleic acid sequence of the library further comprises at least one auxiliary sequence, wherein the at least one auxiliary sequence comprises one or more sample barcode sequences, one or more bead barcode sequences, one or more unique molecular identifier (UMI) sequences, one or more spacer sequences, sequencing primer sequences, and/or complements or combinations thereof.

In some embodiments, the library of nucleic acid sequences is obtained from an NGS device and received at one or more processors for further analysis. In these embodiments, initial NGS data processing may be performed using public tools. Exemplary non-limiting NGS data processing tools are provided below.

a) Create FASTQ format files using bclconvert or bcl2fastq from raw sequencing files. The FASTQ is a text-based sequence file format that is generated from a binary base call (BCL) file (a raw data file generated by an Illumina sequencer), which stores both raw sequence data and quality scores. Alternative processing tools may be used. For example, the Illumina BCL Convert software is a Linux application that converts the BCL files produced by Illumina sequencing systems to FASTQ format files.

b) Obtain quality control statistics using FastQC/MultiQC tools. Quality control on nucleic acid samples from a sequencer is important for further productive mapping. FastQC aims to provide a simple way to do quality control checks on raw sequence data coming from a high-throughput sequencer. It provides a modular set of analyses which one can use to get a quick check on whether the data has any issues of which the user should be aware before doing any further data analysis. MultiQC is a program that creates summaries over all samples for several different types of QC-measures. MultiQC aggregates results from bioinformatics analyses across many samples into a single report. It searches a given directory for analysis logs and compiles a HTML report. MultiQC will automatically look for output files from the supported tools and make a summary of them.

c) Merge paired-end reads with BBMerge. BBMerge is designed to merge two overlapping paired reads into a single read (Bushnell B, Rood J, Singer E. BBMerge—Accurate paired shotgun read merging via overlap. PLoS One. 2017 Oct. 26; 12(10):e0185056). Merging paired-end reads can improve various subsequent bioinformatics processes. BBMerge also provides the ability to merge non-overlapping shotgun read pairs by using k-mer frequency information to assemble the unsequenced gap between reads, achieving a significantly higher merge rate while maintaining or increasing accuracy.

Alternative processing tools may also be used. After initial processing, a plurality of nucleic acid sequences is generated from the library of nucleic acid sequences received from the NGS device. In some embodiments, the plurality of nucleic acid sequences are present in a text-based sequencing data file format, such as FASTQ, that stores both raw sequence data and quality scores. Such format can be used as input for a wide variety of secondary data analysis solutions.

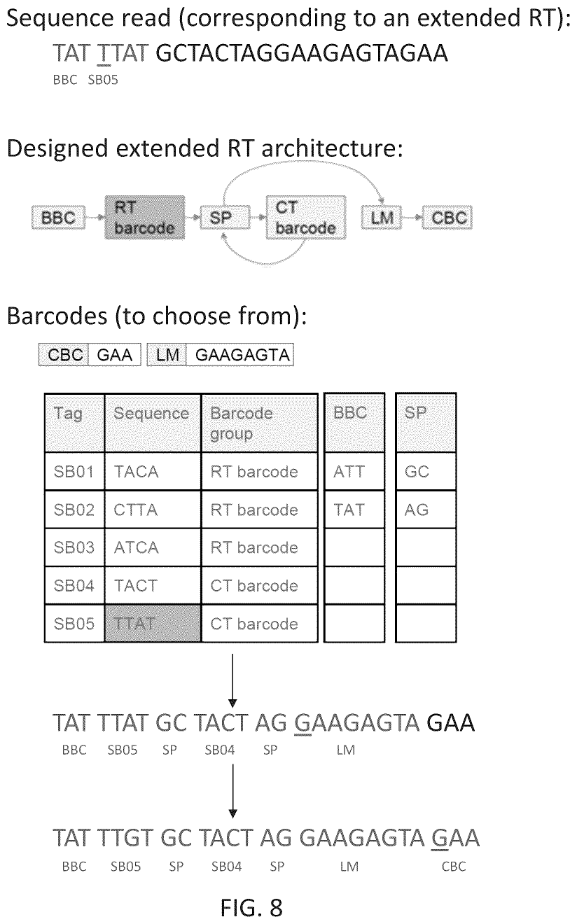

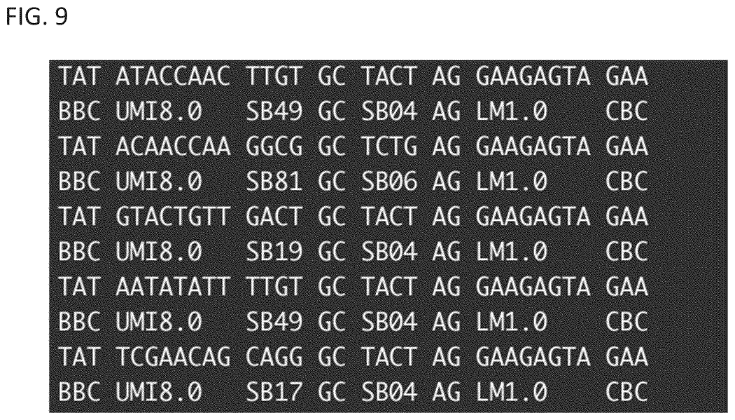

In the next step, a plurality of identifier strings is generated for the plurality of nucleic acid sequences by assigning, using the one or more processors, a binder identifier to each of the series of encoder barcode sequences and, optionally, an auxiliary identifier to at least one of the one or more auxiliary sequences in each of nucleic acid sequences of the plurality. In some embodiments, nucleic acid sequences in FASTQ format provided from the previous processing step are aligned and assigned binder identifiers and auxiliary identifier(s). Exemplary assignment is illustrated in FIG. 8 and FIG. 9. In these embodiments, the designed extended RT architecture comprises a certain sequence of barcodes, spacers and other elements present in each extended RT, and, accordingly, in each nucleic acid sequence of the plurality of nucleic acid sequences generated after initial processing of raw sequencing data (see, e.g., SEQ ID NO: 12-SEQ ID NO: 17). Exemplary barcodes designed to be present in a particular order in extended RT comprise: BBC barcode, RT barcode, CT barcode(s), spacer sequences, LM sequence and CBC barcode attached during the final capping step, which indicates the end of the sequence read. In some embodiments, some of these sequences or barcodes are not present in the extended RTs. In some embodiments, other sequences or barcodes may be present in the extended RTs. During the described step, a binder identifier is assigned to each of the CT barcodes present in each of the analyzed nucleic acid sequences, and an auxiliary identifier is assigned to at least one of the auxiliary sequences in each of the analyzed nucleic acid sequences. In some embodiments, all other barcodes and identifying sequences except for CT barcodes are designated as “auxiliary sequences” for the purpose of the assignment. The key difference between CT barcodes and auxiliary sequences is that CT barcodes comprise identifying information regarding the binding agents that were bound to a particular polypeptide during the above described one or more binding cycles. Thus, given the determined binding profiles of the binding agents, the analysis of CT barcodes that correspond to a particular analyzed polypeptide provides actual structural information about the particular analyzed polypeptide (e.g., identities of particular amino acids within the sequence of the polypeptide). In contrast, auxiliary sequences provide additional information about the analyzed polypeptide, such as which sample the polypeptide originated from, which compartment or fraction the polypeptide originated from, or what polypeptide the polypeptide originated from (e.g., when the polypeptide is formed during polypeptide fragmentation). Also, auxiliary sequences may correspond to one or more optional elements of the extended RT architecture, including spacer sequences (typically, present between CT barcodes), sequences that would be used for extended RT amplification, or sequences that indicate the beginning or end of the sequence read. In some embodiments, an auxiliary identifier is assigned to all of the auxiliary sequences in each of the analyzed nucleic acid sequences. Typically, each auxiliary sequence has a particular use in the polypeptide identification assay; for example, BBC/RT barcode identifies the sample from which the analyzed polypeptide originated from, and spacer sequences are necessary for encoding to occur (in some embodiments).

In some embodiments, the computer model for assigning binder identifiers to each of the encoder barcode sequences and, optionally, assigning one or more auxiliary identifiers to at least one of the auxiliary sequences is a Talon-LUT, which uses a look up table to quickly assign tags (i.e., identifiers) to the various fragments of an analyzed nucleic acid sequence. It uses a ‘grammar’ to know which kind of ‘word’ (i.e., type of barcode or auxiliary sequence) comes next in the designed extended RT architecture. It performs error correction by enumerating all common types of errors of each known barcode or auxiliary sequence. In some embodiments, common types of errors include DNA sequencing errors, such as single nucleotide polymorphisms (SNPs) and indels. An example of such error correction is as follows. For a barcode ACCCCGT, enumerated errors can be assigned by the computer model within a certain probability (such as enumerated errors CCCCCGT, GCCCCGT, AACCCGT . . . ). If the computer model encounters a read that contains CCCCCGT, it's not an exact match to the barcode, but it is an exact match to one of the enumerated errors. Thus, the computer model matches it to the enumerated error sequence, and also to its associated exact barcode.

In some embodiments, the computer model for assigning binder identifiers to each of the encoder barcode sequences and, optionally, assigning one or more auxiliary identifiers to at least one of the auxiliary sequences is a Talon-HMM, which uses a Hidden Markov model (HMM) (or another similar probabilistic model) to model each sequencing read. In some embodiments, the Baum-Welch algorithm is used to find the most likely sequence of tags for each read. The Baum-Welch algorithm is a special case of the expectation-maximization algorithm (EM algorithm). Its purpose is to tune the parameters of the HMM, namely the state transition matrix A, the emission matrix B, and the initial state distribution no, such that the model is maximally like the observed data. Although the Talon-HMM is slower than Talon-LUT, it provides more accuracy to the assignment.

In some embodiments, additional tools that produce sample level statistics for the plurality of nucleic acid sequences are used.

In the next step, the plurality of identifier strings generated in the previous step is provided, using the one or more processors, as input to a computer model. In some embodiments, the plurality of identifier strings may be further processed or converted before providing them as input to the computer model. In some embodiments, the plurality of identifier strings is converted into a plurality of peptidic reads based on determined binding profiles of binders that correspond to binder identifiers assigned in a given binder identifier string. In some embodiments, the computer model reads a plurality of identifier strings produced by Talon-LUT or Talon-HMM, recognizes binder identifiers within the plurality of identifier strings, and produces a “peptidic read” correspond to a series of possible encoding events in the encoding assay. In preferred embodiments, multiple peptidic reads are produced from a given identifier string, depending on complexity of binding profiles of the binders that were bound to the analyzed polypeptide during the encoding assay and in view of probabilistic nature of other steps in the encoding assay (efficiencies of NTF, NTE and NTC steps; see also Example 7 and Example 9 below). In some embodiments, the plurality of identifier strings is not converted into a plurality of peptidic reads before used an as input to the computer model.

In some embodiments, a plurality of binder identifier strings generated in the previous step is evaluated against a plurality of polypeptides that can be present in the analyzed sample(s). In some embodiments, the plurality of binder identifier strings is provided as input to a trained machine learning (ML) model, wherein the ML model is configured to assign binder identifier strings to amino acid sequences of polypeptides of the plurality of polypeptides (all polypeptides that can possible be present in the analyzed sample(s)) and output at least a partial identity and/or quantity for at least one polypeptide of the plurality of polypeptides. Two different workflows that can be used for this step are described below.

1) Bulk analysis algorithm.

The bulk analysis algorithm looks for unique signatures that are correlated with the quantity of a specific polypeptide in a sample. Unique signatures are identified from training data. A statistical threshold can be chosen such that a signature read pattern occurs from the target polypeptide frequently, but from the background—rarely. The output of a bulk analysis algorithm is a sample-level quantification information (e.g., ‘Polypeptide X is at Y peptides per million in this sample’) and may include probabilistic assignments of reads to individual polypeptides. The input of this algorithm comprises two types of training data: a) training data from pure polypeptide samples of interest (e.g., recombinant polypeptides of interest which can produce particular signature reads that are identifiable)—a “foreground” sample; and b) training data from “background” sample, such as a plasma sample. The bulk analysis algorithm needs training data from both foreground and background to analyze real-life polypeptide samples. Using these training data, it chooses unique signatures that are constant in the background, and at the same time can uniquely identify a foreground (a specific polypeptide of interest). For each sample of interest, it uses these identified unique signatures to predict the quantity of each foreground polypeptide. The bulk analysis algorithm does that by correcting the observed amount of the polypeptide (based on the unique signature(s)) with a correction factor calculated from the training data. A correction may be calculated based on the known true positive and false positive rates in the foreground and background samples respectively.

Next, the bulk analysis algorithm uses expectation maximization (EM) to refine the predicted quantifications. EM is an iterative algorithm for refining model predictions. The EM iteration alternates between performing an expectation (E) step, which creates a function for the expectation of the likelihood evaluated using the current estimate for the parameters, and a maximization (M) step, which computes parameters maximizing the expected likelihood found on the E step. These estimates of parameters are then used to determine the distribution of the latent variables in the next E step. In some embodiments, the following two steps are repeated in EM until convergence: 1) Expectation: find the best assignment (in this case, polypeptide label) for each data point (in this case, an input binder identifier string) given current model parameters (current estimate for what polypeptides are in the sample). 2) Maximization: find the best estimates for the model parameters given the current assignments (e.g., current CT barcode assignments).

2) Read-level analysis. The output of read-level analysis are per-read mappings (e.g., ‘Read X comes from Polypeptide Y’), as well as sample-level quantification information (e.g., ‘Polypeptide X is at Y peptides per million in this sample’). The read-level analysis can be performed in a few different ways, described below. A more detailed description is provided in Example 8 below.

(2A) “Top-down” algorithm inspired by Salmon (Patro R, et al., Salmon provides fast and bias-aware quantification of transcript expression. Nat Methods. 2017 April; 14(4):417-419), but differs from Salmon in the details. It uses an HMM-like probabilistic model trained on empirical assay parameters to build an ‘index’ mapping short read fragments (called “k-mers”) to probabilities. For each binder identifier string, it breaks it into k-mers, determines the probabilities for each k-mer, and then determines if it can assign the binder identifier string to a specific polypeptide based on the determined probabilities for the set of k-mer fragments. K-mers are built from a particular binder identifier string as follows: for an exemplary read “A1A2A3A4A5A6A7”, all k-mers of length 5 (5-mers) would be A1A2A3A4A5, A2A3A4A5A6 and A3A4A5A6A7. The assignment of a binder identifier string to a specific polypeptide may be based on the product of the individual probabilities for all k-mers in the set, or on some other functional metric related to the k-mer probabilities.

In some embodiments, the parameters of the computer model are categorized as either “emission probabilities” (the probability of seeing barcode X given that the underlying amino acid is Y; which is, essentially, the encoding probabilities) and “transition probabilities” (the probability of proceeding to the next amino acid after encoding; i.e., the chance that cleavase enzyme would cleave off the NTAA residue after encoding to expose a new NTAA residue of the analyzed polypeptide). In some embodiments, “top-down” algorithm learns the parameters of the encoding system (emission and transition probabilities) from training data, and then applies this knowledge to real-world data.

(2B) Deep learning-based read mapping.

For this algorithm, the same empirical assay parameters are used as for the previously described “top-down” algorithm. The computer model generates multiple simulated encoding data from model polypeptides. The computer model uses probabilities (including the emission probabilities and transition probabilities described above) to create binder identifier strings based on a given input distribution of peptides. In some embodiments, the set of simulated binder identifier strings comprises at least 100,000, at least 500,000 or more simulated binder identifier strings. Accordingly, the generated simulated data is used to train a deep learning model to map from generated raw reads to probabilistic polypeptide assignment.

Next, real-world reads from actual encoding assays (instead of simulated reads) are used to further train the deep learning model, to learn aspects of error model that are not captured in the empirical assay parameters. The advantage of using simulated data first is that one can create orders of magnitude more training data by simulating, rather than from actually performing the assay. The deep learning model is used to fractionally assign each binder identifier string in a sample to polypeptides that may be present in the sample. As an example of fractional assignment: “For read X, the deep learning model predicts that it is 30% likely to come from polypeptide A, and 70% likely to come from polypeptide B”.

Next, the expectation maximization (EM) algorithm is used to refine the predicted read assignments and quantifications in a similar way as described above for the bulk analysis algorithm.

The bioinformatics tools for analyzing biological macromolecules, e.g., peptides, polypeptides, and polypeptides, that are described herein complement recently described methods for polypeptide analysis (i.e., next generation polypeptide assays (NGPAs)) that employ a nucleic acid-based polypeptide encoding technique. See, for example, U.S. Patent Application Publications US 2019/0145982 and US 2023/0136966 A1, each of which are incorporated herein by reference in their entirety. The disclosed bioinformatics methods enable high-throughput processing of nucleic acid-encoded polypeptide data to identify and quantify thousands of peptides and/or polypeptides present in a biological sample using a limited set of binders (e.g., 5-20 binders having imperfect selectivity towards terminal amino acid residues of peptides).

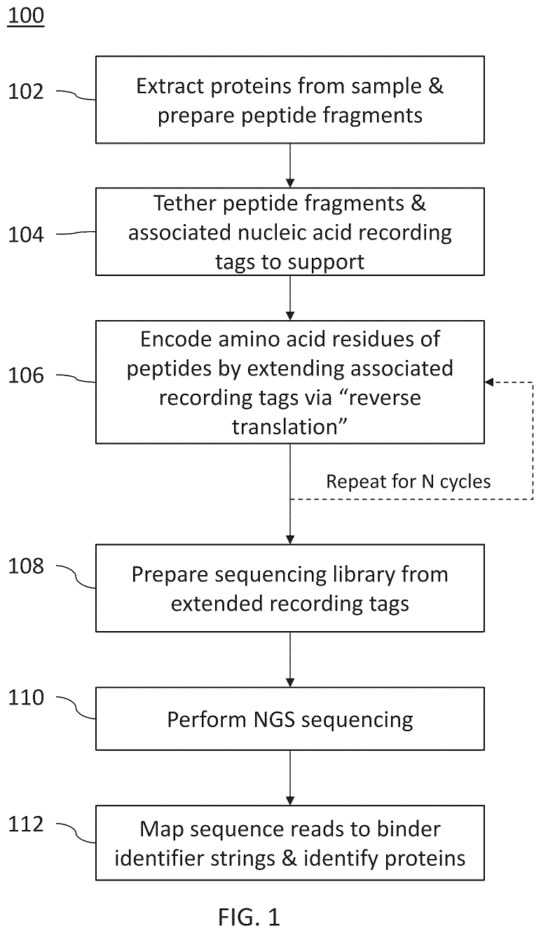

FIG. 1 provides a non-limiting example of a flowchart for a process 100 for identifying and/or quantifying peptides, polypeptides, and polypeptides extracted from a sample, which includes steps of an encoding assay (e.g., 106, 108 and 110).

At step 102 in FIG. 1, peptides, polypeptides, and polypeptides are extracted from a sample and fragmented. In some instances, the sample may be, for example, a tissue sample or cell extract sample. In some instances, the sample may be cell extract sample, and the cell extract sample may be a mammalian cell extract, a plant cell extract, a fungal cell extract, or a bacterial cell extract.

Peptides, polypeptides, and polypeptides may be extracted from the sample using any of a variety of techniques known to those of skill in the art, where the technique chosen may depend on the sample type and whether the extracted polypeptides in the lysate are to be analyzed in a non-denatured or denatured state. For the NGPA assay, either native conformation or denatured polypeptides can be utilized. Examples of the steps that may be combined to perform extraction of peptides and polypeptides include, but are not limited to, homogenization of tissue samples (e.g., by grinding), lysis of cell suspensions (e.g., by sonication or application of shear forces), polypeptide precipitation using acetone (for preparation of denatured lysates), solubilization and/or resuspension in appropriate polypeptide extraction buffer(s), immunoprecipitation, filtration, centrifugation, biomagnetic separation, and/or affinity chromatography.

Polypeptides and polypeptides extracted from a sample may be fragmented using any of a variety of techniques known to those of skill in the art. Examples include, but are not limited to, treatment with a specific protease or endopeptidase (e.g., TEV protease, which is specific for the ENLYFQ\S consensus amino acid sequence), treatment with a non-specific protease or endopeptidase (e.g., polypeptidease K), and/or treatment with a chemical regent that cleaves peptide bonds (e.g., cyanogen bromide (CNBr), hydroxylamine, hydrazine, etc.).

In some instances, fragmentation of polypeptides and/or polypeptides into peptide fragments may be performed prior to attachment of a DNA tag (e.g., a DNA recording tag as described below). In some instances, fragmentation of polypeptides and/or polypeptides may be performed following attachment of a DNA tag (e.g., a DNA recording tag as described below).

In some instances, peptides, polypeptides, and/or polypeptides may be subjected to one or more additional fractionation steps prior to attachment to a solid support. For example, the peptides, polypeptides, and/or polypeptides may be separated by (or enriched for) one or more properties such as cellular location, molecular weight, hydrophobicity, or isoelectric point. Alternatively, or additionally, one or more polypeptide enrichment steps may be used to select for a specific polypeptide or peptide (see, e.g., Whiteaker, et al. (2007), Anal. Biochem. 362:44-54) or to select for a particular post-translational modification (see, e.g., Huang, et al. (2014), J. Chromatogr. A 1372:1-17). Alternatively, a particular class or classes of polypeptides such as immunoglobulins, or immunoglobulin (lg) isotypes such as IgG, can be affinity enriched or selected for analysis. Overly abundant polypeptides can also be subtracted from the sample using standard immunoaffinity methods. Depletion of abundant polypeptides can be useful, e.g., for plasma samples where over 80% of the polypeptide constituent is albumin and immunoglobulins. Several commercial products are available for depletion of plasma samples of overly abundant polypeptides (see, e.g., PROTIA and PROT20 (Sigma-Aldrich, St. Louis, MO)).

At step 104 in FIG. 1, a plurality of peptides, polypeptides, or polypeptide fragments are tethered to a plurality of solid supports (e.g., beads or other substrates) along with corresponding recording tags.

In some instances, the plurality of peptides, polypeptides, or polypeptide fragments may comprise at least 100, 500, 1,000, 1,500, 2,000, 2,500, 5,000, 7,500, or 10,000 different peptides, polypeptides, or polypeptides.

In some instances, the peptides, polypeptides, or polypeptide fragments may be co-localized or co-labeled with a single or multiple recording tags. In some instances, recording tag(s) may comprise one or more universal priming sites (or complements thereof), one or more sequencing primer sites (or complements thereof), one or more barcodes (e.g., sample barcodes, partition barcodes, compartment barcodes, fraction barcodes, bead barcodes) or complements thereof, one or more optional unique molecular identifier (UMI) sequences (or complements thereof), one or more spacer sequences (used in information transfer from a coding tag to an extended recording tag as described below) (or complements thereof), one or more auxiliary sequences (or complements thereof), or any combination thereof. The one or more spacer sequences can be constant across all binding cycles, can be binder specific, or can be binding cycle number specific. The one or more auxiliary sequences may comprise, for example, one or more sample barcode sequences used to identify sample origin of a particular polypeptide or polypeptide analyte in an extended recording tag sequence or may comprise a “landmark” barcode to determine if encoding is complete.

In some instances, the peptides, polypeptides, or polypeptide fragments may be tethered to (or immobilized on) solid supports using a covalent bond. In some instances, the peptides, polypeptides, or polypeptide fragments may be tethered to (or immobilized on) solid supports using a non-covalent bond. Examples of suitable covalent and/or non-covalent techniques for immobilizing peptides, polypeptides, or polypeptide fragments are described in U.S. Patent Application Publication No. US 2019/0145982, which is incorporated herein by reference in its entirety.

In some instances, the solid supports may be, for example, nanoparticles, microspheres, beads (e.g., polystyrene beads, polymer beads, agarose beads, acrylamide beads, glass beads, controlled pore glass beads), solid core beads, porous beads, paramagnetic beads, porous matrices, arrays, glass surfaces, silicon surfaces, plastic or polymer surfaces, filters, membranes, silicon wafer chips, flow cells or microfluidic chips, biochips that include signal transducing electronics, microtiter wells, or ELISA plates, and the like.

At step 106 in FIG. 1, the peptides, polypeptides, or polypeptide fragments are encoded as nucleic acid sequences by performing a cyclic process comprising contacting the immobilized peptides, polypeptides, or polypeptide fragments with a set of binders (each comprising an attached coding tag), and extending the corresponding recording tag using a “reverse translation” process to transfer information from the coding tag attached to each binder to an extended recording tag (see also FIG. 2).

Binders may comprise, e.g., antibodies or fragments thereof, anticalins, N-recognins polypeptides (e.g., ATP-dependent Clp protease adaptor polypeptide (ClpS)), aptamers, etc., and variants or homologues thereof, and may further comprise coding tags that interact with an immobilized peptide, polypeptide, or polypeptide fragment that is co-localized and/or co-labeled with single or multiple recording tags. In some embodiments, binders comprise NTAA binders as disclosed in the patent publications U.S. Ser. No. 10/852,305 B2, US 2022/0283175 A1, US 2023/0220589 A1, incorporated herein by reference.

In some instances, the component of an immobilized peptide, polypeptide, or polypeptide to which the binder binds may comprise a peptide, polypeptide, or portion thereof, obtained by fragmenting a polypeptide molecule. In some instances, the component of an immobilized peptide, polypeptide, or polypeptide to which the binder binds may comprise one or more amino acid residues (e.g., 1, 2, or 3 amino acid residues). In some instances, the component of an immobilized peptide, polypeptide, or polypeptide to which the binder binds may comprise a post-translational modification. In some instances, the component of an immobilized peptide, polypeptide, or polypeptide to which the binder binds may comprise a modified amino acid residue, such as a modified terminal amino acid residue (NTAA or C-terminal amino acid (CTAA) residues).

In some instances, coding tags may comprise an encoder sequence that provides identifying information for the binder used in a given reverse translation cycle (i.e., encoding cycle), an optional UMI, and a spacer sequence that hybridizes to the complementary spacer sequence on the recording tags, thereby facilitating transfer of coding tag information to the recording tag (e.g., through primer extension, also referred to herein as polymerase extension).

As indicated in FIG. 1, the cyclic process of contacting the immobilized peptides, polypeptides, or polypeptide fragments with a binder comprising a coding tag, and performing reverse translation to transfer information about the identity of a portion of an immobilized peptide, polypeptide, or polypeptide fragment to the corresponding extended recording tag, may be repeated for a plurality of cycles, e.g., 2, 3, 4, 5, 6, 7, 8, 9, 10, 20, 30, 40, 50, 60, 70, 80, 90, 100, or more than 100 cycles.

After completion of a cyclic series of binding and encoding reactions, the extended recording tag can be converted into an amplifiable library using, e.g., a capping cycle step where, for example, a capping sequence comprising a universal priming sequence P1′ linked to a universal priming sequence P2 and spacer sequence Sp′ initially anneals to the extended recording tag via complementary P1 and P2′ sequences to bring the cap in proximity to the extended recording tag. The complementary Sp and Sp′ sequences in the extended recording tag and cap anneal, and primer extension adds the second universal primer sequence (P2) to the extended recording tag.

At step 108 in FIG. 1, the extended recording tags are pooled and used to prepare a DNA sequencing library that represents the plurality of immobilized polypeptides, and contain information regarding binding history of polypeptides of the plurality of immobilized polypeptides. The term “binding history” of a polypeptide refers to identities and order of the binding agents that bind and encode one or more components of the polypeptide in the encoding assay.

In some instances, the library may comprise at least 5,000, 10,000, 20,000, 30,000, 40,000, 50,000, 60,000, 70,000, 80,000, 90,000, 100,000, 200,000, 400,000, 600,000, 800,000, 1M, or more than 1M nucleic acid sequences. The library can be appropriately modified using known techniques to enable sequencing on any Next Generation Sequencing (NGS) platform.

At step 110 in FIG. 1, the DNA library is sequenced using, e.g., a next generation sequencing (NGS) technique to generate a plurality of sequence reads.

At step 112 in FIG. 1, the plurality of sequence reads are mapped to a plurality of binder identifier strings, which in turn are used to identify and/or quantify the polypeptides that were extracted from the sample.

In some instances, the plurality of binder identifier strings comprises at least 100,000, 250,000, 500,000, 750,000, 1M, 5M, 10M, 25M, 50M, 75M, 100M, 200M, 300M, 400M, or 500M binder identifier strings.

FIG. 2 depicts an exemplary polypeptide sequencing assay with N-terminal amino acid (NTAA)-specific binding agents. (202) Peptide molecules are each associated with a recording tag (RT) (e.g., a DNA) and attached to beads at a low peptide/RT pair (e.g., a peptide/RT conjugate) density, a sparsity that permits only intra-peptide/RT pair information transfer to occur. For instance, the peptide and its RT can form a construct, and in cases where the peptide is covalently attached to the RT to form a single molecule, the construct sparsity on a bead permits only intramolecular information transfer, and not information transfer between adjacent constructs on the bead. The peptide N-terminal amino acid (NTAA) residues are labeled with an N-terminal modification (NTM). (204) Next, immobilized and labeled peptides are contacted with binding agents specific for labeled NTAA (labeled F-specific binding agent is shown). Each binding agent comprises a coding tag (CT) (e.g., DNA) that comprises identifying information regarding the binder in the binding agent. After binding and washing, the coding tag identifying information is transferred enzymatically to the recording tag (via extension and/or ligation, such as primer extension followed by ligation), generating an extended RT. (206) The labeled NTAA is removed, e.g., by using mild Edman-like elimination chemistry or by a Cleavase enzyme. The cycle 1-2-3 is repeated n times. After n cycles, the extended RT representing the n amino acids of the peptide sequence is formed and can be sequenced by NGS. A representative structure of the extended RT after 7 cycles is shown (208). The exemplary assay described in FIG. 2 can be referred to as next generation peptide sequencing (NGPS).

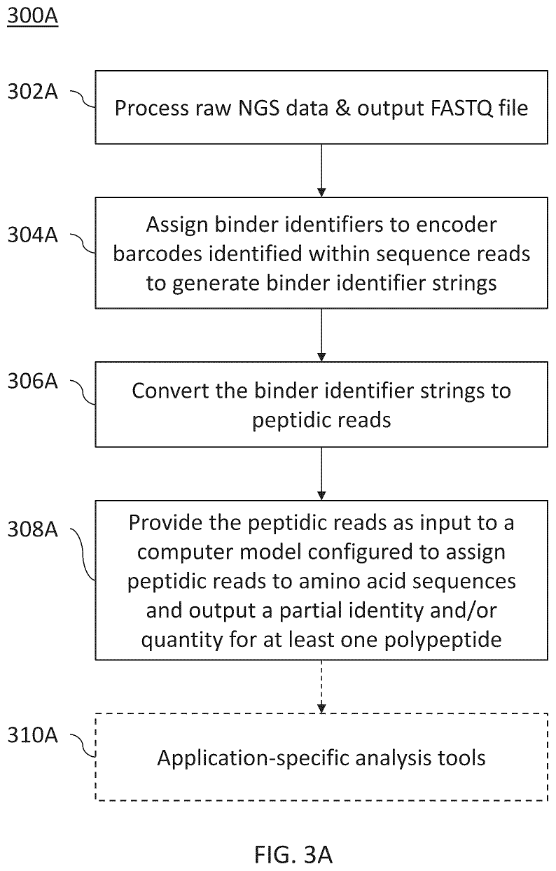

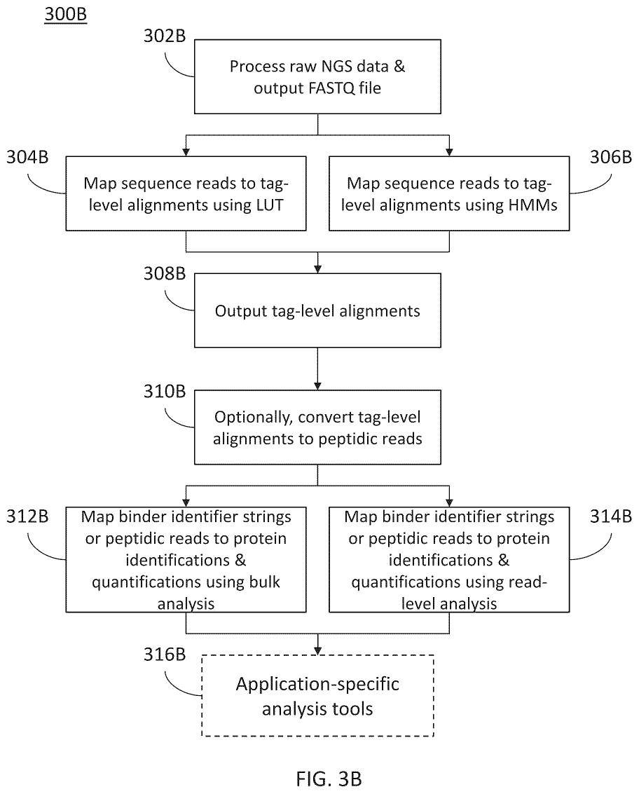

FIG. 3A provides a non-limiting example of a flowchart for a process 300A (corresponding to step 112 in FIG. 1) for processing barcoded nucleic acid sequences to identify (or partially identify, such as identify with certain probability) polypeptides represented therein. Process 300A can be performed, for example, using one or more electronic devices implementing a software platform. In some examples, process 300A is performed using a client-server system, and the blocks of process 300A are divided up in any manner between the server and a client device. In other examples, the blocks of process 300A are divided up between the server and multiple client devices. Thus, while portions of process 300A are described herein as being performed by particular devices of a client-server system, it will be appreciated that process 300A is not so limited. In other examples, process 300A is performed using only a client device or only multiple client devices. In process 300A, some blocks are, optionally, combined, the order of some blocks is, optionally, changed, and some blocks are, optionally, omitted. In some examples, additional steps may be performed in combination with the process 300A. Accordingly, the operations as illustrated (and described in greater detail below) are exemplary by nature and, as such, should not be viewed as limiting.

At step 302A in FIG. 3A, raw NGS data is received (e.g., by one or more processors of a system configured to implement the disclosed methods) for a plurality of polypeptides or fragments thereof (e.g., peptides or polypeptides extracted from a sample) that are encoded in a plurality of nucleic acid sequences (i.e., extended recording tag sequences). The raw NGS data is processed to create and output a sequence read file (e.g., a FASTQ file) which comprises nucleic acid sequence reads corresponding to the plurality of nucleic acid sequences (corresponding to extended recording tag sequences), where each of the plurality of nucleic acid sequences comprises a series of encoder barcode sequences, and optionally one or more auxiliary sequences, and where each barcode sequence of the series of encoder barcode sequences corresponds to a binder that binds to a component of a given polypeptide in the plurality of polypeptides.

FASTQ files may be created using tools such as BCL Convert (Illumina, Inc., San Diego, CA) or bcl2fastq (Illumina, Inc., San Diego, CA). Quality control statistics may be generated using, e.g., FastQC (BaseSpace Labs, Illumina, Inc., San Diego, CA) or MultiQC (Segera Labs, Barcelona, Spain). Overlapping paired end sequence reads may be merged using, e.g., BBMerge (Joint Genome Institute, Lawrence Berkeley National Lab, Berkeley, CA).

As noted above, the binder may comprise, e.g., antibodies or fragments thereof, anticalins, N-recognins polypeptides (e.g., ATP-dependent Clp protease adaptor polypeptide (ClpS)), aptamers, etc., and variants or homologues thereof.

In some instances, the component of the polypeptide to which the binder binds comprises a polypeptide, or portion thereof, obtained by fragmenting the polypeptides of the plurality of polypeptides. In some instances, the component of the polypeptide to which the binder binds comprises one or more amino acid residues. In some instances, the component of the polypeptide to which the binder binds comprises a post-translation modification. In some instances, the component of the polypeptide to which the binder binds comprises a modified amino acid residue (e.g., a modified NTAA or modified CTAA residue).

In some instances, the one or more auxiliary sequences (e.g., 1, 2, 3, 4, 5, or more than 5 auxiliary sequences) may comprise one or more identifier sequences or complements thereof (e.g., 1, 2, 3, 4, 5, or more than 5 identifier sequences or complements thereof), one or more spacer sequences or complements thereof (e.g., 1, 2, 3, 4, 5, or more than 5 spacer sequences or complements thereof), one or more sequencing primer sequences or complements thereof (e.g., 1, 2, 3, 4, 5, or more than 5 sequencing primer sequences or complements thereof), or any combination thereof.

In some instances, the one or more identifier sequences may comprise one or more sample barcode sequences (e.g., 1, 2, 3, 4, 5, or more than 5 sample barcode sequences or complements thereof), one or more bead barcode sequences (e.g., 1, 2, 3, 4, 5, or more than 5 bead barcode sequences or complements thereof), one or more unique molecular identifier (UMI) sequences (e.g., 1, 2, 3, 4, 5, or more than 5 UMI sequences or complements thereof), or any combination thereof.

In some instances, the plurality of nucleic acid sequences may comprise at least 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1M, or more than 1M nucleic acid sequences.

In some instances, the plurality of peptides, polypeptides, polypeptides, or fragments thereof, may comprise at least 100, 500, 1,000, 1,500, 2,000, 2,500, 5,000, 7,500, 10,000, or more than 10,000 different polypeptides.

In some instances, the method may further comprise determining the nucleic acid sequences for the plurality of nucleic acid sequences. In some instances, the nucleic acid sequences may be determined by performing DNA sequencing. In some instances, the DNA sequencing may be performed using, for example, a next generation DNA sequencer.

At step 304A in FIG. 3A, one or more binder identifiers are assigned to each of the series of encoder barcode sequences (and an auxiliary identifier is assigned to at least one of the one or more auxiliary sequences) in each of the plurality of nucleic acid sequences to generate a plurality of binder identifier strings.

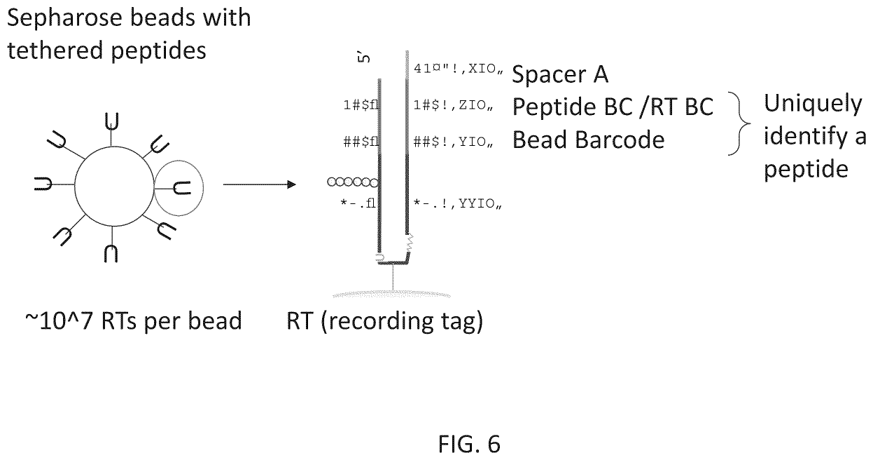

In some instances, the assignment of binder identifiers to encoder barcode sequences may be based on a known architecture for the nucleic acid sequences of the plurality (e.g. a known pattern of sequencing primer sequences, bead barcode sequences, binder barcode sequences, spacer sequences, etc.; see, for example, FIG. 6 and FIG. 8) and a look-up table (LUT) of barcode types. In some instances, the assignment of binder identifiers to encoder barcode sequences may further comprise performing error correction based on an enumeration of error types to account for errors associated with an encoding process used to generate each of the series of encoder barcode sequences. In some instances, accounting for the errors associated with the process used to generate each of the series of encoder barcode sequences may comprise determining: (i) a first probability for reading a specified barcode sequence for a specified underlying polypeptide component, and (ii) a second probability for successfully transitioning from one polypeptide component to the next polypeptide component during the encoding process.

In some instances, the assignment of binder identifiers to encoder barcode sequences may comprise use of a probabilistic model to predict a sequence of binder identifiers for each nucleic acid sequence of the plurality. In some instances, for example, the probabilistic model may comprise a hidden Markov model (HMM). In some instances, the hidden Markov model (HMM) may be trained using one or more training data sets (e.g., 1, 2, 3, 4, 5, or more than 5 training data sets) comprising labeled pairs of binder identifiers and nucleic acid sequences. In some instances, the hidden Markov model (HTMM) may be trained using an iterative Expectation-Maximization (EM) algorithm to determine a set of model parameters that maximize a probability of correctly predicting a sequence of binder identifiers for each nucleic acid sequence of the plurality. In some instances, the Expectation-maximization (EM) algorithm may comprise a Baum-Welch algorithm.

At step 306A in FIG. 3A, the plurality of identifier strings are converted into a plurality of peptidic reads (e.g., probabilistic peptidic reads) based on, for example, an order of binding of the binders in the encoding assay (e.g., the order of binder identifiers in a given binder identifier string) and determined binding profile for the binders (e.g., the known or most probable component of a peptide or polypeptide fragment (e.g., amino acid residue(s), modified amino acid residue(s), post-translation modification, etc.) to which a given binder likely binds) that correspond to binder identifiers assigned to a given identifier string.

In some instances, at least two peptidic reads (e.g., at least 2, 3, 4, 5, or more than 5 peptidic reads) are generated for at least one nucleic acid sequence (e.g., at least 1, 2, 3, 4, 5, 10, 20, 30, 40, 50, or more than 50 nucleic acid sequences) of the plurality of nucleic acid sequences.

At step 308A in FIG. 3A, the plurality of peptidic reads is provided as input to a computer model, where the computer model is configured to assign peptidic reads to amino acid sequences of polypeptides and output an identity, a partial identity, and/or a quantity for at least one polypeptide present in the sample. In some instances, the computer model may output a partial identity for the at least one polypeptide, e.g., in cases where a binder is known to bind to a family of polypeptides, related polypeptides, or homologous polypeptides.

In some instances, the computer model may be configured to identify unique polypeptide signatures in the plurality of peptidic reads as part of assigning peptidic reads to amino acid sequences. In some instances, a given unique polypeptide signature may comprise a set of peptidic reads associated with a single polypeptide. In some instances, the computer model may be trained using a training data set comprising peptidic read data for one or more isolated polypeptide samples that are processed using the same sample preparation protocol as that used to process the plurality of polypeptides. In some instances, the training data set may further comprise peptidic read data for a background sample processed using the same sample preparation protocol as that used to process the plurality of polypeptides. In some instances, the background sample may comprise, for example, a plasma sample, a urine sample, a saliva sample, or a cell extract sample. In some instances, the sample may be a cell extract sample, and the cell extract sample may comprise, for example, a mammalian cell extract, a plant cell extract, a fungal cell extract, or a bacterial cell extract. In some Instances, the computer model may be further configured to correct the quantity output for the at least one polypeptide using a correction factor calculated from the training data set.

In some instances, the computer model may be configured to map each peptidic read of the plurality of peptidic reads to a specific polypeptide as part of assigning peptidic reads to amino acid sequences. In some instances, mapping a peptidic read of the plurality of peptidic reads to a specific polypeptide may comprise: i) generating a set of k-mer fragments for the peptidic read; ii) determining a probability that a given k-mer fragment of the set belongs to a specific polypeptide based on a previously determined probability distribution; and iii) assigning the peptidic read to the specific polypeptide based on the determined probabilities for the set of k-mer fragments. In some instances, the probabilistic model is trained on pre-determined (e.g., empirically determined) assay parameter data. In some instances, the empirically determined assay parameter data may comprise, for each barcode sequence of the series of encoder barcode sequences, a probability of reading a given barcode sequence in a nucleic acid sequence for a given one or more amino acid residue(s) of a corresponding polypeptide to which a binder was bound. In some instances, the empirically determined assay parameter data may comprise, for each potential N-terminal amino acid residue in a polypeptide, a probability of successfully cleaving the N-terminal amino acid residue during a cyclical process used to encode the plurality of polypeptides in the plurality of nucleic acid sequences. In some instances, mapping a peptidic read of the plurality of peptidic reads to a specific polypeptide may comprise providing the plurality of peptidic reads as input to the computer model, where the computer model is configured to fractionally assign a given peptidic read to two or more specific polypeptides.

In some instances, the computer model may be trained on a training data set comprising a set of simulated peptidic reads generated for a given input distribution of polypeptides based on empirically-determined assay parameter data comprising: i) for each barcode sequence of the series of encoder barcode sequences, a probability of reading a given barcode sequence in a nucleic acid sequence for a given one or more amino acid residue(s) of a corresponding polypeptide to which a binder was bound; and ii) for each potential N-terminal amino acid residue in a polypeptide, a probability of successfully cleaving the N-terminal amino acid residue during a cyclical process used to encode the plurality of polypeptides in the plurality of nucleic acid sequences. In some instances, the set of simulated peptidic reads may comprise at least 100,000, 500,000, 1M, 5M, 10M, 25M, 50M, 75M, 100M, 200M, 300M, 400M, or 500M simulated peptidic reads.

In some instances, the computer model may be further configured to output a confidence interval for the partial identity of the at least one polypeptide. In some instances, a level of stringency in the output confidence interval may be selectable by a user. In some instances, for example, the level of stringency in the confidence interval corresponds to a confidence level of 90%, 95%, 98%, or 99%.

In some instances, the computer model may comprise, for example, a trained artificial neural network model, a trained deep learning model, a trained random forest model, or a trained support vector machine.

In some instances, the method may further comprise performing an iterative Expectation Maximization (EM) process to refine the quantity output for the at least one polypeptide of the plurality of polypeptides. In some instances, for example, the iterative EM process may comprise repetitively: i) finding a best assignment of a binder to each barcode sequence in a nucleic acid sequence based on a current estimate of what polypeptides are present in the plurality of polypeptides; ii) finding an updated best estimate of what polypeptides are present in the plurality of polypeptides based on the best assignment of a binder to each different barcode sequence in the nucleic acid sequence; and iii) determining an amount of at least one polypeptide present in the plurality of polypeptides based on the updated best estimate of what polypeptides are present in the plurality of polypeptides. In some instances, steps (i) to (iii) are repeated until a difference between the estimated amount of at least one polypeptide determined in one iteration and the next is less than a specified threshold, or that sum of the squared differences between iterations is less than a specified threshold, or until a specified maximum number of iterations has been reached. In some instances, the specified threshold for the difference between the amount of the at least one polypeptide determined in one iteration and the next may be, for example, a 5%, 1%, 0.1% or 0.01% difference. In some instances, the maximum number of iterations may be, for example, 16, 32, 64, 128, or 256 iterations.

At step 310A in FIG. 3A, the peptidic read data and/or identification and quantification data may optionally be provided as input to any of a variety of application-specific analysis tools, e.g., software packages for performing statistical comparisons of diseased versus normal samples.

Methods (e.g., computer-implemented methods) for generating a plurality of encoding nucleic acid sequences based on the known amino acid sequences for an input plurality of polypeptides or polypeptide fragments are also described herein. Such methods may be useful for, e.g., generating training and/or validation data for the nucleic acid decoding process illustrated in FIG. 3A or FIG. 3B.

In some instances, for example, such a method may comprise: a) receiving (e.g., at one or more processors of a system configured to implement the method) amino acid sequences for a plurality of polypeptides; b) generating a plurality of peptide sequences based on the amino acid sequences; c) generating a plurality of peptidic reads based on the plurality of peptide sequences; d) converting each peptidic read of the plurality of peptidic reads into an identifier string based on an order and determined binding profile for a plurality of binders, where each binder of the plurality binds to a component of a polypeptide and corresponds to a binder identifier, and where the binder identifier corresponds to a barcode sequence; and e) providing the plurality of identifier strings as input to a trained model, where the trained model is configured to convert identifier strings to nucleic acid sequences based on a set of probabilities for binding of the plurality of binders to components of the polypeptides of the plurality of polypeptides and output a plurality of nucleic acid sequences corresponding to the plurality of polypeptides.

In some instances, the method may further comprise comparing the plurality of nucleic acid sequences output by the computer model to a plurality of nucleic acid sequences determined by subjecting the plurality of polypeptides to a reverse translation assay (i.e., encoding assay) and sequencing the resulting extended recording tags. In some instances, the computer model may comprise a statistical model. In some instances, the computer model may comprise a trained machine learning model.

FIG. 3B provides another non-limiting example of a process 300B for processing barcoded nucleic acid sequences to identify peptides and/or polypeptides represented therein. Process 300B can be performed, for example, using one or more electronic devices implementing a software platform. In some examples, process 300B is performed using a client-server system, and the blocks of process 300B are divided up in any manner between the server and a client device. In other examples, the blocks of process 300B are divided up between the server and multiple client devices. Thus, while portions of process 300B are described herein as being performed by particular devices of a client-server system, it will be appreciated that process 300B is not so limited. In other examples, process 300B is performed using only a client device or only multiple client devices. In process 300B, some blocks are, optionally, combined, the order of some blocks is, optionally, changed, and some blocks are, optionally, omitted. In some examples, additional steps may be performed in combination with the process 300B. Accordingly, the operations as illustrated (and described in greater detail below) are exemplary by nature and, as such, should not be viewed as limiting.

At step 302B in FIG. 3B, raw NGS data is received (e.g., by one or more processors of a system configured to implement the disclosed methods) for a plurality of peptides, polypeptides, polypeptides, or fragments thereof (e.g., peptides, polypeptides, or polypeptides extracted from a sample) that are encoded in a plurality of nucleic acid sequences (i.e., extended recording tag sequences).

As noted above with respect to FIG. 3A, the raw NGS data is processed to create and output a sequence read file (e.g., a FASTQ file) which comprises nucleic acid sequence reads corresponding to the plurality of nucleic acid sequences (corresponding to extended recording tag sequences), where each of the plurality of nucleic acid sequences comprises a series of encoder barcode sequences, and optionally one or more auxiliary sequences, and where each barcode sequence of the series of encoder barcode sequences corresponds to a binder (described elsewhere herein) that binds to a component of a given polypeptide in the plurality of polypeptides.

At step 304B in FIG. 3B, one or more binder identifiers are assigned to each of the series of encoder barcode sequences (and an auxiliary identifier is assigned to at least one of the one or more auxiliary sequences) in each of the plurality of nucleic acid sequences using a Lookup Table (LUT) of barcode types to generate a plurality of identifier strings.

The Lookup Table (LUT) is used to quickly assign binder identifiers to the different encoder barcode sequences identified in a sequence read based on a known architecture for the nucleic acid sequences (extended recording tag sequences) as described above in reference to FIG. 3A. In some instances, the assignment of binder identifiers to encoder barcode sequences may further comprise performing error correction based on an enumeration of error types to account for errors associated with an encoding process used to generate each of the series of encoder barcode sequences. In some instances, accounting for the errors associated with the process used to generate each of the series of encoder barcode sequences may comprise determining: (i) a first probability for reading a specified barcode sequence for a specified underlying polypeptide component, and (ii) a second probability for successfully transitioning from one polypeptide component to the next polypeptide component during the encoding process.

Alternatively, at step 306B in FIG. 3B, the assignment of binder identifiers to encoder barcode sequences may comprise use of a probabilistic model to predict a sequence of binder identifiers for each nucleic acid sequence of the plurality. In some instances, for example, the probabilistic model may comprise a hidden Markov model (HMM). In some instances, the hidden Markov model (HMM) may be trained using one or more training data sets (e.g., 1, 2, 3, 4, 5, or more than 5 training data sets) comprising labeled pairs of binder identifiers and nucleic acid sequences. In some instances, the hidden Markov model (HMM) may be trained using an iterative Expectation-Maximization (EM) algorithm to determine a set of model parameters that maximize a probability of correctly predicting a sequence of binder identifiers for each nucleic acid sequence of the plurality. In some instances, the Expectation-maximization (EM) algorithm may comprise a Baum-Welch algorithm.

In some instances, a hidden Markov model (HMM)/Baum-Welch approach may be computationally slower than the LUT approach, but may be more accurate in correctly assigning barcode (and auxiliary sequence) identifiers.

At step 308B in FIG. 3B, the plurality of identifier strings generated in step 304B or step 306B are output.

At step 310B in FIG. 3B, the plurality of identifier strings are optionally converted into a plurality of peptidic reads based on, for example, an order (e.g., the order of binder identifiers in a given identifier string) and determined binding profile for the binders (e.g., the known component of a peptide or polypeptide fragment (e.g., amino acid residue(s), modified amino acid residue(s), post-translation modification, etc.) to which a given binder binds) that correspond to binder identifiers assigned to a given identifier string.

In some instances, at least two peptidic reads (e.g., at least 2, 3, 4, 5, or more than 5 peptidic reads) are generated for at least one nucleic acid sequence (e.g., at least 1, 2, 3, 4, 5, 10, 20, 30, 40, 50, or more than 50 nucleic acid sequences) of the plurality of nucleic acid sequences.

At step 312B in FIG. 3B, the plurality of binder identifier strings or peptidic reads (if converted from binder identifier strings) is provided as input to a computer model, and mapped to polypeptide identifications and quantifications for at least one polypeptide based on a bulk analysis of the binder identifier strings or peptidic reads data. That is, the computer model may be configured to identify unique polypeptide signatures in the plurality of binder identifier strings or peptidic reads as part of assigning binder identifier strings or peptidic reads to amino acid sequences. In some instances, a given unique polypeptide signature may comprise a set of binder identifier strings or peptidic reads associated with a single polypeptide. In some instances, the computer model may be trained using a training data set comprising binder identifier strings or peptidic reads peptidic read data for one or more isolated polypeptide samples that are processed using the same sample preparation protocol as that used to process the plurality of polypeptides. In some instances, the training data set may further comprise binder identifier strings or peptidic reads data for a background sample processed using the same sample preparation protocol as that used to process the plurality of polypeptides. In some instances, the background sample may comprise, for example, a plasma sample, a urine sample, a saliva sample, or a cell extract sample. In some instances, the sample may be a cell extract sample, and the cell extract sample may comprise, for example, a mammalian cell extract, a plant cell extract, a fungal cell extract, or a bacterial cell extract. In some instances, the computer model may be further configured to correct the quantity output for the at least one polypeptide using a correction factor calculated from the training data set.

Alternatively, at step 314B in FIG. 3B, the plurality of binder identifier strings or peptidic reads is provided as input to a computer model, and mapped to polypeptide identifications and quantifications for at least one polypeptide based on a read-level analysis of the binder identifier strings or peptidic reads data. That is, the computer model may be configured to map each binder identifier string or peptidic read of the plurality to a specific polypeptide as part of assigning binder identifier strings or peptidic reads to amino acid sequences. In some instances, mapping a binder identifier string or peptidic read of the plurality to a specific polypeptide may comprise: i) generating a set of k-mer fragments for the binder identifier string or peptidic read; ii) determining a probability that a given k-mer fragment of the set belongs to a specific polypeptide based on a previously determined probability distribution; and iii) assigning the binder identifier string or peptidic read to the specific polypeptide based on the determined probabilities for the set of k-mer fragments. In some instances, the probabilistic model is trained on empirically determined assay parameter data. In some instances, the empirically determined assay parameter data may comprise, for each barcode sequence of the series of encoder barcode sequences, a probability of reading a given barcode sequence in a nucleic acid sequence for a given one or more amino acid residue(s) of a corresponding polypeptide to which a binder was bound. In some instances, the empirically determined assay parameter data may comprise, for each potential N-terminal amino acid residue in a polypeptide, a probability of successfully cleaving the N-terminal amino acid residue during a cyclical process used to encode the plurality of polypeptides in the plurality of nucleic acid sequences. In some instances, mapping a binder identifier string or peptidic read of the plurality to a specific polypeptide may comprise providing the plurality of binder identifier strings or peptidic reads as input to the computer model, where the computer model is configured to fractionally assign a given binder identifier string or peptidic read to two or more specific polypeptides.

The output of the computer model in the bulk analysis and/or read-level analysis approach may comprise the identification and/or quantification of at least one peptide, polypeptide, or polypeptide present in the original sample. In some instances, the quantification of a peptide, polypeptide, or polypeptide present in the sample may be specified, for example, in units of parts per million (ppm), e.g., the number of peptides, polypeptides, or polypeptide molecules of a given species that are present per million peptides, polypeptides, or polypeptide molecules detected in total. In some instances, the output of the computer model may comprise a determination that, e.g., binder identifier string or peptidic read X comes from polypeptide Y, and that polypeptide Y is present at Z parts per million.

In some instances, the computer model may be trained on a training data set comprising a set of simulated binder identifier strings generated for a given input distribution of polypeptides based on empirically-determined assay parameter data comprising: i) for each barcode sequence of the series of encoder barcode sequences, a probability of reading a given barcode sequence in a nucleic acid sequence for a given one or more amino acid residue(s) of a corresponding polypeptide to which a binder was bound; and ii) for each potential N-terminal amino acid residue in a polypeptide, a probability of successfully cleaving the N-terminal amino acid residue during a cyclical process used to encode the plurality of polypeptides in the plurality of nucleic acid sequences. In some instances, the set of simulated binder identifier strings may comprise at least 100,000, 500,000, 1M, 5M, 10M, 25M, 50M, 75M, 100M, 200M, 300M, 400M, or 500M simulated binder identifier strings.

In some instances, the computer model may be further configured to output a confidence interval for the partial identity of the at least one polypeptide. In some instances, a level of stringency in the output confidence interval may be selectable by a user. In some instances, for example, the level of stringency in the confidence interval corresponds to a confidence level of 90%, 95%, 98%, or 99%.

In some instances, the computer model may comprise, for example, a trained artificial neural network model, a trained deep learning model, a trained random forest model, or a trained support vector machine.

In some instances, the method may further comprise performing an iterative Expectation Maximization (EM) process to refine the quantity output for the at least one polypeptide of the plurality of polypeptides. In some instances, for example, the iterative EM process may comprise repetitively: i) finding a best assignment of a binder to each barcode sequence in a nucleic acid sequence based on a current estimate of what polypeptides are present in the plurality of polypeptides; ii) finding an updated best estimate of what polypeptides are present in the plurality of polypeptides based on the best assignment of a binder to each different barcode sequence in the nucleic acid sequence; and iii) determining an amount of the at least one polypeptide present in the plurality of polypeptides based on the updated best estimate of what polypeptides are present in the plurality of polypeptides. In some instances, steps (i) to (iii) are repeated until a difference between the35ephart of the at least one polypeptide determined in one iteration and the next is less than a specified threshold, or until a specified maximum number of iterations has been reached. In some instances, the specified threshold for the difference between the amount of the at least one polypeptide determined in one iteration and the next may be, for example, a 10%, 8%, 6%, 4%, or 2% difference. In some instances, the maximum number of iterations may be, for example, 5, 10, 15, 20, 25, 30, 35, 40, 45, or 50 iterations.

At step 316B in FIG. 3B, the peptidic read data and/or identification and quantification data may optionally be provided as input to any of a variety of application-specific analysis tools, e.g., software packages for performing statistical comparisons of diseased versus normal samples.

As noted above with regard to FIG. 3A, in some instances the computer model may be trained and/or validated using a combination of experimental data and/or simulated data (e.g., a plurality of encoding nucleic acid sequences generated in silico based on the known amino acid sequences for an input plurality of polypeptides or polypeptide fragments).

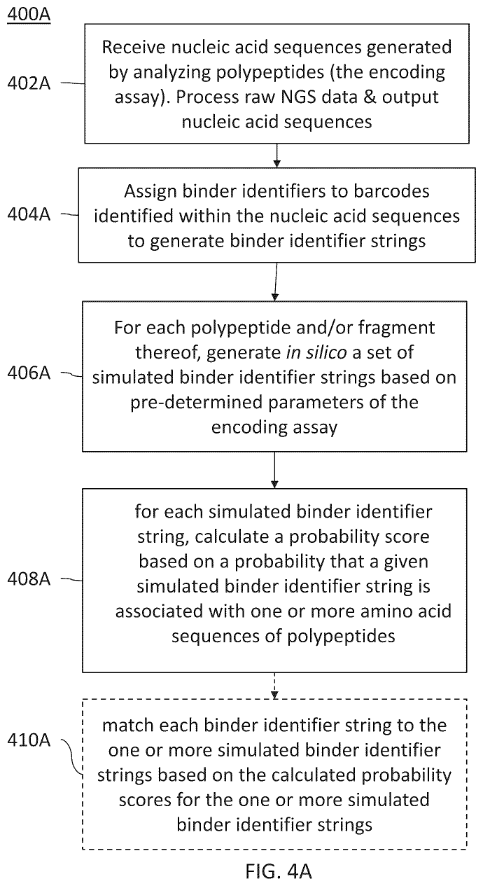

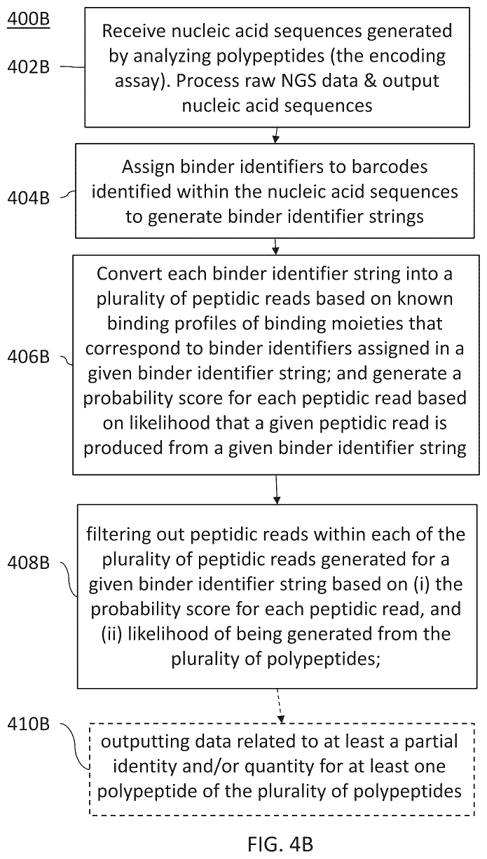

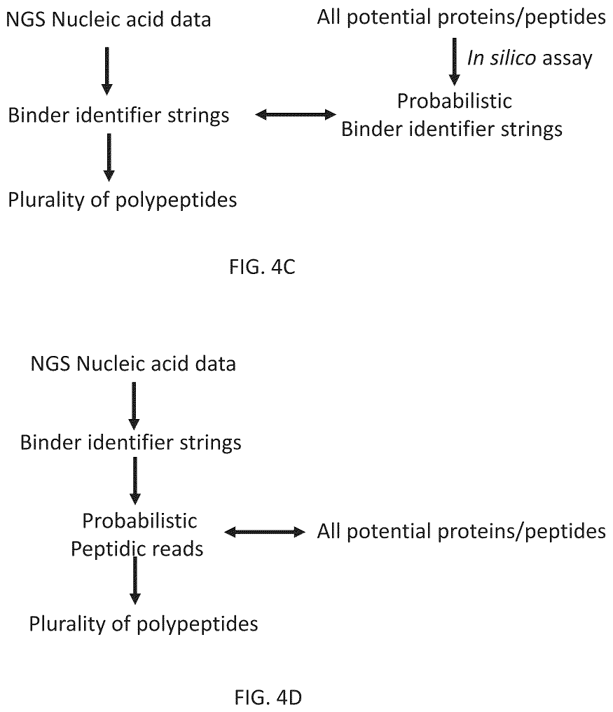

FIG. 4A provides another non-limiting example of a process 400A for processing barcoded nucleic acid sequences to identify polypeptides represented therein. Process 400A can be performed, in part, as described above for processes 300A and 300B. In process 400A, step 402A is the same as step 302B in process 300B; and step 404A is the same as either step 304B or step 306 B in process 300B (see the description of steps 302B, 304B and 306 B above).

At step 406A in FIG. 4A, a set of simulated probabilistic binder identifier strings is generated in silico for each polypeptide and/or polypeptide fragment based on pre-determined parameters of the encoding assay (see also Example 8). When the number of polypeptides needs to be analyzed is limited (e.g., below 100), then probabilistic distribution for the polypeptides to be analyzed may be generated for polypeptides as used in the encoding assay (such as, e.g., tryptic digests of proteins from one or more biological samples). When the number of polypeptides needs to be analyzed exceeds certain threshold (e.g., above 100, above 1000, or above 10000), computational ability for generating probabilistic distribution for the polypeptides to be analyzed may be impaired. Therefore, for analysis of a large number of polypeptides simultaneously (such as, e.g., analysis of a whole eukaryotic proteome), each polypeptide may be broken up to k-mer fragments as described in Example 8, which would reduce requirements for computational power.

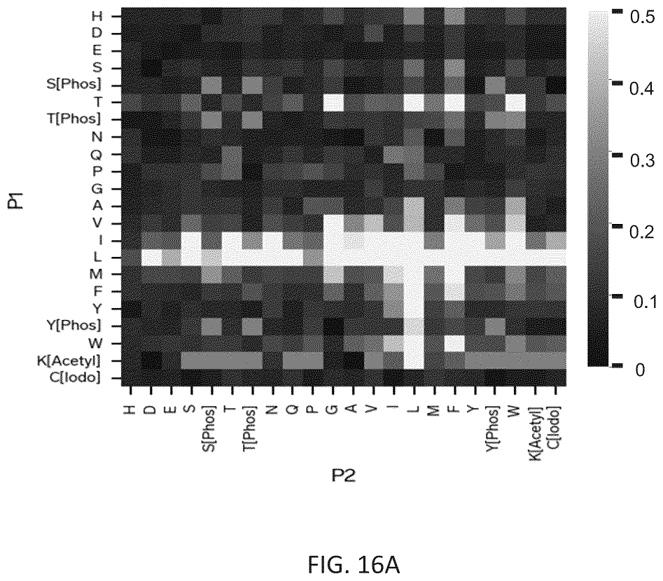

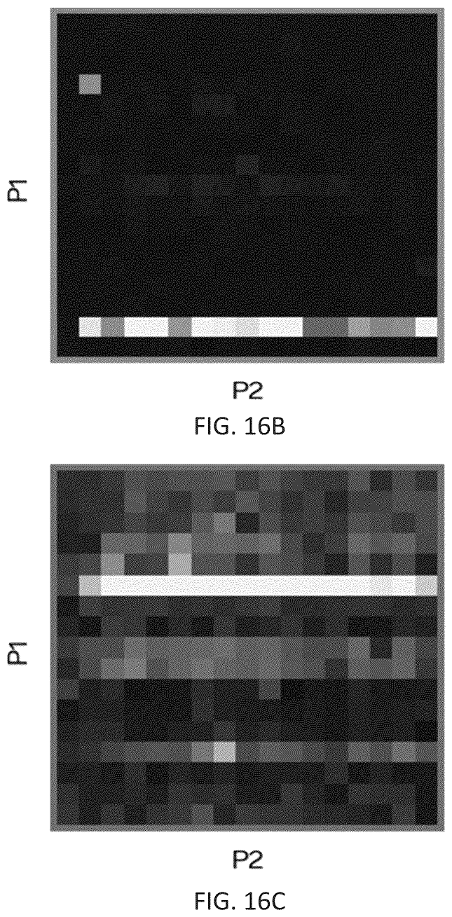

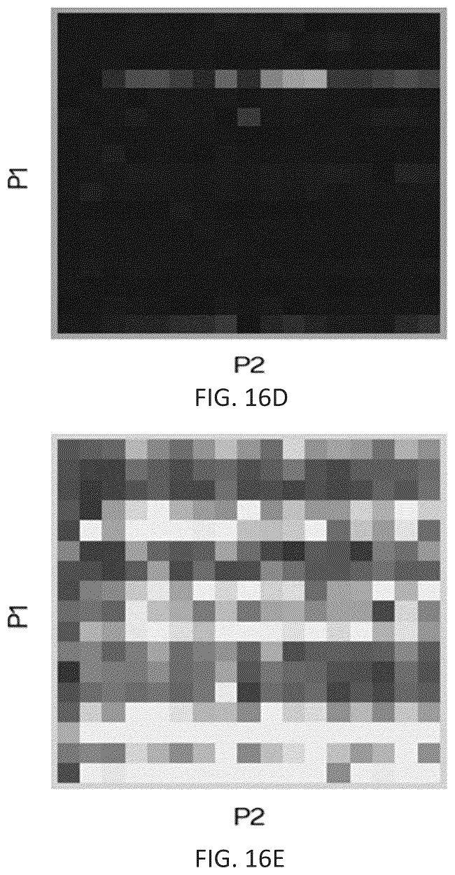

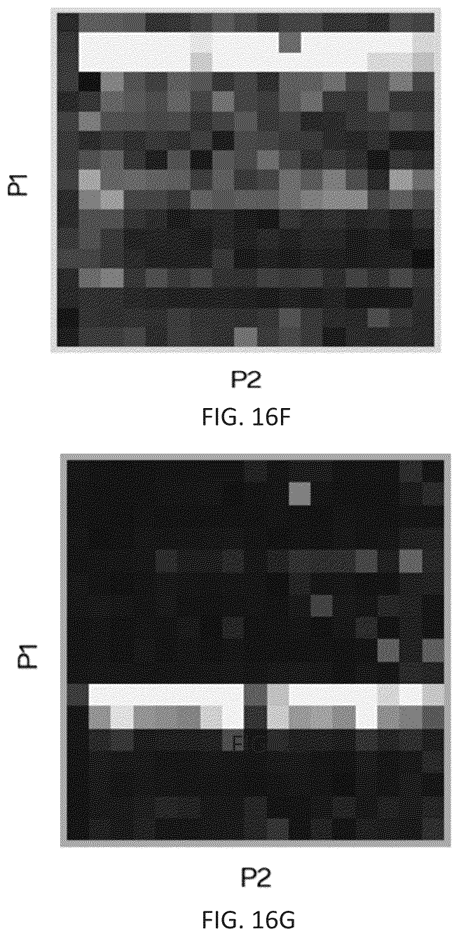

In preferred embodiments, pre-determined parameters of the encoding assay are obtained by performing multiple encoding assays using a panel of known polypeptide analytes. For example, when each binder of a set of binders used in the encoding assay is specific for a particular P1-P2 residues of polypeptide analytes, a model set of polypeptides having all possible combinations of P1-P2 may be utilized for the encoding assay (see, e.g., heatmap encoding data in FIG. 16A-FIG. 16G). In some embodiments, pre-determined parameters of the encoding assay are obtained experimentally and further used to train a computer model, which uses them as input parameters. In some embodiments, parameters of the encoding assay may not be determined in advance but rather calculated within a machine learning (ML) model, when encoding data from a large number of model polypeptides ((such as genetically encoded polypeptides that represent a variety of different amino acid sequences) are inputted into the ML model (see Example 10 for more details), and the model outputs data comprising pluralities of nucleic acid sequences. Millions of encoding assays may be performed in parallel to provide a sufficient dataset for training of a ML model.

In some embodiments, in addition to training on recognition of components of polypeptide analytes by binders, training of the ML model may be provided on efficiency of cleavage of polypeptide components in the encoding assay, such as efficiency of cleavage of an N-terminal amino acid residue after each encoding cycle, which may depend of the neighboring amino acid residues (see Example 7). The training may be performed by providing to the ML model as an input experimental outputs (i.e., data comprising pluralities of nucleic acid sequences) generated from performing multiple encoding assays with pre-determined polypeptide analytes (such as polypeptides that produce all possible NTAA residues at some point during cleavage events), and analyzing encoded binding events to detect presence or absence of a cleavage event depending on neighboring amino acid residues. For example, depending on cleavage methods (e.g., a chemical cleavage or an enzymatic cleavage), efficiency of NTAA cleavage may vary depending on particular NTAA cleaved off. In various embodiments, the efficiency of cleavage may vary from 70% to 100%.

In some embodiments, the pre-determined parameters of the encoding assay comprise: i) probabilities of assigning to at least one component of a given polypeptide or polypeptide fragment one or more binder identifiers either correctly or incorrectly based on determined binding profiles of binders used in the encoding assay; and ii) optionally, for at least one component of a given polypeptide or polypeptide fragment, a probability of successfully cleaving the at least one component after a binder binds to the at least one component in the encoding assay. In preferred embodiments, the pre-determined parameters comprise both i) and ii) elements.

At step 408A in FIG. 4A, using the simulated assay and calculated probabilities for each simulated binder identifier string as disclosed in step 406A, probabilities for each binder identifier string of the plurality of binder identifier strings generated in step 404A (i.e., probabilities that a given binder identifier string is generated from one or more polypeptides or fragments thereof) are calculated. This allows to associate with certain probabilities or directly match experimental encoding data (nucleic acid sequences) with particular polypeptides or fragments thereof that are present in the analyzed sample (or samples). The described process would allow to determine at least a partial identity and/or quantity for at least one polypeptide of the plurality of polypeptide analytes.

In preferred embodiments, as a result of step 408A in FIG. 4A, probabilities for each simulated binder identifier string generated from a given polypeptide or polypeptide fragment are calculated using the pre-determined assay parameters (see Example 8 for exemplary calculations). Data generated during the described simulation may be re-organized, so that for each simulated binder identifier string a probability that this identifier string comes from a particular polypeptide is determined.

In process 400A, step 410A comprises matching each binder identifier string to the one or more simulated binder identifier strings based on the calculated probability scores for the one or more simulated binder identifier strings. The step 410A comprises outputting data related to at least a partial identity and/or quantity for at least one polypeptide of the plurality of polypeptides, similar to step 316B in process 300B (see the description above).

FIG. 4B provides yet another non-limiting example of a process 400B for processing barcoded nucleic acid sequences to identify polypeptides represented therein. Process 400B can be performed, in part, as described above for processes 300A and 300B. In process 400B, step 402B is the same as step 302B in process 300B; and step 404B is the same as either step 304B or step 306 B in process 300B (see the description of steps 302B, 304B and 306 B above).

At step 406B in FIG. 4B, each binder identifier string is converted into a plurality of peptidic reads based on determined binding profiles of binding moieties that correspond to binder identifiers assigned in a given binder identifier string; and a probability score is generated for each peptidic read based on likelihood that a given peptidic read is produced from a given binder identifier string (see Example 9 for further details).

At step 408B in FIG. 4B, peptidic reads are filtered out within each of the plurality of peptidic reads generated for a given binder identifier string based on (i) the probability score for each peptidic read, and (ii) likelihood of being generated from the plurality of polypeptides. This step is optional and may be performed iteratively one or more times while inferring an amino acid sequence of a polypeptide of the plurality of polypeptides from binder identifiers of a binder identifier string. This step allows to simplify association between binder identifier strings generated from the encoding assay data and amino acid sequences of polypeptides of the plurality of polypeptides by filtering out low probability binder identifier strings that are unlikely to be produced from amino acid sequences of the plurality of polypeptides that are potentially present in an analyzed sample or samples. In some embodiments, filtering step can be performed based on pre-defined threshold (see Example 9 for further details).

Finally, the step 410B comprises outputting data related to at least a partial identity and/or quantity for at least one polypeptide of the plurality of polypeptides, similar to step 316B in process 300B (see the description above).

### Machine Learning Methods

Any of a variety of machine learning approaches & algorithms (where a machine learning model, as referred to herein, comprises a trained machine learning algorithm) may be used in implementing the disclosed methods. For example, the machine learning model/algorithm employed may comprise a supervised learning model/algorithm, an unsupervised learning model/algorithm, a semi-supervised learning model/algorithm, a deep learning model/algorithm, or any combination thereof, as will be discussed in more detail below. In some instances, one or more machine learning models (e.g., 1, 2, 3, 4, 5, 6, or more than 6 machine learning models) may be utilized to implement the disclosed methods.

Examples of machine learning algorithms that may be employed include, but are not limited to, artificial neural networks, deep neural networks, deep recurrent neural networks, deep convolutional neural networks, Gaussian process regression algorithms, logistical model tree algorithms, random forest algorithms, fuzzy classifier algorithms, decision tree algorithms, hierarchical clustering algorithms, k-means clustering algorithms, fuzzy clustering algorithms, deep Boltzmann machine learning algorithms, or any combination thereof.

Supervised learning models algorithms: Supervised learning models comprise trained algorithms that rely on the use of labeled training data to infer the relationship between a set of one or more input features (e.g., a plurality of binder identifier strings) and an output prediction (e.g., an identification and/or quantification of at least one polypeptide represented in the plurality of binder identifier strings). The training data comprises one or more sets (e.g., 1, 2, 3, 4, 5, 6, 7, 8, 9, 10, or more than 10 sets) of paired training examples, e.g., where each example comprises a set of input features (e.g., a set of binder identifier strings) and the corresponding output (e.g., identification and/or quantification of a polypeptide corresponding to the set of binder identifier strings).

Unsupervised learning models algorithms: Unsupervised learning algorithms are algorithms used to draw inferences from training datasets consisting of input datasets (e.g., sets of binder identifier strings) that are not paired with labeled output data (e.g., the identification and/or quantification of the polypeptides corresponding to the sets of binder identifier strings). One example of a commonly used unsupervised learning algorithm is cluster analysis, which is often used for exploratory data analysis to find hidden patterns or groupings in multi-dimensional data sets. Other examples of unsupervised learning algorithms include, but are not limited to, artificial neural networks, association rule learning algorithms, hierarchical clustering algorithms, matrix factorization approaches, dimensionality reduction approaches, or any combination thereof.

Semi-supervised learning models algorithms: Semi-supervised learning algorithms are algorithms that make use of both labeled and unlabeled training data for training (typically using a relatively small amount of labeled data with a larger amount of unlabeled data).

Artificial neural networks & deep learning models algorithms: Artificial neural networks (ANNs) and deep learning algorithms are algorithms inspired by the structure and function of the human brain. Specifically, deep learning algorithms are large artificial neural networks comprising many layers of coupled “nodes” that may be used to map input feature data to, e.g., classification decisions. In some instances, the machine learning model/algorithm used for implementing the disclosed methods and systems may be an artificial neural network (ANN) or deep learning model/algorithm that comprises any type of neural network model known to those of skill in the art, such as a feedforward neural network, radial basis function network, recurrent neural network, or convolutional neural network, and the like. In some instances, the disclosed methods and systems may employ a pre-trained ANN or deep learning model. In some embodiment, the disclosed methods and systems may employ a continuous learning ANN or deep learning model, where the model is periodically or continuously updated based on new training data provided by, e.g., a single local operational system, a plurality of local operational systems, or a plurality of geographically-distributed operational systems.

Artificial neural networks generally comprise an interconnected group of nodes organized into multiple layers of nodes. For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to a preferred output value or set of output values. Each layer of the neural network comprises a number of nodes (or neurons). A node receives input data (e.g., binder identifier string data) that comes either directly from the input data nodes or from the output of nodes in previous layers, and performs a specific operation, e.g., a summation operation. In some cases, a connection from an input to a node is associated with a weight (or weighting factor). In some cases, the node may, for example, sum up the products of all pairs of inputs, Xᵢ, and their associated weights, Wᵢ. In some cases, the weighted sum is offset with a bias, b. In some cases, the output of a neuron may be gated using a threshold or activation function, ƒ, which may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, a sigmoid function, or any combination thereof.

The weighting factors, bias values, and threshold values, or other computational parameters of the neural network, can be “taught” or “learned” in a training phase using one or more sets of training data. For example, the parameters may be trained using the input data from a training data set and a gradient descent or backward propagation method so that the output value(s) (e.g., an identification and/or quantification for at least one polypeptide) that the ANN computes are consistent with the examples included in the training data set. The adjustable parameters of the model may be obtained from a back propagation neural network training process that may or may not be performed using the same hardware as that used for performing the disclosed methods.

In general, the number of nodes in the input layer of the ANN or deep learning model/algorithm (which enables input of data for, e.g., a large plurality or binder identifier strings) may range from about 10 to about 10,000 nodes. In some instances, the number of nodes used in the input layer may be at least 10, at least 50, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, or at least 10,000 nodes, or any number of nodes within this range.

In some instances, the total number of layers used in the ANN or deep learning model/algorithm (including input and output layers) may range from about 3 to about 20 layers, or more. In some instances for example, the total number of layers may be at least 3, at least 4, at least 5, at least 6, at least 7, at least 8, at least 9, at least 10, at least 15, or at least 20 layers, or any number of layers within this range.

In some instances, the total number of learnable or trainable parameters, e.g., weighting factors, biases, or threshold values, used in the ANN or deep learning model/algorithm may range from about 1 to about 1,000,000,000. In some instances, the total number of learnable parameters may be at least 1, at least 10, at least 100, at least 500, at least 1,000, at least 2,000, at least 3,000, at least 4,000, at least 5,000, at least 6,000, at least 7,000, at least 8,000, at least 9,000, at least 10,000, at least 100,000, at least 1,000,000, at least 10,000,000, at least 100,000,000, or at least 1,000,000,000 parameters, or any number of parameters within this range.

In some embodiments, ANNs can consist of many more nodes/layers/trainable parameters, such as ANNs can have millions or billions of parameters across suitably designed models.

Hidden Markov models: Hidden Markov Models (HMMs) are probabilistic models that can be used to describe the evolution over time of observable events (symbols) that depend on internal factors (hidden states) which are not directly observable (see, e.g., Yoon (2009), “Hidden Markov Models and their Applications in Biological Sequence Analysis”, Current Genomics 10:402-415; Tamposis, et al. (2019), “Semi-Supervised Learning of Hidden Markov Models for Biological Sequence Analysis”, Bioinformatics 35(13); 2208-2215). They have been widely used in applications such as pattern recognition, speech recognition, digital communication, and computational analysis of biological sequences (e.g., gene prediction, sequence alignment, base-calling, modeling DNA sequencing errors, and predicting polypeptide secondary structure).

The system being modeled is assumed to be governed by a Markov process, i.e., a stochastic process comprising a sequence of possible events in which the probability of each event depends only on the underlying hidden state attained in the previous event. For a sequence of events, x=x₁, x₂, x₃, . . . x_{L}, in a set of observations, O, and an underlying sequence of hidden states, y=y₁, y₂, y₃, . . . y_{L}, in a set of states, S, the sequence of hidden states is assumed to evolve in a time-homogenous manner, i.e., the probability of entering state j in the next time point depends only on the current state I and does not change over time. This fixed probability for making a transition from state I to state j

$$P \{ {y}_{n + 1} = j \vert {y}_{n} = i \} = t ( i , j )$$

for all states I, j∈S and for all n≥1 is called the transition probability. For the initial state, y₁, the initial state probability is denoted as P{y₁=i}=π(i) for all I∈S.

The probability that the n^{th} observation of an event will be x=xₙ depends only on the underlying state y=yₙ=I,

$$P \{ {x}_{n} = x \vert {y}_{n} = i \} = e ( x \vert i )$$

for all possible observations x∈O, all states I∈S, and all n≥1, and is called the emission probability of x at state i. Collectively, the set of three probability measures t(I, j), π(i), and e(x|i) (denoted Θ) completely specify the Hidden Markov Model.

Hidden Markov Models may be trained using a supervised, semi-supervised, or unsupervised learning approach. In a supervised machine learning approach, for example, training data comprising examples of input data (e.g., sets of binder identifier strings) accompanied by labels corresponding to the different output classes (e.g., different polypeptides) are provided as input to the model, and the set of model parameters that maximize the joint probability of correctly mapping input data to the appropriate output class is determined.

Hidden Markov Models are often trained using an iterative Expectation-Maximization (EM) algorithm. Each iteration comprises an “estimation” step and a “maximization” step. In the “maximization” step, one aligns each set of input data values (the set of input data values comprising an observation vector X) with a state S in the model so that a likelihood measure is maximized. In the “estimation” step, for each state, S, one estimates: (i) the parameters of a statistical model for the alignment of the X observation vectors to state S, and (ii) the state transition probabilities. In the following iteration, the maximization step is repeated using the updated statistical model parameters. The process is repeated either for a specified maximum number of iterations or until the change in likelihood measure from one iteration to the next is less that a specified threshold (i.e., the model converges to a stable solution). A UNHMM will typically have a designated “start” state which is aligned to the first set of observations, and has a unidirectional topology so that once the system has transitioned away from a given state, it doesn't return to it.

Training data sets: As noted above, the type of training data used for training a machine learning model/algorithm for use in the disclosed methods and systems will depend on, for example, whether a supervised or unsupervised approach is taken. In some instances, one or more training data sets may be used to train the computer model in a training phase that is distinct from that of the deployment phase. In some instances, the training data may be continuously updated and used to update the machine learning algorithm in real time. In some cases, the training data may be stored in a training database that resides on a local computer or server. In some cases, the training data may be stored in a training database that resides online or in the cloud.

Machine learning software: Any of a variety of commercial or open-source software packages, software languages, or software platforms known to those of skill in the art may be used to implement the machine learning algorithms of the disclosed methods and systems. Examples include, but are not limited to, Shogun (www.shogun-toolbox.org), Mlpack (www.mlpack.rog), R (r-project.org), Weka (www.cs.waikato.ac.nz/ml/weka/), Python (www.python.org), and/or MATLAB (MathWorks, Natick, MA, www.mathworks.com).

### Systems & Software for Deducing the Identities of Encoded Polypeptides

Also disclosed herein are systems designed to implement any of the disclosed bioinformatics methods for deducing the identities of encoded polypeptides. The systems may comprise, e.g., one or more processors, and a memory unit communicatively coupled to the one or more processors and configured to store instructions that, when executed by the one or more processors, cause the system to: receive a plurality of nucleic acid sequences, where each of the plurality of nucleic acid sequences comprises a series of encoder barcode sequences and one or more auxiliary sequences, and where each barcode sequence of the series of encoder barcode sequences corresponds to a binder that binds to a component of a polypeptide of the plurality of polypeptides; assign a binder identifier to each of the series of encoder barcode sequences and an auxiliary identifier to at least one of the one or more auxiliary sequences in each of the plurality of nucleic acid sequences to generate a plurality of identifier strings for the plurality of nucleic acid sequences; convert the plurality of identifier strings into a plurality of binder identifier strings based on an order and determined binding profile for the binders that correspond to binder identifiers assigned to a given identifier string; and provide the plurality of binder identifier strings as input to a computer model, where the computer model is configured to assign binder identifier strings to amino acid sequences of polypeptides of the plurality of polypeptides and output a partial identity and/or quantity for at least one polypeptide of the plurality of polypeptides.

In some instances, the disclosed systems may further comprise a sequencer, e.g., a next generation sequencer. Examples of next generation sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLiD) system, Polonator's G.007 system, Helicos BioSciences' HeliScope Gene Sequencing system, ThermoFisher Scientific's Ion Torrent Genexus system, or Pacific Biosciences' PacBio® RS system.

Software comprising the coded instructions necessary to implement any of the methods described herein is also included in the present disclosure. For example, disclosed herein are non-transitory computer-readable storage media storing one or more programs, the one or more programs comprising instructions, which when executed by one or more processors of a system, cause the system to: receive a plurality of nucleic acid sequences generated from an encoding assay, where each of the plurality of nucleic acid sequences comprises a series of encoder barcode sequences, and where each barcode sequence of the series of encoder barcode sequences corresponds to a binder that binds to a component of a polypeptide of the plurality of polypeptides in the encoding assay; assign a binder identifier to each of the series of encoder barcode sequences in each of the plurality of nucleic acid sequences to generate a plurality of identifier strings for the plurality of nucleic acid sequences; and provide the plurality of binder identifier strings as input to a computer model, where the computer model is configured to infer amino acid sequences of polypeptides of the plurality of polypeptides from binder identifier strings based on determined binding profiles of binders that correspond to binder identifiers in a given identifier string, and output data related to a partial identity and/or quantity for at least one polypeptide of the plurality of polypeptides.

In the next paragraphs below, a non-limiting list of known algorithms is provided, where each of these algorithms can be used to (i) calculate probability scores of an association between one or more binder identifiers of the binder identifier string and one or more amino acid sequences of polypeptides; and/or (ii) infer an amino acid sequence of a polypeptide of the plurality of polypeptides from binder identifiers of a binder identifier string.

In some embodiments, a Bayesian Network (BN) may be implemented to infer polypeptide sequences from given binder identifier strings, in which each binder identifier serves as a node in the network, nodes may be connected sequentially based on the order in which the binder identifiers influence amino acid outcomes, and where a conditional probability table is defined that represents the likelihood of each possible amino acid identity outcome given one or more parent binder identifiers. Inference may be run on the BN optionally using a variable elimination algorithm, belief propagation algorithm, or Gibbs sampling algorithm.

In another non-limiting embodiment, conditional random fields (CRFs) may be used to infer polypeptide sequences from given binder identifier strings, which model conditional dependencies between output labels (i.e., amino acids in the polypeptide sequence) given the input features (i.e., binder identifier transitions), which optionally may be trained using gradient descent with a Limited-memory Broyden-Fletcher-Goldfarb-Shanno (L-BFGS) optimization algorithm, stochastic gradient descent with L2 regularization term optimization algorithm, averaged perceptron optimization algorithm, Passive-Aggressive (PA) optimization algorithm, and/or Adaptive Regularization Of Weight Vector (AROW) optimization algorithm.

In another non-limiting embodiment, Markov random fields (MRFs) may be implemented to predict polypeptide sequences from binder identifier strings, which may optionally involve: treating each position in the binder identifier string as a node; defining potentials for each node representing the probability of producing specific amino acid identities, optionally adding pairwise potentials to represent dependencies between consecutive binder identifiers (e.g., if neighboring binder identifiers have a higher likelihood of producing a pair of amino acid identities, then optionally including these joint probabilities in the model); using inference techniques, including, but not limited to, belief propagation or Gibbs sampling, to compute the most probable polypeptide sequence given the modeled potentials.

In another non-limiting embodiment, a Kalman filter (or linear quadratic estimation) algorithm may be implemented for inferring polypeptide sequences from binder identifier strings, in which the Kalman filter is optionally adapted to incorporate transition probabilities between one or more binder identifiers in a binder identifier string and their likelihoods of resulting in one or more amino acids in the polypeptide sequences. In some instances, the Kalman filter algorithm may involve defining state models in which each state represents a hidden state of the polypeptide sequence linked to a binder identifier or transition between binder identifiers, and may involve defining an observation model that relates each binder identifier to probabilities of amino acid identities in the polypeptide sequence. The Kalman filter components may involve: a state vector, in which each element of the vector represents the probability of an amino acid in the polypeptide sequence at a certain position in the polypeptide sequence; a transition matrix, which models the transitions between binder identifiers in the binder identifier strings, and wherein some transitions are optionally more probable than others based on known polypeptide sequences in the dataset; an observation matrix, which captures the probability of each binder identifier emitting specific amino acid identities; and/or covariance matrices, which define covariances for process noise (i.e., transition variability) and observation noise (i.e., uncertainty in emission probabilities). After processing all binder identifiers in a binder identifier string, the Kalman filter may have an adjusted state vector representing the most probable polypeptide sequence.

In another non-limiting embodiment, a particle filter (or sequential Monte Carlo) algorithm is implemented to infer polypeptide sequences from binder identifier strings, which may involve: initializing particles where each particle represents a possible polypeptide sequence outcome; one or more propagation or prediction steps, in which new possible amino acid identities are generated by extending each particle according to observed probabilities in the dataset of binder identifiers resulting in amino acid identities; one or more weight update or likelihood calculation steps, in which weights are assigned to each particle based on how well the new amino acid matches the aforementioned observed probabilities; resampling particles based on their weights, optionally duplicating particles with high weights and discarding those with lower weights; optionally iterating through propagation, weight update, and resampling steps for each binder identifier; and extracting a polypeptide sequence prediction, wherein the particle with the highest weight can be considered the most probable polypeptide sequence, or the top-N weighted particles may be considered to predict a range of most likely polypeptide sequences, where N is a positive integer greater than zero.

In another non-limiting embodiment, Gaussian process (GP) algorithms may be implemented to infer polypeptide sequences from binder identifier strings, which may involve: representing each binder identifier string as a point in the input space with each binder identifier as categorical or numerical features, and representing one or more individual amino acids in the polypeptide sequences as a point in the output space as categorical or numerical features; use of a kernel function (e.g., radial basis function, or Matern kernel function) to capture similarity between binder identifiers; training the GP algorithm with observed probabilities of mapping one or more binder identifiers to one or more amino acids as labels for the GP; and running inference on the trained model, wherein given a new binder identifier string, one or more amino acid identities are predicted by drawing from the GP posterior, and optionally obtaining a mean prediction and uncertainty (or confidence interval) of the prediction.

In another non-limiting embodiment, an autoregressive integrated moving average (ARIMA) model is implemented to forecast the next amino acid identity in a polypeptide sequence by considering previously determined amino acid identities in the polypeptide sequence and a binder identifier string.

In another non-limiting embodiment, a dynamic time warping (DTW) algorithm may be implemented to find the optimal alignment between binder identifier strings and polypeptide sequences, which may account for inefficiencies in encoding assay steps, such as one or more skipped N-terminal modifications and/or one or more skipped NTAA-modified cleavage steps and/or one or more unmodified NTAA cleavage steps.

In another non-limiting embodiment, a neural autoregressive model may be implemented to infer polypeptide sequences from binder identifier strings, which involves generating amino acid samples in an output sequence autoregressively given in an input sequence which may involve a binder identifier string.

In another non-limiting embodiment, a variational autoencoder (VAE) algorithm may be implemented to infer polypeptide sequences from binder identifier strings, which may be trained to learn a latent space representation of a dataset mapping binder identifier strings to polypeptide sequences, and may be used in inference to generate a polypeptide sequence given a previously unseen binder identifier string.

In another non-limiting embodiment, a generative adversarial network (GAN) algorithm may be implemented to infer polypeptide sequences from binder identifier strings, which uses a generator model (that takes an input binder identifier string and outputs a polypeptide sequence) and a discriminator model (that classifies polypeptide sequences produced by the generator model as real polypeptide sequences or fake polypeptide sequences) to learn the probability distribution of amino acid identities given binder identifiers in an adversarial framework. The generator model may be trained to generate a polypeptide sequence conditioned on a binder identifier string to minimize the probability that the discriminator model correctly identifies its sequences as fake, and the discriminator model is trained to maximize the probability of classifying real polypeptide sequences as real and minimize the probability of classifying fake polypeptide sequences as real. Optionally, a conditional GAN (cGAN) approach may be taken where both the generator and discriminator are conditioned on the binder identifier input (Mirza, M. (2014). Conditional generative adversarial nets. arXiv preprint arXiv:1411.1784).

### Computer Devices and Systems

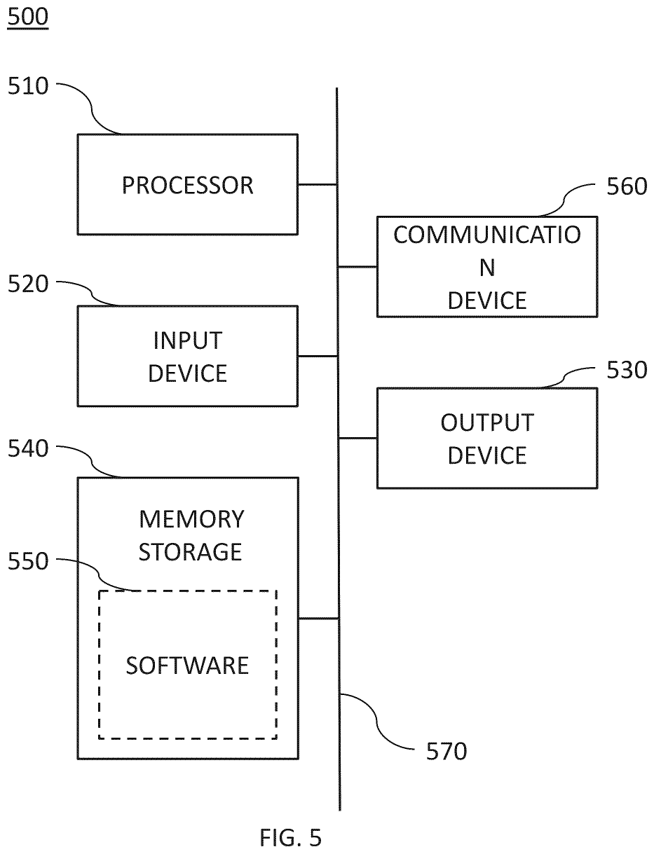

FIG. 5 illustrates an example of a computing device in accordance with one or more examples of the disclosure. Device 500 can be a host computer connected to a network. Device 500 can be a client computer or a server. As shown in FIG. 5, device 500 can be any suitable type of microprocessor-based device, such as a personal computer, workstation, server, or handheld computing device (portable electronic device), such as a phone or tablet. The device can include, for example, one or more of processor 510, input device 520, output device 530, memory storage 540, and communication device 560. Input device 520 and output device 530 can generally correspond to those described above, and they can either be connectable or integrated with the computer.

Input device 520 can be any suitable device that provides input, such as a touch screen, keyboard or keypad, mouse, or voice-recognition device. Output device 530 can be any suitable device that provides output, such as a touch screen, haptics device, or speaker.

Memory storage 540 can be any suitable device that provides storage, such as an electrical, magnetic, or optical memory including a RAM, cache, hard drive, or removable storage disk. Communication device 560 can include any suitable device capable of transmitting and receiving signals over a network, such as a network interface chip or device. The components of the computer can be connected in any suitable manner, such as via a physical bus or wirelessly.

Software 550, which can be stored in memory storage 540 and executed by processor 510, can include, for example, the programming that embodies the functionality of the present disclosure (e.g., as embodied in the methods described above).

Software 550 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1240, that can contain or store programming for use by or in connection with an instruction execution system, apparatus, or device.

Software 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 500 may be connected to a network, which can be any suitable type of interconnected communication system. The network can implement any suitable communications protocol and can be secured by any suitable security protocol. The network can comprise network links of any suitable arrangement that can implement the transition and reception of network signals, such as wireless network connections, T1 or T3 lines, cable networks, DSL, or telephone lines.

Device 500 can implement any operating system suitable for operating on the network. Software 550 can be written in any suitable programming language, such as C, C++, Java, or Python. In various embodiments, application software embodying the functionality of the present disclosure can be deployed in different configurations, such as in a client/server arrangement or through a web browser as a web-based application or web service, for example.

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, the term “analyte” refers to a substance whose chemical constituents are being identified and/or measured. In preferred embodiments, “analyte” refers to “polypeptide analyte”, and at least partial amino acid sequence and/or identity of the polypeptide analyte is/are determined by the methods disclosed herein. In some embodiments, one, two or more amino acid residues of a polypeptide analyte each are individually determined with a certain probability, which may be sufficient for determining identity of the polypeptide analyte. Polypeptide analyte are substrates of binders disclosed herein.

As used herein, the term “sample” refers to anything which may contain an analyte for which an analyte assay is desired. As used herein, a “sample” can be a solution, a suspension, liquid, powder, a paste, aqueous, non-aqueous or any combination thereof. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like.

As used herein, the term “macromolecule” encompasses large molecules composed of smaller subunits. Examples of macromolecules include, but are not limited to peptides, polypeptides, polypeptides, nucleic acids, carbohydrates, lipids, macrocycles, or a combination or complex thereof.

As used herein, the term “polypeptide” encompasses peptides, polypeptides and proteins, referring to a molecule comprising a chain of three or more amino acids joined by peptide bonds. In some embodiments, a polypeptide comprises 3 to 50 amino acid residues and is used interchangeably with the term “peptide”. In some embodiments, the term “peptide” refers to a polypeptide fragment. In some embodiments, a peptide does not comprise a secondary, tertiary, or higher structure. In some embodiments, the polypeptide is a protein. In some embodiments, a protein comprises more than 50 amino acid residues, and, in addition to a primary structure, has a secondary, tertiary, or higher structure. In some embodiments, polypeptides are formed by fragmenting proteins with a protease or by other means. The amino acids of the polypeptides are most typically L-amino acids, but may also be D-amino acids, modified amino acids, amino acid analogs, amino acid mimetics, or any combination thereof. Polypeptides may be naturally occurring, synthetically produced, or recombinantly expressed, or produced by a combination of these methodologies. Polypeptides may also comprise additional groups modifying the amino acid chain, for example, functional groups added via post-translational modification. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a detectable label.

As used herein, the term “amino acid” refers to an organic compound, which serves as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized.

As used herein, the term “binding agent” or “binder” refers to a nucleic acid molecule, a peptide, a polypeptide, a polypeptide, carbohydrate, or a small molecule that binds to, associates, unites with, recognizes, or combines with a binding target, e.g., a polypeptide or a component or feature of a polypeptide (e.g., a modified terminal amino acid residue). A binding agent may form a covalent association or non-covalent association with the polypeptide or component or feature of a polypeptide. A binding agent may also be a chimeric binding agent, composed of two or more types of molecules, such as a nucleic acid molecule-peptide chimeric binding agent or a carbohydrate-peptide chimeric binding agent. A binding agent may be a naturally occurring, synthetically produced, or recombinantly expressed molecule. A binding agent may bind to a single monomer or subunit of a polypeptide (e.g., a single amino acid of a polypeptide) or bind to a plurality of linked subunits of a polypeptide (e.g., a di-peptide, tri-peptide, or higher order peptide of a longer peptide, polypeptide, or polypeptide molecule). A binding agent may bind to a linear molecule or a molecule having a three-dimensional structure (also referred to as conformation). A binding agent may bind to an N-terminal peptide, a C-terminal peptide, or an intervening peptide of a peptide, polypeptide, or polypeptide molecule. A binding agent may bind to an N-terminal amino acid, C-terminal amino acid, or an intervening amino acid of a peptide molecule. A binding agent may preferably bind to a chemically modified or labeled amino acid (e.g., an amino acid that has been labeled by a chemical reagent) over a non-modified or unlabeled amino acid. For example, a binding agent may preferably bind to an amino acid that has been labeled or modified over an amino acid that is unlabeled or unmodified. A binding agent may bind to a post-translational modification of a peptide molecule. A binding agent may exhibit selective binding to a component or feature of a polypeptide (e.g., a binding agent may selectively bind to one of the 20 possible natural amino acid residues and bind with very low affinity or not at all to the other 19 natural amino acid residues). A binding agent may exhibit less selective binding, where the binding agent is capable of binding or configured to bind to a plurality of components or features of a polypeptide (e.g., a binding agent may bind with similar affinity to two or more different amino acid residues). A binding agent may comprise a coding tag, which may be joined to the binding agent by a linker.

The term “specific binding” as used herein refers to a binding reaction between an engineered binder and a cognate peptide (e.g., a peptide having a particular NTAA residue to which the binder binds) or a portion thereof, which occurs more readily than a similar reaction between the engineered binder and a random, non-cognate peptide. The term “specificity” is used herein to qualify the relative affinity by which an engineered binder binds to a cognate (e.g., suitable for binding based on the designed affinity) peptide. Specific binding typically means that an engineered binder binds to a cognate peptide at least twice more likely that to a random, non-cognate peptide (a 2:1 ratio of specific to non-specific binding). Specific binding to a particular modified NTAA residue of a peptide means that a binder binds to the modified NTAA residue with higher affinity compared to the same, but unmodified NTAA residue, and compared to other (structurally different) modified NTAA residues (modification of NTAA residue increases binding affinity between the binder and the peptide). In some embodiments, specific binding is not strictly selective, such as a binder specifically bind to two or more different modified NTAA residues compared to other modified NTAA residues. For example, a binder may specifically bind to both D and E modified NTAA residues compared to other modified NTAA residues (dual specificity). In another example, a binder may specifically bind to V, I and L modified NTAA residues compared to other modified NTAA residues (multi-specificity). Non-specific binding refers to background binding, and is the amount of signal that is produced in a binding assay between an engineered binder and an N-terminally modified peptide when the modified NTAA residue cognate for the engineered binder is not present at the N-terminus of the peptide. In some embodiments, specific binding refers to binding between an engineered metalloprotein binder and an N-terminally modified target peptide with a dissociation constant (Kd) of 500 nM or less.

In some embodiments, binding specificity between an engineered binder and an N-terminally modified target peptide is predominantly or substantially determined by interaction between the engineered binder and the modified NTAA residue of the N-terminally modified target peptide, which means that there is only minimal or no interaction between the engineered binder and the penultimate terminal amino acid residue (P2) of the target peptide, as well as other residues of the target peptide. In some embodiments, the engineered binder binds with at least 5-fold higher binding affinity to the modified NTAA residue of the target peptide than to any other region of the target peptide. In some embodiments, the engineered binder has a substrate binding pocket with certain size and/or geometry matching the size and/or geometry of the modified NTAA residue of the N-terminally modified target peptide, to which the engineered binder specifically binds to. In such embodiments, the modified NTAA residue occupies a volume encompassing a substrate binding pocket of the engineered binder that effectively precludes the P2 residue of the target peptide from entering into the substrate binding pocket or interacting with affinity-determining residues of the engineered binder. In some embodiments, the engineered binder specifically binds to N-terminally modified target peptides, wherein the target peptides share the same modified NTAA residue that interacts with the engineered binder, but have different P2 residues. In some embodiments, the engineered binder is capable of specifically binding to each N-terminally modified target peptide from a plurality of N-terminally modified target peptides, wherein the plurality of N-terminally modified target peptides contains at least 3, at least 5, or at least 10 N-terminally modified target peptides that were modified with the same N-terminal modifier agent, have the same modified NTAA residue, and have different P2 residues. Thus, in preferred embodiments, the engineered binder possesses binding affinity towards the modified NTAA residue of the N-terminally modified target peptide, but has little or low affinity towards P2 or other residues of the target peptide.

As used herein, the term “selectivity” refers to the ability of a binder to preferentially bind to one or to several amino acid residues of a peptide analyte, optionally modified with a chemical modification. In preferred embodiments, “selectivity” describes preferential binding of a binder to a single N-terminal amino acid residue, or to a small group of NTAA residues (e.g., structurally related), optionally covalently modified with a modification or label. In some embodiments, a binder may exhibit selective binding to a particular amino acid residue. In some embodiments, a binder may exhibit selective binding to a particular class or type of amino acid residues. In some embodiments, a binder may exhibit particular binding kinetics (e.g., higher association rate constant and/or lower dissociation rate constant) to a particular class or type of amino acid residues or modified amino acid residues, compared to other amino acid residues or modified amino acid residues. In some embodiments, a binder may exhibit selective binding to a component of a peptide analyte. In other embodiments, binder may exhibit less selective binding, where the binder is capable of binding or configured to bind to a plurality of components of a peptide analyte (e.g., a binder may bind with similar affinity to two or more different NTAA residues). In some embodiments, selectivity of each binder towards NTAA resides of peptide analytes is determined in advance, before performing contacting steps of the disclosed methods.

As used herein, the terms “encoding assay” and “reverse translation assay” are used interchangeably herein to refer to a process where polypeptides are encoded as nucleic acid sequences by performing a cyclic process comprising contacting the immobilized polypeptides with binders from a set of binders (individually or as a mixture), and, following the binding, extending a recording tag associated with each polypeptide by transferring nucleic acid information which corresponds to the binding event. Each cycle of the encoding assay (“encoding cycle”) results in extension of recording tags of polypeptide analytes. Typically, encoding assay has from 3 to 20 encoding cycles. Non-limited examples of encoding assays are disclosed in U.S. Ser. No. 11/513,126 B2, U.S. Ser. No. 11/782,062 B2, US 2019/0145982 A1, US 2023/0136966 A1, US 2024/0294981 A1, and US 2024/0053350 A1, each of which is incorporated herein by reference.

As used herein, the term “linker” refers to one or more of a nucleotide, a nucleotide analog, an amino acid, a peptide, a polypeptide, a polymer, or a non-nucleotide chemical moiety that is used to join two molecules. A linker may be used to join a binder with a coding tag, a recording tag with a polypeptide, a polypeptide with a support, a recording tag with a solid support, etc. In certain embodiments, a linker joins two molecules via enzymatic reaction or chemistry reaction (e.g., click chemistry).

As used herein, the term “barcode” refers to a nucleic acid molecule of about 3 to about 30 bases (e.g., 3, 4, 5, 6, 7, 8, 9, 10, 11, 12, 13, 14, 15, 16, 17, 18, 19, 20, 21, 22, 23, 24, 25, 26, 27, 28, 29 or 30 bases) providing a unique identifier tag or origin information for a polypeptide, a binder, a set of binders from one encoding cycle (when sets are changed between cycles), a sample polypeptides, a set of samples, polypeptides within a compartment (e.g., droplet, bead, or separated location), polypeptides within a set of compartments, a fraction of polypeptides, a spatial region or set of spatial regions, or a library of polypeptides. A barcode can be an artificial sequence or a naturally occurring sequence. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error-correcting or error-tolerant barcodes. Hamming distance, Lee distance, asymmetric Lee distance, Reed-Solomon, Levenshtein-Tenengolts, or similar methods for error-correction may be employed. Barcodes can be used to computationally deconvolute the multiplexed sequencing data and identify sequence reads derived from an individual polypeptide, sample, library, etc. A barcode can also be used for deconvolution of a collection of polypeptides that have been distributed into small compartments for enhanced mapping. For example, rather than mapping a peptide back to the proteome, the peptide is mapped back to its originating polypeptide molecule or polypeptide complex. In some embodiments, the term “barcode” also refers to a peptide molecule (e.g., peptide barcode).

As used herein, the term “encoder barcode” refers to a nucleic acid molecule of about 2 bases to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) in length that provides identifying information for its associated binding agent. The encoder barcode may uniquely identify its associated binding agent. During the described methods, an encoder barcode is first present in the coding tag attached to the associated binding agent. During the encoding cycle, when the binding agent binds to a polypeptide analyte, the encoder barcode of a complement thereof is transferred to a recording tag associated with the polypeptide. In certain embodiments, an encoder barcode provides identifying information for its associated binding agent and for the binding cycle in which the binding agent is used. In some embodiments, encoder barcode may correspond to an absence of a binding event within the encoding cycle (“null” encoder barcode sequence). In other embodiments, an encoder barcode is combined with a separate binding cycle-specific barcode (i.e., a unique sequence used to identify a library of binding agents used within a particular binding cycle) within a coding tag. In some embodiments, the encoder barcode may identify its associated binding agent as belonging to a member of a set of two or more different binding agents. In some embodiments, the encoder barcode sequence may correspond to two or more binder from a set of binders, and this level of identification is sufficient for the purposes of analysis. For example, in some embodiments involving a binding agent that binds to an amino acid, it may be sufficient to know that a peptide comprises one of two possible amino acids at a particular position, rather than definitively identify the amino acid residue at that position. In other embodiments, where an encoder barcode identifies a set of possible binding agents, a sequential decoding approach can be used to produce unique identification of each binding agent. This is accomplished by varying encoder barcodes for a given binding agent in repeated cycles of binding (see, Gunderson et al., 2004, Genome Res. 14:870-7). The partially identifying coding tag information from each binding cycle, when combined with coding information from other cycles, produces a unique identifier for the binding agent, e.g., the particular combination of coding tags rather than an individual coding tag (or encoder barcode) provides the uniquely identifying information for the binding agent.

As used herein, the term “coding tag” or “CT” refers to a polynucleotide with any suitable length, e.g., a nucleic acid molecule of about 3 bases to about 100 bases, including any integer including 3 and 100 and in between, that comprises identifying information for its associated binder. A “coding tag” may also be made from a “sequenceable polymer” (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety). A coding tag may comprise an encoder sequence (e.g., barcode that comprises identifying information regarding the binder), which is optionally flanked by one spacer on one side or optionally flanked by a spacer on each side. A coding tag may also be comprised of an optional UMI and/or an optional encoding cycle-specific barcode. A coding tag may be single stranded or double-stranded. A double-stranded coding tag may comprise blunt ends, overhanging ends, or both. A coding tag may refer to the coding tag that is directly attached to a binder, to a complementary sequence hybridized to the coding tag directly attached to a binder (e.g., for double-stranded coding tags), or to coding tag information present in an extended recording tag.

As used herein, the term “recording tag” or “RT” refers to a moiety, e.g., a nucleic acid molecule, or a sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) to which identifying information of a coding tag can be transferred, or from which identifying information about the polypeptide (e.g., UMI information) associated with the recording tag can be transferred to the coding tag. Identifying information can comprise any information characterizing a molecule such as information pertaining to sample, fraction, partition, spatial location, interacting neighboring molecule(s), cycle number, etc. Additionally, the presence of UMI information can also be classified as identifying information. In certain embodiments, after a binder binds to a polypeptide, information from a coding tag linked to a binder can be transferred to the recording tag associated with the polypeptide while the binder is bound to the polypeptide. In other embodiments, after a binder binds to a polypeptide, information from a recording tag associated with the polypeptide can be transferred to the coding tag linked to the binder while the binder is bound to the polypeptide. A recording tag may be directly linked to a polypeptide, linked to a polypeptide via a multifunctional linker, or associated with a polypeptide by virtue of its proximity (or co-localization) on a support. A recording tag may be linked via its 5′ end or 3′ end or at an internal site, as long as the linkage is compatible with the method used to transfer coding tag information to the recording tag or vice versa. A recording tag may further comprise other functional components, e.g., a universal priming site, unique molecular identifier, a barcode (e.g., a sample barcode, a fraction barcode, spatial barcode, a compartment tag, etc.), a spacer sequence that is complementary to a spacer sequence of a coding tag, or any combination thereof. The spacer sequence of a recording tag is preferably at the 3′-end of the recording tag in embodiments where polymerase extension is used to transfer coding tag information to the recording tag.

As used herein, the term “spacer” (Sp) refers to a nucleic acid molecule of about 1 base to about 20 bases (e.g., 1, 2, 3, 4, 5, 6, 7, 8, 9, 10, 11, 12, 13, 14, 15, 16, 17, 18, 19, or 20 bases) in length that is present on a terminus of a recording tag or coding tag. In certain embodiments, a spacer sequence flanks an encoder sequence of a coding tag on one end or both ends. Following binding of a binder to a polypeptide, annealing between complementary spacer sequences on their associated coding tag and recording tag, respectively, allows transfer of binding information through a primer extension reaction or ligation to the recording tag, coding tag, or a di-tag construct. Preferably, spacer sequences within a set of binders possess the same number of bases. A common (shared or identical) spacer may be used in a set of binders. A spacer sequence may have a “cycle specific” sequence in order to track binders used in a particular encoding cycle (i.e., contacting-transferring-releasing steps of the methods disclosed herein form “encoding cycle”). The spacer sequence (Sp) can be constant across all encoding cycles, be specific for a particular class of polypeptides, or be encoding cycle number specific. In some embodiments, only the sequential binding of correct cognate pairs of RT and CT results in interacting spacer elements and effective primer extension. A spacer sequence may comprise sufficient number of bases to anneal to a complementary spacer sequence in a recording tag to initiate a primer extension (also referred to as polymerase extension) reaction, or provide a “splint” for a ligation reaction, or mediate a “sticky end” ligation reaction. A spacer sequence may comprise a fewer number of bases than the encoder sequence within a coding tag.

As used herein, the term “primer extension”, also referred to as “polymerase extension” and “extension”, refers to a reaction catalyzed by a nucleic acid polymerase (e.g., DNA polymerase) whereby a nucleic acid molecule (e.g., oligonucleotide primer, spacer sequence) that anneals to a complementary strand is extended by the nucleic acid polymerase, using the complementary strand as template. Various polymerases capable of performing the extension are known in the art.

As used herein, the term “unique molecular identifier” or “UMI” refers to a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases) in length providing a unique identifier tag for each polypeptide, polypeptide or binder to which the UMI is linked. A polypeptide UMI can be used to computationally deconvolute sequencing data from a plurality of extended recording tags to identify extended recording tags that originated from an individual polypeptide. A polypeptide UMI can be used to accurately count originating polypeptide molecules by collapsing NGS reads to unique UMIs. A binder UMI can be used to identify each individual molecular binder that binds to a particular polypeptide. For example, a UMI can be used to identify the number of individual binding events for a binder specific for a single amino acid that occurs for a particular peptide molecule. It is understood that when UMI and barcode are both referenced in the context of a binder or polypeptide, that the barcode refers to identifying information other that the UMI for the individual binder or polypeptide (e.g., sample barcode, compartment barcode, encoding cycle barcode).

As used herein, the term “universal priming site” or “universal primer” or “universal priming sequence” refers to a nucleic acid molecule, which may be used for analysis, amplification, and/or for sequencing of extended recording tags. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. Universal priming sites can be used for other types of amplification, including those commonly used in conjunction with next generation digital sequencing. For example, extended recording tag molecules may be circularized and a universal priming site used for rolling circle amplification to form DNA nanoballs that can be used as sequencing templates (Drmanac et al., 2009, Science 327:78-81). Alternatively, recording tag molecules may be circularized and sequenced directly by polymerase extension from universal priming sites (Korlach et al., 2008, Proc. Natl. Acad. Sci. 105:1176-1181).

As used herein, the term “extended recording tag” refers to a recording tag to which information of at least one binder's coding tag (or its complementary sequence) has been transferred following binding of the binder to a polypeptide. Information of the coding tag may be transferred to the recording tag directly (e.g., ligation) or indirectly (e.g., primer extension). Information of a coding tag may be transferred to the recording tag enzymatically or chemically (e.g., by chemical ligation). An extended recording tag may comprise binder information of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50 or more coding tags. The base sequence of an extended recording tag may reflect the temporal and sequential order of binding of the binders identified by their coding tags, may reflect a partial sequential order of binding of the binders identified by the coding tags, or may not reflect any order of binding of the binders identified by the coding tags. In certain embodiments, the coding tag information present in the extended recording tag represents with at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity the polypeptide sequence being analyzed. In certain embodiments where the extended recording tag does not represent the polypeptide sequence being analyzed with 100% identity, errors may be due to off-target binding by a binder, or to a “missed” encoding cycle (e.g., because a binder fails to bind to a polypeptide during a encoding cycle, because of a failed primer extension reaction), or both.

As used herein, the term “solid support” or “support” refers to any solid material, including porous and non-porous materials, to which a polypeptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, a PTFE membrane, a PTFE membrane, a nitrocellulose membrane, a nitrocellulose-based polymer surface, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, a microparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. For example, when solid surface is a bead, the bead can include, but is not limited to, a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, a controlled pore bead, a silica-based bead, or any combinations thereof. A bead may be spherical or an irregularly shaped. A bead's size may range from nanometers, e.g., 10 nm, to millimeters, e.g., 1 mm. In certain embodiments, beads range in size from about 0.2 micron to about 200 microns, or from about 0.5 micron to about 5 micron. In certain embodiments, “a bead” solid support may refer to an individual bead or a plurality of beads. In certain embodiments, the nanoparticles range in size from about 10 nm to about 500 nm in diameter.

As used herein, the term “nucleic acid” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), γPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In some embodiments, the nucleic acid molecule or oligonucleotide is a DNA with pseudo-complementary bases. In some embodiments, the nucleic acid molecule or oligonucleotide is backbone modified, sugar modified, or nucleobase modified.

As used herein, “nucleic acid sequencing” means the determination of the order of nucleotides in a nucleic acid molecule or a sample of nucleic acid molecules. And refer to any possible sequencing method from a variety of sequencing methods known in the art. Examples of sequencing methods include, without limitation, next generation sequencing, sequencing by synthesis, sequencing by ligation, sequencing by hybridization, polony sequencing, ion semiconductor sequencing, nanopore sequencing, single molecule sequencing and pyrosequencing. Further examples of nucleic acid sequencing technologies include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays (See e.g., Service, Science (2006) 311:1544-1546). Some sequencing methods rely on amplification to clone many nucleic acid (e.g., DNA) molecules in parallel for sequencing in a phased approach. Single molecule sequencing interrogates single molecules of DNA and does not require amplification or synchronization. Examples of single molecule sequencing methods include single molecule real-time sequencing (Pacific Biosciences), nanopore-based sequencing (Oxford Nanopore), duplex interrupted nanopore sequencing, and direct imaging of DNA using advanced microscopy.

As used herein, “analyzing” the polypeptide means to identify, detect, quantify, characterize, distinguish, or a combination thereof, all or a portion of the components of the polypeptide. For example, analyzing a peptide, polypeptide, or polypeptide includes determining all or a portion of the amino acid sequence (contiguous or non-continuous) of the peptide. Analyzing a polypeptide also includes partial identification of a component of the polypeptide (e.g., NTAA of the polypeptide). For example, partial identification of an amino acid residue in the polypeptide sequence can identify an amino acid in the polypeptide as belonging to a subset of possible amino acid residues. In preferred embodiments, polypeptide analysis typically begins with analysis of the n NTAA, and then proceeds to the next amino acid residue of the polypeptide (i.e., n−1, n−2, n−3, and so forth). This is accomplished by removal of the n NTAA, thereby converting the n−1 amino acid residue of the polypeptide to a N-terminal amino acid (referred to herein as the “n−1 NTAA”). Analyzing the polypeptide may also include determining the presence and frequency of post-translational modifications on the peptide, which may or may not include information regarding the sequential order of the post-translational modifications on the polypeptide. Analyzing the peptide may also include determining the presence and frequency of epitopes in the polypeptide, which may or may not include information regarding the sequential order or location of the epitopes within the polypeptide. Analyzing the polypeptide may include combining different types of analysis, for example obtaining epitope information, amino acid sequence information, post-translational modification information, or any combination thereof.

As used herein, the term “detectable label” refers to a substance which can indicate the presence of another substance when associated with it. The detectable label can be a substance that is linked to or incorporated into the substance to be detected. In some embodiments, a detectable label is suitable for allowing for detection and also quantification, for example, a detectable label that emitting a detectable and measurable signal. Detectable labels include any labels that can be utilized and are compatible with the provided peptide analysis assay format and include, but not limited to, a bioluminescent label, a biotin/avidin label, a chemiluminescent label, a chromophore, a coenzyme, a dye, an electro-active group, an electrochemiluminescent label, an enzymatic label (e.g. alkaline phosphatase, luciferase or horseradish peroxidase), a fluorescent label, a latex particle, a magnetic particle, a metal, a metal chelate, a phosphorescent dye, a polypeptide label, a radioactive element or moiety, and a stable radical. When attached to a binder, a detectable label may indicate a binding event between the binder and a polypeptide analyte.

The term “unmodified” (also “wild-type” or “native”) as used herein is used in connection with biological materials such as nucleic acid molecules and polypeptides, refers to those which are found in nature and not modified by human intervention.

The term “modified”, or “engineered”, or “variant”, or “mutant” as used in reference to nucleic acid molecules and polypeptide molecules, e.g., engineered binders, implies that such molecules are created by human intervention and/or they are non-naturally occurring. The variant, mutant or engineered binder is a polypeptide having an altered amino acid sequence, relative to an unmodified or wild-type polypeptide, such as starting scaffold, or a portion thereof. An engineered binder is a polypeptide which differs from a wild-type scaffold sequence, or a portion thereof, by one or more amino acid substitutions, deletions, additions, or combinations thereof. Sequence of an engineered binder can contain 1, 2, 3, 4, 5, 6, 7, 8, 9, 10, 20, 30, 50, 100 or more amino acid differences (e.g., mutations) compared to the sequence of starting scaffold. An engineered binder generally exhibits at least 30%, 40%, 50%, 60%, 70%, 80%, 85%, 86%, 87%, 88%, 89%, 90%, 91%, 92%, 93%, 94%, 95%, 96%, 97%, 98%, 99% or more sequence identity to a corresponding wild-type starting scaffold. Non-naturally occurring amino acids as well as naturally occurring amino acids are included within the scope of permissible substitutions or additions. An engineered binder is not limited to any engineered binders made or generated by a particular method of making and includes, for example, an engineered binder made or generated by genetic selection, polypeptide engineering, chemical synthesis, directed evolution, de novo recombinant DNA techniques, or combinations thereof. The term “variant” in the context of variant or engineered binder is not to be construed as imposing any condition for any particular starting composition or method by which the variant or engineered binder is created. Thus, variant or engineered binder denotes a composition and not necessarily a product produced by any given process.

The term “modified amino acid residue” as used herein refers to an amino acid residue within a polypeptide that comprises a modification that distinguish it from the corresponding original, or unmodified, amino acid residue. In some embodiments, the modification can be a naturally occurring post-translational modification of the amino acid residue. In other embodiments, the modification is a non-naturally occurring modification of the amino acid residue; such modified amino acid residue is not naturally present in peptides of living organisms (represents an unnatural amino acid residue). Such modified amino acid residue can be made by modifying a natural amino acid residue within the polypeptide by a modifying reagent, such as N-terminal modifier agent, or can be chemically synthesized and incorporated into the peptide during peptide synthesis.

In some embodiments, variants of an engineered polypeptide displaying only non-substantial or negligible differences in structure can be generated by making conservative amino acid substitutions in the engineered polypeptide. By doing this, engineered polypeptide variants that comprise a sequence having at least 80% (85%, 90%, 91%, 92%, 93%, 94%, 95%, 96%, 97%, 98%, and 99%) sequence identity with the engineered polypeptide sequences can be generated, retaining at least one functional activity of the engineered polypeptide, e.g., ability to specifically bind an N-terminal amino acid (NTAA) residue of the polypeptide analyte. Examples of conservative amino acid changes are known in the art. Examples of non-conservative amino acid changes that are likely to cause major changes in polypeptide structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histidine, for (or by) an electronegative residue, e.g., glutamic acid or aspartic acid; or (d) a residue having a bulky side chain, e.g., phenylalanine, for (or by) one not having a side chain, e g., glycine. Methods of making targeted amino acid substitutions, deletions, truncations, and insertions are generally known in the art. For example, amino acid sequence variants can be prepared by mutations in the DNA. Methods for polynucleotide alterations are well known in the art, for example, Kunkel et al. (1987) Methods in Enzymol. 154:367-382; U.S. Pat. No. 4,873,192 and the references cited therein.

As used herein, “identifying” a peptide means to predict identity of the peptide with a certain probability. It can be done by identifying a component (e.g., one or more amino acid residues) of the peptide. It can also be done by predicting certain amino acid residues of the peptide and their positions with certain probability, thus creating a peptide signature, and then matching bioinformatically the resulted peptide signature with corresponding signatures of peptides that may be present in the sample (e.g., by matching the peptide signature with peptide sequences from a proteomic or genomic database). For example, in some embodiments, existing selectivity of a binder is not enough to determine the NTAA residue to which the binder is bound with certainty. In these cases, identity of the NTAA residue can be determined with certain probability (such as being D, E or H and not A, G, I or L). Subsequent similar determination of adjacent amino acid residues creates an array of possible variants for the peptide based on variants in the assayed amino acid residues, and by matching this array of variants with theoretical possibilities determined from a proteomic or genomic database, it can be narrowed down to a particular sequence, if enough amino acid residues were assayed.

The term “sequence identity” is a measure of identity between polypeptides at the amino acid level, and a measure of identity between nucleic acids at nucleotide level. The polypeptide sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. “Sequence identity” means the percentage of identical subunits at corresponding positions in two sequences when the two sequences are aligned to maximize subunit matching, i.e., taking into account gaps and insertions. For example, the BLAST algorithm (NCBI) calculates percent sequence identity and performs a statistical analysis of the similarity and identity between the two sequences. The software for performing BLAST analysis is publicly available through the National Center for Biotechnology Information (NCBI) website.

The terms “corresponding to position(s)” or “position(s) . . . with reference to position(s)” of or within a polypeptide or a polynucleotide, such as recitation that nucleotides or amino acid positions “correspond to” nucleotides or amino acid positions of a disclosed sequence, such sequence set forth in the Sequence Listing, refers to nucleotides or amino acid positions identified in the polynucleotide or in the polypeptide upon alignment with the disclosed sequence using a standard alignment algorithm, such as the BLAST algorithm (NCBI). One skilled in the art can identify any given amino acid residue in a given polypeptide at a position corresponding to a particular position of a reference sequence, such as set forth in the Sequence Listing, by performing alignment of the polypeptide sequence with the reference sequence (for example, by using BLASTP publicly available through the NCBI website), matching the corresponding position of the reference sequence with the position in polypeptide sequence and thus identifying the amino acid residue within the polypeptide.

The term “joining” or “attaching” one substance to another substance means connecting or linking these substances together utilizing one or more covalent bond(s) and/or non-covalent interactions. Some examples of non-covalent interactions include hydrogen bonding, hydrophobic binding, and Van der Waals forces. Joining can be direct or indirect, such as via a linker or via another moiety. In preferred embodiments, joining two or more substances together would not impair structure or functional activities of the joined substances. The term “associated with” (e.g., one substance is associated with to another substance) means bringing two substances together, so they can coordinately participate in the methods described herein. In preferred embodiments, association of two substances preserves their structures and functional activities. Association can be direct or indirect. When one substance is directly associated with another substance, it is equivalent to one substance being joined or attached to another substance. Indirect association means that two substances are brought together by means other than direct joining or attachment. In some embodiments, indirect association implies that two substances are co-localized with each other, or located in a close proximity with each other.

As used herein, the term “macromolecule comprises a component” refers to a situation where the component is either a part of the macromolecule, or directly attached to the macromolecule by means of one or more covalent bond(s), which unite them into a single molecule. Instead, the term “macromolecule associated with a component” indicates that the component may or may not be directly attached to the macromolecule by means of one or more covalent bond(s), but instead can be associated, or co-localized, with the macromolecule by means of non-covalent interactions, or, alternatively, be associated indirectly through a solid support (for example, when the macromolecule is attached to the solid support, and the component is independently attached to the solid support in a proximity to the macromolecule. For example, “macromolecule is associated with a recording tag” encompasses various possible ways for association between the macromolecule and the recording tag (either direct, covalent or non-covalent association, or indirect association, such as association via a linker or via another object, such as via solid support). The terms “attaching” and “joining” are used interchangeably and refer to either covalent or non-covalent attachment.

The term “peptide bond” as used herein refers to a chemical bond formed between two molecules (such as two amino acids) when the carboxyl group of one molecule reacts with the amino group of the other molecule, releasing a water molecule (H₂O).

The term “binding profile” of a binder (also known as a binder's specificity profile) conveys information about binding and/or encoding activity of the binder in an encoding assay across two or more polypeptide analytes. In some embodiments, a binding profile is referred to as binding and/or encoding activity of a binder at a given concentration for various NTAA-modified polypeptides at their ultimate and penultimate amino acid residues (e.g., a representative exemplar binding profile is given in FIG. 16A-FIG. 16G). In these examples, the binding profile of a binder towards P1-P2 residues of immobilized peptides conveys information how well the binder binds and encodes different modified NTAA residues of peptides (the binding profile depends on P2 residue of peptides). In other embodiments, binding profiles convey information about binding and/or encoding activity in an encoding assay at a given concentration of a single binder for entire polypeptide sequences in a sample. Encoding activity is characterized by encoding yield (or encoding rate) which is a fraction of recording tags encoded at each cycle (a fraction of recording tags that were extended with an encoder barcode transferred from a coding tag of the binder in a given encoding cycle). Encoding yield of a binder depends on binder concentration in the encoding assay. Encoding yield of a binder may also change when encoding assay parameters are changing, such as binder incubation time, ligase incubation time, buffer conditions, washing steps, etc.

Binding profile of a binder may change when the binder is mixed with other binders that can compete with the binder for substrates (such as compete for modified NTAA residues of peptides). In a given set of binders used in an encoding assay, the binding profile of each binder of the set may be determined from its encoding activity even when other binders are present around (see FIG. 16A-FIG. 16G). Binding profile of a binder may change when concentrations of binders in the binder set are altered. In the methods of inferring amino acid sequences of polypeptides from binder identifier strings produced in an encoding assay, binding profiles of each binder of the set of binders used in the encoding assay may be determined in advance (predetermined from previously generated encoding datasets obtained using essentially identical encoding assay parameters, at one or several different binder concentrations), or may be determined concurrently while performing other method steps, for example, by including a ground truth sample within the peptide analytes. In some embodiments, a computer model may calculate (determine) binding profiles of each binder of the set of binders used in the encoding assay when either previously generated encoding datasets or actual encoding data are inputted into the model, allowing the model to infer binder specificity and encoding activity. In some embodiments, binding profiles of each binder may be determined from encoding activity of the binder implicitly using a training dataset with known polypeptide sequences, such as the disclosed algorithms may internally learn a representation of binding profiles of the set of binders. In some embodiments, the encoding activity of a binder is implicit in the underlying dataset and may be determined through training a machine learning model on the dataset, and although the binding profiles may not be analyzed directly, the binding profile information may be captured and learned through updating the parameters of the machine learning model during training. In a non-limiting example, in the trained naive Bayes model approach (see Example 10), the conditional probabilities are calculated that a given amino acid identity in the polypeptide sequence at index i co-occurs with a binder token at index j. Thus, implicit within the trained model is the learned binding profiles for each binder (represented as a binder token) for each amino acid identity (represented as an amino acid token) at a given position in the polypeptide, such that a binding profile is learned from the training dataset by the model, although no one explicitly inputs supplementary binding profile information stemming from other encoding assay datasets into the computer model during training. Instead, the binding profiles are learned from the same underlying dataset during the training process, and then the learned implicit representations of binding profiles can be utilized to decode polypeptide sequences on the testing dataset or additional datasets.

The term “probability score” in general refers to a value that indicates the likelihood of an entity being classified into a specific class. In preferred embodiments, probability scores are between 0 and 100%. In some embodiments, “probability score” may refer to either empirical probability (also known as experimental probability) or theoretical probability, and derivations thereof including logits, log-probabilities, likelihoods, log-likelihoods, odds, log-odds, frequency, maximum likelihood estimate, and/or any metric that can be arrived at through statistical inference to represent likeliness or expectation of correct association between one or more binder identifiers and one or more amino acid sequences of polypeptides, as disclosed in the methods herein. In some embodiments, the disclosed machine learning models may generate probability scores as logits, which can be transformed into probabilities using a softmax function. Conversely, probabilities in the polypeptide decoding algorithm may be transformed into logits using the logit function. In some embodiments, when decoding binder identifier strings to infer polypeptide sequences, any metric that can be arrived at through statistical inference and that can be transformed into a probability that a given polypeptide sequence is inferred from, or associated with, a given binder identifier string is encompassed in the meaning of the “probability score” that may be produced by decoding algorithms described herein.

A “numerical representation” (also known as an “embedding”) of a binder identifier string, polypeptide sequence, or other amino acid sequence of a binder, Cleavase enzyme, or ligase enzyme is defined as an N-dimensional tensor, where N is a positive rational number, containing real numbers at each element of the tensor. In some embodiments, the information in the input sequence string (e.g., containing alphanumerical characters or amino acid identities) is transformed into a tensor of real numbers where the information in the sequence is present in a latent space that can be or further transformed by functions and/or input into a machine learning model, deep learning model, or artificial intelligence model.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

Other objects, advantages and features of the present invention will become apparent from the following specification taken in conjunction with the accompanying drawings.

### Description of Exemplary Embodiments

Provided herein is a computer-implemented method for analyzing a plurality of polypeptides encoded in a plurality of nucleic acid sequences by an encoding assay, the method comprising: (a) receiving, at one or more processors, the plurality of nucleic acid sequences generated from the encoding assay, wherein each of the plurality of nucleic acid sequences comprises a series of encoder barcode sequences, and wherein each encoder barcode sequence of a given series of encoder barcode sequences corresponds to a binder, from a set of binders, that binds to one or more components of a polypeptide of the plurality of polypeptides in the encoding assay; (b) generating a binder identifier string for each nucleic acid sequence of the plurality of nucleic acid sequences based on a corresponding series of encoder barcode sequences, thereby generating a plurality of binder identifier strings corresponding to the plurality of nucleic acid sequences; (c) inferring, using the one or more processors, an amino acid sequence of a polypeptide of the plurality of polypeptides from binder identifiers of a binder identifier string of the plurality of binder identifier strings based on (i) binding profiles of the binders from the set of binders that correspond to the binder identifiers of the binder identifier string, and (ii) calculated probability scores of an association between one or more binder identifiers of the binder identifier string and one or more amino acid sequences of polypeptides of the plurality of polypeptides; and (d) based on the calculated probability scores and the inferred amino acid sequences, outputting data related to at least a partial identity and/or quantity for at least one polypeptide of the plurality of polypeptides.

Provided herein is also a computer-implemented method for analyzing a computer-implemented method for analyzing a plurality of polypeptides encoded in a plurality of nucleic acid sequences by an encoding assay, the method comprising: a) receiving, at one or more processors, the plurality of nucleic acid sequences generated from the encoding assay, wherein each of the plurality of nucleic acid sequences comprises a series of encoder barcode sequences, and wherein each encoder barcode sequence of a given series of encoder barcode sequences corresponds to a binder that binds to a component of a polypeptide of the plurality of polypeptides in the encoding assay; b) assigning, using the one or more processors, a binder identifier to each of the series of encoder barcode sequences in each of the plurality of nucleic acid sequences to generate a plurality of binder identifier strings for the plurality of nucleic acid sequences; c) for each polypeptide and/or polypeptide fragment of the plurality of polypeptides, generating in silico a set of simulated binder identifier strings based on pre-determined parameters of the encoding assay, wherein the pre-determined parameters comprise: i) probabilities of assigning to at least one component of a given polypeptide or polypeptide fragment one or more binder identifiers either correctly or incorrectly based on binding profiles of binders used in the encoding assay; and ii) optionally, for at least one component of a given polypeptide or polypeptide fragment, a probability of successfully cleaving the at least one component after a binder binds to the at least one component in the encoding assay, thereby determining probabilities for each simulated binder identifier string generated from a given polypeptide or polypeptide fragment; d) for each binder identifier string of the plurality of binder identifier strings generated in (b), calculating probabilities that a given binder identifier string is generated from one or more polypeptides or fragments thereof based on probabilities for simulated binder identifier strings determined in (c), thereby determining at least a partial identity and/or quantity for at least one polypeptide of the plurality of polypeptides.

Provided herein is also a computer-implemented method, comprising: a) receiving, at one or more processors, amino acid sequences for a plurality of polypeptides; b) generating, using the one or more processors, a plurality of peptide sequences based on the amino acid sequences; c) generating, using the one or more processors, a plurality of peptidic reads based on the plurality of peptide sequences; d) converting, using the one or more processors, each peptidic read of the plurality of peptidic reads into an identifier string based on an order and determined binding profile for a plurality of binders, wherein each binder of the plurality binds to a component of a polypeptide and corresponds to a binder identifier, and wherein the binder identifier corresponds to a barcode sequence; and e) providing, using the one or more processors, the plurality of identifier strings as input to a trained model, wherein the trained model is configured to convert identifier strings to nucleic acid sequences based on a set of probabilities for binding of the plurality of binders to components of the polypeptides of the plurality of polypeptides and output a plurality of nucleic acid sequences corresponding to the plurality of polypeptides.

Various embodiments apply equally to the aspects provided herein but will for the sake of brevity be recited only once. Thus, various of the following embodiments apply equally to aspects recited below.

In some embodiments of the disclosed methods, the component of the polypeptide to which the binder binds comprises a polypeptide, or portion thereof, obtained by fragmenting the polypeptides of the plurality of polypeptides.

In some embodiments of the disclosed methods, the component of the polypeptide to which the binder binds comprises one or more amino acid residues.

In some embodiments of the disclosed methods, the component of the polypeptide to which the binder binds comprises a post-translation modification.

In some embodiments of the disclosed methods, the component of the polypeptide to which the binder binds comprises a modified amino acid residue.

In some embodiments of the disclosed methods, inferring amino acid sequences of polypeptides comprises: (i) for each of the plurality of binder identifier strings, converting a given binder identifier string into one or more peptidic reads based on the binding profiles of the binders of the set of binders that correspond to binder identifiers present in a given binder identifier string, and (ii) calculating a probability score for each of the one or more peptidic reads, wherein the probability score is indicative of a probability that a given peptidic read produces a given binder identifier string.

In some embodiments, the disclosed methods further comprise: for each of the plurality of binder identifier strings, filtering out peptidic reads of the one or more peptidic reads generated for a given binder identifier string based on (i) the probability score for each peptidic read, and/or (ii) a probability that a given peptidic read was generated from amino acid sequences of the plurality of polypeptides.

In some embodiments of the disclosed methods, inferring amino acid sequences of polypeptides comprises: (i) for each amino acid sequence of the plurality of polypeptides, generating one or more simulated binder identifier strings using one or more parameters of the encoding assay; (ii) for each of the one or more simulated binder identifier strings, or for each of one or more amino acid sequences of polypeptides of the plurality of polypeptides, calculating a probability score based on a probability that a given simulated binder identifier string is associated with one or more amino acid sequences of polypeptides of the plurality of polypeptides; and (iii) matching each of the plurality of binder identifier strings to the one or more simulated binder identifier strings based on the calculated probability scores for the one or more simulated binder identifier strings

In some embodiments of the disclosed methods, the one or more parameters of the encoding assay comprise an efficiency of a functionalization of N-terminal amino acid (NTAA) residues of polypeptides of the plurality of polypeptides, an efficiency of a cleavage of NTAA residues of polypeptides of the plurality of polypeptides, an efficiency of an encoding of NTAA residues of polypeptides of the plurality of polypeptides, or any combination thereof.

In some embodiments of the disclosed methods, inferring amino acid sequences of polypeptides comprises inputting the binder identifier strings into a trained machine learning model, wherein the trained machine learning model is trained on empirically determined encoding assay data.

In some embodiments of the disclosed methods, the trained machine learning model is trained using a training data set comprising binder identifier strings data, or numerical representations thereof, for one or more isolated polypeptide samples, or numerical representations thereof, that are subjected to the encoding assay using a same sample preparation protocol as that used to process the plurality of polypeptides.

In some embodiments of the disclosed methods, the trained machine learning model is configured to (i) map each binder identifier string of the plurality of binder identifier strings to a specific polypeptide sequence, or (ii) to fractionally assign a given binder identifier string to two or more specific polypeptide sequences, as part of inferring amino acid sequences of polypeptides from binder identifiers.

In some embodiments of the disclosed methods, the empirically determined assay parameter data comprises: (i) probabilities of assigning one or more binder identifiers from the set of binders to potential N-terminal amino acid (NTAA) residues of a polypeptide based on binding profiles of binders used in the encoding assay; and (ii) for each potential N-terminal amino acid residue in a polypeptide, a probability of successfully cleaving the N-terminal amino acid residue after the N-terminal amino acid residue is encoded in the encoding assay.

In some embodiments of the disclosed methods, inferring amino acid sequences of polypeptides from binder identifier strings comprises inputting the binder identifier strings, or numerical representations thereof, into a trained machine learning model, wherein the trained machine learning model is trained on a training data set comprising a set of simulated binder identifier strings generated for a given input distribution of polypeptides, or numerical representations thereof, based on empirically determined assay parameter data comprising:
(i) probabilities of assigning one or more binder identifiers from the set of binders to potential N-terminal amino acid (NTAA) residues of a polypeptide based on binding profiles of binders used in the encoding assay;
(ii) for each potential NTAA residue in a polypeptide, a probability of cleaving the NTAA residue after the NTAA is encoded in the encoding assay, and (iii) a probability of modifying the NTAA residue.

In some embodiments of the disclosed methods, the set of binders comprises at least 5 different binders, and wherein the one or more components of polypeptides to which each binder binds each comprises an NTAA residue of polypeptides of the plurality of polypeptides or an NTAA residue modified by a N-terminal modifier agent.

In some embodiments of the disclosed methods, the set of binders comprises at least 5 different binders, and wherein the one or more components of polypeptides to which each binder binds each comprises a terminal amino acid or a terminal dipeptide of polypeptides of the plurality of polypeptides.

In some embodiments of the disclosed methods, the trained machine learning model is trained on a training data set generated by performing multiple encoding assays with pre-determined analytes and by inputting the series of encoder barcode sequences or numerical representations thereof generated during each encoding assay and encoding assay parameters to the machine learning model.

In some embodiments of the disclosed methods, a computer model infers two or more amino acid sequences of polypeptides of the plurality of polypeptides from each binder identifier string, and outputs probabilities that a given binder identifier string of the plurality is derived from one of the two or more amino acid sequences of polypeptides inferred from the binder identifier string.

In some embodiments of the disclosed methods, (i) each of the plurality of nucleic acid sequences further comprises one or more auxiliary sequences; (ii) the method further comprises assigning, using the one or more processors, an auxiliary identifier to at least one of the one or more auxiliary sequences in each of the plurality of nucleic acid sequences to generate a plurality of binder identifier strings comprising the auxiliary identifier; and (iii) the one or more auxiliary sequences comprise one or more identifier sequences or complements thereof, one or more spacer sequences or complements thereof, one or more sequencing primer sequences or complements thereof, or any combination thereof. In some embodiments, the one or more identifier sequences comprise one or more sample barcode sequences, one or more bead barcode sequences, one or more unique molecular identifier (UMI) sequences, or any combination thereof.

In some embodiments of the disclosed methods, inferring amino acid sequences of polypeptides from binder identifier strings comprises inputting the binder identifier strings and encoding assay data to the computer model. In some embodiments, the computer model is configured to identify unique binder identifier signatures in the plurality of binder identifier strings as part of inferring amino acid sequences of polypeptides from binder identifiers, and wherein a given unique binder identifier signature comprises a set of binder identifier strings associated with a single polypeptide of the plurality of polypeptides. In some embodiments, the computer model computes binding profiles of binders of the set of binders based on the inputted encoding assay parameter data.

In some embodiments of the disclosed methods, inferring amino acid sequences of polypeptides from binder identifiers comprises inputting one or more corresponding numerical representations of the binder identifier strings and optionally one or more corresponding numerical representations of encoding assay parameter data to a computer model.

In some embodiments of the disclosed methods, inferring amino acid sequences of polypeptides from binder identifiers comprises outputting one or more corresponding numerical representations of the polypeptide sequences from a computer model. In some embodiments, the corresponding numerical representations of the binder identifier strings are binder sequence embeddings generated from a trained protein language model (pLM), protein foundation model or natural language processing (NLP) model. In some embodiments, the corresponding numerical representations of the polypeptide sequences are polypeptide sequence embeddings generated from a protein language model, protein foundation model, or natural language processing model.

In some embodiments of the disclosed methods, the corresponding numerical representations of encoding assay parameter data includes: (i) the concentrations of one or more binders in the set of binders; (ii) thermodynamic parameters of binders, including association rate constants and dissociation rate constants for components of one or more polypeptides; (iii) the incubation time of binders; (iv) the wash time of binders after binder incubation; (v) the ligase concentration; (v) the ligase reaction time; (vi) thermodynamic parameters of the ligase, including Michaelis constant and catalytic turnover rate; (vii) estimated polypeptide concentrations; (viii) the concentration of cleavase enzymes; and (ix) buffer conditions for each step of the encoding assay, including enzymatic substrate concentrations, salt identities, salt concentrations, and pH.

In some embodiments of the disclosed methods, inferring amino acid sequences of polypeptides from binder identifiers of the plurality of binder identifier strings comprises: (c1) converting the plurality of binder identifier strings into a plurality of peptidic reads based on determined binding profiles of binders that correspond to binder identifiers assigned in a given binder identifier string; and (c2) providing, using the one or more processors, the plurality of peptidic reads as input to the computer model, wherein the computer model is configured to assign peptidic reads to amino acid sequences of polypeptides of the plurality of polypeptides and output data related to at least a partial identity and/or quantity for at least one polypeptide of the plurality of polypeptides.

In some embodiments of the disclosed methods, the output of the computer model comprises either (i) the partial identity and/or quantity for at least one polypeptide of the plurality of polypeptides, or (ii) data from which the partial identity and/or quantity for at least one polypeptide of the plurality of polypeptides are derived by a user.

In some embodiments of the disclosed methods, the output of the computer model comprises one or more amino acid sequences of polypeptides of the plurality of polypeptides for each of the plurality of nucleic acid sequences generated by the encoding assay and received at one or more processors. In some embodiments of the disclosed methods, the computer model outputs several amino acid sequences for a given nucleic acid sequence of the plurality of nucleic acid sequences generated by the encoding assay, and optionally calculates probability scores for each of the several amino acid sequences. These probability scores can be used to predict what polypeptides are present in the analyzed sample. In some embodiments of the disclosed methods, the computer model outputs one or more amino acid sequences that provide at least partial identities of polypeptides of the plurality of polypeptides. The term “partial identity” of a polypeptide refers to an amino acid sequence obtained through the described decoding methods that is homologous or identical to a portion of the polypeptide amino acid sequence, and from which the full polypeptide amino acid sequence can be inferred with certain probability. For example, the disclosed computer model may output several amino acid sequences for a given nucleic acid sequence of the plurality of nucleic acid sequences generated by the encoding assay, and one or more of the several amino acid sequences are each at least 50% identical, at least 60% identical, at least 70% identical, at least 80% identical, or at least 90% identical to a portion of one or more amino acid sequences of polypeptides of the plurality of polypeptides. For example, outputted amino acid sequences for a given nucleic acid sequence may correspond to one or more homologs of a polypeptide of the plurality of polypeptides. In some embodiments of the disclosed methods, the computer model outputs “imperfect” or “partial” amino acid sequences, which are amino acid sequences in which one or more amino acid residues are not defined (i.e. designated as Z, which can be any amino acid residue). Imperfect or partial amino acid sequences can still be used to identify polypeptides of the plurality of polypeptides with some degree of confidence.

In some embodiments of the disclosed methods, the output of the computer model comprises data related to relative quantity for one or more polypeptide of the plurality of polypeptides. In some embodiments, relative quantity for one or more polypeptides may be calculated or estimated based on amounts of specific binder identifier strings (or nucleic acid sequences that comprise a specific series of encoder barcode sequences) generated from the encoding assay.

Binder identifiers within a given binder identifier string may be nucleic acid sequences, abstract binder identifiers (e.g., binder names), or other objects that correspond to encoder barcode sequences in a giving nucleic acid sequence of the plurality of nucleic acid sequences generated from the encoding assay and received at the one or more processors. In some embodiments of the disclosed methods, each binder identifier is assigned to a particular encoder barcode sequence within a nucleic acid sequence of the plurality of nucleic acid sequences by inferring identifying information regarding the corresponding binder for the encoder barcode sequence. In some embodiments of the disclosed methods, the assigned binder identifier for each nucleic acid sequence of the plurality of nucleic acid sequences are then organized in a series of binder identifiers (i.e., binder identifier string) which reflect the identify and the order of binders used during encoding of a particular polypeptide in the encoding assay. In other words, each binder identifier string of plurality of binder identifier strings comprises information regarding the identify and the order of binders used during encoding of a particular polypeptide of the plurality of polypeptides in the encoding assay. The plurality of binder identifier strings comprises information regarding the identify and the order of binders used to encode the plurality of polypeptides into the plurality of nucleic acid sequences in the encoding assay.

In some embodiments of the disclosed methods, a binder identifier string is generated for each nucleic acid sequence of the plurality of nucleic acid sequences generated from the encoding assay based on a corresponding series of encoder barcode sequences. In some embodiments, the computer model generates a binder identifier string in advance before starting the inferring step of the disclosed methods. In other embodiments, each binder identifier string of the plurality of binder identifier strings is generated while performing the inferring step of the disclosed methods. In these embodiments, the computer model starts the inferring step by analyzing a single binder identifier, then takes into account the next binder identifier detected in a given series of encoder barcode sequences, and so on. At the end of the inferring process for the given series of encoder barcode sequences, a binder identifier string is defacto generated, because all binder identifiers are detected and analyzed in a particular order during the inferring process, which is determined by the order of particular encoding events that have occurred for a particular polypeptide in the encoding assay (the order of binders that encoded a particular polypeptide in the encoding assay).

In some embodiments of the disclosed methods, the computer model is trained based on pre-determined parameters of the encoding assay, wherein the pre-determined parameters comprise: i) probabilities of assigning to at least one component of a given polypeptide or polypeptide fragment one or more binder identifiers either correctly or incorrectly based on determined binding profiles of binders used in the encoding assay; and ii) optionally, for at least one component of a given polypeptide or polypeptide fragment, a probability of successfully cleaving the at least one component.

In some embodiments, in order to infer amino acid sequences of polypeptides from binder identifiers, binding profiles of binders are determined experimentally by testing individual binders for binding to a variety of known peptides (e.g., testing binding affinities of binders using an array of different peptides). In other embodiments, binding profiles of binders are predicted based on computer modeling of binders' structures and analyzing in silico interactions between binders and a variety of known peptides. In yet other embodiments, a combinational approach (both computational and experimental) may be utilized to determine binding profiles of binders.

In some embodiments, the described decoding algorithms infer an amino acid sequence of a polypeptide of the plurality of polypeptides from binder identifiers of a binder identifier string of the plurality of binder identifier strings based on (i) binding profiles of the binders from the set of binders that correspond to the binder identifiers of the binder identifier string, and (ii) calculated probability scores of an association between one or more binder identifiers of the binder identifier string and one or more amino acid sequences of polypeptides of the plurality of polypeptides.

In some embodiments, the described decoding algorithms calculate probability scores of an association between one or more binder identifiers of the binder identifier string and one or more amino acid sequences of polypeptides using (i) binding profiles of the binders from the set of binders that correspond to the one or more binder identifiers; and (ii) one or more parameters of the encoding assay. In some embodiments, the one or more parameters of the encoding assay comprise an efficiency of a functionalization of N-terminal amino acid (NTAA) residues of polypeptides of the plurality of polypeptides, an efficiency of a cleavage of NTAA residues of polypeptides of the plurality of polypeptides, an efficiency of an encoding of NTAA residues of polypeptides of the plurality of polypeptides, or any combination thereof.

In some embodiments, the term “association” between one or more binder identifiers and one or more amino acid sequences of polypeptides refers to the fact that the one or more amino acid sequences of polypeptides generate the one or more binder identifiers during the encoding assay and subsequent post-encoding assay analysis of the plurality of nucleic acid sequences (the output of the encoding assay). The calculated probability scores account for possibilities of “correct” association. For example, when the probability score is 100%, one is 100% confident that the computer model correctly associates one or more amino acid sequences of polypeptides of the plurality of polypeptides to one or more binder identifiers of the plurality of binder identifier strings. If the probability score is 70%, then there is 70% chance that the association is correct and 30% chance that the association is incorrect. In some embodiments, even when the probability scores are less than 100%, the computer model can still infer one or more amino acid sequences of polypeptides from binder identifiers of a given binder identifier string. In some embodiments, the computer model outputs certain amino acid sequences of polypeptides of the plurality of polypeptides associated to one or more binder identifiers of the plurality of binder identifier strings, and further provides probability scores for each amino acid sequences to be present within the plurality of polypeptides.

In some embodiments, the methods disclosed above further comprise the following steps performed after (a) and (b): (d) for each polypeptide and/or polypeptide fragment of the plurality of polypeptides, generating a set of binder identifier strings based on pre-determined parameters of the encoding assay, wherein the pre-determined parameters comprise: i) probabilities of assigning to at least one component of a given polypeptide or polypeptide fragment one or more binder identifiers either correctly or incorrectly based on binding profiles of binders used in the encoding assay; and ii) optionally, for at least one component of a given polypeptide or polypeptide fragment, a probability of successfully cleaving the at least one component, thereby determining probabilities for each binder identifier string generated from a given polypeptide or polypeptide fragment; and (e) for each binder identifier string of the plurality of binder identifier strings generated in (b), calculate probabilities that a given binder identifier string is generated from one or more polypeptides or fragments thereof based on the probabilities for binder identifier strings determined in (d), thereby determining at least a partial identity and/or quantity for at least one polypeptide of the plurality of polypeptides.

In some embodiments, a set of binder identifier strings based on pre-determined parameters of the encoding assay is generated not only for each polypeptide and/or polypeptide fragment of the plurality of polypeptides, but also for other polypeptides and/or polypeptide fragments that could potentially be present in at least one analyzed sample. For example, when a sample is a human blood plasma, all polypeptides that could potentially be present in a human blood plasma can be processed to generate a set of binder identifier strings and determine probabilities for each binder identifier string generated from a given polypeptide or polypeptide fragment. Each individual plasma sample may contain only a fraction of plasma proteins (other proteins may, for example, be degraded), and it is not known which protein are present in the sample, and which are not. Thus, a set of binder identifier strings needs to be generated for all polypeptides potentially present in the sample, creating a large table having (simulated) binder identifier strings and probabilities for each (simulated) binder identifier string to be generated from a given polypeptide or polypeptide fragment. Later, the data generated from the encoding assay are matched against this table, and polypeptides of a particular sample is determined or at least predicted with a certain probability.

In some embodiments, a set of binder identifier strings based on pre-determined parameters of the encoding assay is generated in silico, so that it is a set of simulated binder identifier strings, and probabilities are determined for each simulated binder identifier string. Using simulated binder identifier strings allows to generate much more data than generated experimentally, which is very useful for training purposes.

In some embodiments, the set of binder identifier strings is a set of simulated binder identifier strings generated in silico in (d), and wherein probabilities for each simulated binder identifier string are determined in (d). In other embodiments, the set of binder identifier strings is produced experimentally by the encoding assay. In yet other embodiments, a combinational approach (both computational and experimental) may be utilized to generate a set of binder identifier strings based on pre-determined parameters of the encoding assay.

In some embodiments of the disclosed methods, the plurality of polypeptides encoded in the plurality of nucleic acid sequences by the encoding assay comprises at least 1000, at least 10,000, at least 100,000, at least 1,000,000, or more polypeptides.

In some embodiments of the disclosed methods, the plurality of polypeptides encoded in the plurality of nucleic acid sequences by the encoding assay comprises at least 500, at least 1000, at least 2000, at least 5000, at least 10,000, at least 20,000 or more different polypeptides. In some embodiments, the different polypeptides come from 2, 5, 10, 50, 100, 200 or more different biological samples analyzed in the single encoding assay.

In some embodiments of the disclosed methods, the series of encoder barcode sequences comprises at least two different encoder barcode sequences. In some embodiments of the disclosed methods, the series of encoder barcode sequences comprises no more than 30 different encoder barcode sequences. In some embodiments of the disclosed methods, the series of encoder barcode sequences comprises from 4 to 20 different encoder barcode sequences (each unique barcode sequence of the series of encoder barcode sequences corresponds to a unique binder that binds to a component of a polypeptide of the plurality of polypeptides in the encoding assay). In some embodiments, no more than 30 unique binders are necessary to fully characterize at least 1000, at least 10,000, at least 100,000, at least 1,000,000, or more polypeptides.

In some embodiments of the disclosed methods, the component of the polypeptide to which the binder binds consists of a terminal amino acid residue modified with an N-terminal modifier agent.

In some embodiments of the disclosed methods, the pre-determined parameters are obtained by training a machine learning model, which uses as an input output data generated from performing multiple encoding assays with pre-determined analytes, thereby generating the computer model.

In some embodiments of the disclosed methods, the computer model outputs probabilities for each binder identifier string to be derived from the two or more amino acid sequences of polypeptides to which a given binder identifier string is associated.

In some embodiments of the disclosed methods, i) each of the plurality of nucleic acid sequences further comprises one or more auxiliary sequences; ii) the method further comprises assigning, using the one or more processors, an auxiliary identifier to at least one of the one or more auxiliary sequences in each of the plurality of nucleic acid sequences to generate a plurality of identifier strings for the plurality of nucleic acid sequences; and iii) the one or more auxiliary sequences comprise one or more identifier sequences or complements thereof, one or more spacer sequences or complements thereof, one or more sequencing primer sequences or complements thereof, or any combination thereof.

In some embodiments of the disclosed methods, the one or more identifier sequences comprise one or more sample barcode sequences, one or more bead barcode sequences, one or more unique molecular identifier (UMI) sequences, or any combination thereof.

In some embodiments of the disclosed methods, the assignment of binder identifiers to encoder barcode sequences is based on a specific order of components in each of the plurality of nucleic acid sequences and a look-up table (LUT) of binder identifiers.

In some embodiments of the disclosed methods, the assignment of binder identifiers to encoder barcode sequences further comprises performing error correction based on an enumeration of error types to account for errors associated with an encoding process used to generate each of the series of encoder barcode sequences.

In some embodiments of the disclosed methods, accounting for the errors associated with the process used to generate each of the series of encoder barcode sequences comprises determining: (i) a first probability for reading a specified barcode sequence for a specified underlying polypeptide component, and (ii) a second probability for successfully transitioning from one polypeptide component to the next polypeptide component during the encoding process.

In some embodiments of the disclosed methods, the assignment of binder identifiers to encoder barcode sequences in (b) comprises use of a probabilistic model to predict a sequence of binder identifiers for each nucleic acid sequence of the plurality.

In some embodiments of the disclosed methods, the probabilistic model comprises a hidden Markov model (HMM). In some embodiments, the hidden Markov model (HMM) is trained using one or more training data sets comprising labeled pairs of binder identifiers and nucleic acid sequences. In some embodiments, the hidden Markov model (HMM) is trained using an iterative Expectation-Maximization (EM) algorithm to determine a set of model parameters that maximize a probability of correctly predicting a sequence of binder identifiers for each nucleic acid sequence of the plurality.

In some embodiments of the disclosed methods, in the Expectation-maximization (EM) algorithm comprises a Baum-Welch algorithm.

In some embodiments of the disclosed methods, the computer model is configured to identify unique binder identifier signatures in the plurality of binder identifier strings as part of inferring amino acid sequences of polypeptides from binder identifier strings, and wherein a given unique binder identifier signature comprises a set of binder identifier strings associated with a single polypeptide.

In some embodiments of the disclosed methods, the computer model is trained using a training data set comprising binder identifier string data for one or more isolated polypeptide samples that are processed using a same sample preparation protocol as that used to process the plurality of polypeptides.

In some embodiments of the disclosed methods, the training data set further comprises binder identifier string data for a background sample processed using a same sample preparation protocol as that used to process the plurality of polypeptides.

In some embodiments of the disclosed methods, the background sample comprises a plasma sample, a urine sample, a saliva sample, or a cell extract sample.

In some embodiments of the disclosed methods, the sample is a cell extract sample, and the cell extract sample comprises a mammalian cell extract sample, a plant cell extract, a fungal cell extract, or a bacterial cell extract sample.

In some embodiments of the disclosed methods, the computer model is further configured to correct the quantity output for the at least one polypeptide using a correction factor calculated from the training data set.

In some embodiments of the disclosed methods, the computer model is configured to map each binder identifier string of the plurality of binder identifier strings to a specific polypeptide as part of assigning binder identifier strings to amino acid sequences.

In some embodiments of the disclosed methods, mapping a binder identifier string of the plurality of binder identifier strings to a specific polypeptide comprises: i) generating a set of k-mer fragments for the binder identifier string; ii) determining a probability that a given k-mer fragment of the set belongs to a specific polypeptide based on a previously determined probability distribution; and iii) assigning the binder identifier string to the specific polypeptide based on the determined probabilities for the set of k-mer fragments.

In some embodiments of the disclosed methods, the probabilistic model is trained on empirically-determined assay parameter data.

In some embodiments of the disclosed methods, the empirically-determined assay parameter data comprises, for each barcode sequence of the series of encoder barcode sequences, a probability of reading a given barcode sequence in a nucleic acid sequence for a given one or more amino acid residue(s) of a corresponding polypeptide to which a binder was bound.

In some embodiments of the disclosed methods, the empirically-determined assay parameter data comprises, for each potential N-terminal amino acid residue in a polypeptide, a probability of successfully cleaving the N-terminal amino acid residue during a cyclical process used to encode the plurality of polypeptides in the plurality of nucleic acid sequences.

In some embodiments of the disclosed methods, mapping a binder identifier string of the plurality of binder identifier strings to a specific polypeptide comprises providing the plurality of binder identifier strings as input to the computer model, wherein the computer model is configured to fractionally assign a given binder identifier string to two or more specific polypeptides.

In some embodiments of the disclosed methods, the computer model is trained on a training data set comprising a set of simulated binder identifier strings generated for a given input distribution of polypeptides based on empirically-determined assay parameter data comprising: i) for each barcode sequence of the series of encoder barcode sequences, a probability of reading a given barcode sequence in a nucleic acid sequence for a given one or more amino acid residue(s) of a corresponding polypeptide to which a binder was bound; and ii) for each potential N-terminal amino acid residue in a polypeptide, a probability of successfully cleaving the N-terminal amino acid residue during a cyclical process used to encode the plurality of polypeptides in the plurality of nucleic acid sequences.

In some embodiments of the disclosed methods, the set of simulated binder identifier strings comprises at least 100,000, 500,000, 1M, 5M, 10M, 25M, 50M, 75M, 100M, 200M, 300M, 400M, or 500M simulated binder identifier strings.

In some embodiments of the disclosed methods, the computer model is further configured to output a confidence interval for the partial identity of the at least one polypeptide.

In some embodiments of the disclosed methods, a level of stringency in the output confidence interval is selectable by a user.

In some embodiments of the disclosed methods, the level of stringency in the confidence interval corresponds to a confidence level of 90%, 95%, 98%, or 99%.

In some embodiments of the disclosed methods, the computer model comprises a trained artificial neural network model, a trained deep learning model, a trained random forest model, or a trained support vector machine.

In some embodiments, the disclosed methods further comprise performing an iterative Expectation Maximization (EM) process to refine the quantity output for the at least one polypeptide of the plurality of polypeptides.

In some embodiments of the disclosed methods, the iterative EM process comprises repetitively: (i) finding a best assignment of a binder to each barcode sequence in a nucleic acid sequence based on a current estimate of what polypeptides are present in the plurality of polypeptides; (ii) finding an updated best estimate of what polypeptides are present in the plurality of polypeptides based on the best assignment of a binder to each different barcode sequence in the nucleic acid sequence; and (iii) determining an amount of the at least one polypeptide present in the plurality of polypeptides based on the updated best estimate of what polypeptides are present in the plurality of polypeptides. In some embodiments, the described steps (i) to (iii) are repeated until a difference between the amount of the at least one polypeptide determined in one iteration and the next is less than a specified threshold, or until a specified maximum number of iterations has been reached.

In some embodiments, disclosed methods further comprise determining the nucleic acid sequences for the plurality of nucleic acid sequences. In some embodiments, the nucleic acid sequences are determined by performing DNA sequencing. In some embodiments, the DNA sequencing is performed using a next generation DNA sequencer.

In some embodiments of the disclosed methods, the plurality of nucleic acid sequences comprises at least 10,000, 100,000, or 1M nucleic acid sequences.

In some embodiments of the disclosed methods, the plurality of polypeptides comprises at least 100, 500, 1,000, 2,000, 5,000, or 10,000 different polypeptides.

In some embodiments of the disclosed methods, at least two binder identifier strings are generated for at least one nucleic acid sequence of the plurality of nucleic acid sequences.

In some embodiments, disclosed methods further comprise comparing the plurality of nucleic acid sequences output by the trained model to a plurality of nucleic acid sequences determined by subjecting the plurality of polypeptides to a reverse translation assay and sequencing the resulting extended recording tags.

In some embodiments of the disclosed methods, the computer model comprises a machine statistical model. In other embodiments, the computer model is a trained machine learning model.

In preferred embodiments, the disclosed methods are for analysis and/or sequencing of multiple polypeptide analytes simultaneously (multiplexing).

Multiplexing as used herein refers to analysis of a plurality of polypeptide analytes in the same assay. The plurality of polypeptide analytes can be derived from the same sample or different samples. The plurality of polypeptide analytes can be derived from the same subject or different subjects. The plurality of polypeptide analytes that are analyzed can be different polypeptide analytes, or the same polypeptide analyte derived from different samples. A plurality of polypeptide analytes includes 2 or more polypeptide analytes, 5 or more polypeptide analytes, 10 or more polypeptide analytes, 50 or more polypeptide analytes, 100 or more polypeptide analytes, 500 or more polypeptide analytes, 1000 or more polypeptide analytes, 5,000 or more polypeptide analytes, 10,000 or more polypeptide analytes, 50,000 or more polypeptide analytes, 100,000 or more polypeptide analytes, 500,000 or more polypeptide analytes, or 1,000,000 or more polypeptide analytes.

In preferred embodiments, the disclosed methods are for identifying a large number of polypeptides (e.g., at least 1000, 10000, 100000, 1000000 or more polypeptide molecules which comprise molecules of at least 100, 1000, 10000 or more different polypeptides) in a single assay.

In some embodiments of the disclosed methods, the molecules comprise two or more molecules of the same polypeptide analyte and two or more molecules of different polypeptide analytes.

In some embodiments of the disclosed methods and compositions, any two or more of the molecules are each independently associated with the same nucleic acid recording tag or different nucleic acid recording tags.

In some embodiments of the disclosed methods and compositions, any two or more of the different polypeptide analytes are each independently associated with the same nucleic acid recording tag or different nucleic acid recording tags.

In some embodiments, provided herein is a method of identifying a large plurality of polypeptide analytes (e.g., at least 1000, 10000, 100000, 1000000 or more polypeptide molecules which comprise molecules of at least 100, 1000, 10000 or more different polypeptides) in a single assay, using one or more deconvolution methods based on the determined binding properties of the binders to match the group of the binders to a sequence of a polypeptide for each of the plurality of polypeptide analytes, thereby determining the identity of each of the plurality of polypeptide analytes. In preferred embodiments, both known specificities (binding profiles) of binders for NTAA residues and their order of binding to the polypeptide analyte are used to decode identify of the polypeptide analyte.

In preferred embodiments, the methods provided herein are able to simultaneously identify multiple different polypeptide analytes (such as at least 100, 1000, 10000 or more different polypeptides) within a single sample. In some embodiments, polypeptides from a sample can be fractionated into a plurality of fractions, and polypeptides in each plurality of fractions can be fragmented to polypeptides followed by barcoding of the polypeptides (e.g., by introducing a sample barcode into an associated recording tag for each polypeptide). Then, barcoded polypeptides from different fractions each conjugated to a recording tag can be pooled together and analyzed using methods and compositions disclosed herein. Fractionation, barcoding and pooling techniques are beneficial for analysis of complex biological samples, such as samples having polypeptides of vastly different abundances (e.g., plasma). Techniques for fractionation, barcoding and pooling are known in the art and disclosed, for example, in US 20190145982 A1, incorporated by reference herein.

In some embodiments of the disclosed methods, given that selectivities of each of the binders towards NTAA residues are known, information regarding identity of the NTAA residue of the analyzed immobilized polypeptide is encoded in a unique nucleic acid barcode present in the extended recording tag. This nucleic acid barcode may be used to decode the identity of the NTAA residue by using known information regarding binding kinetics and/or specificity of the binders bound to the polypeptide at a given binding cycle. In some embodiments, the nucleic acid barcode may be used as an input to a probabilistic neural network which was trained to relate the sequence of the barcode to amino acid identity. Training can be performed by testing each binder individually (optionally, conjugated to a coding tag) against a panel of polypeptides each having a different NTAA residue and an associated recording tag, collecting sequence information of the recording tags extended after the binding, and feeding the collected information to the probabilistic neural network. Alternatively, training can be performed by testing a set of binders (optionally, each conjugated to a coding tag) against the panel of polypeptides, collecting sequence information of the recording tags extended after the binding, and feeding the collected information to the probabilistic neural network.

In some embodiments, during each encoding cycle, only single amino acid residue of the analyzed polypeptide gets encoded into the recording tag (each time it is a NTAA residue, which gets cleaved off at the end of each binding cycle). In other embodiments, a dipeptide gets encoded into the recording tag and dipeptides are cleaved between encoding cycles (e.g., by use of a dipeptidyl carboxypeptidase).

In some embodiments, the generated DNA barcodes on the extended recording tag of each polypeptide analyte are input to a probabilistic neural network (PNN) which will learn to relate the sequence of a DNA barcode to an amino acid identity. Probabilistic neural networks (Mohebali, B., et al., Chapter 14—Probabilistic neural networks: a brief overview of theory, implementation, and application, in Handbook of Probabilistic Models, P. Samui, et al., Editors. 2020, Butterworth-Heinemann. P. 347-367) can approach Bayes optimal classification for multiclass problems such as amino acid identification from DNA barcodes (Klocker, J., et al., Bayesian Neural Networks for Aroma Classification. Journal of Chemical Information and Computer Sciences, 2002. 42(6): p. 1443-1449). A classifier based on PNN is guaranteed to learn and converge to an optimal classifier as the size of the representative data set increases. Probabilistic neural networks have parallel structure such that data from any amino acid residue are used to learn/predict most other amino acid residues.

In some embodiments, the disclosed methods are used for polypeptide sequence determination based on probabilistic neural network ensembles. The machine learning method is characterized in that the sequence determination can be realized by the following steps: i) the polypeptide fragments of polypeptides are encoded using a set of binders into stretches of DNA sequences based on the structural properties of amino acid residues; ii) a group of probabilistic neural network sub-classifiers are established, polypeptide fragments of polypeptides with known sequence are used to perform amino acid classification training and obtain a group of trained amino acid classification models; iii) the obtained models are utilized to determine polypeptide amino acid sequences in the test data sets; iv) the classification results output by the models are counted to generate amino acid candidate sets; v) the methods showing highest accuracy are combined to determine the amino acid sequence of polypeptide fragment; and vi) the algorithmic amino acid determination result is verified through k-fold cross-validation, where k is an integer.

In some embodiments, k-fold cross-validation operates as follows. In k-fold cross-validation, the dataset is shuffled and divided into k groups randomly with no overlap and replacements. This means each group is unique and is used for model evaluation only once. The data groups are carried through the following steps to perform the k-fold cross-validation:
1) A unique group is taken as a test data set;
2) The remaining (k−1) groups are used as a training data set;
3) A model is built using the training set and is evaluated using the test set;
4) The evaluation score is retained and the model is discarded;
5) Step 1-4 are repeated until all k groups are used for model evaluation;
6) The mean of the k evaluation scores is output as the k-fold cross-validated model performance score.

In some embodiments, the nucleic acid barcodes on the extended recording tag of each polypeptide analyte are input to a probabilistic neural network (PNN), which will learn to relate the DNA sequence of a barcode to an amino acid identity of the analyzed polypeptide. In other embodiments, other statistical models (e.g., hidden Markov models) and machine learning methods (e.g., random forest models) can be used for classifying a NGS read from each extended recording tag into a specific amino acid residue (or amino acid residue type, if binder is not selective to particular NTAA residues).

In some embodiments of the disclosed methods, the N-terminal amino acid residue of the polypeptide analyte is not modified or removed.

In some embodiments of the disclosed methods, the N-terminal amino acid (NTAA) residues of polypeptide analytes are modified by an N-terminal modifier agent. In some embodiments of the disclosed methods, the N-terminal modifier agent is a compound of a formula selected from the group consisting of:

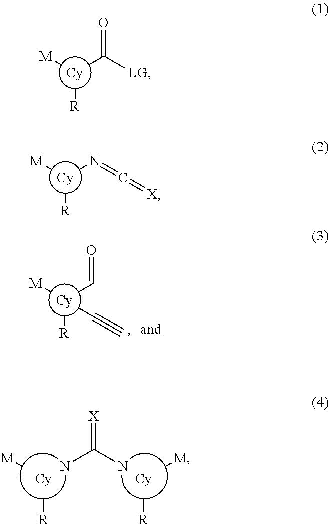

wherein M is a metal binding group that comprises sulfonamide, hydroxamic acid, sulfamate, or sulfamide; the group

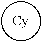

is a 5 or 6 membered aromatic ring containing up to three heteroatoms selected from N, O, and S as ring members, and is optionally substituted by R; R represents one or two optional substituents selected from the group consisting of F, Cl, CH₃, CF₂H, CF₃, OH, OCH₃, OCF₃, NH₂, N(CH₃)₂, NO₂, SCH₃, SO₂CH₃, CH₂OH, B(OH)₂, CN, CONH₂, CO₂H, CN₄H, and CONHCH₃; LG is OH, ORQ, or OCC, each RQ is independently aryl or heteroaryl, each of which is optionally substituted with one or more groups selected from halo, nitro, cyano, sulfonate, carboxylate, alkylsulfonyl, and N of heteroaryl is optionally oxidized; or RQ can be —C(═O)R or —C(═O)—OR; CC is a cationic counterion; X is one of the following: O, S, Se, or NH. In some embodiments of the disclosed methods, the N-terminal modifier agent is a compound of Formula (5):

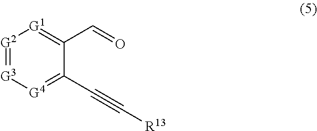

wherein:
G¹-G⁴ are each independently selected from CH, CJ, and N, provided not more than 3 of G¹-G⁴ are N;
J at each occurrence is independently selected from H, C₁-C₂ alkyl, NO₂, C₁-C₂ haloalkyl, C₁-C₂ haloalkoxy, halo, —OR⁸, —N(R⁸)₂, —SR⁸, —S(O)ₙR⁸, —NR⁸SO₂R⁸, —SO₂N(R⁸)₂, SO₃R⁸, —B(OR⁸)₂, C(═O)R⁸, CN, CON(R⁸)₂, —COOR⁸, —C(═O)Ar, and tetrazole, where Ar represents a phenyl or 5-6 membered heteroaryl ring that is optionally substituted with one or two groups selected from halo, CN, R⁸ and OR⁸;
each R⁸ is independently selected from H, C₁-C₂ alkyl, and C₁-C₂ haloalkyl;
n at each occurrence is independently 1 or 2; and
R¹³ is selected from H, C₁-C₂ alkyl, C₁-C₂ alkoxy, C₁-C₂ haloalkyl, and C₁-C₂ haloalkoxy. In some embodiments of the disclosed methods, the N-terminal modifier agent is a compound described in the following patent publications: US 2023/0220589 A1 and US 2022/0283175 A1, incorporated by reference herein.

Coding tag information associated with a specific binder may be transferred to a recording tag using a variety of methods. In any of the disclosed embodiments, the transfer of information (e.g., from a coding tag to a recording tag, or from a recording tag to a coding tag) can be accomplished by ligation (e.g., an enzymatic or chemical ligation, a splint ligation, a sticky end ligation, a single-strand (ss) ligation such as a ssDNA ligation, or any combination thereof), a polymerase-mediated reaction (e.g., primer extension of single-stranded nucleic acid or double-stranded nucleic acid), or any combination thereof.

In some embodiments, a DNA polymerase that is used for primer extension during information transfer possesses strand-displacement activity and has limited or is devoid of 3′-5 exonuclease activity. Several of many examples of such polymerases include Klenow exo-(Klenow fragment of DNA Pol 1), T4 DNA polymerase exo−, T7 DNA polymerase exo (Sequenase 2.0), Pfu exo−, Vent exo−, Deep Vent exo−, Bst DNA polymerase large fragment exo−, Bca Pol, and Phi29 Pol exo−. In a preferred embodiment, the DNA polymerase is active at room temperature and up to 45° C. In another embodiment, a “warm start” version of a thermophilic polymerase is employed such that the polymerase is activated and is used at about 40° C.-50° C. An exemplary warm start polymerase is Bst 2.0 Warm Start DNA Polymerase (New England Biolabs).

Coding tag information associated with a specific binder may be transferred to a recording tag associated with the immobilized polypeptide via ligation. Ligation may be a blunt end ligation or sticky end ligation. Ligation may be an enzymatic ligation reaction. Examples of ligases include, but are not limited to CV DNA ligase, T4 DNA ligase, T7 DNA ligase, T3 DNA ligase, Taq DNA ligase, E. coli DNA ligase. Alternatively, a ligation may be a chemical ligation reaction. In one embodiment, a spacer-less ligation is accomplished by using hybridization of a “recording helper” sequence with an arm on the coding tag. The annealed complement sequences are chemically ligated using standard chemical ligation or “click chemistry” (Gunderson et al., Genome Res (1998) 8(11): 1142-1153; Litovchick et al., Artif DNA PNA XNA (2014) 5(1): e27896; Roloff et al., Methods Mol Biol (2014) 1050:131-141).

Various aspects of coding tag and recording tag compositions, as well as aspects of transferring identifying information from a coding tag to a recording tag are disclosed in the published applications US 2019/0145982 A1 and US 2023/0136966 A1, incorporated herein by reference.

In some embodiments, the coding tags within a set of binding agents share a common spacer sequence used in an assay (e.g. the entire library of binding agents used in a multiple binding cycle method possess a common spacer in their coding tags). In another embodiment, the coding tags are comprised of a binding cycle-specific barcodes, identifying a particular binding cycle. In other embodiments, the coding tags within a set of binding agents have a binding cycle-specific spacer sequence. For example, a coding tag for binding agents used in the first binding cycle comprise a “cycle 1” specific spacer sequence, a coding tag for binding agents used in the second binding cycle comprise a “cycle 2” specific spacer sequence, and so on up to “n” binding cycles. In further embodiments, coding tags for binding agents used in the first binding cycle comprise a “cycle 1” specific spacer sequence and a “cycle 2” specific spacer sequence, coding tags for binding agents used in the second binding cycle comprise a “cycle 2” specific spacer sequence and a “cycle 3” specific spacer sequence, and so on up to “n” binding cycles. This embodiment is useful for subsequent PCR assembly of non-concatenated extended recording tags after the binding cycles are completed. In some embodiments, a spacer sequence comprises a sufficient number of bases to anneal to a complementary spacer sequence in a recording tag or extended recording tag to initiate a primer extension reaction or sticky end ligation reaction.

In certain embodiments, an ensemble of recording tags may be employed per polypeptide to improve the overall robustness and efficiency of coding tag information transfer. The use of an ensemble of recording tags associated with a given polypeptide rather than a single recording tag improves the efficiency of library construction due to potentially higher coupling yields of coding tags to recording tags, and higher overall yield of libraries. The yield of a single concatenated extended recording tag is directly dependent on the stepwise yield of concatenation, whereas the use of multiple recording tags capable of accepting coding tag information does not suffer the exponential loss of concatenation.

In some embodiments of the disclosed methods, the N-terminal amino acid residue of each polypeptide analyte is joined to the support or the nucleic acid recording tag associated with the polypeptide analyte.

In some embodiments of the disclosed methods, the analyzing step comprises determining identities of the NTAA residue and the penultimate terminal amino acid of the polypeptide analyte.

Binders in the disclosed methods and compositions do not need to be strictly selective and may recognize, for example, functional classes of NTAA residues, such as negatively charged residues, positively charged residues, small hydrophobic residues, aromatic residues, and so on, or recognize other NTAA residue types. In some embodiments, at least some of binders of the set of binders are degenerate (each can bind more than one structure or more than one component of polypeptide). In some embodiments, degenerate binders have specificity towards two or more NTAA residues. In some embodiments, specificity of each of binders towards particular NTAA is not high. Use of degenerate binders may reduce the overall number of binders needed for successful polypeptide identification. In some embodiments, no more than 5, 6, 7, 8, 9 or 10 binders having different NTAA specificities are needed for identification of at least 90% of polypeptide analytes present in a sample.

In some embodiments, polypeptide analytes may be mixed, spotted, dropped, pipetted, flowed, or otherwise applied to the support. In some embodiments, support has been functionalized with a chemical moiety such as an NHS ester or other amine-specific reagent before polypeptide analytes are applied to the support. This allows to use immobilization of polypeptide analytes to the support through N-terminus (see also Example 3 below).

In preferred embodiments, selectivity of each binder used during the encoding assay towards NTAA resides of polypeptide analytes is determined in advance, before performing contacting steps of the disclosed methods. Each binder may be tested against a panel of peptides each having a different NTAA reside and an associated recording tag to characterize selectivity and, optionally, binding kinetics of the binder for each of the 20 natural NTAA resides. When multiple alternative binders exist, a set comprising minimum number of binders may be selected that would cover all or a maximum number of the 20 natural NTAA resides.

### Attachment to the Support

In some embodiments, the polypeptide is joined to a support before performing the binding/cleaving reaction. In some cases, it is desirable to use a support with a large carrying capacity to immobilize a large number of polypeptides. In some embodiments, it is preferred to immobilize the polypeptides using a three-dimensional support (e.g., a porous matrix or a porous bead). For example, the preparation of the polypeptides including joining the polypeptide to a support is performed prior to performing the binding reaction. In some examples, the preparation of the polypeptide including joining the polypeptide to nucleic acid molecule or a oligonucleotide may be performed prior to or after immobilizing the polypeptide. In some embodiments, a plurality of polypeptides are attached to a support prior to the binding reaction and contacting with a binder.

In some embodiments, the support may comprise any suitable solid material, including porous and non-porous materials, to which a polypeptide, e.g., a polypeptide, can be associated directly or indirectly, by any means known in the art, including covalent and non-covalent interactions, or any combination thereof.

Various reactions may be used to attach the polypeptide analytes to a support, or to associate each polypeptide analytes (directly or indirectly, such as through support) with a recording tag. The polypeptides may be attached directly or indirectly to the support. In some cases, the polypeptides are attached to the support via a nucleic acid (e.g., via a nucleic acid recording tag). Exemplary reactions include click chemistry reactions, such as the copper catalyzed reaction of an azide and alkyne to form a triazole (Huisgen 1,3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2]cycloaddition, alkene and tetrazine inverse electron demand Diels-Alder (IEDDA) reaction (e.g., m-tetrazine (mTet) or phenyl tetrazine (pTet) and trans-cyclooctene (TCO); or pTet and an alkene), alkene and tetrazole photoreaction, Staudinger ligation of azides and phosphines, and various displacement reactions, such as displacement of a leaving group by nucleophilic attack on an electrophilic atom (Horisawa 2014, Knall, Hollauf et al. 2014). Exemplary displacement reactions include reaction of an amine with: an activated ester; an N-hydroxysuccinimide ester; an isocyanate; an isothioscyanate, an aldehyde, an epoxide, or the like. In some embodiments, iEDDA click chemistry is used for immobilizing polypeptides to a support since it is rapid and delivers high yields at low input concentrations. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction. In one case, a polypeptide is labeled with a bifunctional click chemistry reagent, such as alkyne-NHS ester (acetylene-PEG-NHS ester) reagent or alkyne-benzophenone to generate an alkyne-labeled polypeptide. In some embodiments, an alkyne can also be a strained alkyne, such as cyclooctynes including Dibenzocyclooctyl (DBCO). Other suitable examples of a covalent conjugation between two moieties are disclosed in US 2021/0101930 A1, incorporated by reference herein.

Similar methods (e.g., click chemistry reactions, bioorthogonal reactions) can be used to attach the polypeptide analyte to the associated nucleic acid recording tag, or to attach the binder to the associated nucleic acid coding tag. Such attachments can be achieved by introducing reactive moiety or moieties on one or on both attachment partners.

In some embodiments of the disclosed methods, a plurality of different polypeptides is immobilized on a solid support, wherein each polypeptide of the plurality of different polypeptides is associated with a nucleic acid recording tag. Various possible ways exist for association between an immobilized polypeptide and the associated nucleic acid recording tag. A recording tag may be directly linked to the polypeptide, linked to a polypeptide via a linker, via a multifunctional linker, or associated with a polypeptide by virtue of its proximity (or co-localization) on the support. In some embodiments, the recording tag is attached to the support, and the polypeptide is immobilized on the support via the recording tag. In some embodiments, a linker is attached to the support, and the polypeptide and the recording tag are independently attached to the linker, thereby generating immobilization on the support and association of the polypeptide with the recording tag. Other immobilization and association variants are possible.

In some embodiments, at least one recording tag is associated or co-localized directly or indirectly with the polypeptide. In another embodiment, multiple recording tags are attached to the polypeptide, such as to the lysine residues or peptide backbone. In some embodiments, a polypeptide labeled with multiple recording tags is fragmented or digested into smaller peptides, with each peptide labeled on average with one recording tag. A recording tag may be single stranded, or partially or completely double stranded. In some embodiments, the recording tag may comprise a unique molecular identifier, a compartment tag, a partition barcode, sample barcode, a fraction barcode, a spacer sequence, a universal priming site, or any combination thereof. In some embodiments, the recording tag may comprise a blocking group, such as at the 3′-terminus of the recording tag. In some cases, the 3′-terminus of the recording tag is blocked to prevent extension of the recording tag by a polymerase.

In some embodiments, the recording tag can include a sample identifying barcode. A sample barcode is useful in the multiplexed analysis of a set of samples in a single reaction vessel or immobilized to a single solid support (e.g., a bead or a planar substrate) or collection of solid supports. For example, polypeptides from many different samples can be labeled with recording tags with sample-specific barcodes, and then all the samples pooled together prior to immobilization to a support, cyclic binding of the binder, and recording tag analysis. In certain embodiments, a recording tag comprises an optional unique molecular identifier (UMI), which provides a unique identifier tag for each polypeptide to which the UMI is associated with. A UMI can be used to de-convolute sequencing data from a plurality of extended recording tags to identify sequence reads from individual polypeptides. In some embodiments, within a library of polypeptides, each polypeptide is associated with a single recording tag, with each recording tag comprising a unique UMI. In other embodiments, multiple copies of a recording tag are associated with a single polypeptide, with each copy of the recording tag comprising the same UMI. In certain embodiments, a recording tag comprises a universal priming site, e.g., a forward or 5′ universal priming site. A universal priming site is a nucleic acid sequence that may be used for priming a library amplification reaction and/or for sequencing. A universal priming site may include, but is not limited to, a priming site for PCR amplification, flow cell adaptor sequences that anneal to complementary oligonucleotides on flow cell surfaces (e.g., Illumina next generation sequencing), a sequencing priming site, or a combination thereof. In some embodiments, a universal priming site comprises an Illumina P5 primer or an Illumina P7 primer for NGS.

In some embodiments, identifying components of a coding tag or recording tag, e.g., barcode, UMI, compartment tag, partition barcode, sample barcode, spatial region barcode, cycle specific sequence or any combination thereof, is subject to Hamming distance, Lee distance, asymmetric Lee distance, Reed-Solomon, Levenshtein-Tenengolts, or similar methods for error-correction. Hamming distance refers to the number of positions that are different between two strings of equal length. It measures the minimum number of substitutions required to change one string into the other. Hamming distance may be used to correct errors by selecting encoder sequences that are reasonable distance apart. Thus, in the example where the encoder sequence is 5 bases, the number of useable encoder sequences is reduced to 256 unique encoder sequences (Hamming distance of 1→4⁴ encoder sequences=256 encoder sequences). In another embodiment, the encoder sequence, barcode, UMI, compartment tag, cycle specific sequence, or any combination thereof is designed to be easily read out by a cyclic decoding process (Gunderson, 2004, Genome Res. 14:870-7). In another embodiment, barcode, UMI, compartment tag, partition barcode, spatial barcode, sample barcode, cycle specific sequence, or any combination thereof is designed to be read out by low accuracy nanopore sequencing, since rather than requiring single base resolution, words of multiple bases (^{˜}5-20 bases in length) need to be read.

In certain embodiments where multiple polypeptides are immobilized on the same support, the polypeptide molecules can be spaced appropriately to accommodate methods of performing the binding reaction and any downstream analysis steps to be used to assess the polypeptide. For example, it may be advantageous to space the polypeptide molecules that optimally to allow a nucleic acid-based method for assessing and sequencing the polypeptides to be performed. To control spacing of the immobilized polypeptides on the support, the density of functional coupling groups for attaching the polypeptide (e.g., TCO or carboxyl groups (COOH)) may be titrated on the support surface. In some embodiments, multiple polypeptide molecules are spaced apart on the surface or within the volume (e.g., for porous supports) of a support such that adjacent molecules are spaced apart at a distance of about 50 nm to about 500 nm. Further details on these methods can be found in the published applications US 2019/0145982 A1 and US 2023/0136966 A1, incorporated herein by reference.

### Exemplary Embodiments

The following enumerated exemplary embodiments represent certain embodiments and examples of the invention:

1. A computer-implemented method for analyzing a plurality of polypeptides encoded in a plurality of nucleic acid sequences by an encoding assay, the method comprising:
(a) receiving, at one or more processors, the plurality of nucleic acid sequences generated from the encoding assay, wherein each of the plurality of nucleic acid sequences comprises a series of encoder barcode sequences, and wherein each encoder barcode sequence of a given series of encoder barcode sequences corresponds to a binder, from a set of binders, that binds to one or more components of a polypeptide of the plurality of polypeptides;
(b) generating a binder identifier string for each nucleic acid sequence of the plurality of nucleic acid sequences based on a corresponding series of encoder barcode sequences, thereby generating a plurality of binder identifier strings corresponding to the plurality of nucleic acid sequences;
(c) inferring an amino acid sequence of a polypeptide of the plurality of polypeptides from binder identifiers of a binder identifier string of the plurality of binder identifier strings based on (i) binding profiles of the binders from the set of binders that correspond to the binder identifiers of the binder identifier string, and (ii) calculated probability scores of an association between one or more binder identifiers of the binder identifier string and one or more amino acid sequences of polypeptides of the plurality of polypeptides; and
(d) based on the calculated probability scores and the inferred amino acid sequences, outputting data related to at least a partial identity and/or quantity for at least one polypeptide of the plurality of polypeptides.

2. The computer-implemented method of embodiment 1, wherein inferring amino acid sequences of polypeptides comprises: (i) for each of the plurality of binder identifier strings, converting a given binder identifier string into one or more peptidic reads based on the binding profiles of the binders of the set of binders that correspond to binder identifiers present in a given binder identifier string, and (ii) calculating a probability score for each of the one or more peptidic reads, wherein the probability score is indicative of a probability that a given peptidic read produces a given binder identifier string.

3. The computer-implemented method of embodiment 2, further comprising: for each of the plurality of binder identifier strings, filtering out peptidic reads of the one or more peptidic reads generated for a given binder identifier string based on (i) the probability score for each peptidic read, and/or (ii) a probability that a given peptidic read was generated from amino acid sequences of the plurality of polypeptides.

4. The computer-implemented method of embodiment 1, wherein inferring amino acid sequences of polypeptides comprises:
(i) for each amino acid sequence of the plurality of polypeptides, generating one or more simulated binder identifier strings using one or more parameters of the encoding assay;
(ii) for each of the one or more simulated binder identifier strings, or for each of one or more amino acid sequences of polypeptides of the plurality of polypeptides, calculating a probability score based on a probability that a given simulated binder identifier string is associated with one or more amino acid sequences of polypeptides of the plurality of polypeptides; and
(iii) matching each of the plurality of binder identifier strings to the one or more simulated binder identifier strings based on the calculated probability scores for the one or more simulated binder identifier strings.

5. The computer-implemented method of embodiment 4, wherein the one or more parameters of the encoding assay comprise an efficiency of a functionalization of N-terminal amino acid (NTAA) residues of polypeptides of the plurality of polypeptides, an efficiency of a cleavage of NTAA residues of polypeptides of the plurality of polypeptides, an efficiency of an encoding of NTAA residues of polypeptides of the plurality of polypeptides, or any combination thereof.

6. The computer-implemented method of any one of embodiments 1-5, wherein inferring amino acid sequences of polypeptides comprises inputting the binder identifier strings into a trained machine learning model, wherein the trained machine learning model is trained on empirically determined encoding assay data.

7. The computer-implemented method of embodiment 6, wherein the trained machine learning model is trained using a training data set comprising binder identifier strings data, or numerical representations thereof, for one or more isolated polypeptide samples, or numerical representations thereof, that are subjected to the encoding assay using a same sample preparation protocol as that used to process the plurality of polypeptides.

8. The computer-implemented method of embodiment 6, wherein the trained machine learning model is configured to (i) map each binder identifier string of the plurality of binder identifier strings to a specific polypeptide sequence, or (ii) to fractionally assign a given binder identifier string to two or more specific polypeptide sequences, as part of inferring amino acid sequences of polypeptides from binder identifiers.

9. The computer-implemented method of embodiment 6, wherein the empirically determined assay parameter data comprises: (i) probabilities of assigning one or more binder identifiers from the set of binders to potential N-terminal amino acid (NTAA) residues of a polypeptide based on binding profiles of binders used in the encoding assay; and (ii) for each potential N-terminal amino acid residue in a polypeptide, a probability of successfully cleaving the N-terminal amino acid residue after the N-terminal amino acid residue is encoded in the encoding assay.

10. The computer-implemented method of any one of embodiments 1-9, wherein inferring amino acid sequences of polypeptides from binder identifier strings comprises inputting the binder identifier strings, or numerical representations thereof, into a trained machine learning model, wherein the trained machine learning model is trained on a training data set comprising a set of simulated binder identifier strings generated for a given input distribution of polypeptides, or numerical representations thereof, based on empirically determined assay parameter data comprising:
(i) probabilities of assigning one or more binder identifiers from the set of binders to potential N-terminal amino acid (NTAA) residues of a polypeptide based on binding profiles of binders used in the encoding assay;
(ii) for each potential NTAA residue in a polypeptide, a probability of cleaving the NTAA residue after the NTAA is encoded in the encoding assay, and
(iii) a probability of modifying the NTAA residue.

11. The computer-implemented method of any one of embodiments 1-10, wherein the set of binders comprises at least 5 different binders, and wherein the one or more components of polypeptides to which each binder binds each comprises an NTAA residue of polypeptides of the plurality of polypeptides or an NTAA residue modified by a N-terminal modifier agent.

12. The computer-implemented method of any one of embodiments 1-11, wherein the set of binders comprises at least 5 different binders, and wherein the one or more components of polypeptides to which each binder binds each comprises a terminal amino acid or a terminal dipeptide of polypeptides of the plurality of polypeptides.

13. The computer-implemented method of embodiment 11 or embodiment 12, wherein the N-terminal modifier agent is a compound of a formula selected from the group consisting of.

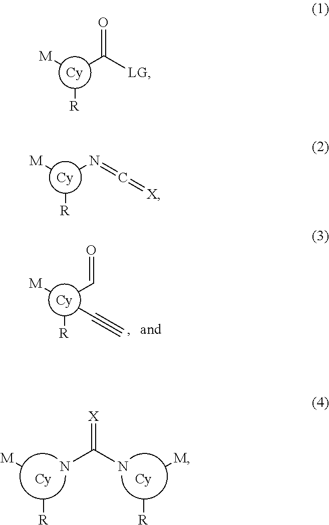

wherein M is a metal binding group that comprises sulfonamide, hydroxamic acid, sulfamate, or sulfamide; the group

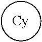

is a 5 or 6 membered aromatic ring containing up to three heteroatoms selected from N, O, and S as ring members, and is optionally substituted by R; R represents one or two optional substituents selected from the group consisting of F, Cl, CH₃, CF₂H, CF₃, OH, OCH₃, OCF₃, NH₂, N(CH₃)₂, NO₂, SCH₃, SO₂CH₃, CH₂OH, B(OH)₂, CN, CONH₂, CO₂H, CN₄H, and CONHCH₃; LG is OH, ORQ, or OCC, each RQ is independently aryl or heteroaryl, each of which is optionally substituted with one or more groups selected from halo, nitro, cyano, sulfonate, carboxylate, alkylsulfonyl, and N of heteroaryl is optionally oxidized; or RQ can be —C(═O)R or —C(═O)—OR; CC is a cationic counterion; X is one of the following: O, S, Se, or NH.

14. The computer-implemented method of any one of embodiments 1-13, wherein the plurality of nucleic acid sequences are generated in an encoding assay that comprises contacting the plurality of polypeptides with the set of binders.

15. The computer-implemented method of any one of embodiments 1-14, wherein the series of encoder barcode sequences comprises at least three different encoder barcode sequences.

16. The computer-implemented method of any one of embodiments 1-15, wherein the series of encoder barcode sequences comprises from 4 to 20 different encoder barcode sequences.

17. The computer-implemented method of embodiment 6, wherein the trained machine learning model is trained on a training data set generated by performing multiple encoding assays with pre-determined analytes and by inputting the series of encoder barcode sequences or numerical representations thereof generated during each encoding assay and encoding assay parameters to the machine learning model.

18. The computer-implemented method of any one of embodiments 1-17, wherein a computer model infers two or more amino acid sequences of polypeptides of the plurality of polypeptides from each binder identifier string, and outputs probabilities that a given binder identifier string of the plurality is derived from one of the two or more amino acid sequences of polypeptides inferred from the binder identifier string.

19. The computer-implemented method of any one of embodiments 1-18, wherein
(i) each of the plurality of nucleic acid sequences further comprises one or more auxiliary sequences;
(ii) the method further comprises assigning, using the one or more processors, an auxiliary identifier to at least one of the one or more auxiliary sequences in each of the plurality of nucleic acid sequences to generate a plurality of binder identifier strings comprising the auxiliary identifier; and
(iii) the one or more auxiliary sequences comprise one or more identifier sequences or complements thereof, one or more spacer sequences or complements thereof, one or more sequencing primer sequences or complements thereof, or any combination thereof.

20. The computer-implemented method of embodiment 19, wherein the one or more identifier sequences comprise one or more sample barcode sequences, one or more bead barcode sequences, one or more unique molecular identifier (UMI) sequences, or any combination thereof.

21. The computer-implemented method of embodiment 19, wherein generating a binder identifier string is based on (i) a specific order of encoder barcode sequences in each of the plurality of nucleic acid sequences and, optionally, (ii) a look-up table (LUT) of binder identifiers.

22. The computer-implemented method of embodiment 21, wherein generating a binder identifier string further comprises performing error correction based on an enumeration of error types to account for errors associated with an encoding process used to generate each of the series of encoder barcode sequences.

23. The computer-implemented method of embodiment 21 or embodiment 22, wherein generating a binder identifier string comprises use of a probabilistic model to predict a sequence of binder identifiers for each nucleic acid sequence of the plurality.

24. The computer-implemented method of any one of embodiments 1-23, wherein the plurality of polypeptides encoded in the plurality of nucleic acid sequences by the encoding assay comprises at least 1000, at least 10,000, at least 100,000, at least 1,000,000, or more polypeptides.

25. The computer-implemented method of any one of embodiments 1-24, wherein the plurality of polypeptides encoded in the plurality of nucleic acid sequences by the encoding assay comprises at least 500, at least 1000, at least 2000, at least 5000, at least 10,000, at least 20,000 or more different polypeptides.

26. The computer-implemented method of any one of embodiments 1-25, wherein the binding profiles of the binders are determined experimentally.

27. The computer-implemented method of any one of embodiments 1-25, wherein the binding profiles are determined computationally.

28. The computer-implemented method of any one of embodiments 18-26, wherein inferring amino acid sequences of polypeptides from binder identifier strings comprises inputting the binder identifier strings and encoding assay data to the computer model.

29. The computer-implemented method of embodiment 28, wherein the computer model is configured to identify unique binder identifier signatures in the plurality of binder identifier strings as part of inferring amino acid sequences of polypeptides from binder identifiers, and wherein a given unique binder identifier signature comprises a set of binder identifier strings associated with a single polypeptide of the plurality of polypeptides.

30. The computer-implemented method of embodiment 28, wherein the computer model computes binding profiles of binders of the set of binders based on the inputted encoding assay parameter data.

31. The computer-implemented method of any one of embodiments 1-30, wherein inferring amino acid sequences of polypeptides from binder identifiers comprises inputting one or more corresponding numerical representations of the binder identifier strings and optionally one or more corresponding numerical representations of encoding assay parameter data to a computer model.

32. The computer-implemented method of any one of embodiments 1-31, wherein inferring amino acid sequences of polypeptides from binder identifiers comprises outputting one or more corresponding numerical representations of the polypeptide sequences from a computer model.

33. The computer-implemented method of embodiment 32, wherein the corresponding numerical representations of the binder identifier strings are binder sequence embeddings generated from a trained protein language model (pLM), protein foundation model or natural language processing (NLP) model.

34. The computer-implemented method of embodiment 32, wherein the corresponding numerical representations of the polypeptide sequences are polypeptide sequence embeddings generated from a protein language model, protein foundation model, or natural language processing model.

35. The computer-implemented method of embodiment 31, wherein the corresponding numerical representations of encoding assay parameter data includes: (i) the concentrations of one or more binders in the set of binders; (ii) thermodynamic parameters of binders, including association rate constants and dissociation rate constants for components of one or more polypeptides; (iii) the incubation time of binders; (iv) the wash time of binders after binder incubation; (v) the ligase concentration; (v) the ligase reaction time; (vi) thermodynamic parameters of the ligase, including Michaelis constant and catalytic turnover rate; (vii) estimated polypeptide concentrations; (viii) the concentration of cleavase enzymes; and (ix) buffer conditions for each step of the encoding assay, including enzymatic substrate concentrations, salt identities, salt concentrations, and pH.

36. The computer-implemented method of any one of embodiments 1-35, wherein the binder identifier string for each nucleic acid sequence is generated by inferring identifying information regarding the corresponding binder for each encoder barcode sequence within a given nucleic acid sequence.

37. The computer-implemented method of embodiments 22, wherein accounting for the errors associated with the process used to generate each of the series of encoder barcode sequences comprises determining: (i) a first probability for reading a specified encoder barcode sequence for a specified underlying polypeptide component, and (ii) a second probability for successfully transitioning from one polypeptide component to the next polypeptide component during the encoding process.

38. The computer-implemented method of embodiment 23, wherein the probabilistic model comprises a hidden Markov model (HMM), and optionally wherein the hidden Markov model (HMM) is trained using one or more training data sets comprising labeled pairs of binder identifiers and nucleic acid sequences.

39. The computer-implemented method of embodiment 38, wherein the hidden Markov model (HMM) is trained using an iterative Expectation-Maximization (EM) algorithm to determine a set of model parameters that maximize a probability of correctly predicting a sequence of binder identifiers for each nucleic acid sequence of the plurality, and optionally wherein the Expectation-maximization (EM) algorithm comprises a Baum-Welch algorithm.

40. The computer-implemented method of any one of embodiments 1-39, wherein the one or more components of the polypeptide to which the binding moiety binds comprises a post-translation modification of at least one amino acid residue.

41. The computer-implemented method of embodiment 29, wherein the trained machine learning model is trained using a training data set comprising binder identifier string data for one or more isolated polypeptide samples that are processed using a same sample preparation protocol as that used to process the plurality of polypeptides.

42. The computer-implemented method of embodiment 41, wherein the training data set further comprises binder identifier string data for a background sample processed using a same sample preparation protocol as that used to process the plurality of polypeptides, and wherein optionally the background sample comprises a plasma sample, a urine sample, a saliva sample, or a cell extract sample.

43. The computer-implemented method of any one of embodiments 18-42, wherein the computer model is further configured to correct the quantity output for the at least one polypeptide using a correction factor calculated from a training data set.

44. The computer-implemented method of embodiment 8, wherein mapping a binder identifier string of the plurality of binder identifier strings to a specific polypeptide comprises:
i) generating a set of k-mer fragments for the binder identifier string;
ii) determining a probability that a given k-mer fragment of the set belongs to a specific polypeptide based on a previously determined probability distribution; and
iii) assigning the binder identifier string to the specific polypeptide based on the determined probabilities for the set of k-mer fragments.

45. The computer-implemented method of embodiment 44, wherein the probabilistic model is trained on empirically determined assay parameter data.

46. The computer-implemented method of embodiment 45, wherein the empirically-determined assay parameter data comprises, for each barcode sequence of the plurality of barcode sequences, a probability of reading a given barcode sequence in a nucleic acid sequence for a given one or more amino acid residue(s) of a corresponding polypeptide to which a binding moiety was bound.

47. The computer-implemented method of embodiment 45 or embodiment 46, wherein the empirically determined assay parameter data comprises, for each potential N-terminal amino acid residue in a polypeptide, a probability of successfully cleaving the N-terminal amino acid residue during a cyclical process used to encode the plurality of polypeptides in the plurality of nucleic acid sequences.

48. The computer-implemented method of embodiment 8, wherein mapping a binder identifier string of the plurality of binder identifier strings to a specific polypeptide comprises providing the plurality of binder identifier strings as input to the trained machine learning model, wherein the trained machine learning model is configured to fractionally assign a given binder identifier string to two or more specific polypeptides.

49. The computer-implemented method of embodiment 48, wherein the trained machine learning model is trained on a training data set comprising a set of simulated binder identifier strings generated for a given input distribution of polypeptides based on empirically-determined assay parameter data comprising:
i) for each barcode sequence of the plurality of barcode sequences, a probability of reading a given barcode sequence in a nucleic acid sequence for a given one or more amino acid residue(s) of a corresponding polypeptide to which a binding moiety was bound; and
ii) for each potential N-terminal amino acid residue in a polypeptide, a probability of successfully cleaving the N-terminal amino acid residue during a cyclical process used to encode the plurality of polypeptides in the plurality of nucleic acid sequences.

50. The computer-implemented method of embodiment 49, wherein the set of simulated binder identifier strings comprises at least 100,000, 500,000, 1M, 5M, 10M, 25M, 50M, 75M, 100M, 200M, 300M, 400M, or 500M simulated binder identifier strings.

51. The computer-implemented method of any one of embodiments 1 to 50, wherein the method is further configured to output a confidence interval for the partial identity of the at least one polypeptide.

52. The computer-implemented method of embodiment 51, wherein a level of stringency in the output confidence interval is selectable by a user.

53. The computer-implemented method of embodiment 52, wherein the level of stringency in the confidence interval corresponds to a confidence level of 90%, 95%, 98%, or 99%.

54. The computer-implemented method of embodiment 6, wherein the trained machine learning model comprises a trained artificial neural network model, a trained deep learning model, a trained random forest model, or a trained support vector machine

55. The computer-implemented method of any one of embodiments 1 to 54, further comprising performing an iterative Expectation Maximization (EM) process to refine the quantity output for the at least one polypeptide of the plurality of polypeptides.

56. The computer-implemented method of embodiment 55, wherein the iterative EM process comprises repetitively:
i) finding a best assignment of a binding moiety to each barcode sequence in a nucleic acid sequence based on a current estimate of what polypeptides are present in the plurality of polypeptides;
ii) finding an updated best estimate of what polypeptides are present in the plurality of polypeptides based on the best assignment of a binding moiety to each different barcode sequence in the nucleic acid sequence; and
iii) determining an amount of the at least one polypeptide present in the plurality of polypeptides based on the updated best estimate of what polypeptides are present in the plurality of polypeptides.

57. The computer-implemented method of embodiment 56, wherein steps (i) to (iii) are repeated until a difference between the amount of the at least one polypeptide determined in one iteration and the next is less than a specified threshold, or until a specified maximum number of iterations has been reached.

58. The computer-implemented method of any one of embodiments 1 to 57, further comprising determining the nucleic acid sequences for the plurality of nucleic acid sequences, optionally wherein the nucleic acid sequences are determined by performing DNA sequencing.

59. The computer-implemented method of any one of embodiments 1 to 58, further comprising determining amino acid sequences for each polypeptide of the plurality of polypeptides.

60. The computer-implemented method of any one of embodiments 1 to 59, wherein the plurality of nucleic acid sequences comprises at least 10,000, 100,000, or 1M nucleic acid sequences.

61. The computer-implemented method of any one of embodiments 1 to 60, wherein the plurality of polypeptides comprises at least 100, 500, 1,000, 2,000, 5,000, or 10,000 different polypeptides.

62. The computer-implemented method of embodiment 2, wherein at least two peptidic reads are generated for at least one nucleic acid sequence of the plurality of nucleic acid sequences.

63. A computer-implemented method comprising:
a) receiving, at one or more processors, amino acid sequences for a plurality of polypeptides;
b) generating, using the one or more processors, a plurality of peptide sequences based on the amino acid sequences;
c) converting, using the one or more processors, each peptide sequence of the plurality into a binder identifier string based on an order of amino acid residues in the peptide sequence and a binding profile for a plurality of binding moieties, wherein each binding moiety of the plurality binds to a component of a polypeptide and corresponds to a binder identifier, and wherein the binder identifier corresponds to an encoder barcode sequence; and
d) providing, using the one or more processors, the plurality of binder identifier strings as input to a trained model, wherein the trained model is configured to convert binder identifier strings to nucleic acid sequences based on a set of probabilities for binding of the plurality of binding moieties to components of the polypeptides of the plurality of polypeptides and output a plurality of nucleic acid sequences corresponding to the plurality of polypeptides.

64. The computer-implemented method of embodiment 63, further comprising comparing the plurality of nucleic acid sequences output by the trained model to a plurality of nucleic acid sequences determined by subjecting the plurality of polypeptides to an encoding assay and sequencing the resulting extended recording tags.

65. A system comprising:
one or more processors; and
a memory communicatively coupled to the one or more processors and configured to store instructions that, when executed by the one or more processors, cause the system to perform the computer-implemented method of any one of embodiments 1 to 64.

66. A non-transitory computer-readable storage medium storing one or more programs, the one or more programs comprising instructions, which when executed by one or more processors of a system, cause the system to perform the computer-implemented method of any one of embodiments 1 to 64.

### EXAMPLES

The following examples are offered to illustrate but not to limit the methods, compositions, and uses provided herein. Certain aspects of the present invention, including, but not limited to, embodiments for the Encoding polypeptide sequencing assay, methods of information transfer between coding tags and recording tags, methods of modifying (functionalizing) terminal amino acid residues of peptides, methods of generating specific binders recognizing modified terminal amino acid residues of peptides, methods of cleaving modified terminal amino acid residues of peptides, methods of making nucleotide-polypeptide conjugates, methods for attachment of nucleotide-polypeptide conjugates to a support, methods of generating barcodes, methods for analyzing extended recording tags were disclosed in earlier published applications, e.g., US 2019/0145982 A1, US 2020/0348308 A1, US 2020/0348307 A1, US 2021/0208150 A1, US 2022/0049246 A1, US 2022/0283175 A1, US 2022/0144885 A1, US 2022/0227889 A1, US 2021/0214701 A1, US 2024/0294981 A1, US 2024/0053350 A1, and US 2023/0136966 A1, the contents of which are incorporated herein by reference in their entireties.

### Example 1—Encoding Assay

The bioinformatics tools described herein complement recently described methods for polypeptide analysis that employ a nucleic acid-based polypeptide encoding technique (i.e., a reverse translation assay).

FIG. 6 depicts an exemplary design of a solid support (such as 113epharose bead) configured for use in a polypeptide analysis/reverse translation assay. The bead comprises multiple covalently attached polypeptides, each associated with a barcoded nucleic acid recording tag (RT). About 10\hat{ }7 RTs per bead is used. Two barcodes are shown: BBC—bead barcode which allows for sample multiplexing (each polypeptide sample would be incubated with a set of beads sharing a unique bead barcode); and PBC—peptide barcode, which may uniquely identify the attached polypeptide (also called RT barcode), or identify a fraction or a compartment from which the attached polypeptide is originated. ILM is used for polypeptide attachment as well as for amplification of extended RTs produced during encoding. Spacer A (Sp_A) is required for transfer of information (encoding) to occur, forming a mutual complementary region between RT and a coding tag (CT) of a binding agent that binds to the polypeptide associated with the RT and allowing ligation between RT and CT to occur.

FIG. 7A and FIG. 7B depict two exemplary polypeptide analysis assays involving information transfer. The left panels of FIG. 7A and FIG. 7B show a polypeptide attached to a solid support and associated with a barcoded nucleic acid recording tag (RT), similar to shown in FIG. 6. Next, a cycle of encoding (information transfer) is shown, which comprises either 3 steps (binding, ligation and digestion with the restriction enzyme (RE), see FIG. 7A), or 4 steps (binding, ligation, extension and digestion with the restriction enzyme (RE), see FIG. 7B). During the binding step, a binding agent binds to the polypeptide (e.g., a modified NTAA of the polypeptide), bringing a coding tag (CT) attached to the binding agent into proximity with the RT. In some embodiments, the binding agent can be attached or joined to the coding tag in locations other than depicted (e.g., at the loop region of the CT or others). The binding agent as shown is attached to the CT by a linker. The coding tag may contain a binding agent-specific barcode (SB), a 2 bp spacer, and a type IIS restriction enzyme site (RS). While embodiment of FIG. 7A utilizes only double-stranded ligation (ligation of both strands of or connected to the double-stranded region of the CT to the strands of or connected to the double-stranded region of the RT) during information transfer, embodiment of FIG. 7B utilizes both ligation on one strand of double-stranded region (ligation of a single strand of or connected to the double-stranded region of the CT to a strand of or connected to the double-stranded region of the RT; also called “single-stranded ligation”) and extension (polymerase extends the 3′ (non-ligated) end of the RT). Both embodiments utilize the digestion step (cleaving the double-stranded extended recording tag with a nucleic acid cleaving reagent, such as RE), which allows for binding agent release and generates a terminus of the double-stranded extended recording tag suitable for further encoding cycles (makes the terminus of the extended RT compatible with the terminus of a new coding tag of the next binding agent). Suitable terminus of the extended RT may contain a short single-stranded overhang, or may be a blunt end. Next, in some embodiments, after a cycle of information transfer, a portion of the polypeptide for analysis (e.g., one or two terminal amino acid residues) can be removed from the polypeptide. The cycle of binding/ligation/digestion may be repeated one or more times with additional binding agents and coding tags to further extend the recording tag associated with the polypeptide. Thus, multiple binding events for the polypeptide may be encoded into the extended recording tag.

### Example 2—Decoding Encoded Polypeptide Information

FIG. 8 depicts an exemplary sequence read (SEQ ID NO: 12) corresponding to an extended RT obtained by an encoding method (see e.g., the method described in FIG. 2 or FIG. 7A and FIG. 7B) is shown. The architecture of the extended RT is designed before the assay and comprises two separate barcodes located in the original (non-extended) RT (BBC and RT barcode), then one or more spacer-CT barcode pairs, wherein each pair is added to the RT or extended RT during a single encoding cycle (CT barcode is transferred to the RT from CT of the binding agent bound to the polypeptide during the current encoding cycle), and wherein additional spacer-CT barcode pairs are generated from additional encoding cycles. Finally, the extended RT further comprises LM sequence (used for the extended RT amplification) and CBC barcode attached during the final capping step, which indicates the end of the sequence reads. An algorithm is used to generate barcode-level alignments of nucleic acid sequence reads based on the shown look-up table and accounting for error correction. In this example, only two binding agents were used, and there are only two corresponding CT barcodes in the look-up table (SB04 and SB05). The computer model generates as an output one or more identifier strings comprising binder identifiers and auxiliary identifiers for each analyzed sequence read.

FIG. 9 provides a non-limiting example of the output of the computer model that generates one or more binder identifier string for each analyzed nucleic acid sequence read as described in FIG. 8. The first row, for example, indicates the nucleic acid sequence for a sequence read or portion thereof, and the second row indicates the corresponding binder identifiers that constitute an identifier string corresponding to the sequence read. BBC=bead barcode. UMI8.0=unique molecular identifier (8 nucleotides in length). SB49=RT barcode. GC=GC spacer. SB04=CT barcode. AG=AG spacer. LM1.0=amplification primer sequence. CBC=capping barcode.

### Example 3—Proteome Analysis System

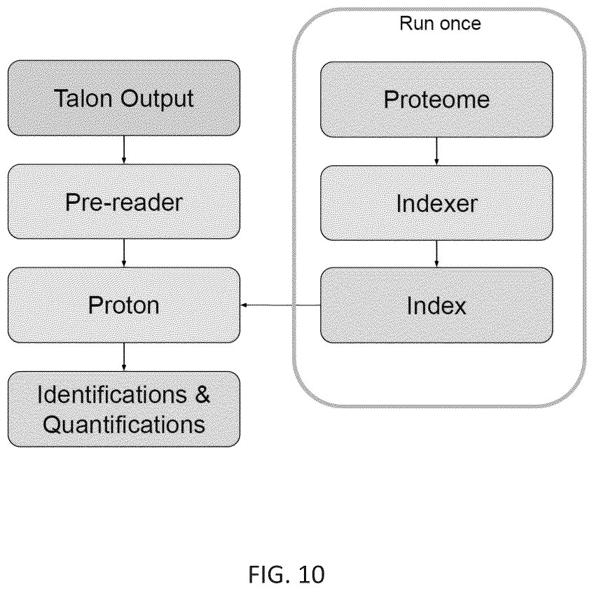

FIG. 10 provides an exemplary schematic illustration of a proteome analysis system, according to one implementation as described herein, that enables high-throughput analysis of polypeptides (e.g., peptides and polypeptides, or fragments thereof) based on amino acid sequence data that has been encoded in the form of barcoded nucleic acid sequences. FIG. 10 illustrates an exemplary flow diagram of how the identifier strings may undergo preprocessing before being processed using a polypeptide aligner program (“top-down” algorithm) in order to make polypeptide level identifications and quantifications. The methods described herein are used to analyze sequence read data obtained by sequencing the barcoded nucleic acid sequences, and enable correction of data for short (e.g., corresponding to 10-20 amino acid sequence fragments of a polypeptide) and error prone sequence reads (e.g., comprising substitutions, deletions, and phase shifts) that are often not amenable to traditional “seed and extend” methods of sequence alignment to identify peptides or polypeptides from which the reads were derived.

The disclosed methods are based on several underlying concepts, e.g., that one can pre-calculate the frequency of occurrence of high probability proteome encoding error events (e.g., errors in converting amino acid sequences to nucleic acid sequences, and errors in correctly transitioning from one amino acid residue to the next during the encoding process) and that one can use empirical error models to correct for these events. As a result, the disclosed methods provide for direct detection and quantification of peptides and polypeptides rather than relying on alignment to reference sequences.

As indicated in FIG. 10, an index of a given proteome of interest is prepared as part of a preprocessing step. Reference proteome data for a proteome of interest (e.g., for a given organism) is obtained (e.g., obtained experimentally and/or downloaded from appropriate proteomics databases (polypeptide abundances may be obtained, for example, from PaxDb (see, e.g., Huang, et al. (2023), “PaxDb 5.0: Curated Polypeptide Quantification Data Suggests Adaptive Proteome Changes”, Biorxiv, doi: 10.1101/2023.04.16.536357v1), and polypeptide sequences may be obtained from, e.g., UniProt (see, e.g., The UniProt Consortium (2021), “UniProt: the universal polypeptide knowledgebase in 2021”, Nucleic Acids Research 49(D1): D480-D489)) and processed using an Indexer to generate an index of the proteome that allows for fast sequence lookups. In some instances, the Indexer may generate the index by, for example, synthetically fragmenting the polypeptide sequences in the reference proteome, and performing sample preparation and encoding assay steps in silico to generate a look-up table of short binder identifier strings and their probabilities of arising from corresponding amino acid sequences derived from a plurality of different polypeptides in the proteome (see, e.g., Example 8 below). In some embodiments, the look-up table comprise of abbreviated codes each reflecting binder specificities for particular terminal amino acid residues. In some embodiments, the look-up table comprise of short nucleic acid sequences instead of short binder identifier strings.

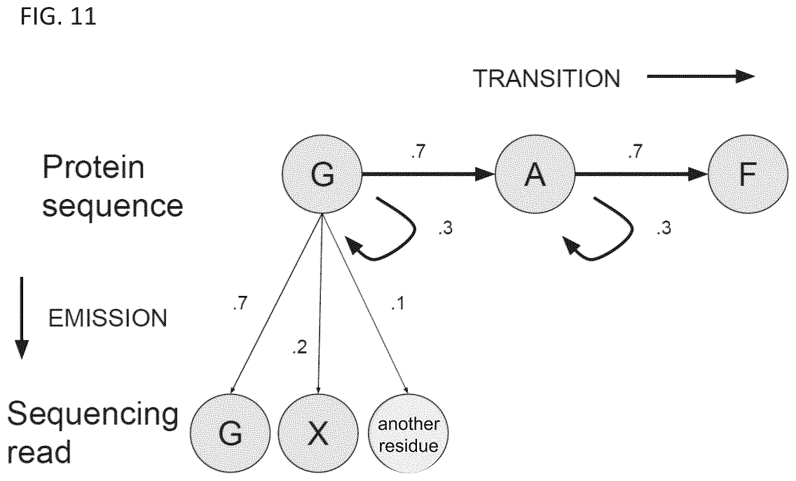

FIG. 11 provides an exemplary schematic illustration of potential errors that may occur when encoding polypeptide sequence information as barcoded nucleic acid sequences. This illustrates a probabilistic model, consisting of transitions between polypeptide states in subsequent encoding cycles (upper level) and emissions within a single encoding cycle that simulate the predicted encoding output based on use of the terminal amino acid (TAA)-specific binders. The indicated errors include transition errors (and associated probabilities) and emission errors (and associated probabilities). Transition errors comprise errors (and associated probabilities) of the encoding assay proceeding from one encoding cycle to another (e.g., from one amino acid residue to the next), such as errors caused by non-cleavage of a terminal amino acid (TAA) residue of the analyzed polypeptide after the encoding event in order to generate a newly formed terminal amino acid residue. In FIG. 11, a current TAA of the polypeptide (G) is cleaved with certain probability (indicated as 0.7 for both G and A residues in the non-limiting example of FIG. 11), thereby exposing A as a next TAA of the polypeptide, which is then cleaved to generate F as a next TAA of the polypeptide. In other embodiments, a terminal dipeptide may be cleaved during transition from one encoding cycle to another. In some instances, the probability of correctly transitioning from one amino acid residue to the next may be at least about 0.5, 0.55, 0.6, 0.65, 0.7, 0.75, 0.8, 0.85, 0.9, 0.95 or 0.99, with the corresponding probability of not correctly transitioning from one amino acid residue to the next (indicated as 0.3 for both G and A residues in the non-limiting example of FIG. 11) may be at most about 0.5, 0.45, 0.4, 0.35, 0.3, 0.25, 0.2, 0.15, 0.1, 0.05 or 0.01. Emission errors comprise errors (and associated probabilities) in correct recognition of polypeptide components, such as TAA residues, terminal oligopeptides (e.g., dipeptides, tripeptides) or other polypeptide components, within an encoding cycle. In FIG. 11, G residue (TAA of the polypeptide) is correctly recognized (and encoded) as G residue by a cognate binder with certain probability (indicated as 0.7 in the non-limiting example of FIG. 11). However, during the encoding cycle when the polypeptide is contacted with a set of different binders, G residue may incorrectly be recognized (and encoded) as X residue by a non-cognate binder with the probability of 0.2. Finally, G residue may incorrectly be recognized (and encoded) as any other residue (except for G and X) with the probability of 0.1 (exemplary values). In some instances, the probability of correctly encoding a given terminal amino acid residue (or other structure identified by a binder) as a corresponding nucleic acid sequence during the encoding cycle may be at least about 0.2, 0.3, 0.4, 0.5, 0.55, 0.6, 0.65, 0.7, 0.75, 0.8, 0.85, or 0.9, with a corresponding combined probability of not encoding or incorrectly encoding a given amino acid (or other structure identified by a binder) as a corresponding nucleic acid sequence may be at most about 0.8, 0.7, 0.6, 0.5, 0.45, 0.4, 0.35, 0.3, 0.25, 0.2, 0.15, or 0.1.

Returning to FIG. 10, Talon is a tool that maps raw nucleic acid sequence data to barcode-level alignments (i.e., tag sequences or identifier strings) using either a look-up table (LUT) approach or a hidden Markov model (HMM)-based approach, as described elsewhere herein. The LUT approach makes use of a “grammar” (i.e., the known structure of the extended recording tag sequences to identify next “words” (i.e., encoder barcode sequences) in the extended recording tag based on an exact match. The HMM approach relies on experimentally-determined probabilities that a given tag (binder identifier sequence) is associated with a given nucleic acid sequence (e.g., due to the possibility that a given binding agent binds to more than one amino acid residue (or set of amino acid residues) with different probabilities). The HMM model is used to determine the most likely order of tags (binder identifier sequences) in the extended reporting tag sequence. In some instances, the HMM approach may be more accurate than the LUT approach because it can correct for unexpected errors, e.g., sequencing errors.

The output from Talon is processed by a Pre-Reader to convert the tag sequences into binder identifier strings based on, e.g., on the order of binding and binding profiles for the binders that correspond to binder identifiers assigned in a given tag sequence (identifier string).

The output from the Pre-Reader is then processed by the “top-down” algorithm to assign each binder identifier string to a specific polypeptide. As described elsewhere herein, the assignment of binder identifier string to specific polypeptides may be accomplished using, e.g., a bulk analysis approach or a read-level analysis approach to identify specific polypeptides or polypeptides present in the sample and/or to quantify the polypeptides or polypeptides present in the sample.

In one non-limiting example, the polypeptides encoded by the nucleic acid sequences generated in a reverse translation assay may be identified by: (i) loading the proteome index generated for the sample type, (ii) reading each binder identifier string, (iii) breaking the binder identifier string into k-mers, (iv) scoring each k-mer based on a probability that it was derived from a given polypeptide present in the sample type, (v) combining the scores for the individual k-mers derived from each binder identifier string (e.g., by multiplication), and (vi) identifying the polypeptide from which the binder identifier string was derived, e.g., by comparing a ratio of probabilities (P_{Top Hit}/P_{Second Hit}) to a predetermined threshold. Exemplary ranges for the threshold are 10/1, 100/1, or 1000/1.

In another non-limiting example, the polypeptides encoded by the nucleic acid sequences generated in a reverse translation assay may be identified and quantified by (i) (i) loading the proteome index generated for the sample type, (ii) reading each binder identifier string, (iii) breaking the binder identifier string into k-mers, (iv) scoring each k-mer based on a probability that it was derived from a given polypeptide present in the sample type, (v) combining the scores for the individual k-mers derived from each binder identifier string (e.g., by multiplication), (vi) weighting the combined score according to a current quantification model, (vii) updating the current quantification model based on the latest set of weighting factors, and (viii) repeating steps (iii)-(vii) for each binder identifier string until, e.g., a specified minimal change in relative polypeptide quantities and/or a maximum number of iterations is reached. The determined probabilities of assignment for each binder identifier string and their determined frequencies of occurrence can then be used to identify and quantify the polypeptides present, e.g., by comparing a ratio of probabilities and frequencies ((P_{Top Hit}*Top Hit Frequency)/(P_{Second Hit}*Second Hit Frequency)) to a predetermined threshold. Exemplary ranges for the threshold are 10/1, 100/1, or 1000/1. Polypeptides may be quantified in terms of, e.g., parts per million (ppm) for all known/detected gene products.

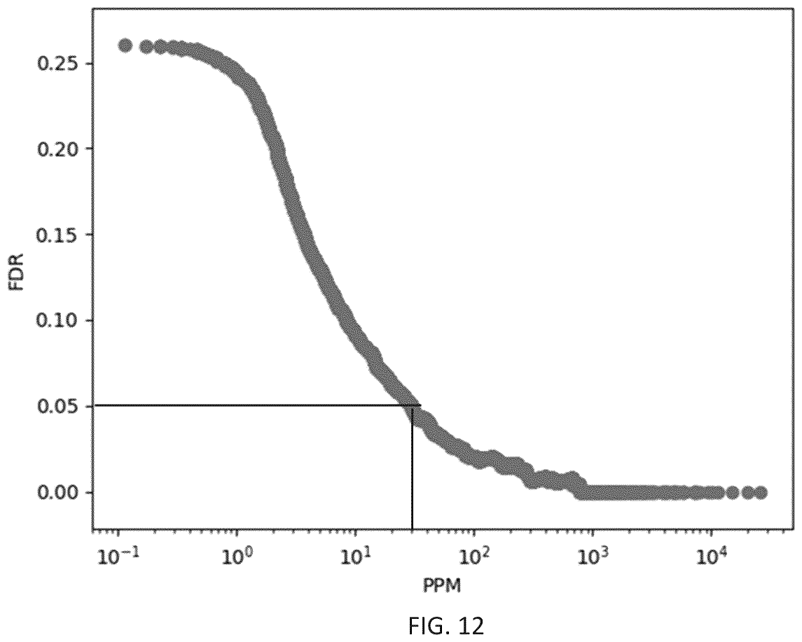

FIG. 12 provides a non-limiting example of false discovery rate (FDR) data as a function of polypeptide concentration (relative abundance) in a sample analyzed using the disclosed assay and bioinformatics techniques. For read level analysis and quantification, FIG. 12 provides an example of how the false discovery rate is affected by the polypeptide abundance in parts per million (PPM). To generate the data shown in FIG. 12, approximately 11 million peptidic reads were simulated based on a set of polypeptide sequences taken from UniProt and polypeptide abundances taken from PaxDb as described elsewhere herein. The polypeptide sequences were synthetically fragmented, filtered to account for expected sample preparation losses, and used to generate the nucleic sequences expected to be generated by performing the reverse translation assay described elsewhere herein. The resulting nucleic acid sequences were then processed using the techniques described elsewhere herein and illustrated in FIG. 10 to identify and quantify polypeptides. The output was then compared to the known ground truth data used to generate the synthetic nucleic acid sequences. FIG. 12 shows a plot of the FDR (i.e., the number of false positives detected, divided by the sum of the number of false positives and true positives) as a function of the relative abundances (in ppm) of polypeptides for a maximum error per quantification of 1. It answers the question: what is the lowest PPM above which are quantifications are correct? The data indicate that, for this simulation, the limit of quantification was 24 ppm.

### Example 4—Read-Level Analysis Vs. Bulk Analysis

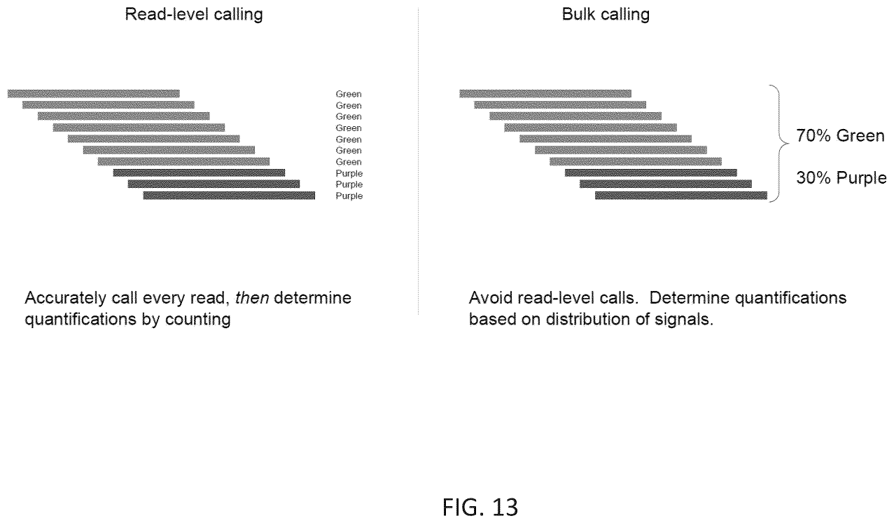

FIG. 13 provides an exemplary schematic illustration of the difference between read-level analysis (left panel) and bulk-level analysis (right panel).

In read-level analysis, the objective is to accurately assign each binder identifier string to a polypeptide, and then quantify the polypeptides present by counting the number of assigned reads for each polypeptide. Exemplary output would include, for example, that binder identifier string X was derived from polypeptide Y, and that polypeptide Y is present at a concentration of Z ppm.

In bulk-level analysis, the objective is to quantify the polypeptides present based on a distribution of polypeptide signatures (e.g., a set of binder identifier strings (or signals) associated with a polypeptide) without assigning individual binder identifier strings to specific polypeptides. In some instances, for example, a quantitative machine learning models may be trained for analysis of both foreground and background signals. The foreground model is trained on data for a specific polypeptide, while the background model is trained on data for a given sample type, e.g., plasma. Signatures detected in a sample are then used to determine what polypeptides are present and what their relative abundances are. In some instances, the quantification of polypeptides may be corrected using correction factors calculated from the training data sets. For example, a signature may be present at a level of 80% in a pure polypeptide sample, and at a level of only 2% in the background (e.g., plasma) sample. The predicted abundance for a given polypeptide in the test sample may then be adjusted accordingly, e.g., by linearly scaling the predicted abundance to comply with a range of signature levels that runs from 2% (no polypeptide present) to 80% (for the given polypeptide in a pure sample). Exemplary output would include, for example, that polypeptide Y is present at a concentration of Z ppm.

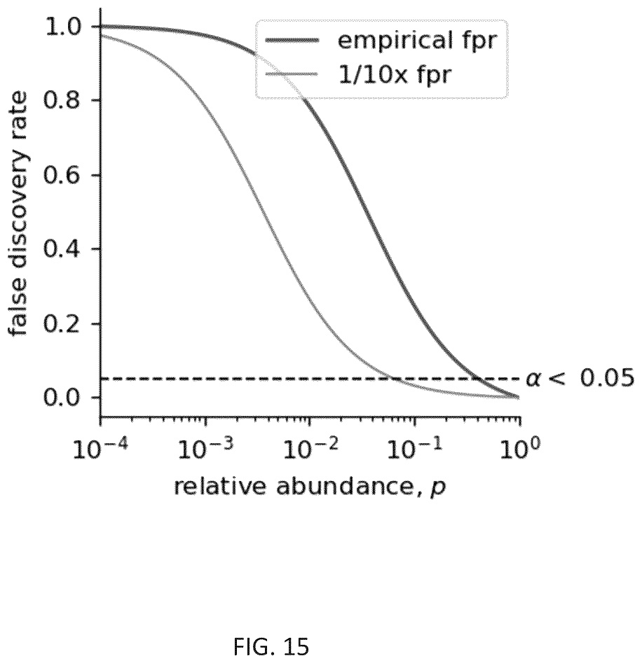

For read level analysis, FIG. 15 illustrates an example of how the false positive rate (fpr) affects the false discovery rate (FDR) based on the actual relative abundance of a polypeptide, p, providing a method to statistically threshold the confidence in making a quantitative assessment of the presence of that polypeptide. The less abundant is a polypeptide, the higher is FDR. The smaller is false positive rate, the smaller is FDR (the curves are shown for empirical fpr and for 1/10^{th} fpr. For single read level analysis, as abundance of a polypeptide drops, the measurements must improve in order to correctly detect the polypeptide.

### Example 5—Use of Expectation-Maximization Techniques to Refine Predictions for Polypeptide Abundance

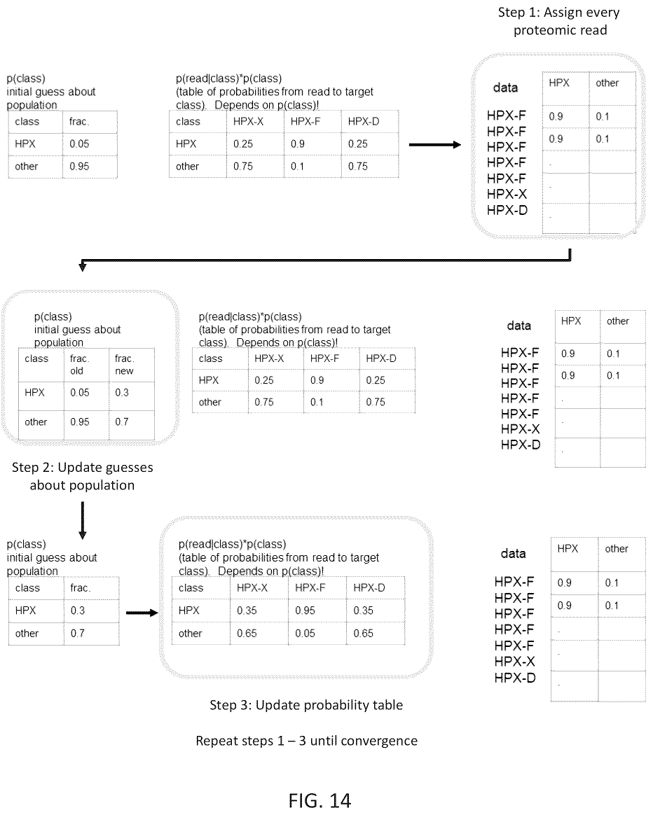

In some instances, an Expectation-Maximization (EM) technique may then be used to refine the predictions for polypeptide quantitation/abundance obtained through the use of the disclosed methods, as illustrated in FIG. 14 for binder identifier strings derived from a sample comprising Hemopexin (HPX). The process starts with an initial guess for the probability distribution of binder identifier strings in the sample, which can be used to generate a table of probabilities that a given read maps to a given target polypeptide. At step 1 in FIG. 14, each binder identifier string is assigned to a specific polypeptide (the expectation step). At step 2, the parameters describing the probability distribution of binder identifier strings are updated based on the current binder identifier string assignments to maximize the fit between the current assignments and the observed distribution of binder identifier strings (the maximization step). At step 3, the probability table is updated based on the revised probability distribution parameters. Steps 1-3 are then repeated until (i) the change in binder identifier string assignments and predicted polypeptide abundances from one iteration to the next is less than a specified threshold (e.g., less than 0.1%, 0.5%, 1%, 2%, 3%, 4%, or 5%), or (ii) a specified maximum number of iterations is reached (e.g., a maximum of 20, 40, 60, 80, 100, 200, 300, 400, or 500 iterations), or (iii) the difference in the sum of the predicted abundances (e.g. squared Euclidean distance, or Euclidean distance, or Manhattan distance) is less than some amount, e.g. 1e-3, 1e-5, 1e-7.

### Example 6—Evaluation of Binding Efficiencies of Binders Specific to Modified NTAA Residues of Polypeptides Via the Multiplex Encoding Assay

A high-throughput polypeptide sequencing assay (a variant of an encoding assay) as described in the published US patent applications US 2019/0145982 A1 and US 2023/0136966 A1, see also FIG. 2 and FIG. 7A-FIG. 7B, requires a set of at least 4, 5, 6, 10 or more different binders, wherein each of at least 4, 5, 6, 10 or more different binders is (i) configured to bind to a N-terminal amino acid (NTAA) residue, C-terminal amino acid (CTAA) residue, modified NTAA residue or modified CTAA residue of immobilized polypeptide molecules, and (ii) at least partially selective for a different peptide component, wherein the different peptide component is a different NTAA residue or a different CTAA residue of immobilized polypeptide molecules. In some embodiments, a set of binders contains at least 4 different binders, wherein is each binder is at least partially selective for and configured to bind to a different modified NTAA residue of immobilized polypeptide molecules. The described binding set may be generated as described in the following patent publications: US 2023/0220589 A1 and US 2022/0283175 A1, incorporated by reference herein. These applications also recite exemplary synthesis and installation methods of N-terminal modifier agents (NTMs) on NTAA residues of polypeptides. Brief schematics of installation of exemplary NTMs are shown below (details are provided in US 2021/0214701 A1, US 2023/0220589 A1 and US 2022/0283175 A1, incorporated by reference).

### N-Terminal Modifier Agent M15:

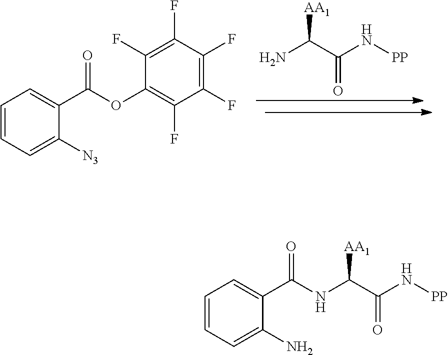

### N-Terminal Modifier Agent M17:

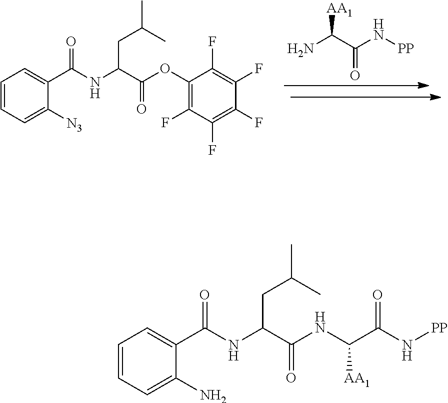

### N-Terminal Modifier Agent M64:

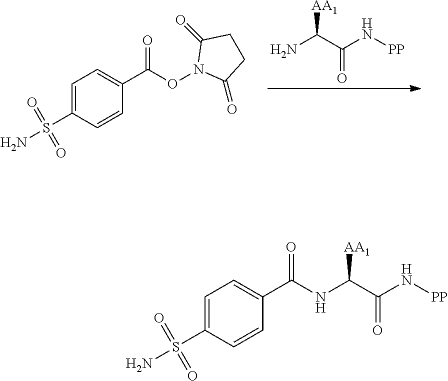

### N-Terminal Modifier Agent M103:

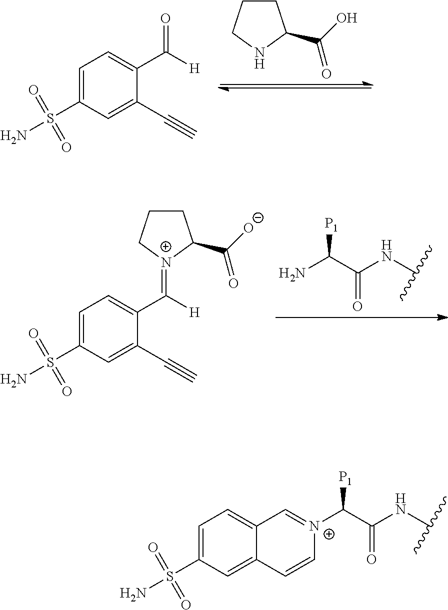

To select and/or analyze engineered NTM-NTAA specific binders in a high-throughput manner, binders were assayed on a set of at least 289 peptides (17×17 combination of different P1 and P2 residues) modified with a specific N-terminal modifier agent and immobilized on a solid support associated with an individual nucleic acid recording tag. For immobilization, the peptides containing C-terminally attached 6-Azido-L-lysine were reacted with DBCO-C2-modified 17 nt oligonucleotides in 100 mM HEPES, pH=7.0 at 60° C. for 1 hour. Each NTAA peptide-oligonucleotide conjugate was ligated to two different 15 nt DNA fragments containing a 7 nt barcode and an 8 nt spacer sequence using splint DNA and T4 DNA ligase to generate a peptide-recording tag conjugate with two different barcodes. A total of 576 peptide-recording tag conjugates were prepared and pooled for ligation and immobilization on short hairpin capture DNAs attached to the beads (NHS-Activated Sepharose High Performance, Cytiva, USA). The capture DNAs were attached to the beads using trans-cyclooctene (TCO) and methyltetrazine (mTet)-based click chemistry. TCO-modified short hairpin capture DNAs (16 basepair stem, 4 base loop, 17 base 5′ overhang) were reacted with mTet-coated beads. The peptide-recording tag pools (20 nM) were annealed to the hairpin capture DNAs attached to the beads in 0.5 M NaCl, 50 mM sodium citrate, 0.02% SDS, pH 7.0, and incubated for 30 minutes at 37° C. The beads were washed once with 1× phosphate buffer, 0.1% Tween 20 and resuspended in 1× Quick ligation solution (New England Biolabs, USA) with T4 DNA ligase. After a 30 min incubation at 25° C., the beads were washed once with 1× phosphate buffer, 0.1% Tween 20, three times with 0.1 M NaOH, 0.1% Tween 20, three times with 1× phosphate buffer, 0.1% Tween 20, and resuspended in 50 μL of PBST.

Each binding agent was conjugated to a corresponding nucleic acid coding tag comprising barcode with identifying information regarding the binding agent. The coding tag specific for the binding agent was attached to SpyTag via a PEG linker, and the resulting fusions were reacted with binding agent-SpyCatcher fusion polypeptide via SpyTag-SpyCatcher interaction, essentially as described in US 2021/0208150 A1. Briefly, amine-functionalized oligonucleotide coding tags were conjugated to a heterobifunctional linker containing an NHS ester, PEG24 linker and maleimide. Excess linker was removed by acetone purification, and excess linker in solution was removed by centrifugation. Purified oligonucleotide-PEG24-maleimide was incubated overnight with SpyTag peptide forming a conjugate via a cysteine residue. The sample was spun down to remove precipitate and the supernatant was transferred to a 10k molecular weight filter to remove excess SpyTag peptide. After multiple washes, the final bioconjugate of SpyTag peptide containing a PEG24 linker and coding tag oligonucleotide was obtained and subsequently combined with the binder/SpyCatcher fusion polypeptide spontaneously forming the final binder-fused coding tag conjugate.

Before the encoding assay, the beads with immobilized target peptide-recording tag conjugates were treated with an N-terminal modifier agent to modify the N-terminal of the immobilized peptides. The modified beads with peptide conjugates were washed once with 70% Ethanol, washed once with water and resuspended in PBST. The coding tags attached to the binding agents form a loop with 12 bp duplex and 9 nt spacer at the 3′, which is complementary to the 3′ spacer of the recording tag on the beads.

The cycle of the encoding assay described in this example consists of contacting the immobilized peptides with a set of binder-coding tag conjugates. For this, each binder (50 nM) was incubated with the recording tag-peptide conjugates immobilized on the beads for 30 min at 25° C., followed by washing twice with 1× phosphate buffer, pH 7.3, 500 mM NaCl, 0.1% Tween 20. This was followed by transferring information of the coding tag to the recording tags associated with the target peptides by a primer extension reaction after partial hybridization between the coding tag and the recording tag through a shared spacer region using a DNA polymerase having 5′-to-3′ polymerization activity and having substantially reduced 3′-to-5′ exonuclease activity. Extension was performed by addition of 50 mM Tris-HCl, pH 7.5, 2 mM MgSO₄, 50 mM NaCl, 1 mM DTT, 0.1 mg/mL BSA, 0.1% Tween 20, dNTP mixture (125 uM of each) and 0.125 U/uL of Klenow fragment (3′->5′ exo−) (MCLAB, USA) at 25° C. for 15 min, followed by one wash of 1× phosphate buffer, 0.1% Tween 20, twice with 0.1 M NaOH+0.1% Tween 20, and twice with 1× phosphate buffer, 0.1% Tween 20. After the recording tag extension, the binder-coding tag conjugate was washed away, and the sample was capped by introducing with primer binding site for PCR and NGS with incubation of 400 nM of an end capping oligo with 0.125 U/uL of WT Klenow fragment (3′->5′ exo-), dNTPs (each at 125 uM), 50 mM Tris-HCl (pH, 7.5), 2 mM MgSO₄, 50 mM NaCl, 1 mM DTT, 0.1% Tween 20, and 0.1 mg/mL BSA at 25° C. for 10 min. The beads were washed once with 1× phosphate buffer, 0.1% Tween 20, twice with 0.1 M NaOH+0.1% Tween 20, and twice with 1× phosphate buffer, 0.1% Tween 20. Then, the extended recording tags were amplified and analyzed by nucleic acid sequencing.

Sequencing of recording tags after the encoding cycle was used to estimate fractions of the recording tags being extended (encoded) during primer extension reactions. The efficiencies of the encoding reactions were evaluated based on yield (based on fractions of recording tag reads contained barcode information of the coding tag (encoded)) and background signal (fractions of recording tag reads contained barcode information that are associated with a non-cognate peptide).

By performing the described assay, a heatmap array may be generated for each binder, where each cell of the array represents an encoding efficiency of the given binder that binds to a specific combination of P1-P2 residues of the target peptide. The encoding data (fractions of the recording tags being encoded) were collected in parallel for the immobilized set of at least 289 peptides (17×17 combination of different P1 and P2 residues) and plotted as two-dimensional matrix for diverse P1-P2 combinations. An example of heatmap data for a representative “IL” binder is shown on FIG. 16A, which shows results of multiple parallel encoding reactions for the binder engineered from a metalloenzyme scaffold to specifically recognize M64-modified I and L NTAA residues of target peptides. Other examples of heatmap data for representative binders are shown on FIG. 16B-FIG. 16G, which were designed to specifically recognize as a mixture most M64-modified NTAA residues of target peptides. Encoding efficiencies are shown as black/white gradient, wherein the more intense white color represents higher encoding efficiency. To generate FIG. 16A-FIG. 16G, the encoding assay was performed essentially as described above in the Example by using the mixture of binders having sequences set forth in SEQ ID NOs: 1-7. Binders have the following specificities and concentrations in the encoding assay: SEQ ID NO: 1 shown in FIG. 16A—200 nM, SEQ ID NO: 2 shown in FIG. 16B—300 nM, SEQ ID NO: 3 shown in FIG. 16C—299 nM, SEQ ID NO: 4 shown in FIG. 16D—299 nM, SEQ ID NO: 5 shown in FIG. 16E—192 nM, SEQ ID NO: 6 shown in FIG. 16F—300 nM and SEQ ID NO: 7 shown in FIG. 16G—300 nM. More details about generation of the binders can be found in US 2022/0283175 A1, incorporated by reference. FIG. 16A-FIG. 16G illustrate “imperfectness” of recognition of modified NTAA residues by engineered binders. For example, the encoding data show multiple encoding signals outside the desired specificity of individual P1 residues generated from recognition of certain P1-P2-peptides, where encoding signal of P1 (efficiency of encoding) depends on P2 residue.

In some embodiments, using such imperfect binders may generate a large probabilistic variety of peptidic reads converted from sets of binder identifier strings, when conversion of each individual binder identifier string generates a large number of peptidic reads (1 to multiple conversion ratio) due to ambiguous binding profiles for the binders that correspond to binder identifiers assigned in a given binder identifier string. Alternatively, in some embodiments, there is no need for conversion of binder identifier strings to peptidic reads. In these embodiments, binder identifier strings are provided as input to a computer model, wherein the computer model is configured to infer amino acid sequences of polypeptides of the plurality of polypeptides from binder identifier strings (see Examples 8, 9 and 10 below).

### Example 7. Evaluating Efficiency of Encoding Assay Parameters

This Example shows exemplary methods for how common encoding assay parameters, such as the efficiencies of NTAA functionalization (NTF), NTAA cleavage (NTC) and NTAA encoding (NTE) may be determined. Depending on the experimental conditions and particular reagents used in the encoding assay, these parameters may depend on the P1 (i.e., NTAA) residue of polypeptide analytes, may depend on P1-P2 terminal dipeptide of polypeptide analytes, or may depend on the entire amino acid sequence of polypeptide analytes. In preferred embodiments, experimental conditions and reagents used in the encoding assay are engineered so that the dependence of NTF, NTC and NTE parameters on amino acid sequences beyond the P1-P2 terminal dipeptide of polypeptide analytes is minimized. For example, binding agents specific for modified terminal residues of polypeptide analytes can be engineered so that they predominantly bind to modified P1-P2 residues, but not to residues outside of the P1-P2 terminal dipeptide. Engineering of such binding agents is described in US 2023/0220589 A1 and US 2022/0283175 A1, which is incorporated herein by reference. Also, the experimental conditions of the encoding assay may be adjusted to include, e.g., a protease treatment, a solid support attachment method, a mild detergent, an increased temperature, or other conditions that would at least partially unfold the polypeptide analyte structures and make polypeptide termini accessible to modification, interaction with a binding agent and subsequent cleavage.

In some embodiments, particular experiments or sets of peptides are used to experimentally determine the efficiency of the NTF, NTC and NTE processes that encompass the encoding assay. The calculated parameters may then be applied as an input to a computer model and optionally used for training of a machine learning model. In other embodiments, a large number of diverse peptides of known sequences are utilized and data about the output of the encoding system are collected, without any particular mechanistic model, to instead model the encoding system by a neural network or other similar structure, where the training of the model utilizes the data to learn how to predict the output of the encoding system (e.g., binder identifier string) based on the peptide sequence (see more in Example 10 below).

In some embodiments when binding agents predominantly bind to modified P1-P2 residues, efficiencies of NTAA encoding (NTE) can be determined by calculating binding agent encoding efficiencies for individual P1/P2 combinations on model peptides, essentially as described in Example 6 above. To perform this for this example, a set of at least 289 peptides (17×17 combinations of different P1 and P2 residues) modified with a specific N-terminal modifier agent was synthesized and immobilized on a solid support associated with an individual nucleic acid recording tag that comprises a UMI sequence (a polypeptide barcode). These peptides were assessed against a set of 7 binding agents (binders) mixed together, where each binding agent (i) is engineered from a human carbonic anhydrase (hCA) scaffold as described in US 2023/0220589 A1, (ii) has specificity towards one or more particular NTAA residues, and (iii) is attached to a nucleic acid coding tag that comprises identifying information regarding the binding agent. When the set of binders is allowed to bind the set of peptides, binders compete with each other for the target modified NTAA residues present on the immobilized peptides. Upon binding of a binder to a particular peptide, the coding tag of the binder come to proximity with the recording tag attached to the peptide, and upon addition of a joining enzyme (e.g., ligase or topoisomerase), barcodes from the coding tag and the recording tag may be combined in a single di-tag construct, such as an extended recording tag (transfer of identifying information), followed by amplification and analysis. In addition to the transfer of coding tag barcodes, a null barcode was transferred to recording tag that did not undergo the extension during specific binding events. The process of binding and encoding (transfer of identifying information) has been repeated at least 3 times. All the extended recording tags are sequenced by NGS, and the encoder barcode sequences are decoded to obtain information regarding the binding events. For each P1-P2 combination (which correspond to a particular peptide), one or more barcodes (binder identifiers) may be recovered from nucleic acid sequences of extended recording tags. In some cases, there was no specific binding strong enough to support the encoding event, and thus, a blank binder identifier is inferred from nucleic acid sequences of extended recording tags. In other cases, there was a specific binding event that resulted in the encoding event, and the corresponding binder identifier is inferred from nucleic acid sequences of extended recording tags. In cases where two or more binders compete for the same P1-P2 dipeptide, more than one binder identifier may be identified when the encoding is repeated a few times or more. Analysis of nucleic acid sequences of extended recording tags can be used to associate each binder identifier inferred from the nucleic acid sequences with amino acid identities of P1 and P2 residues, and thus, heatmaps of data for each binder may be created demonstrating relative binder specificities and affinities towards P1-P2 terminal dipeptides. FIG. 16A-FIG. 16G show exemplary heat map data obtained as described in (see). Importantly, these relative specificities may be changed when binders are tested individually, at different concentrations, or within a different set of binders, because different binder combinations may generate a different competitive landscape for each of the P1-P2 dipeptides. Encoding yield has been calculated for each binder and each peptide (that differs from other peptides by a unique P1-P2 dipeptide) as the number of times a binder identifier is seen attached to a peptide barcode (in the plurality of nucleic acid recording tag sequences) divided by the number of times the peptide barcode is seen in the nucleic acid recording tag sequences. The brighter the color in FIG. 16A-FIG. 16G the higher the encoding yield is for a specific P1-P2 terminal dipeptide. The figures demonstrate the encoding-linked binding specificities of each binder towards a plurality of P1-P2 terminal dipeptides in polypeptides.

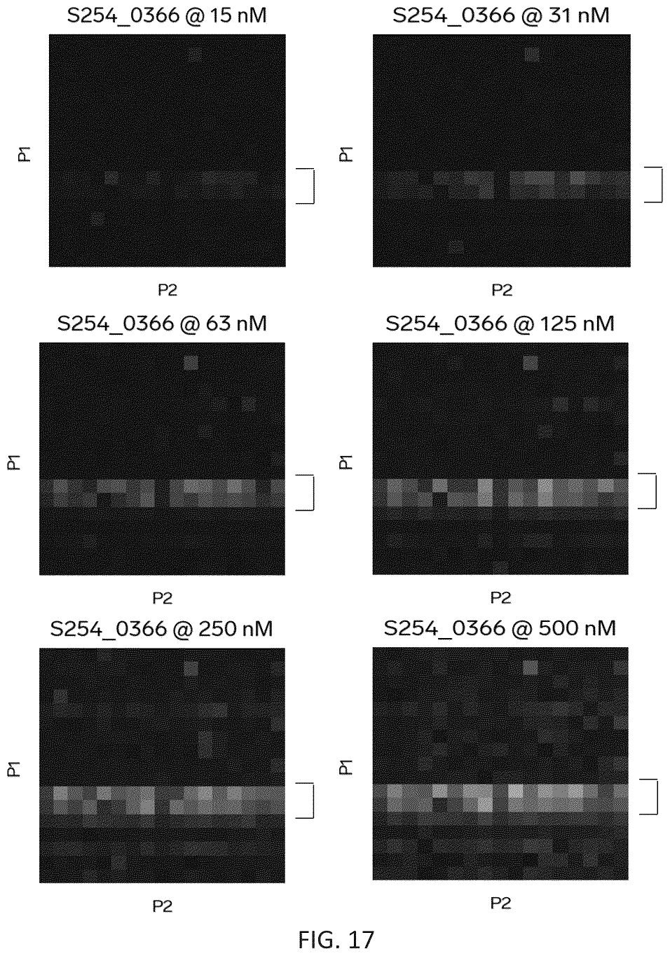

As an alternative method, instead of employing a set of binders simultaneously, each binder may be tested individually at different concentrations, the encoding signals recorded, and based on their intensity, a set of binders may be assembled to cover most of the P1-P2 dipeptides at comparable levels. An example of a binder titration experiment is shown in FIG. 17, where it is shown how the encoding signal is changing depending on the binder concentration. To generate FIG. 17, the encoding assay was performed multiple times under essentially the same conditions (as described in the Example 6 above) by using the individual binder having amino acid sequence set forth in SEQ ID NO: 7 at different concentrations indicated in the panels of FIG. 17.

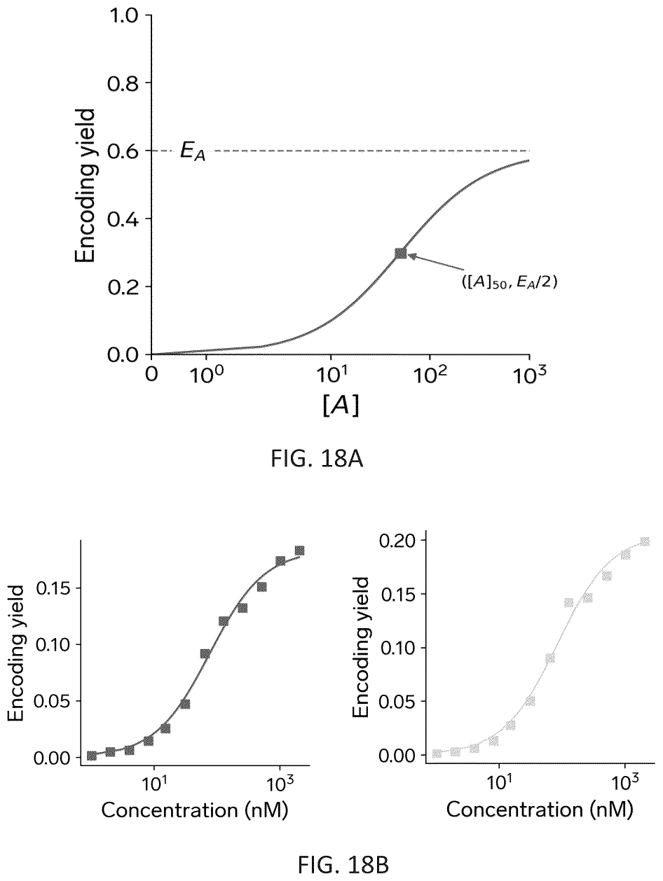

In some embodiments, a set of binders can be modeled computationally. In some embodiments, a set of binders may be devised based on individual binder binding profiles. For example, by fitting kinetic equations to data from the individual binder titration experiments, a mixture can be computationally devised by calculating the encoding for each target using a competitive variant of the Hill equation—where the concentrations of all the binders considered are the parameters of the model and the encoding yields are the outputs. Then it is a matter of finding which combination(s) of particular binders and their concentrations perform optimally given the desired outcome, such as the highest yield for particular targets while minimizing off-target binding. In some embodiments, encoding yield can be fit with a kinetic equation (e.g., the Hill equation). For example, from the titration series of a binder (see, e.g., FIG. 17), the values can be fit to a suitable kinetic equation using, for example, a least squares method in a standard numerical optimization library. In a particular example, FIG. 18A shows modeling of an encoding yield for an individual binder depending on the binder concentration [A]. The encoding yield for an individual binder may be determined based on the following equation:

$${e}_{A} ( [ A ] ) = \frac{{E}_{A}}{1 + \frac{{[ A ]}_{50}}{[ A ]}}$$

Experimental data for individual binders obtained by the encoding assay as described in Example 6 are compatible with predictions produced by the described model—see FIG. 18B, which shows experimental data for concentration-dependent encoding yields of two individual exemplary binders that fit with the described equation.

Summarizing the above, binder specificity profiles (or simply binding profiles) may be determined based on data produced by performing multiple encoding assays using a reproducible set of experimental conditions and a predetermined set of binders. Importantly, the binder specificity profile may change where composition of the set of binders is changed, or where concentration of individual binders within the set is changed. In some embodiments, binder specificity profiles may be determined in advance (i.e., predetermined) and these parameters are inputted into a machine learning model together with binder identifiers of the plurality of binder identifier strings in order to infer amino acid sequences of polypeptides from the binder identifiers. In other embodiments, binder specificity profiles may not be determined in advance, and instead may be computed within the machine learning model if sufficient encoding data are used to train the model. In some embodiments, such encoding data can include “ground truth” data on model polypeptides together with encoding data on the model polypeptides, which allows for validating the computer model and computing binder specificity profiles and other encoding assay parameters, such as efficiencies of NTF and NTC (see more discussion below). In some particular embodiments, nucleic acid sequences of the plurality of nucleic acid sequences each contain a polypeptide barcode (UMI) in addition to the series of encoder barcode sequences, and the identity of the polypeptide can be inferred from nucleic acid sequence of the polypeptide barcode. Thus, the polypeptide identity inferred from the polypeptide barcode may be compared with one or more polypeptide identities predicted from analysis of the encoder barcode sequences. This can be used to evaluate or validate a machine learning model used to predict identities of polypeptides present in a sample. This can also be used in training of the machine learning model, where the machine learning model can be trained using data on model peptides which are either separately processed or included within a sample of unknown polypeptides (see Example 10 for further details).

In some embodiments, in addition to determining binder specificity profiles, efficiencies of the NTF step are also determined. In some of these embodiments, each binder of the set of binders used in the encoding assay binds to a terminal amino acid (e.g., NTAA) residue modified with an N-terminal modifier agent. In other embodiments, each binder of the set of binders used in the encoding assay binds to a terminal dipeptide (e.g., the P1-P2 residues) modified with an N-terminal modifier agent. The efficiency of such modification reactions (e.g., functionalization of the terminal amino acid to provide better affinity for a specific binder) may depend on the particular P1 residue, depend on the P1-P2 terminal dipeptide of polypeptide analytes, or may depend on entire amino acid sequence of polypeptide analytes. In some embodiments, NTF efficiencies may depend mostly on P1-P2 terminal dipeptide of polypeptide analytes.

NTF efficiencies for a particular N-terminal modifier (NTM) agent acting on a plurality of peptides having various P1-P2 terminal residues may be determined by various methods. In one example, an antibody may be obtained to recognize the structure of N-terminal modifier (NTM) agent installed on the terminus of a peptide. Then, this antibody may be used to quantify conjugation rates of the NTM agent to different P1-P2 peptides, whereas synthesized NTM agent-peptides are used as normalization controls.

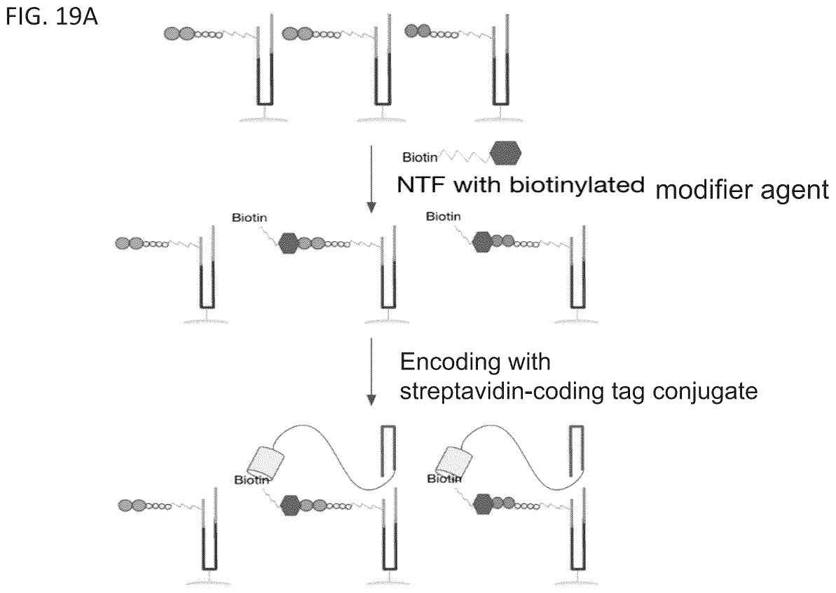

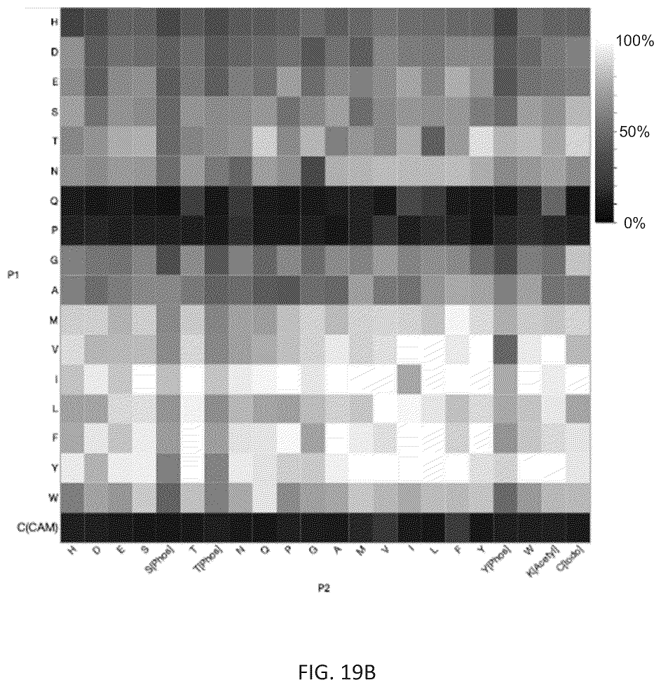

In another example, conjugation rates of the NTM agent to different P1-P2 peptides are determined based on a variation of the encoding assay using model peptides each immobilized on a solid support (see FIG. 19A-FIG. 19B). An NTM agent conjugated with a biotin via a flexible linker is attached to different P1-P2 peptides, each associated with a nucleic acid recording tag and immobilized on beads as described in Example 6 above. In this example, NTM agent was allowed to interact with P1 residues of the peptides for 1 h and unbound molecules were washed away. Next, a streptavidin-coding tag conjugate was applied and upon binding of streptavidin to biotin, coding tag interacted with a recording tag located in proximity forming an extended recording tag (FIG. 19A). All recording tag constructs formed during the binding step were collected and sequenced via NGS and corresponding “encoding yield”, which in this case is a fraction of extended recording tags compared to all recording tags collected for a given P1-P2 dipeptide. FIG. 19B shows correspondent NTF P1-P2 profiling (heatmap) data collected in this experiment (only K and R residues were excluded from the experiment based on the particular peptide sample preparation method utilizing a trypsin digestion of proteins under the assumption that these residues are likely to be present only at peptide C-termini). Low NTF efficiency was observed for some P1-P2 combinations. In particular, P1 residues such as Pro, Glu and Cys(CAM) act as a poor substrate for this NTM agent. Glu may form pyroglutamate, which may affect NTF efficiency.

In summary, using the described approaches, NTF efficiency of a particular NTM agent may be assessed and used as an input to a machine learning model to perform inference of amino acid sequences of polypeptides based on binder identifier strings obtained by a given encoding assay (see Examples 8, 9 and 10).

The P1-P2 peptide arrays described here and produced as shown in Example 6 above may be used not only to estimate NTF efficiency, but also to estimate efficiencies of cleavage of the modified NTAA residues at the end of each encoding cycle (also called NTC efficiency). In some embodiments, after encoding, modified NTAA residues are cleaved by a set of engineered Cleavase enzymes, thereby exposing new NTAA residues of the immobilized peptides (see U.S. patent Ser. No. 11/427,814 B2 and 11,788,080 B2). In other embodiments, a chemical cleavage of modified NTAA residues may be employed (see US 2022/0227889 A1, incorporated by reference). In most cases, efficiency of the modified NTAA residues cleavage varies depending on P1-P2 residues of peptide substrates. The advantage of using engineered Cleavase enzymes is that these enzymes may be evolved to recognize different sets of modified NTAA residues, so that a set of Cleavase enzymes may be assembled that work uniformly across all or most P1-P2 residues of peptide substrates (see U.S. patent Ser. No. 11/427,814 B2 and 11,788,080 B2). In addition, these enzymes may be evolved to recognize and cleave only modified NTAA residues but not native NTAA residues of peptide substrates (see U.S. patent Ser. No. 11/427,814 B2). This allows one to control the cleavage rate of these enzymes so as to cleave a single amino acid residue at a time. After the cleavage of a modified NTAA residue, a Cleavase enzyme may stop and not proceed with cleavage of a next residue (e.g., the former P2 residue that becomes a new NTAA residue after cleavage) until this next residue undergoes modification in the next encoding cycle.

As an example of an NTC efficiency assessment for a particular Cleavase enzyme, two (identical) P1-P2 peptide arrays were prepared as described above and the P1 residues of the immobilized peptides were modified with an NTM agent. Peptides on the first array were incubated in 25 mM EPPS (pH=8.5) buffer (a Good's buffer) at 45° C. for 1 h (control) and then encoded with a mixture of 400 nM binding agents that are configured to bind to most modified NTAA residues of the immobilized peptides (see, e.g., FIG. 16A-FIG. 16G). Peptides on the second array were first cleaved by a Cleavase enzyme (10 nM) in 25 mM EPPS pH=8.5 buffer at 45° C. for 1 h, which is configured to cleave off the NTM-NTAA moiety, and then encoded with a mixture of the same binding agents (400 nM) that are configured to bind to most modified NTAA residues of the immobilized peptides. Encoding yields for each P1P2 peptide on the first array and on the second array were assessed. If the cleavase enzyme cleaves a particular P1P2 peptide efficiently, then the encoding yields for this peptide on the second array is expected to be negligible, since binding agents would not recognize unmodified terminal residues efficiently enough to produce an encoding event. The Cleavase enzyme efficiency (which is a loss of encoding) may be calculated by assessing two samples (with and without performing cleavage) using the following approach:

$$\begin{matrix}( no cleave ) \text{?} = \text{?} P \vert ij 〉 \\ = \text{?} ( {P}_{ij} \vert + ij 〉 + ( 1 - {P}_{ij} ) \vert ij 〉 ) \\ = \text{?} {P}_{ij}\end{matrix}$$

$$\begin{matrix}( cleave ) \text{?} = \text{?} CP \vert ij 〉 \\ = \text{?} C ( {P}_{ij} \vert + ij 〉 + ( 1 - {P}_{ij} ) \vert ij 〉 ) \\ = \text{?} {C}_{ij} {P}_{ij} \vert jA 〉 + ( 1 - {C}_{ij} ) {P}_{ij} \vert + ij 〉 ) \\ = \text{?} ( 1 - {C}_{ij} ) {P}_{ij}\end{matrix}$$

$$\text{?} \text{indicates text missing or illegible when filed}$$

In these equations, P indicates a function that reflects modification efficiency of a particular P1-P2 peptide (NTF efficiency); index “i” indicates P1 residue; index “j” indicates P2 residue; C indicates a function that reflects cleavage efficiency of a particular P1-P2 peptide (NTC efficiency); E indicates a function that reflects encoding probability for a particular P1-P2 peptide (NTE efficiency); and e indicates experimental (actual) encoding yield of a particular P1-P2 peptide. Based on the equations, the NTC efficiency can be calculated as

$${C}_{ij} = 1 - \frac{\text{?}}{\text{?}}$$

$$\text{?} \text{indicates text missing or illegible when filed}$$

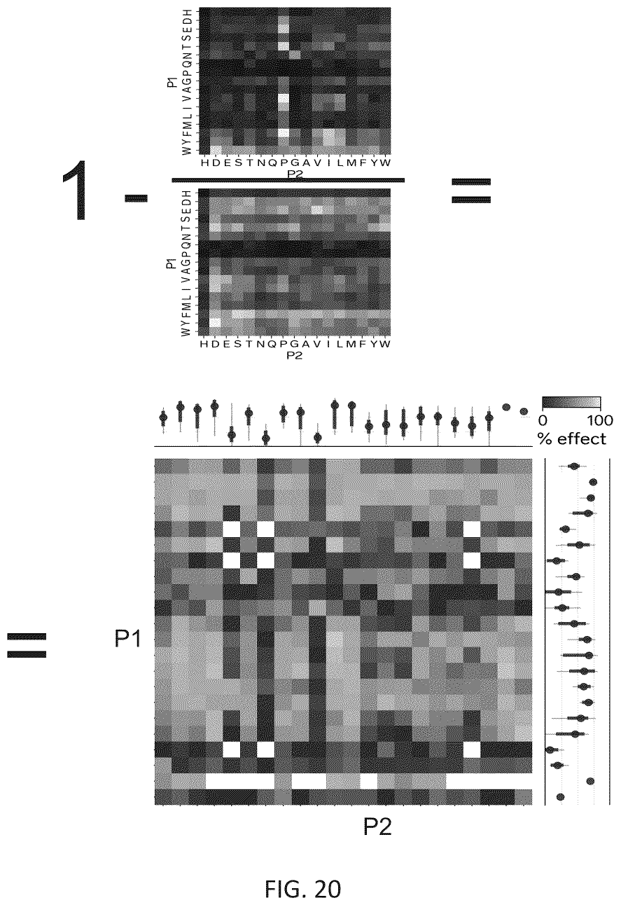

Actual encoding data from two P1-P2 peptide arrays described above were collected and, based on these data, the NTC efficiencies of P1-P2 peptides were calculated (see FIG. 20, where the upper heatmap corresponds to the experimental encoding yields corresponding to different combinations of P1 and P2 residues performed with the Cleavase enzyme cleavage, the middle heatmap corresponds to experimental encoding yields when the assay was performed under the “no cleavage” condition, and the larger heatmap at the lower right shows data for efficiencies of the Cleavase enzyme used the assay for P1-P2 residues calculated according to the Formula above (which calculates ratios between “with the cleavage” and “without the cleavage” encoding yields for P1-P2 residues)). Bright color indicates a higher NTC efficiency. A similar approach may be implemented for determining cleavage efficiencies for a set of Cleavase enzymes applied as a mixture.

In summary, using the described approaches, NTC efficiencies of a given Cleavase enzyme (or a given set of Cleavase enzymes) towards a variety of P1-P2 peptides may be assessed and used as an input to a computer model to perform inference of amino acid sequences of polypeptides from binder identifier strings obtained by a given encoding assay (see Examples 8, 9 and 10 below).

### Example 8—Assigning Binder Identifier Strings to Amino Acid Sequences of Polypeptides Potentially Present in the Analyzed Sample(s) “Top-Down” Approach

This Example describes exemplary algorithms that may be utilized for inferring amino acid sequences of polypeptides of the plurality of polypeptides (all polypeptides that can potentially be present in the analyzed sample(s)) from binder identifier strings generated during analysis of a plurality of nucleic acid sequences received at one or more processors. As described above, the plurality of nucleic acid sequences can be generated by performing an encoding assay which comprises n encoding cycles, wherein n is typically between 3 and 20, and preferably between 6 and 10, inclusive. In preferred embodiments, during each cycle, terminal amino acids of polypeptide analytes that are bound by binders conjugated with identifying coding tags are encoded into nucleic acid sequences attached to recording tags, each associated with a corresponding polypeptide analyte. Encoding occurs by transferring identifying information regarding the corresponding binder from the coding tag to the recording tag associated with the polypeptide analyte. In preferred embodiments, after performing n cycles of the assay, the recording tag associated with the polypeptide analyte has been extended n times (extended in each cycle with a corresponding sequence comprising the identifying information for the binder). Also, after performing each encoding cycle, the terminal amino acids of polypeptide analytes are cleaved, thereby generating newly formed terminal amino acids of the polypeptide analytes. The above description is a variant of an encoding assay (such as the encoding assay for polypeptide analysis described elsewhere herein).

An exemplary binder identifier string association algorithm suitable for analysis of a large number of polypeptide analytes (such as at least 100, at least 1000, at least 5000, at least 10000, at least 100000, at least 1000000 or more polypeptide analytes) encoded in a plurality of nucleic acid sequences by an encoding assay is described below.

First, the plurality of nucleic acid sequences generated from the encoding assay is received as an output of the encoding assay (i.e., as nucleic acid sequences determined by sequencing the extended recording tag molecules that are generated during the encoding assay), where each of the plurality of nucleic acid sequences comprises a series of encoder barcode sequences, and where each barcode sequence of the series of encoder barcode sequences corresponds to a binder that binds to a component of a polypeptide (e.g., one or more amino acid residue(s) or modification thereof) of the plurality of polypeptides in the encoding assay;

Next, for each barcode sequence within a given nucleic acid sequence, the identifying information regarding corresponding binder is extracted and stored. In some embodiments, a binder identifier is assigned to each of the series of encoder barcode sequences in each of the plurality of nucleic acid sequences. In preferred embodiments, a plurality of corresponding binder identifier strings is generated for the plurality of nucleic acid sequences. Alternatively, rather than generating binder identifier strings in advance, each binder identifier can be analyzed separately and in view of the order in which encoder barcode sequences are present in the nucleic acid sequences. The order of encoder barcode sequences is important to take into account since it provides information about the order of the encoded amino acid residues in the analyzed polypeptides.

The plurality of generated binder identifier strings then needs to be matched with the amino acid sequences of polypeptides of the plurality of polypeptides in order to identify and/or quantify at least some polypeptides present in the plurality of polypeptides. The problem with simple matching is that the specificity of each binding agent is not absolute. In fact, most, if not all, binding agents will bind with some probability to one or more non-cognate components of a polypeptide (e.g., to one or more NTAA residues other than one it is configured to bind). Also, for a multicycle assay, a newly formed NTAA residue of a polypeptide is encoded in each binding-transferring cycle, and the transition from one cycle to another cycle may be impaired if the encoded NTAA is not cleaved after the completion of the current cycle. Finally, if modification (functionalization) of the NTAA residue is required before binding and/or cleavage, the efficiency of the modification reaction needs to be taken into account for each residue. The methods described this Example and in Example 9 below are suitable to address all of these issues, and can be used to perform inference of amino acid sequences of polypeptides present in the plurality of polypeptides based on binder identifier strings obtained via an encoding assay.

In order to infer amino acid sequences from binder identifiers, the computer model described in this Example performs the following steps using amino acid sequences present in the plurality of polypeptides, (i) generating a plurality of simulated binder identifier strings using one or more parameters (e.g., the efficiencies of NTAA functionalization (NTF), NTAA cleavage (NTC) and NTAA encoding (NTE)) of the encoding assay; (ii) for one or more simulated binder identifier strings of the plurality, generating a probability score based on a probability that a given simulated binder identifier string is associated with one or more amino acid sequences present in the plurality of polypeptides; (iii) matching each of the plurality of binder identifier strings to the one or more simulated binder identifier strings based on the calculated probability scores for the one or more simulated binder identifier strings, and (iv) based on the generated probability scores for the simulated binder identifier strings, outputting data related to at least a partial identity and/or quantity for at least one polypeptide of the plurality of polypeptides (see FIG. 4C). These steps are described in more details below.

In some embodiments, the first step performed by the computer model (Indexing) to generate a plurality of simulated binder identifier strings is to simulate the encoding assay in silico using a collection of input polypeptides (e.g., polypeptides, tryptic digests of polypeptides, peptides, etc.) to generate a large lookup index table comprising k-mer fragments for each polypeptide sequence and probabilistic distribution of binder identifier strings that may be generated from each k-mer fragment during the simulated encoding assay.

First, a database of polypeptides or components thereof (e.g., tryptic peptides) is selected, which encompasses the amino acid sequences of all polypeptides potentially present in the analyzed sample(s). For each polypeptide in the database, a set of k-mer fragments is created. K-mer fragments (or k-mers) are built from a particular polypeptide sequence as follows: for an exemplary amino acid sequence “A1A2A3A4A5A6A7”, all k-mers of length 5 (5-mers) would be A1A2A3A4A5, A2A3A4A5A6 and A3A4A5A6A7. The parameter k should be equal to or less than n (where n is a number of cycles performed in the encoding assay which generated the analyzed plurality of nucleic acid sequences); k is typically between 3 and 8, and preferably between 4 and 6, inclusive.

Next, considering the generated k-mer fragments as an input, the encoding assay is simulated in silico using the empirically determined (or predicted by molecular modeling) parameters of the “real” encoding assay that was used to generate the analyzed plurality of nucleic acid sequences. Among the empirically determined parameters (or those predicted by molecular modeling) of the encoding assay are at least the following: 1) probabilities of encoding each potential N-terminal amino acid residue in a given k-mer fragment either correctly or incorrectly (i.e., “emission” probabilities) based on the binding profiles of the binders used in the encoding assay (thereby assigning for a given amino acid position in the given k-mer fragment either a binder identifier for the same amino acid residue or, optionally, at least one different binder identifier, with a certain probability; this would correspond to generating one or another encoder barcode sequence in the extended recording tag associated with a peptide in a real encoding assay); and 2) for each potential N-terminal amino acid residue in a given k-mer fragment, a probability of successfully cleaving the N-terminal amino acid residue after a binder binds to the N-terminal amino acid residue, thereby exposing a newly formed N-terminal amino acid residue in the given k-mer fragment (i.e., “transition” probabilities). As discussed above, there are inherent ambiguities during recognition of NTAA residues by binders; also, NTAA cleavage efficiencies depend on P1 and P2 residues of the given k-mer fragment. Thus, the conditions of the simulated encoding assay can closely mimic the conditions of the real encoding assay used to produce the analyzed plurality of nucleic acid sequences.

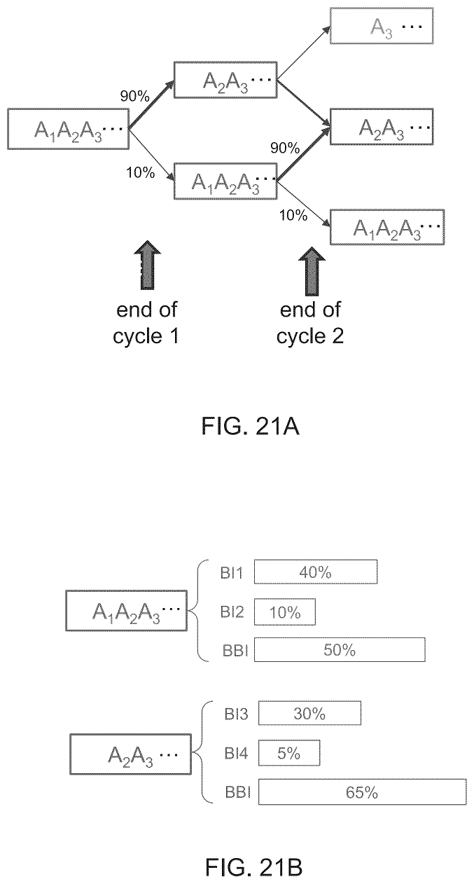

In preferred embodiments, k cycles of the encoding assay are simulated for each generated k-mer fragment. Based on the previously determined (or estimated) probabilities for each binding or cleavage event, each starting k-mer fragment will produce at least one binder identifier assignment for the current NTAA residue during each cycle of the simulated encoding assay (see, e.g., FIG. 21A-B). Each assignment would incorporate a probability of the described binding event. For example, in a first encoding cycle (as illustrated in FIG. 21B), when a plurality of less selective binders are employed during the encoding assay, a first NTAA residue of a particular k-mer may be bound either by binder1 (with 40% probability), or by binder2 (with 10% probability). Finally, with 50% probability (listed probabilities are arbitrary numbers used for illustration purposes only) no binding occurs in the first encoding cycle. Binding by binder1 would assign binder identifier 1 (BI1) to the first NTAA residue. Binding by binder2 would assign binder identifier 2 (BI2) to the first NTAA residue. No binding would assign a blank binder identifier (BBI) to the first NTAA residue. Therefore, the first cycle would produce 3 independent assignments in the simulated encoding assay, where each assignment has a certain probability that may be calculated or estimated empirically in advance. Further steps of the assay would include cleavage of the first NTAA residue (as illustrated in FIG. 21A), which could occur with 90% probability. Therefore, in the next cycle, the same NTAA residue would remain in this k-mer fragment with 10% probability, whereas with 90% probability a new (second) NTAA residue will be formed in this k-mer fragment (e.g., the former P2 residue of the k-mer). Then, the assignment based on current NTAA-binder interactions will continue for the next encoding cycle, similar to described above. In the scenario where no cleavage occurred during the first cycle, the first NTAA would again interact with either binder1 (with 40% probability), or binder2 (with 10% probability), or no binding occurs (with 50% probability). In the scenario where the cleavage the first NTAA occurred, the second NTAA would interact with either binder3 (with 30% probability), or binder4 (with 5% probability), or no binding occurs (with 65% probability) (listed probabilities are again arbitrary numbers). Binding by binder3 would assign binder identifier 3 (BI3) to the second NTAA residue, and binding by binder4 would assign binder identifier 4 (BI4) to the second NTAA residue. Therefore, after the second encoding cycle, the following assignments are generated during the simulation (accounting for both the first NTAA residue cleavage and no first NTAA residue cleavage scenarios): for NTAA cleavage: BBI-BBI (32.3% probability), BBI-BI3 (13.5% probability), BBI-BI4 (2.25% probability), BI1-BBI (23.4% probability), BI1-BI3 (10.8% probability), BI1-BI4 (1.8% probability), BI2-BBI (5.85% probability), BI2-BI3 (2.7% probability), and BI2-BI4 (0.45% probability), BBI-BI1 (2% probability), BBI-BI2 (0.5% probability), BI1-BBI (2.6% probability), BI1-BI1 (1.6% probability), BI1-BI2 (0.4% probability), BI2-BBI (0.5% probability), BI2-BI1 (0.4% probability), and BI2-BI2 (0.1% probability). See FIG. 21A-FIG. 21B for illustration of the described simulation.

The second step of the assay would include cleavage of the second NTAA residue, which could occur with NN % probability (which may be different from the probability of the first NTAA residue cleavage, since the cleavage efficiency may vary depending on amino acid residue side chain). The simulated encoding assay continues until all k encoding cycles are evaluated, thereby generating a plurality of simulated binder identifier strings having an associated probability distribution, where each simulated binder identifier string has a length of k binder identifiers. In some embodiments, instead of binder identifiers, nucleic acids corresponding to barcodes or other corresponding codes may be employed, but the described analysis would be similarly performed.

This example simulates one exemplary encoding process to allow generating probability distributions for a model peptide processing. Other variations of encoding assay steps are possible and will affect the calculation of probability distributions. In some embodiments, the first step may include potential outcomes for functionalization of a N-terminal amino acid residue prior the encoding cycle. Other steps may include potential outcomes for cleavage of an N-terminal amino acid residue after the encoding cycle has been performed. The described simulation process may be combined with the potential outcomes for assigning different NTAA variants due to imperfect binding.

In some embodiments, an explicit probability of occurrence for each binder identifier string may be determined. By using a probability cut-off, low probability binder identifier strings may be removed from further analysis. In some examples, the probability cut-off may be 10⁻⁶, 10⁻⁷, or 10⁻⁸. In other embodiments, the generated plurality of simulated binder identifier strings may be sampled, preferably multiple times, in order to generate estimates of the variance of the kmer:binder identifier string relationships.

Based on the determined probabilities of occurrence for at least a portion of the plurality of simulated binder identifier strings generated for a given k-mer, a large look-up table is assembled, where rows indicate different generated k-mer fragments, and columns indicate different binder identifier strings for each k-mer fragment in the index table. The table can also indicate the probabilities (e.g., expressed as percentages or fractions) of given binder identifier strings being produced for each k-mer fragment in the simulated encoding assay.

In some embodiments, the generated look-up table is inversed, i.e., rearranged by reciting probabilities for each binder identifier string being generated from each of the k-mer fragments in the table.

In some embodiments, after all kmers for a given polypeptide are processed, the probabilities of occurrence for the simulated binder identifier strings are divided by the number of kmers for that polypeptide, so that probability values are correctly normalized across all polypeptides, thereby associating simulated binder identifier strings with polypeptides.

In some embodiments, for computational efficiency, only a certain number of polypeptides associated with a given simulated binder identifier string may be kept in the look-up table. This number could be, for example, no more than 10, no more than 50, no more than 100, or no more than 200. In some embodiments, only polypeptides associated with simulated binder identifier strings having probability values that meet a specified threshold (e.g., at least 1/threshold times that current most probable hit) are kept. In some embodiments, polypeptides that are kept in the look-up table may be determined by rank-ordering “the top N” (e.g., the top 10, 50, 100, or 200 based on the rank-ordered probabilities), but for further efficiency this list may be further be pruned so that entries that fall below some level such as the (“top N”)/(a specified threshold) (e.g., the (top 10,000)/1000) are removed.

In the second step performed by the matching algorithm (Aligning), each binder identifier string generated by processing the plurality of nucleic acid sequences generated from a real encoding assay is split into binder identifier k-mers of the same length as used in the indexing step.

Next, the binder identifier k-mers are compared to the look-up table to identify probabilities that a given binder identifier k-mer came from a particular peptidic k-mer fragment.

In some embodiments, each binder identifier string can be associated with several polypeptides. When probabilities that a particular binder identifier k-mer came from more than one polypeptide are identified from the look-up table, these probabilities can be merged. There are a few different modes in which these probabilities can be merged, including, for example: i) they can be multiplied together for each binder identifier k-mer; or ii) they can be multiplied together in a weighted manner based on the current estimated relative abundances of the different polypeptides in the sample.

Next, an alignment of a given binder identifier k-mer to a particular peptidic k-mer fragment is called if a particular threshold of probabilities is met. This can include, for example: i) that the most probable polypeptide computed in the previous step has a probability that is some multiple of the probability for the second most probable polypeptide, e.g., 10×, 100×, 1000× more likely; and ii) the most probable polypeptide computed in the previous step has a probability that is some multiple of the probability for all other polypeptides, e.g., 10×, 100×, 1000× more likely.

In some embodiments of the disclosed “top-down” method, a simplified version of the computer model may be employed. This may be useful for analyzing binder identifier string data for less complicated starting samples, such as samples containing 1000 or less polypeptides. The same steps may be employed as described in the present Example above, including simulation of the encoding assay, but without fragmenting polypeptides and binder identifier strings to k-mer fragments. Using a lower number of starting polypeptides would allow the system to simulate the encoding assay and associate binder identifier strings generated from polypeptides with amino acid sequences of polypeptides of the plurality of polypeptides on the level of polypeptides, and not k-mer fragments.

In one specific example, a protein sample was analyzed by the disclosed algorithm that contain some of the 200 most abundant proteins in human plasma. An encoding assay was performed under conditions that were tested in advance and where key parameters of the encoding assay (NTF, NTC and NTE) were experimentally determined as described in Example 7; namely, a) for each standard NTAA (i.e., P1) residue, efficiency of the NTM agent installation was determined; b) for each standard P1-P2 pair, efficiency of cleaving the modified P1 was determined; and c) for each standard P1-P2 pair, encoding rates for each binder of the set of six binders used in the encoding assay (as well as the rate of no encoding (null binder identifier)) were determined. Using these determined encoding assay parameters, an in silico encoding assay was performed, using the same conditions as in physical encoding assay, which included tryptic digestion of proteins present in the sample. Next, simulated binder identifier strings from the in silico assay were generated for each potential peptide that might be present in the sample after the trypsin digestion (derived from a protein present in the sample), and probabilities for each binder identifier string were calculated based on the determined encoding assay parameters. For example, for the (hypothetical) peptide “FLFVVAAATGVQSQVQLVQSGAEVK” (SEQ ID NO: 28), the 15 most probable binder identifier strings produced in the in silico encoding assay were:
ZZZZZZ 0.248736
FZZZZZ 0.082883
ZZFZZZ 0.066128
ZZZFZZ 0.052042
ZFZZZZ 0.048576
ZZZZFZ 0.030728
ZZZZZV 0.024058
FZFZZZ 0.023911
ZZZZVZ 0.021605
FZZFZZ 0.015958
ZZZZZF 0.015446
FFZZZZ 0.012543
ZFZFZZ 0.011553
ZZZVZZ 0.010773
ZFFZZZ 0.010542,

where Z corresponds to a null binder identifier (no encoding event). ZZZZZZ was the most probable binder identifier string because the encoding yields of the binders used in the encoding assay were all well below 50%.

Next, using simulated binder identifier strings produced in the in silico encoding assay based on all proteins potentially present in the sample and the calculated probability scores of these strings, the “real” binder identifier strings produced in the physical encoding assay were matched to the simulated binder identifier strings to infer amino acid sequences of peptides derived from proteins in the sample. For example, for the binder identifying string “FFZFVV” (SEQ ID NO: 29) the computer model returns the following peptides (SEQ ID NO: 28, SEQ ID NO: 30-SEQ ID NO: 33) that may produce this binder identifying string, as well as their probabilities (proteins that the peptides are originated from are indicated in the parentheses):

| | | |
|---|---|---|
| FLFVVAAATGVQSQVQ | (IGHV1-69) | 1.328863e−05 |
| LVQSGAEVK | | |
| FLVNLVK | (AFM) | 2.592956e−06 |
| FLVGPDGIPIMR | (GPX3) | 2.225403e−06 |
| LILVDYILFK | (SERPINA10) | 1.664864e−06 |
| FIQVGVISWGVVDVCK | (CFB) | 1.190012e−06 |

In this case, the top hit is about 5× more likely than the second hit, which gives a prediction of a protein present in the sample. Additional data may be used to confirm this prediction, for example, if the computer model returns other peptides of the same protein (IGHV1-69) based on analysis of other “real” binder identifier strings (i.e., strings produced by the actual encoding assay).

The disclosure presented in this Example so far illustrates only some specific ways how assigning binder identifier strings may work. Other ways are also possible and some of them are discuss below in Example 9.

### Example 9—Assigning Binder Identifier Strings to Amino Acid Sequences of Polypeptides by a “Bottom-Up” Approach

This Example describes exemplary algorithms that may be utilized for inferring amino acid sequences of polypeptides from binder identifier strings generated during analysis of a plurality of nucleic acid sequences received at one or more processors. As described above, the plurality of nucleic acid sequences is generated as a product of an encoding assay, which comprises n cycles, wherein n is typically between 3 and 20, and preferably between 6 and 10, inclusive. This example utilizes the same encoding assay elements as described in Examples 6-8, namely, utilizing a set of binders (binding agents), wherein each binder of the set binds to modified (functionalized) NTAA residues of polypeptide analytes with certain probabilities. Following binding of a binder to a modified NTAA residue of a polypeptide, this modified NTAA residue is encoded into a recording tag attached to the polypeptide by transferring an encoder barcode sequence of a coding tag attached to the binder to the recording tag. After encoding, the modified NTAA residue is cleaved by a set of Cleavase enzymes configured to cleave modified NTAA residues (generation of these enzymes are described in detail in U.S. patent Ser. No. 11/427,814 B2 and 11,788,080 B2, which is incorporated herein by reference), thereby exposing a new NTAA residue of the polypeptide. When such enzymes have efficiency biases towards the cleavage of particular modified NTAA residues, a set of the engineered enzymes can be employed that to cover the cleavage reaction for all (or most) standard NTAA residues (see U.S. Pat. No. 11,427,814 B2). In this example, an M64-specific cleavase set is used, comprising 3 engineered enzymes having amino acid sequences set forth in SEQ ID NO: 9-SEQ ID NO: 11, which were engineered from dipeptidyl peptidase from Thermomonas hydrothermalis (sequence set forth in SEQ ID NO: 8) as described in U.S. Pat. No. 11,427,814 B2.

Modification of NTAA residues of polypeptide analytes is beneficial to limit cleavage of terminal residues to just a single residue per single encoding cycle. In preferred embodiments, Cleavase enzymes are evolved/tailored to accommodate specific N-terminal modifications along with the NTAA residue in their substrate binding pocket, thereby preventing progressive cleavage of the penultimate terminal amino acid residue of the polypeptide analyte, unless the N-terminal modification is also attached to the terminal residue formed after cleavage of the original NTAA by a Cleavase enzyme (see U.S. Pat. No. 11,427,814 B2). Therefore, in preferred embodiments, terminal amino acid residues of the polypeptide analytes are modified before the next cycle of cleavage can occur.

In other embodiments, instead of using a Cleavase enzyme, the N-terminal amino acid may be removed using any of the chemical methods as described in in US 2020/0348307 A1, US 2022/0227889 A1, and U.S. Pat. No. 11,499,979 B2, all of which are incorporated herein by reference.

Next, the new NTAA residue is modified (functionalized) as shown in Example 6, then encoded using the set of binders, and the reaction cycle comprising of NTAA cleavage (NTC), NTAA functionalization (NTF) and NTAA encoding (NTE) is repeated one or more times. It can be seen that after N cycles, the following assay steps occur NTF-NTE-(NTC-NTF-NTE)\hat{ }N−1, which produce one or more nucleic acid sequences that each corresponds to a set of binding-encoding events on individual polypeptides, and that can further be referred to as “reads”. Each read comprises a plurality of encoder barcode sequences, where each barcode sequence corresponds to a binder that binds to a component of a polypeptide of the plurality of polypeptides in the encoding assay. In a computer-implemented method comprising the use of one or more processors, a plurality of binder identifier strings is generated from the plurality of nucleic acid sequences, where each binder identifier string consists of N binder identifiers, which reflects that, after performing the encoding reaction for N cycles, the recording tag associated with the polypeptide is extended N times (in optimal embodiments). Importantly, the probability of successful completion of each step (NTF, NTE and NTC) is typically less than 100%. For example, depending on the particular N-terminal modifier agent and particular NTAA residue, the efficiency of the NTAA modification (functionalization) reaction may be between 80% and 99.9%. Also, following binding of a binder to a modified NTAA residue of a polypeptide, an encoder barcode sequence may or may not be transferred to the recording tag associated with the polypeptide. The efficiency of such transfer depends on i) the residency time of the binder near the modified NTAA residue, which brings the coding tag of the binder in proximity to the terminus of the recording tag, ii) the stability and integrity of the binder-coding tag conjugate employed during the encoding step, and iii) particular molecular architectures of the coding tag and the recording tag, which affect the reaction rate for conjugating the encoder barcode to the recording tag. For example, if the coding tag and recording tag share complementary single-stranded overhangs at their termini, this would typically increase the efficiency of the transferring reaction in a manner somewhat proportional to the length of the overhang. Depending on binder affinity and the particular encoding assay molecular architecture, the probability of successful completing the NTE step varies from about 5% to 90%. To avoid situations which would cause the multicycle encoding assay to stop and prevent from moving to the next NTAA residue (e.g., where NTE efficiency is low due to a lack of specific binders for a particular modified NTAA residue), a cycle cap reaction is used to conjugate a “null” encoder barcode sequence after the NTE step to the recording tag, but only if the recording tag was not extended at the given NTE step. Thus, each generated binder identifier string consists of N binder identifiers, and the “null” encoder barcode sequence is designated as binder identifier “Z”, which reflects the absence of a true encoding event in a given cycle. In preferred embodiments, information regarding the absence of an encoding event at a given step is recorded and used during the analysis, since such information may also provide an insight about the probability of occurrence of a particular NTAA residue at a given position within a polypeptide and the corresponding binder identifier string. Finally, depending on the particular Cleavase enzyme and modified NTAA residue, the probability of performing a successful NTC step is between about 70% and 100%. In preferred embodiments of the assay, a set of Cleavase enzymes is employed that will efficiently cleave all 20 standard modified NTAA residues (see, e.g., U.S. Ser. No. 11/427,814 B2, which is incorporated herein by reference).

The goal of the computer model of the present Example is to determine what peptide sequence or sequences are most likely to produce a read generated during the encoding assay described above. Similar to other mapping algorithms, it requires knowing the probabilities that particular peptides will be able to be N-terminally cleaved, N-terminally functionalized or N-terminally encoded, and such parameters can be determined experimentally and/or via computer modeling using structural information about specific enzymes involved in each step of the encoding assay (see also Example 7). The approach is illustrated schematically in FIG. 4D. Further details of the computer model are provided below.

The key steps of the computer model include: (a) receiving (or determining) the plurality of nucleic acid sequences generated from the encoding assay (e.g., by using an NGS DNA sequencer to sequence the oligonucleotides generated by the assay), where each of the plurality of nucleic acid sequences comprises a series of encoder barcode sequences, and where each encoder barcode sequence of a given series of encoder barcode sequences corresponds to a binder (or binding agent) of the set of binders used to perform the assay; (b) generating a binder identifier string for each nucleic acid sequence of the plurality based on a corresponding series of encoder barcode sequences within a given nucleic acid sequence, thereby generating a plurality of binder identifier strings for the plurality of nucleic acid sequences; (c) for each of the plurality of binder identifier strings, converting a given binder identifier string into a plurality of peptidic reads based on the binding profiles of the binders that correspond to the binder identifiers present in a given binder identifier string; (d) scoring one or more peptidic reads based on the probability that a given peptidic read is derived from a given binder identifier string, thereby producing a probability score (e.g., a numerical or qualitative score) for each of the one or more peptidic reads of the plurality of peptidic reads; (e) optionally, for each of the plurality of binder identifier strings, filtering out peptidic reads within the plurality of peptidic reads generated for a given binder identifier string based on (i) the probability score for each peptidic read, and (ii) a probability that a given peptidic read was generated from amino acid sequences of the plurality of polypeptides; and (f) based on the generated probability scores, outputting data related to at least a partial identity and/or quantity for at least one polypeptide of the plurality of polypeptides.

In some embodiments, the term “peptidic read” refers to an amino acid sequence that could produce a given binder identifier string during the encoding assay. In some embodiments, some peptidic read comprise non-canonical amino acid residues, such as amino acid residues modified with post-translational modifications. In some embodiments, a probability score is calculated to each peptidic read of the plurality of peptidic reads generated from a given binder identifier string. In some embodiments, the calculated probability score is indicative of a probability that a given peptidic read produces a given binder identifier string.

In some embodiments, the term “peptidic read” refers to an amino acid identifier string which comprise two or more identifiers where each identifier identifies a component of polypeptide analytes, such as an amino acid residue, dipeptide, tripeptide, and so on. In these embodiments, each peptidic read may not comprise an amino acid sequence, but instead comprise amino acid identifiers, where each identifier associates with, or corresponds to, a component of polypeptides. In some embodiments, the computer model implements amino acid identifier strings generated from non-amino acid subunits comprising two or more subunits, wherein each subunit provides association with, or corresponds to, a component of polypeptides, such as amino acid residue, dipeptide, etc.

A given peptidic read of the plurality of peptidic reads is built in steps with information regarding each cycle's binder identifier and with additional information regarding the encoding assay parameters and the composition of the peptidic read. For example, binding specificity for a given binder may depend not only the modified NTAA residue but may also depend on the P2 (penultimate) or P3 (antepenultimate) residues of a peptide, which may be taken into account by adjusting the probabilities that a particular binder generates a signal across different combinations of amino acid residues.

In another example, if a given binder identifier is “Z”, which corresponds to a “null” encoder barcode sequence (absence of the encoding event at a given cycle), it is possible that (i) the peptide was not N-terminally modified (i.e., the modification rate can depend on the particular modified NTAA, and can be determined experimentally before performing the assay), or (ii) there was no specific binder in the binder set for a given modified NTAA residue. If a binder identifier is not “Z” (mod=0), the encoding event at a given cycle occurred, and the peptide was definitely N-terminally modified (mod=1).

If the peptide is N-terminally modified, then the posterior probability that any peptide or dipeptide is produced in the next encoding cycle is proportional to the modification rate (i.e., efficiency of NTAA functionalization) multiplied by the encoding rate (i.e., efficiency of NTAA encoding) for that [di]peptide for the binder observed.

For the second (and subsequent) binder identifier, each peptidic read and associated probability from of the previous step(s) is transformed in the following manner:
a) if the previous peptidic read was N-terminally modified, cleavage may occur with a probability based on the sequence of that peptidic read (e.g., based on P1-P2 residues of the peptidic read). In this case, the next encoding step (i.e., the binder identifier under consideration) extends the imputed sequence of the peptidic read, where now the information is added to the C-terminus of the peptidic read, and the probability is multiplied by a factor depending on the sequence and binder profile (exactly the same as for the base case, but now the computer model starts with a prior version of the peptidic read sequence rather than an “empty” sequence). Alternatively, cleavage step may not have occurred, in which case, the encoding step does not extend the peptidic read but the probability does multiply in the same manner (this is equivalent to “double” or “triple” reading the same NTAA of the peptidic read);
b) if the previous peptide was not modified, no cleavage step could have happened and therefore there is no extension to the peptidic read sequence, but the probabilities get multiplied in a manner consistent with the above description.

In some embodiments, a probability score for each peptidic read is generated, which is a probability that a given peptidic read is produced from a given binder identifier string. In some embodiments, all generated peptidic reads are scored. In some embodiments, some scored peptidic reads are then filtered out based on a pre-determined set of criteria.

In some embodiments, for computational efficiency, filtering may be applied to non-complete peptidic reads before every binder identifier in the binder identifier string being analyzed is converted to an amino acid residue based on known or learned binding profiles of the binders. For example, filtering may be applied to non-complete peptidic reads produced before transition from one binder identifier in a binder identifier string to another (which mimics the encoding cycle, since each encoder barcode sequence is installed at the completion of a single encoding cycle). The filtering may include features such as: a) excluding peptidic reads that fall below a particular probability threshold in absolute or relative terms; b) keeping only the top K peptidic reads (where K is one of 10, 100, 1000, 10k, 100k etc.); c) applying a “biological sequence filter” that only keeps peptidic reads that are expected to be in the sample. For example, based on a tryptic digestion of a human proteome sample during the sample preparation before performing the encoding assay, only tryptic fragments of polypeptides should be present in the sample.

In some embodiments, for computational efficiency, only a certain number of peptidic reads associated with a given binder identifier string are kept. This number could be, for example, no more than 10, no more than 50, no more than 100, no more than 200 or no more than 1000. In some embodiments, only peptidic reads associated with a given binder identifier string having probability scores that meet a specified threshold are kept. In some embodiments, a threshold may be arbitrarily chosen before implementation of the disclosed method.

In some embodiments, the described filtering step may be repeated one or more times throughout the process of inferring one or more peptidic reads from a given binder identifier string.

After the steps in the above algorithm are complete, a list of peptidic reads (potential peptide sequences) and corresponding proteins potentially present in the sample may be generated. The more cycles of binding/encoding used in the encoding assay, the more likely one or more peptidic reads may differentiate from the other peptidic reads generated from a given binder identifier string. The number of binding/encoding cycles usually corresponds to the length of the binder identifier string (but could be different if the P1P2-dependency of at least some binders is strong and is accounted for during the analysis). In preferred embodiments, the number of binding/encoding cycles used in the encoding assay, and the number of binder identifiers in the binder identifier strings, can be, for example, 4 or more, 5 or more, 6 or more, 7 or more, 8 or more, 9 or more, or 10 or more.

In some embodiments, the top hit in the list of generated peptidic reads may be attributed to any peptide fragments (which derive from one or more proteins) that share the same sequence. Thresholds, such as requiring the top (most probable) hit to be at least 10×, 100×, or 1000× more probable than the second highest (second most probable) hit within the list of generated peptidic reads, can also be used. As can grouping generated peptidic reads produced by the computer model with corresponding peptide fragments from the proteome of interest, summing the probabilities (as multiple sequences produced by the computer model may have the same peptide origin), and then applying the probability threshold described immediately above.

Alternatively, if the threshold for hit probability is set but not met for a single peptidic read, it may be possible to identify a set of peptidic reads that each have higher probability compared to the rest of the generated peptidic reads. Additional steps may be employed to further differentiate a set of several generated “high probability” peptidic reads. For example, if more than one peptide fragment of a protein is analyzed, each fragment generated a binder identifier string through the encoding assay, and some of generated binder identifier strings are ambiguous such as they map to multiple distinct proteins, the correct protein may be predicted based on the identification of more than one protein fragment (or peptidic read) with reasonable probability that maps to the protein. Optionally, the origin of each protein fragment may be tracked using a UMI sequence installed in the recording tag associated with the protein fragment (such as each peptide originated from the same protein share the same UMI) in the encoding assay, and the protein may be identified when peptidic reads generated from two or more different binder identifier strings correspond to two or more components of a single protein molecule.

In some embodiments, a list of generated peptidic reads with their calculated probabilities is outputted to a user. In some embodiments, the inferred amino acid sequences of polypeptides of the plurality of polypeptides are outputted to a user. In some embodiments, data related to at least a partial identity and/or quantity for at least one polypeptide of the plurality of polypeptides are outputted to a user.

To illustrate performance of the computer model, the computer model is applied, for simplicity, to a nucleotide derived sequence (so there is an alphabet of only 4 (“a”, “c”, “g”, “t”) elements instead of ˜20 for polypeptide analytes) to illustrate association of binder identifiers with (“a”, “c”, “g”, “t”) sequences of “polypeptides”. One can setup a table for the probabilities of the components of the model system that has only 2 “binders” (“A” binder and “C” binder) as shown in Table 1 below.

TABLE 1
showing hypothetical “encoding assay” parameters.

| Action | a nucleotide | c nucleotide | g nucleotide | t nucleotide |
|---|---|---|---|---|
| “A” binder | 60% | 10% | 10% | 20% |
| “C” binder | 10% | 60% | 15% | 15% |
| Cleave | 80% | 80% | 60% | 80% |
| Functionalization | 90% | 80% | 90% | 90% |

Let's say the computer model receives the binder identifier string “AAC”. Then, potential “peptidic reads” for the first binder identifier “A” are based on the encoding profile of the A binder, and are listed in Table 2 below (rank-ordered by probability).

TABLE 2
showing rank-ordered “peptidic reads”
for the first binder identifier “A”.

| | seq | mod | prob |
|---|---|---|---|
| 0 | a | True | 0.6 |
| 1 | t | True | 0.2 |
| 2 | c | True | 0.1 |
| 3 | g | True | 0.1 |

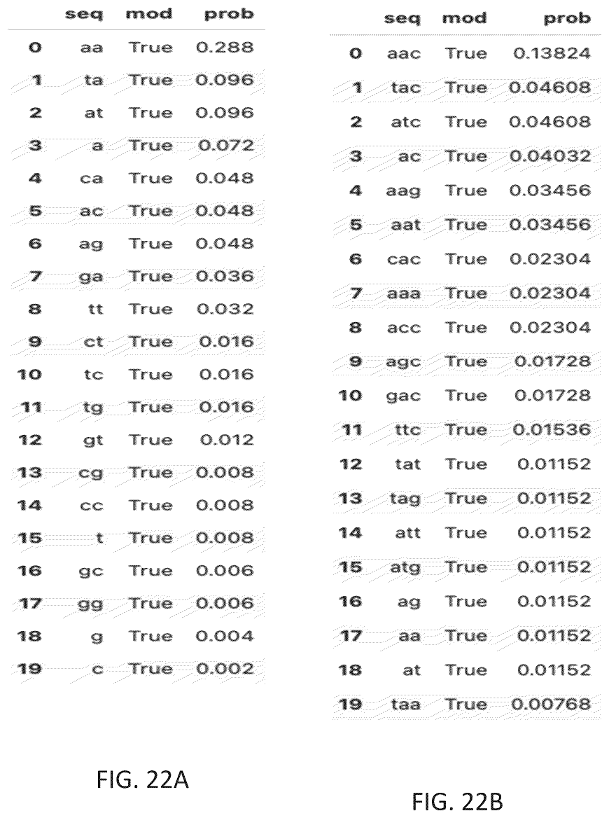

Next, examples of the probabilities of potential “peptidic reads” being identified after analyzing the binder identifiers “AA” are shown in FIG. 22A. Particular examples of probabilities are determined as follows: i) for sequence 0, 0.288=0.6 (probability the first binder identifier corresponds to “a”)*0.8 (cleavage rate for binder identifier “a”)*0.6 (encoding profile for A binder); ii) for sequence 3, 0.072=0.6*(1−0.8) (the situation where there is no cleavage of a)*0.6 (encoding profile for A binder). Other entries are similarly calculated based on whether the cleavage of a particular element happens or not, and on the encoding profile (efficiency of the encoding step) of the binder.

Finally, examples of the probabilities of potential “peptidic reads” being identified after analyzing the binder identifier string “AAC” are peptidic read shown in FIG. 22B. Amongst the list of probabilities, there is 19.5% (normalized probability; the “raw” probability is 0.138) that the “peptide” sequence was “aac”, with the next best candidates only about one third as likely due to the imprecision of the A binder (it is ⅓ as likely to encode the “t” “amino acid” as to encode the “a” “amino acid”).

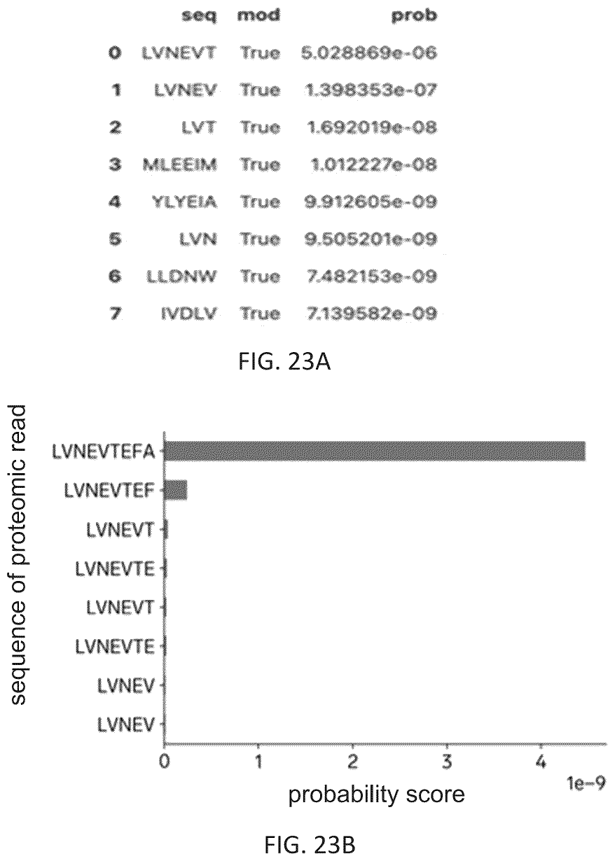

FIG. 23A-FIG. 23B show another example of calculating probabilities of peptidic reads based on a given binder identifier string “LVNDVZDF” (SEQ ID NO: 18) that was generated from experimental encoding assay data (inferred from specific combinations of encoder barcodes present in extended recording tags generated during the encoding assay), and actual assay performance parameters that were inputted to a computer model to calculate probabilities of potential peptidic reads (e.g., candidate peptidic reads) inferred from the given binder identifier string. Generated peptidic reads contain amino acid sequences as set forth in SEQ ID NO: 19-SEQ ID NO: 27. Each binder identifier in the string usually corresponds to a particular binder that encoded an amino acid residue in a particular encoding cycle (such as, e.g., binder identifier “L” corresponding to an L-binder which encoded an amino acid residue in the first encoding cycle in this example). The binder identifier “Z” corresponds to a blank (null) binder identifier, which indicates that no encoding event occurred in the 6th encoding cycle. In this variant of an encoding assay, each encoding cycle “adds” one and only one binder identifier to a resulting binder identifier string, because even without an encoding event the recording tag attached to a peptide analyte is extended each cycle. Thus, each potential peptidic read that could produce a given binder identifier string may be built sequentially by analyzing one binder identifier of a given binder identifier string at a time. For example, after 5 encoding cycles, the corresponded binder identifier string is “LVNDV” (contains only first 5 binder identifiers), and the probabilities of potential peptidic reads for this binder identifier string that were determined using the computer model described above are shown in FIG. 23A. Importantly, the more encoding cycles used in the encoding assay (and the longer the resulting binder identifier strings), the more chances exist to differentiate the “true” peptidic read from the rest of the potential peptidic reads based on the probabilities of occurrence. For example, as shown in FIG. 23A, after 5 encoding cycles, the top probability scores (for “LVNEVT” (SEQ ID NO: 19) and “LVNEV” (SEQ ID NO: 20)) are already significantly higher than the probabilities of the next most probable potential peptidic reads. If another three binder identifiers are included into the analysis (a total of 8 encoding cycles, which produces the binder identifier string “LVNDVZDF”), then all of the top probability scores correspond to essentially the same peptidic read sequence (FIG. 23B).

In examples shown in FIG. 23A and FIG. 23B, the length of the most probable peptidic read is longer than the length of the binder identifier string by one. The reason for that is that in these embodiments, the binders used for the encoding step in corresponding encoding cycles provided information not only about a current NTAA (P1) residue of a peptide analyte, but about both P1 and P2 residues. The nature of P2 may affect P1 binding and encoding, as shown in heatmap data (see, e.g., FIG. 16A-FIG. 16G). Accordingly, the fifth binder identifier in “LVNDV” corresponds to a V-binder which has preferential specificity not only for the primary amino acid residue (such as V), but also for the neighboring amino acid residue, and the highest probability of binding is when the neighboring amino acid residue is T. Thus, peptidic read LVNEVT has the highest probability score among all of the potential peptidic reads, because this read has the highest probability to generate binder identifier string “LVNDV” during the encoding assay. Similarly, peptidic read “LVNEVTEFA” (SEQ ID NO: 25) has the highest probability to generate binder identifier string “LVNDVZDF” during the encoding assay (see FIG. 23B). It is also possible that other potential peptidic reads that have been generated based on the determined binding profiles of the binders may have been filtered out based on a probability of being generated from the amino acid sequences present in a given plurality of polypeptides.

In summary, in preferred embodiments, the computer model described in this Example determines the potential amino acid sequences (called “peptidic reads”) that could produce a given binder identifier string (a specific combination of encoder barcodes) by performing a given encoding assay. The computer model may include optional filtering steps to only allow peptidic reads that are expected for a given sample or sample type (e.g., peptidic reads from a tryptic digestion of the sample). In some embodiments, the computer model can determine the most likely amino acid sequence that could have produced a given binder identifier string in the absence of biological foreknowledge (i.e., when the amino acid sequences of polypeptides in a sample are unknown). In contrast, in some embodiments, the computer model of Example 8 determines a set of binder identifier strings that the encoding assay could produce given one or more polypeptide sequences from polypeptides that are present or potentially present in a sample (e.g., where the polypeptides that are present or potentially present in a sample are known or are included in a database, such as tryptic peptides from the human proteome). In some embodiments, the mapping part of the computer model operates to find a binder identifier string that is probabilistically likely to arise from one or more polypeptides compared to the other polypeptides in the sample.

### Example 10—Use of Machine Learning, Deep Learning, and Artificial Intelligence for Inferring Amino Acid Sequences of Polypeptides from Binder Identifiers

This Example shows how to use and train machine learning models in order to decode amino acid sequences of polypeptides from binder identifier strings.

### Part I. General Considerations for Training of Machine Learning Models and Methods for Preparing Large Datasets to be Used in the Training.

Different machine learning models can be used to decode polypeptides from binder identifier strings. In order to train a machine learning model that generalizes beyond the training dataset to perform polypeptide sequence decoding with high accuracy on the test dataset (and on novel datasets as they are generated), the training dataset should be large (e.g., >1 million samples), high-quality (e.g., generated under optimized encoding assay conditions), and contain diverse examples of data covering a broad range of possible combinations of features (e.g., binder identifier strings) and labels (e.g., polypeptide sequences) to capture the complex relationships of input parameters/variables in the encoding assay system (such as binder sequences, binder concentrations, buffer conditions, coding tag sequences, recording tag sequences, cleavase sequences, cleavase concentrations, NTAA functionalization (NTF) efficiencies, number of cycles, polypeptide sequences in the sample, etc.) and output data (e.g., potential peptidic reads and/or relative abundances thereof). In order to generate such a large, high-quality dataset, it becomes important to be able to synthesize a large number of exemplar polypeptide sequences that may reasonably represent the P1/P2 (and optionally P3 onward) probability distribution at any given encoding cycle (i.e., between the NTF-NTE-NTC steps) in an actual unknown sample, and obtain a large number of exemplar binder identifier strings through next-generation deep sequencing platforms (e.g., using a NovaSeq X Plus platform (Illimina, San Diego, CA) with 20 billion single reads or 40 billion paired-ends reads per run). In order to acquire such a dataset, the low-to-medium throughput of solid-phase peptide synthesis workflows may not be sufficient enough to generate a large dataset of binder identifier strings for known polypeptide sequences on which to train a machine learning model that could be generalized to inferring different polypeptide sequences. One reason that low-to-medium throughput solid-phase peptide synthesis workflows may not be sufficient to generate adequate training data is that while the previously described decoding algorithms (see Example 8 and Example 9) assume identical NTF and NTC efficiencies across different polypeptide backbones (i.e., P3 onward) that share the same P1/P2 sequence, it is possible that NTF and NTC efficiencies depend on the entire polypeptide sequence and not just the P1/P2 sequences. For example, polypeptides may be partially folded when covalently attached to the recording tag in the Encoding assay, in which case the NTAA may be partially sequestered away from reactants in the flow cell solution and unable to react to become N-terminally modified. Therefore, depending on the entire polypeptide backbone sequence, the effective NTAA concentration able to undergo the N-terminal modification reaction may be apparently lower than the total NTAA concentration for a given NTAA, and the degree to which the NTAA is sequestered could be different for polypeptides with the same P1/P2 sequence but different backbone sequences. Similarly, NTC efficiency may also depend on the entire polypeptide sequence (i.e. P1, P2, and P3 onward) because engineered cleavase enzymes used to cleave N-terminally modified P1 residue may interact with amino acid residues outside of P1-P2. Further, as discussed above, if a polypeptide is partially folded when attached to the recording tag in the Encoding assay, the polypeptide sequence outside P1-P2 may contribute to the NTC efficiency. Indeed, the Gibbs free energy of a certain NTAA-modified polypeptide folding may be given by ΔΔG_{folding}, and the Gibbs free energy of the NTAA-modified polypeptide substrate binding to a given cleavase enzyme may be given by ΔΔG_{binding}, which is related to the Michaelis constant, K_{M}, of the cleavase enzyme with an N-terminally modified polypeptide substrate by K_{M}=e\hat{ }(ΔΔG_{binding}/(R·T)), where R is the gas constant and T is the temperature of the cleavase reaction. In some embodiments, the NTAA-modified polypeptide can only bind to the cleavase enzyme pocket in the unfolded state (e.g., where P1 through at least P8 are unfolded), and the ΔΔG_{binding} needs to overcome ΔΔG_{folding} in order to bind, which is a prerequisite for cleavase activity. Thus, the effective Michaelis constant would be expected to be higher and substrate binding less favorable when the polypeptide has a sequence that promotes it to be folded under the cleavage conditions in the Encoding assay (i.e., K_{M}=e\hat{ }((ΔΔG_{binding}−ΔΔG_{folding})/(R·T)). In addition, the polypeptide backbone sequence may associate non-covalently with other elements of the encoding assay system, such as the bead surface or recording tag surface (e.g., an arginine residue at position P3 may interact with DNA phosphate backbone of the recording tag, partially sequestering the NTAA from reacting with NTF reactants or increasing the effective K_{M} of the cleavase enzyme for the NTAA-modified polypeptides). For at least these reasons (and potentially for other, previously uncharacterized reasons), it is advantageous to train a robust machine learning model that captures such known and unknown effects of the Encoding assay system in a polypeptide decoding algorithm to achieve a more accurate decoding of polypeptide sequences from binder identifier strings with fewer underlying assumptions than required in the previously described decoding algorithms (see Example 8 and Example 9 above).

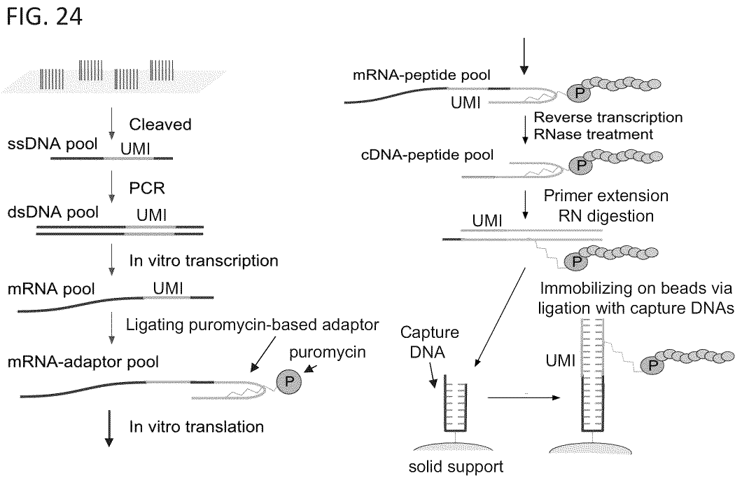

In order to generate a large, high-quality dataset for machine learning, deep learning, and artificial intelligence models to capture the complex relationships between the input parameters (e.g., binder identifier strings and parameters of the encoding assay used to generate them) and the output (e.g., potential peptidic reads and their associated probabilities, the relative abundance of predicted peptides and/or proteins, etc.) we engineered an immobilized large-scale array of genetically encoded polypeptides, each covalently conjugated with polynucleotide-based recording tags that include a unique molecular identifying (UMI) barcode for each polypeptide (i.e., a polypeptide barcode). In one embodiment, the attachment of recording tags to polypeptides occurs via ribosome or mRNA/cDNA display in which the polypeptide barcode (UMI) is contained within the mRNA sequence. Custom polynucleotides each encoding a polypeptide sequence and further containing a UMI sequence were synthesized on a DNA microarray and used for dsDNA pool production via PCR (see FIG. 24). The PCR products are transcribed in vitro, and the resulting mRNA pools are ligated to a puromycin-containing adaptor. As a result, each mRNA has a puromycin molecule attached near its 3′ end via a linker. In some embodiments, in addition to the UMI sequence, other functional elements such as a universal primer site and/or spacer sequence(s) for downstream information transfer is engineered to be present in each mRNA transcript. In one embodiment, a unique restriction site is also included, such as at the 5′ terminus of the UMI sequence and/or at other DNA recording tag functional elements.

During in vitro translation of the mRNA-puromycin pools, puromycin (P), an analogue of tyrosyl-tRNA, incorporates into the growing polypeptide strand when the ribosome nears the 3′ end of mRNA constructs, terminates translation, and effectively creates an mRNA-polypeptide fusions linking the RNA transcript with the UMI sequence to its corresponding translated polypeptide (see, e.g., US 2012/0258871 A1 and U.S. Ser. No. 12/129,463 B2, all of which are incorporated herein by reference). After that, the resulting mRNA-polypeptide pool is treated with reverse transcriptase and RNase to generate a cDNA-polypeptide pool. Double-stranded DNA-polypeptide constructs were generated using primer extension, which were then digested by a restriction endonuclease enzyme (RN) to create an overhang, which was then used to ligate DNA-polypeptide constructs to capture DNAs immobilized to a solid support, such as beads (see FIG. 24). As a result, a large number (e.g., 1 million or more, 10 million or more, 100 million or more) of polypeptide molecules may be covalently attached to beads and each attached to a recording tag comprising a customizable UMI sequence (polypeptide barcode). The customizable UMI sequence represents the attached polypeptide, and following next-generation sequencing of the polypeptide barcodes in an encoding assay can serve as an identifier for the ground-truth polypeptide sequence when used in training machine learning, deep learning, and artificial intelligence algorithms.

In some embodiments, rather than employing short polypeptide barcodes as UMI sequences to represent polypeptides, the entire gene sequence of the genetically-encoded polypeptide can represent the polypeptide for the polypeptide identification. One advantage of using the entire gene sequence of the genetically-encoded polypeptide as the UMI is that it is less prone to misidentification during next-generation sequencing as an encoding assay readout compared to a short UMI barcode. For example, if there is a DNA mutation (e.g., single nucleotide polymorphism) introduced during PCR amplification of recording tags extended during encoding events, or there is a sequencing error introduced during sequencing of the extended recording tags, the correct polypeptide sequence can be inferred from the surrounding sequence context in the polypeptide's gene sequence, as opposed to the case where a mutation or sequencing error occurs in a polypeptide-representing UMI barcode resulting in a loss of information. However, error correction methods (based on, e.g., Hamming distances) may be also employed in UMI sequences to mitigate spontaneous error problems. Following next-generation sequencing of extended recording tags, the ground-truth polypeptide sequences can be bioinformatically decoded from the aforementioned polypeptide UMIs by using a simple look-up database of the UMI that was originally associated with the genetically-encoded polypeptide gene, or in the non-limiting additional embodiment of the polypeptide gene itself acting as the UMI, the UMI can be simply translated using a standard genetic codon table.

Other strategies may be employed to attach a large number of custom polypeptide amino acid sequences of interest to a DNA-based recording tag with a UMI barcode for generating encoding data using an encoding assay, thus enabling high-throughput, high-content, multiplexed datasets that can be utilized for training robust machine learning, deep learning, and artificial intelligence algorithms, as described in more detail in the following sections.

In the following examples, it is assumed that large-scale, high-content, high-quality binder identifier string datasets (i.e., generated using fresh, highly pure reagents under optimized Encoding assay conditions (e.g., using optimized temperatures and reaction times during the various assay steps) are generated through the encoding assay system that provide: (i) a list of binder identifier strings (i.e., features for machine learning algorithms) generated from the next-generation sequencing of extended recording tags followed by bioinformatic preprocessing to convert nucleic acid sequences into binder identifier strings, and (ii) a corresponding list of ground-truth polypeptide sequences generated from the next-generation sequencing of extended recording tags followed by bioinformatic preprocessing to convert the aforementioned polypeptide UMIs to ground-truth polypeptide sequences (i.e., labels or classes for machine learning algorithms). These large, high-quality datasets (which importantly include the ground-truth polypeptide sequences) may also be leveraged to evaluate the performance of the computer models described in Example 8 and Example 9 above. In some embodiments, instead of providing pre-determined encoding assay parameters (such as NTF, NTE and NTC efficiencies; see Example 7) as an input to a computer model while assuming no or little polypeptide backbone sequence effects, information on NTF efficiency, NTC efficiency, and binding agent encoding efficiencies that may depend on entire polypeptide backbone sequences may be holistically integrated (i.e., assimilated) into the underlying dataset used for training the models, and the complex relationships of the input parameters of the encoding assay can then be learned through appropriate choices of machine learning model architectures and hyperparameters.

In some embodiments, following an encoding assay, the bioinformatically preprocessed dataset of binder identifier strings and their corresponding ground-truth polypeptide sequences are split into three datasets, as per standard machine learning practices: the training dataset, the validation dataset, and the test dataset, in a ratio of, for example, 8:1:1, for training:validation:test datasets, respectively (Chollet, F. (2021). Deep learning with Python. Simon and Schuster). The training dataset is used to update the weights (and/or biases), also called learned parameters of the model during training; the validation dataset is used to evaluate the model performance during training; and the test dataset is used to evaluate model performance on unseen data after training is completed. As a general rule, the closer the training dataset is to the unknown, not previously characterized sample of interest, the higher the accuracy of polypeptide sequence identification that can be delivered by the model. In some embodiments, the model is prevented from overfitting to the training dataset by careful monitoring its performance on the validation dataset during training, and training is stopped when the performance gap between the training dataset and validation dataset reaches a certain value or threshold. For example, if the goal is to identify polypeptides present in a human blood plasma sample, then a machine learning model would be trained (or fine-tuned from a model trained on more general data from diverse protein samples) on ground-truth polypeptide samples that were generated by using large-scale pools of genetically-encoded polypeptides covalently attached to recording tag immobilized on beads with corresponding UMI barcodes (generated as described above), where the polypeptides are the same fragments that would be obtained by a protease (e.g., trypsin) digestion of human plasma samples, and preferably with abundance ratios that mimic the abundance ratio present in the analyzed (unknown) sample. Actual polypeptide abundances may be estimated using existing technologies such as tandem mass-spectrometry (MS/MS) on prepared human blood plasma samples.

In some embodiments, the learned weights (and/or biases) of the machine learning model depend on the exact experimental input parameters (e.g., binding agent concentrations, NTF reaction time, temperature, etc.) and the experimental reproducibility of the encoding assay is paramount to accurate polypeptide identification (Rapp, J. T., Bremer, B. J., & Romero, P. A. (2024). Self-driving laboratories to autonomously navigate the protein fitness landscape. Nature chemical engineering, 1(1), 97-107; Whang, S. E., Roh, Y., Song, H., & Lee, J. G. (2023). Data collection and quality challenges in deep learning: A data-centric ai perspective. The VLDB Journal, 32(4), 791-813). For example, pre-training a machine learning model on a dataset that utilized high-quality, fresh reagents could generate polypeptide mis-identifications if applying the same pre-trained machine learning model to experimental encoding data generated under different assay conditions such as with partially unfolded or disintegrated binding agents (e.g., due to mishandling) such that the effective (folded) binding agent concentrations are different than those stemming from the training dataset that the model was trained on and therefore that the model expects since it has learned from high-quality datasets. In order to mitigate experimental reproducibility differences in each encoding assay, in another non-limiting embodiment, re-training a new machine learning model or fine-tuning existing trained models on new datasets may be performed by spiking ground-truth polypeptides (associated to their corresponding UMI barcodes, as described above) into the sample with unknown polypeptides. This may be accomplished by preparing beads containing known sample(s) side-by-side with beads containing unknown sample(s), and mixing the two beads sets (at a ratio of, for example, but not limited to, 10:1, 5:1, 2:1, or 1:1 unknown:known sample bead sets) prior to running the encoding assay on the same instrument. Thus, a new model may be trained (or an existing model fine-tuned) using training data from known polypeptide samples, and subsequently model inference may be run on the unknown polypeptide samples in a single-use experiment, in order to decode polypeptides on an experiment-by-experiment basis. This approach can also mitigate batch- or lot-dependent effects between encoding assay reagents, for example, mitigating any drifts in manufacturability over time, such as binding agents stored at the wrong temperature by the user or using reagents that are past their expiration dates, or for example if the binding agents used to collect datasets under which the original model was trained were to be purified under different experimental conditions such as different buffers or pH, and thus the binding agents have slightly different binding properties to the NTAA-modified polypeptides (e.g., thermodynamic dissociation rate, and/or thermodynamic association rate) or are partially unfolded/degraded during the encoding assay. Such re-training or fine-tuning of a machine learning model on an experiment-by-experiment basis can mitigate and reduce the occurrence of misidentification of polypeptide sequences in unknown samples.

Part II. Supervised Learning of Amino Acid Sequences of Polypeptides from Binder Identifier Strings by Trained Naive Bayes Models.

In one embodiment, an application of machine learning to probabilistic relationships between a plurality of binder identifier strings and a plurality of polypeptide sequences, a categorical naive Bayes supervised learning algorithm can be employed and trained. Naive Bayes methods are probabilistic classifier methods based on Bayes' theorem that assumes that every pair of input features are conditionally independent given the value of the class variable, which is the core “naive” assumption (McCallum, A., & Nigam, K. (1998, July). A comparison of event models for naive bayes text classification. In AAAI-98 workshop on learning for text categorization (Vol. 752, No. 1, pp. 41-48)). Thus, for a categorical naive Bayes method, independence between the input features is assumed (i.e., between each binder identifier (BI) in the binder identifier strings, such that BI1 is independent of BI2, BI2 is independent of BI3, and so forth) for predicting each of the classes (i.e., each of the amino acid identities in the polypeptide sequence). The advantage of this method is that naive Bayes supervised learning algorithms train quickly on large datasets such as high-content datasets generated by multiplexed encoding assays, and they also provide a baseline model against which to compare the performance of more complex neural network architectures and language models as described later (which may utilize, e.g., attention mechanisms, transformers, or recurrent neural network layers). In a simplest categorical naive Bayes supervised learning method, training, validation, and test datasets are first preprocessed to tokenize each barcode in the binder identifier string so that each encoding event barcode is identified by a token, i.e., BI1, BI2, and upward to BI″N″ where N is the total number of binding events in the encoding assay that may or may not correspond to the total number of the encoding cycles, since not all binding events result in actual encoding events. To account for that, an additional binder token “BBI” may also be included as a blank binder identifier (BI) when no encoding event has occurred in a certain cycle (but where instead a cycle cap barcode has been transferred to the recording tag in place of a binding agent's encoder barcode from the coding tag). In another non-limiting embodiment, nucleic acid barcode sequences may be directly processed into binder tokens during model training (i.e., as part of the model training procedure), rather than being bioinformatically preprocessed before model training. In another non-limiting embodiment, nucleic acid barcode sequences may be directly processed into binder tokens during running inference on the trained model (i.e., as part of running inference on the trained model). In these additional non-limiting embodiments, the bioinformatic preprocessing steps may be performed within the polypeptide decoding algorithm, rather than separately from running model training or model inference. There is no need to preprocess each residue in the polypeptide sequence into tokens since the amino acid identity itself may act as a token, i.e. methionine is M, alanine is A, etc. In some embodiments, both the j-th element in the input binder identifier string, X, and the i-th element in the polypeptide sequence, Y (the output polypeptide sequence) are categorized into tokens, where j refers to the index (or position) in the binder identifier string, and where i refers to the index (or position) in the polypeptide sequence. Next, for each polypeptide token in the polypeptide sequence, the prior probabilities of each class (i.e., of each amino acid identity) are calculated by counting how often each amino acid residue appear across the training dataset, which gives the probability distribution of amino acid identities (i.e., polypeptide tokens or amino acid identity tokens) in the dataset, which is given by: P(Y=yᵢ)=(number of occurrences of amino acid identity yᵢ)/(total number of amino acid identity occurrences in the dataset). Next, conditional probabilities are calculated that a given amino acid identity in the polypeptide sequence at index i co-occurs with a binder token at index j, as given by: P(X=xⱼ|Y=yᵢ)=(number of binder token xⱼ occurring with amino acid identity yᵢ)/(total occurrences of amino acid identity yᵢ). This is calculated for each index j in the binder identifier string and each index i in the polypeptide sequence, treating each binder identifier token position and amino acid identity token position independently. Then, the amino acid identity can be determined (predicted) at index i of the polypeptide sequence using Bayes theorem:

$$P ( Y = {y}_{i} \vert X ) ∝ P ( Y = {y}_{i} ) \cdot {\prod }_{j = 1}^{n} P ( {X}_{j} = {x}_{j} \vert Y = {y}_{i} )$$

Subsequently, the argmax function of this computed posterior probability is taken such that the predicted amino acid at each index i of the polypeptide sequence is the one with the highest posterior probability:

$${\hat{Y}}_{ι} = arg max ( P ( Y = {y}_{i} \vert X ) )$$

This computation is then repeated for each index i in the polypeptide sequence, which returns the amino acid identity token at each position, and once combined across all positions in Y ultimately provides the highest probability polypeptide sequence given a binder identifier string. For example, if the binder identifier string input is “BI3-BI4-BI1-BBI-BI1” (where binder tokens are separated by dashes), then the output polypeptide sequence could be “X3-X4-X1-X5-X1” (where peptide tokens are separated by dashes), which is a decoded polypeptide sequence. Implicit in the model is the learned NTF efficiency, NTE efficiency, and NTC efficiencies, which is factored into the posterior probabilities as part of the conditional probabilities P(X=xⱼ|Y=yᵢ), since the binder token BBI might be encountered at index j due to a skipped NTF event on certain NTAAs more often than on other NTAAs, which we refer to as a phasing issue. This might warrant implementing a frame-shift algorithmic step in the decoded polypeptide sequence to align binder token j+1 with peptide token i. Similarly, if a cleavase NTAA removal event is skipped due to insufficient cleavase reaction time, this might warrant implementing a frame-shift algorithmic step in the decoded polypeptide sequence to align binder token j with peptide token i−1. However, these NTF, NTE and NTC efficiencies are implicitly learned as the probability distributions from the underlying data (i.e., they are holistically integrated or assimilated into the training dataset). When the encoding assay is operating efficiently with high NTF efficiencies, high NTE efficiencies, and high NTC efficiencies, and the training dataset has a sufficient number of samples, the phasing issue of frame-shift effects becomes less and less probable, and a correct polypeptide sequence is decoded from a given binder identifier string. The accuracy (or performance) of the trained model can be evaluated by identifying polypeptide sequences from binder identifier strings in the test dataset using evaluation metrics such as, but not limited to, the accuracy per binder token between the predicted peptide token and ground-truth peptide token, or the edit distance of the predicted polypeptide sequence to the ground-truth polypeptide sequence.

Part III. Supervised Learning of Amino Acid Sequences of Polypeptides from Binder Identifier Strings by Trained n-Gram Naive Bayes Models.

This embodiment shows how to use n-gram naive Bayes supervised learning algorithms (Raschka, S. (2014). Naive bayes and text classification i-introduction and theory. arXiv preprint arXiv:1410.5329) to decode polypeptide sequences from given binder identifier strings. For example, in a bigram (n=2) naive Bayes model, two input features (i.e., two consecutive binder identifier tokens at indices j and j+1) are used to predict a single polypeptide residue (i.e., an amino acid identity at position i). A bigram model can usually capture more information than a unigram model (as in the previously described categorical naive Bayes supervised learning algorithm), because it uses pairs of consecutive binder tokens instead of single binder tokens, and thus captures more context for each peptide token prediction, such as skipped NTF events, skipped NTC events, and skipped NTE events. In another non-limiting embodiment, a categorical naive Bayes model is implemented in which the joint probability of two consecutive amino acid tokens (e.g., yᵢ and yᵢ₊₁) is computed based on the probability of one binder token at a time (e.g., xⱼ), which has the advantage of factoring into the learned probability distributions the P1- and P2-binding specificities of each binding agent. In yet another embodiment, the n-gram naive Bayes supervised learning algorithm could be implemented where n is the number of cycles in the encoding assay, to compute either the amino acid identity at index i, or amino acid identities at positions i and i+1, or amino acid identities at all positions between i and n simultaneously. In this case, the joint probability distribution of the entire polypeptide sequence given an entire binder identifier string is learned, and the joint probability P(Y₁,Y₂, . . . , Yₙ|X₁,X₂, . . . , Xₙ) is computed, where the class-conditional probability P(X₁,X₂, . . . , Xₙ|Y₁,Y₂, . . . , Yₙ) depends on all positions of both the binder identifier string and the polypeptide sequence, which is the full joint distribution across the entire binder identifier string and polypeptide sequence conditioned on the amino acid identity tokens. There may be an issue with computational memory limitations due to the exponentially increasing number of possible binder token combinations and peptide token combinations (i.e., with 20 canonical amino acid identities and 5 cycles, we are already approaching 20⁵=3.2 million possible classes to predict, which becomes intractable as the number of cycles, n, increases). With further development in quantum hardware technologies, the problem may become more tractable when using quantum computing with quantum Bayes classifiers (Wang, M. M., & Zhang, X. Y. (2024). Quantum Bayes classifiers and their application in image classification. Physical Review A, 110(1), 012433).

Part IV. Learning Amino Acid Sequences of Polypeptides from Binder Identifier Strings by a Trained Large Language Model.

In the context of large language models, translation of binder identifier strings into polypeptide sequences could be implemented with a sequence-to-sequence (e.g., seq2seq) language model that performs sequence transduction (Shi, T., Keneshloo, Y., Ramakrishnan, N., & Reddy, C. K. (2021). Neural abstractive text summarization with sequence-to-sequence models. ACM Transactions on Data Science, 2(1), 1-37), analogous to a generative artificial intelligence model that translates, for example, English sentences into Japanese sentences. These large language model neural network architectures utilize an encode-transmit-decode process, where the encoder may be a residual neural network (RNN) using long short-term memory (Hochreiter, S. (1997). Long Short-term Memory. Neural Computation MIT-Press) or several self-attention transformer blocks, and the decoder may be an RNN or several cross-attention causally-masked transformer blocks (Yin, Q., He, X., Zhuang, X., Zhao, Y., Yao, J., Shen, X., & Zhang, Q. (2024). StableMask: Refining Causal Masking in Decoder-only Transformer. arXiv preprint arXiv:2402.04779). Given the grammatical nature of polypeptide sequences that protein language models such as ProteinBERT (Brandes, N., Ofer, D., Peleg, Y., Rappoport, N., & Linial, M. (2022). ProteinBERT: a universal deep-learning model of protein sequence and function. Bioinformatics, 38(8), 2102-2110), ESM2 (Lin, Z., Akin, H., Rao, R., Hie, B., Zhu, Z., Lu, W., . . . & Rives, A. (2022). Language models of protein sequences at the scale of evolution enable accurate structure prediction. BioRxiv, 2022, 500902; Rives, A., Meier, J., Sercu, T., Goyal, S., Lin, Z., Liu, J., . . . & Fergus, R. (2021). Biological structure and function emerge from scaling unsupervised learning to 250 million protein sequences. Proceedings of the National Academy of Sciences, 118(15), e2016239118), ESM3 (Hayes, T., Rao, R., Akin, H., Sofroniew, N. J., Oktay, D., Lin, Z., . . . & Rives, A. (2024). Simulating 500 million years of evolution with a language model. bioRxiv, 2024-07), SaProt (Su, J., Han, C., Zhou, Y., Shan, J., Zhou, X., & Yuan, F. (2023). Saprot: Protein language modeling with structure-aware vocabulary. bioRxiv, 2023-10), ProstT5 (Heinzinger, M., Weissenow, K., Sanchez, J. G., Henkel, A., Mirdita, M., Steinegger, M., & Rost, B. (2023). Bilingual language model for protein sequence and structure. bioRxiv, 2023-07), ProGen (Madani, A., McCann, B., Naik, N., Keskar, N. S., Anand, N., Eguchi, R. R., . . . & Socher, R. (2020). Progen: Language modeling for protein generation. arXiv preprint arXiv:2004.03497), and ProtGPT2 (Ferruz, N., Schmidt, S., & HOcker, B. (2022). ProtGPT2 is a deep unsupervised language model for protein design. Nature communications, 13(1), 4348) have learned after training on millions to billions of naturally occurring polypeptide sequences, it is possible for such models to pick up signals on the corresponding grammatical nature of binder identifier strings which, with the appropriate mixture of binding agents that show high P1 or P1/P2 encoding specificity in the encoding assay, could effectively learn a comparable representation of the polypeptide language probability distributions that protein language models have been shown to learn, i.e., that there is a probability distribution for the full or partial polypeptide sequence given the full or partial binder identifier string. Therefore, it is reasonable that an appropriately trained or fine-tuned large language model could estimate the complex relationships between binder identifier strings and polypeptide sequences by using a sufficiently sized, high-quality training dataset. In another non-limiting embodiment, a temperature parameter (Holtzman, A., Buys, J., Du, L., Forbes, M., & Choi, Y. (2019). The curious case of neural text degeneration. arXiv preprint arXiv:1904.09751) of the large language model may be implemented which affects the randomness or variability of the generated polypeptide sequence output by the model given the input binder identifier string, and may be set to low value near zero (e.g., 0.1) so that the model chooses the highest probability polypeptide sequence given the input binder identifier string, or may be set to a higher value from zero (e.g., 1.0) so that the model chooses lower probability polypeptide sequences given the input binder identifier string (which may or may not result in higher accuracy of the model on the test dataset).

In another non-limiting embodiment, a nucleus sampling parameter (also called top-p) value may be implemented, which also affects the randomness or variability of the generated polypeptide sequence output by the model given an input binder identifier string. The nucleus sampling parameters sets a threshold probability of polypeptide sequences that can be generated by the model. For example, given an input binder identifier string, a high nucleus sampling value allows the model to possibly generate more polypeptide sequences, and a low nucleus sampling value only allows the model to generate a few possible polypeptide sequences. In a non-limiting example, using a nucleus sampling value of 0.1 means that the model may only generate polypeptide sequences with probabilities that together add up to at least 10% of the probability mass of possible polypeptide sequences given an input binder identifier string. In another non-limiting embodiment, a top-k parameter may be implemented, which involves the model sampling from the top k most probable polypeptide sequences given an input binder identifier string. In a non-limiting example, if k=3, then the large language model generates one polypeptide sequence from the 3 most probable polypeptide sequences given an input binder identifier string. Implementing a top-k parameter reduces the chances of the model generating improbable polypeptide sequences, which may or may not result in higher accuracy of the model on the test dataset.

In another non-limiting embodiment, the large language model may be trained to translate nucleic acid barcode sequences representing binder identifiers directly into polypeptide sequences, rather than bioinformatically preprocessing the nucleic acid barcode sequences into binder identifier strings that the model then learns to translate into polypeptide sequences (i.e., the model performs sequence transduction from nucleic acid barcode sequences into polypeptide sequences). In another non-limiting embodiment, the large language model may be trained to translate nucleic acid barcode sequences representing binder identifiers directly into nucleic acid barcode sequences representing polypeptide identifiers, rather than bioinformatically preprocessing the nucleic acid barcode sequences into binder identifier strings and bioinformatically preprocessing the nucleic acid barcode sequences into polypeptide identifiers (i.e., the model performs sequence transduction from nucleic acid barcode sequences into different nucleic acid barcode sequences). It is important to note that the bioinformatic preprocessing steps may be accomplished within the polypeptide decoding algorithm, rather than separately from running model training or model inference.

Part V. Learning Amino Acid Sequences of Polypeptides from Binder Identifier Strings by a Trained Feed-Forward Neural Network Model.

A feed-forward neural network model can also be used to map binder identifier strings to polypeptide sequences. In one embodiment, in order to featurize binder identifier strings from categories represented by individual binder identifiers into a continuous-valued embedding space, one or more protein language models (pLMs), including, but not limited to, ESM2 (Lin, Z., et al. (2022). Language models of protein sequences at the scale of evolution enable accurate structure prediction. BioRxiv, 2022, 500902; Rives, A., et al. (2021). Biological structure and function emerge from scaling unsupervised learning to 250 million protein sequences. Proceedings of the National Academy of Sciences, 118(15), e2016239118), ProstT5 (Heinzinger, M., et al. (2023). Bilingual language model for protein sequence and structure. bioRxiv, 2023-07) and SaProt (Su, J., et al., (2023). Saprot: Protein language modeling with structure-aware vocabulary. bioRxiv, 2023-10), are used to embed the amino acid sequence of each binding agent into a 2-dimensional matrix of embeddings, with the number of binding agent amino acid positions along the first dimension and the feature representation along the second dimension. The advantage of embedding the binding agent amino acid sequences with one or more protein language model (pLM) embeddings is to capture physicochemical and structural representations of the binding agents, which includes structural features of the pocket residues that interact directly with NTAA-modified targets, and thus the feed-forward neural network model could prospectively learn a latent space representation of NTAA-modified target binding specificity and strength (e.g., affinity, thermodynamic association rate and/or thermodynamic dissociation rate) for the entire polypeptide sequence. Because binding agent amino acid sequences vary in length, once the amino acid sequences are tokenized using a protein language model (pLM) tokenizer, they are padded with a special padding token along the first dimension such that all tokenized sequences conform to the same sized output embedding matrix when fed through the aforementioned pLM. For a given binder identifier string, in consecutive order of the binder identifiers the corresponding 2-dimensional embeddings for each binding agent are stacked into a third dimension representing the encoding cycles, with one embedded binding agent amino acid sequence per rank on the third dimension. Thus, for each binder identifier string, a three-dimensional tensor of continuous-valued embeddings is generated. Optionally, mean pooling (or maximum pooling) of the 2-dimensional embeddings for each binding agent is performed along the first dimension (representing residue position) to reduce the embeddings to a 1-dimensional array per binding agent, and then these arrays are stacked in consecutive order of binder identifiers from the binder identifier string into a second dimension representing the encoding cycles, with one embedded binding agent amino acid sequence per rank on the second dimension. The advantage of reducing (i.e., compressing) the binding agent sequence embeddings along the residue position dimension is to achieve a compact representation of each binding agent to fit into random access memory during feed-forward neural network model training. In some embodiments, the polypeptide sequences (i.e., the labels) are preprocessed, where each is transformed into a 2-dimensional matrix of one-hot encoded canonical amino acid identities (along the first dimension) and at each position in the polypeptide (along the second dimension). For this preprocessing step, a look-up table for canonical amino acid identities with corresponding indices may be established:

TABLE 3
Look-up table for one-hot encoded canonical polypeptide amino acid identities.

| Index | 0 | 1 | 2 | 3 | 4 | 5 | 6 | 7 | 8 | 9 | 10 | 11 | 12 | 13 | 14 | 15 | 16 | 17 | 18 | 19 |
|---|---|---|---|---|---|---|---|---|---|---|---|---|---|---|---|---|---|---|---|---|
| Identity | A | C | D | E | F | G | H | I | K | L | M | N | P | Q | R | S | T | V | W | Y |

Because the feed-forward neural network model is trained to perform a classification task of predicting the correct class (e.g., amino acid identity) per encoding cycle, an appropriate loss function may be implemented to calculate the loss during training between the predicted polypeptide sequence and the ground-truth polypeptide sequence, in order to backpropagate the loss through the gradients of the weights and biases of the model, and update the weights and biases using an appropriate optimizer (e.g., the Adam algorithm (Kingma, D. P. (2014). Adam: A method for stochastic optimization. arXiv preprint arXiv:1412.6980)) during training, including, but not limited to, categorical cross entropy loss for each encoding cycle, with optional further amalgamation of losses over encoding cycles, such as L1 loss or mean squared error of categorical cross entropy losses over all encoding cycles (i.e., over all positions in the polypeptide sequence). Next, a feed-forward neural network model is trained to map one three-dimensional tensor (or one two-dimensional matrix) to categorically distributed data representing the polypeptide sequence. In some embodiments, a series of feed-forward neural network layers are employed, including (but not limited to) 2-dimensional convolutional layers, 1-dimensional convolutional layers, fully-connected linear layers, average pooling layers, maximum pooling layers, dropout layers, normalization layers such as 2-dimensional or 1-dimensional batch normalization layers (Ioffe, S. (2015). Batch normalization: Accelerating deep network training by reducing internal covariate shift. arXiv preprint arXiv:1502.03167), layer normalization, and instance normalization layers, transformer layers such as encoder and decoder layers, attention layers such as multi-head attention layers and Performer layers (Choromanski, K., Likhosherstov, V., Dohan, D., Song, X., Gane, A., Sarlos, T., . . . & Weller, A. (2020). Rethinking attention with performers. arXiv preprint arXiv:2009.14794), non-linear activation function layers such as rectified linear unit (ReLU) layers, leaky ReLU layers, Gaussian error linear unit (GELU) layers, exponential linear unit (ELU) layers, hyperbolic tangent function layers, softplus function layers, and softmax function layers. In another embodiment, the neural network model may incorporate layers with bi-directional information flow, including recurrent layers such as long short-term memory cells and gated recurrent unit cells. Using one or more of these layers the model is trained to map the 3-dimensional tensor (or 2-dimensional matrix) embeddings of the binding agent sequence over a plurality of encoding cycles (while optionally reducing the dimensionality) to learn meaningful continuous-valued latent space representations of the polypeptide sequence. As the final layer in the neural network model, a softmax function is applied to transform the predictions in continuous-valued numerical space to nearly categorical space represented by amino acid probabilities per encoding cycle. Optionally, the loss can be computed on the output of the softmax layer. Optionally, a final argmax function is applied across the second dimension to obtain categorically distributed data in the output array, represented by the indices of the predicted amino acid identities based on the look-up table in Table 3 per position in the polypeptide sequence. Using the look-up table such as Table 3, the output of the argmax function may be postprocessed back into one-hot encoded polypeptide sequences in order to discretize the neural network output, and optionally to compute the loss on the discretized outputs. When the trained model is used for inference to predict polypeptide sequences, the output of the argmax function is simply decoded back into a string of amino acid identities (where each position represents a single encoding cycle in the encoding assay) using the look-up table such as Table 3. For example, the output of the argmax function along the second dimension could be [5,2,0,3,15], which corresponds to a 5-cycle encoding assay with a predicted polypeptide sequence of G-D-A-E-S (where the predicted amino acid identities in the polypeptide are separated by a dash), using Table 3. Using this expounded methodology, we effectively adapt the polypeptide sequence identification problem from one of categorical mappings (as in the naive Bayes supervised learning approach) to a regression problem followed by an optional argmax function, outlined by the following algorithmic design: transform the binder identifier strings in discrete, categorical space into continuous-valued space to learn the predicted polypeptide sequence embeddings in continuous-valued space, which are then transformed back into discrete, categorical space. In another non-limiting embodiment, hyperparameters of the neural network model, such as learning rate, learning decay rate, batch size(s) of the training dataset and validation dataset, number of training epochs, hidden layer sizes, and number of hidden layers, are optimized in order to improve the accuracy of the model on the test dataset. In another non-limiting embodiment, k-fold cross validation or nested k-fold cross validation algorithms (Raschka, S. (2018). Model evaluation, model selection, and algorithm selection in machine learning. arXiv preprint arXiv:1811.12808) are implemented during training to optimize hyperparameters to improve the performance of the trained model on the test dataset. In order to evaluate the trained model performance on unseen data in the test dataset, inference on the model is run to calculate evaluation metrics such as, but not limited to, the accuracy of the amino acid identity at each position between the predicted polypeptide sequence to the ground-truth polypeptide sequence, or the edit distance of the predicted polypeptide sequence to the ground-truth polypeptide sequence.

In another non-limiting embodiment, nucleic acid barcode sequences representing binder identifiers may be directly processed into binder identifier strings and further embedded/transformed during model training (i.e., as part of the model training procedure), rather than being bioinformatically preprocessed before model training. Similarly, nucleic acid barcode sequences representing polypeptide identifiers may be directly processed into polypeptide sequences and further one-hot encoded during model training (i.e., as part of the model training procedure), rather than being bioinformatically preprocessed before model training. In another non-limiting embodiment, nucleic acid barcode sequences representing binder identifiers may be directly processed into binder identifier strings and further embedded/transformed during running inference on the trained model (i.e., as part of running inference on the trained model). Similarly, nucleic acid barcode sequences representing polypeptide identifiers may be directly processed into polypeptide sequences and further one-hot encoded during running inference on the trained model (i.e., as part of running inference on the trained model). In these additional non-limiting embodiments, the bioinformatic preprocessing steps may be performed within the polypeptide decoding algorithm, rather than separately from running model training or model inference.

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for identifying at least one polypeptide present in a sample comprising a plurality of polypeptides, the method comprising:
(a) performing an encoding assay using the plurality of polypeptides and a set of binders, wherein each binder of the set of binders is attached to a nucleic acid coding tag that comprises an encoder barcode that comprises identifying information regarding the binder, the encoding assay comprising:
(i) attaching the plurality of polypeptides or fragments thereof to a solid support, wherein each polypeptide or a fragment thereof is associated with a nucleic acid recording tag, thereby generating a plurality of immobilized polypeptides;
(ii) contacting the plurality of immobilized polypeptides with binders of the set of binders;
(iii) following binding of a binder of the set of binders to an immobilized polypeptide of the plurality of immobilized polypeptides, transferring an encoder barcode from a nucleic acid coding tag attached to the binder to a nucleic acid recording tag associated with the immobilized polypeptide; and
(iv) repeating steps (ii)-(iii) at least one time, thereby generating a plurality of extended nucleic acid recording tags associated with the plurality of immobilized polypeptides;
(b) determining a nucleic acid sequence of each extended nucleic acid recording tag of the plurality of extended nucleic acid recording tags or complements thereof, thereby generating, at one or more processors, a plurality of nucleic acid sequences, wherein each of the plurality of nucleic acid sequences comprises a series of encoder barcode sequences;
(c) generating, using the one or more processors, a binder identifier string for each nucleic acid sequence of the plurality of nucleic acid sequences based on a corresponding series of encoder barcode sequences, thereby generating a plurality of binder identifier strings corresponding to the plurality of nucleic acid sequences;
(d) inferring, using one or more processors, an amino acid sequence of a polypeptide of the plurality of polypeptides from binder identifiers of a binder identifier string of the plurality of binder identifier strings based on (i) binding profiles of the binders from the set of binders that correspond to the binder identifiers of the binder identifier string, and (ii) calculated probability scores of an association between one or more binder identifiers of the binder identifier string and one or more amino acid sequences of polypeptides of the plurality of polypeptides; and
(e) determining, using the one or more processors, at least one of:
(i) at least a partial identity of at least one polypeptide present in the sample, or
(ii) a quantity of at least one the at least one polypeptide present in the sample, based on the calculated probability scores and the inferred amino acid sequences.

2. A computer-implemented method for identifying a polypeptide present in a sample, the method comprising:
(a) receiving, at one or more processors, the plurality of nucleic acid sequences generated by performing an encoding assay for a plurality of polypeptides present in the sample, wherein each of the plurality of nucleic acid sequences comprises a series of encoder barcode sequences, and wherein each encoder barcode sequence of a given series of encoder barcode sequences corresponds to a binder, from a set of binders, that binds to one or more components of a polypeptide of the plurality of polypeptides;
(b) generating, using the one or more processors, a binder identifier string for each nucleic acid sequence of the plurality of nucleic acid sequences based on a corresponding series of encoder barcode sequences, thereby generating a plurality of binder identifier strings corresponding to the plurality of nucleic acid sequences;
(c) inferring, using the one or more processors, an amino acid sequence of a polypeptide of the plurality of polypeptides from binder identifiers of a binder identifier string of the plurality of binder identifier strings based on (i) binding profiles of the binders from the set of binders that correspond to the binder identifiers of the binder identifier string, and (ii) calculated probability scores of an association between one or more binder identifiers of the binder identifier string and one or more amino acid sequences of polypeptides of the plurality of polypeptides; and
(d) determining, using the one or more processors, at least one of:
(i) at least a partial identity of a polypeptide present in the sample, or
(ii) a quantity of the polypeptide present in the sample,
based on the calculated probability scores and the inferred amino acid sequences.

3. The computer-implemented method of claim 2, wherein inferring amino acid sequences of polypeptides comprises: (i) for each of the plurality of binder identifier strings, converting a given binder identifier string into one or more peptidic reads based on the binding profiles of the binders of the set of binders that correspond to binder identifiers present in a given binder identifier string, and (ii) calculating a probability score for each of the one or more peptidic reads, wherein the probability score is indicative of a probability that a given peptidic read produces a given binder identifier string.

4. The computer-implemented method of claim 3, further comprising: for each of the plurality of binder identifier strings, filtering out peptidic reads of the one or more peptidic reads generated for a given binder identifier string based on (i) the probability score for each peptidic read, and/or (ii) a probability that a given peptidic read was generated from amino acid sequences of the plurality of polypeptides.

5. The computer-implemented method of claim 2, wherein inferring amino acid sequences of polypeptides comprises:
(i) for each amino acid sequence of the plurality of polypeptides, generating one or more simulated binder identifier strings using one or more parameters of the encoding assay;
(ii) for each of the one or more simulated binder identifier strings, or for each of one or more amino acid sequences of polypeptides of the plurality of polypeptides, calculating a probability score based on a probability that a given simulated binder identifier string is associated with one or more amino acid sequences of polypeptides of the plurality of polypeptides; and
(iii) matching each of the plurality of binder identifier strings to the one or more simulated binder identifier strings based on the calculated probability scores for the one or more simulated binder identifier strings.

6. The computer-implemented method of claim 5, wherein the one or more parameters of the encoding assay comprise an efficiency of a functionalization of N-terminal amino acid (NTAA) residues of polypeptides of the plurality of polypeptides, an efficiency of a cleavage of NTAA residues of polypeptides of the plurality of polypeptides, an efficiency of an encoding of NTAA residues of polypeptides of the plurality of polypeptides, or any combination thereof.

7. The computer-implemented method of claim 2, wherein inferring amino acid sequences of polypeptides comprises inputting the binder identifier strings into a trained machine learning model, wherein the trained machine learning model is trained on empirically determined encoding assay parameter data.

8. The computer-implemented method of claim 7, wherein the trained machine learning model is trained using a training data set comprising binder identifier strings data, or numerical representations thereof, for one or more isolated polypeptide samples, or numerical representations thereof, that are subjected to the encoding assay using a substantially same sample preparation protocol as that used to process the plurality of polypeptides.

9. The computer-implemented method of claim 7, wherein the trained machine learning model is configured to (i) map each binder identifier string of the plurality of binder identifier strings to a specific polypeptide sequence, or (ii) to fractionally assign a given binder identifier string to two or more specific polypeptide sequences, as part of inferring amino acid sequences of polypeptides from binder identifiers.

10. The computer-implemented method of claim 7, wherein the empirically determined assay parameter data comprises: (i) probabilities of assigning one or more binder identifiers from the set of binders to potential N-terminal amino acid (NTAA) residues of a polypeptide based on binding profiles of binders used in the encoding assay; and (ii) for each potential N-terminal amino acid residue in a polypeptide, a probability of successfully cleaving the N-terminal amino acid residue after the N-terminal amino acid residue is encoded in the encoding assay.

11. The computer-implemented method of claim 2, wherein inferring amino acid sequences of polypeptides from binder identifier strings comprises inputting the binder identifier strings, or numerical representations thereof, into a trained machine learning model, wherein the trained machine learning model is trained on a training data set comprising a set of simulated binder identifier strings generated for a given input distribution of polypeptides, or numerical representations thereof, based on empirically determined assay parameter data comprising:
(i) probabilities of assigning one or more binder identifiers from the set of binders to potential N-terminal amino acid (NTAA) residues of a polypeptide based on binding profiles of binders used in the encoding assay;
(ii) for each potential NTAA residue in a polypeptide, a probability of cleaving the NTAA residue after the NTAA is encoded in the encoding assay, and
(iii) optionally, a probability of modifying the NTAA residue.

12. The computer-implemented method of claim 2, wherein the series of encoder barcode sequences comprises from 4 to 20 different encoder barcode sequences.

13. The computer-implemented method of claim 2, wherein a computer model infers two or more amino acid sequences of polypeptides of the plurality of polypeptides from each binder identifier string, and outputs probabilities that a given binder identifier string of the plurality is derived from one of the two or more amino acid sequences of polypeptides inferred from the binder identifier string.

14. The computer-implemented method of claim 2, wherein the plurality of polypeptides encoded in the plurality of nucleic acid sequences by the encoding assay comprises at least 100,000 polypeptides.

15. The computer-implemented method of claim 2, wherein the plurality of polypeptides encoded in the plurality of nucleic acid sequences by the encoding assay comprises at least 500 different polypeptides.

16. The computer-implemented method of claim 2, wherein the binding profiles of the binders are determined experimentally.

17. The computer-implemented method of claim 13, wherein inferring amino acid sequences of polypeptides from binder identifier strings comprises inputting the binder identifier strings and encoding assay parameter data to the computer model.

18. The computer-implemented method of claim 17, wherein the computer model is configured to identify unique binder identifier signatures in the plurality of binder identifier strings as part of inferring amino acid sequences of polypeptides from binder identifiers, and wherein a given unique binder identifier signature comprises a set of binder identifier strings associated with a single polypeptide of the plurality of polypeptides.

19. The computer-implemented method of claim 2, wherein inferring amino acid sequences of polypeptides from binder identifiers comprises inputting one or more corresponding numerical representations of the binder identifier strings and optionally one or more corresponding numerical representations of encoding assay parameter data to a computer model.

20. The computer-implemented method of claim 19, wherein inferring amino acid sequences of polypeptides from binder identifiers comprises outputting one or more corresponding numerical representations of the polypeptide sequences from the computer model.

21. The computer-implemented method of claim 19, wherein the corresponding numerical representations of encoding assay parameter data includes one or more of the following: (i) the concentrations of one or more binders in the set of binders; (ii) thermodynamic parameters of binders, including association rate constants and dissociation rate constants for components of one or more polypeptides; (iii) the incubation time of binders; (iv) the wash time of binders after binder incubation; (v) the ligase concentration; (v) the ligase reaction time; (vi) thermodynamic parameters of the ligase, including Michaelis constant and catalytic turnover rate; (vii) estimated polypeptide concentrations; (viii) the concentration of cleavase enzymes; and (ix) buffer conditions for each step of the encoding assay, including enzymatic substrate concentrations, salt identities, salt concentrations, and pH.

22. The computer-implemented method of claim 2, wherein the one or more components of the polypeptide to which the binding moiety binds comprises a post-translation modification of at least one amino acid residue.

23. The computer-implemented method of claim 13, wherein the computer model is further configured to correct the quantity output for the at least one polypeptide using a correction factor calculated from a training data set.

24. The computer-implemented method of claim 9, wherein mapping a binder identifier string of the plurality of binder identifier strings to a specific polypeptide comprises:
i) generating a set of k-mer fragments for the binder identifier string;
ii) determining a probability that a given k-mer fragment of the set belongs to a specific polypeptide based on a previously determined probability distribution; and
iii) assigning the binder identifier string to the specific polypeptide based on the determined probabilities for the set of k-mer fragments.

25. The computer-implemented method of claim 9, wherein mapping a binder identifier string of the plurality of binder identifier strings to a specific polypeptide comprises providing the plurality of binder identifier strings as input to the trained machine learning model, wherein the trained machine learning model is configured to fractionally assign a given binder identifier string to two or more specific polypeptides.

26. The computer-implemented method of claim 2, wherein the method is further configured to output a confidence interval for the partial identity of the at least one polypeptide.

27. The computer-implemented method of claim 2, further comprising performing an iterative Expectation Maximization (EM) process to refine the quantity output for the at least one polypeptide of the plurality of polypeptides, wherein the iterative EM process comprises repetitively:
(i) finding a best assignment of a binding moiety to each barcode sequence in a nucleic acid sequence based on a current estimate of what polypeptides are present in the plurality of polypeptides;
(ii) finding an updated best estimate of what polypeptides are present in the plurality of polypeptides based on the best assignment of a binding moiety to each different barcode sequence in the nucleic acid sequence; and
(iii) determining an amount of the at least one polypeptide present in the plurality of polypeptides based on the updated best estimate of what polypeptides are present in the plurality of polypeptides.

28. The computer-implemented method of claim 2, further comprising determining amino acid sequences for each polypeptide of the plurality of polypeptides.

29. The computer-implemented method of claim 2, wherein the plurality of nucleic acid sequences comprises at least 100,000 nucleic acid sequences.

30. A system comprising:
one or more processors; and
a memory communicatively coupled to the one or more processors and configured to store instructions that, when executed by the one or more processors, cause the system to perform the computer-implemented method of claim 2.


SEQUENCE LISTING (33 sequences)

SEQ ID NO: 1 - protein, 259 aa, synthetic construct
SHHWGYGKHNGPEHWHKDFPIAKGERQSPVDIDTHTAKYDPSLKPLSVSYDQATSLRILNNGHVFGVEFDDSQDKAVLKGGPLDGTYRLNQFHFHWGSLDGQGSEHTVDKKKYAAELHLVHWNTKYGDLGKAVQQPDGIAILGIFLKVGSAKPGLQKVVDVLDSIKTKGKSADFTNFDPRGLLPESLDYWTYPGSLTIPPLLESVTWIVLKEPISVSSEQVLKFRKLNFNGEGEPEELMVDNWRPAQPLKNRQIKASFK

SEQ ID NO: 2 - protein, 259 aa, synthetic construct
SHHWGYGKHNGPEHWHKDFPIAKGERQSPVDIDTHTAKYDPSLKPLSVSYDQATSLRILNDGHAFKVEFDDSQDKAVLKGGPLDGTYRLEQFHFHWGSLDGQGSEHTVDKKKYAAELHLVHWNTKYGDLGKAMQQPDGLAILGIFLKVGSAKPGLQKVVDVLDSIKTKGKSADFTNFDPRGLLPESLDYWTYPGSQTIPPLIESVTWIVLKEPISVSSEQVLKFRKLNFNGEGEPEELMVDNWRPAQPLKNRQIKASFK

SEQ ID NO: 3 - protein, 259 aa, synthetic construct
SHHWGYGKHNGPEHWHKDFPIAKGERQSPVDIDTHTAKYDPSLKPLSVSYDQATSLRILNDGHAFAVEFDDSQDKAVLKGGPLDGTYRLFQFHFHWGSLDGQGSEHTVDKKKYAAELHLVHWNTKYGDFGKAKQQPDGLAVLGIFLKVGSAKPGLQKVVDVLDSIKTKGKSADFTNFDPRGLLPESLDYWTYPGSQTVPPLLESVTWIVLKEPISVSSEQVLKFRKLNFNGEGEPEELMVDNWRPAQPLKNRQIKASFK

SEQ ID NO: 4 - protein, 259 aa, synthetic construct
SHHWGYGKHNGPEHWHKDFPIAKGERQSPVDIDTHTAKYDPSLKPLSVSYDQATSLRILNNGHAFDVEFDDSQDKAVLKGGPLDGTYRLHQFHFHWGSLDGQGSEHTVDKKKYAAELHLVHWNTKYGDYGKAAQQPDGIAILGIFLKVGSAKPGLQKVVDVLDSIKTKGKSADFTNFDPRGLLPESLDYWTYPGSMTIPPLLESVTWIVLKEPISVSSEQVLKFRKLNFNGEGEPEELMVDNWRPAQPLKNRQIKASFK

SEQ ID NO: 5 - protein, 259 aa, synthetic construct
SHHWGYGKHNGPEHWHKDFPIAKGERQSPVDIDTHTAKYDPSLKPLSVSYDQATSLRILNNGHTFIVEFDDSQDKAVLKGGPLDGTYRLVQFHFHWGSLDGQGSEHTVDKKKYAAELHLVHWNTKYGDLGKALQQPDGLAILGIFLKVGSAKPGLQKVVDVLDSIKTKGKSADFTNFDPRGLLPESLDYWTYPGSWTVPPLLESVTWIVLKEPISVSSEQVLKFRKLNFNGEGEPEELMVDNWRPAQPLKNRQIKASFK

SEQ ID NO: 6 - protein, 260 aa, synthetic construct
ASPDWGYDDKNGPEQWSKLYPIANGNNQSPVDIKTSETKHDTSLKPISVSYNPATAKEIINVGHSFRVNFEDNDNRSVLKGGPFSDSYRLYQFHFHWGSTNEHGSEHTVDGVKYSAELHVVHWNSAKYSSLAEALSKADGIAVIGVLMKVGEANPKLQKVLDALQAIKTKGKRAPFTNFDPSTLLPSSLDFWTYPGSWTAPPLYEIVTWIILKESISVSSEQLAQFRSLLSNVEGDNAVPMQHNNRPTQPLKGRTVRASF

SEQ ID NO: 7 - protein, 260 aa, synthetic construct
ASPDWGYDDKNGPEQWSKLYPIANGNNQSPVDIKTSETKHDTSLKPISVSYNPATAKEIINVGHSFWVNFEDNDNRSVLKGGPFSDSYRLFQFHFHWGSTNEHGSEHTVDGVKYSAELHVAHWNSAKYSSLAEAQSKADGVAIIGVLMKVGEANPKLQKVLDALQAIKTKGKRAPFTNFDPSTLLPSSLDFWTYPGSLTVPPLFESVTWIILKESISVSSEQLAQFRSLLSNVEGDNAVPMQHNNRPTQPLKGRTVRASF

SEQ ID NO: 8 - protein, 700 aa, Thermomonas hydrothermalis
DEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDNWMWPRHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNNTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFDGTLESVASNWVFDPVLTRMISVDQRYMRWIMQEVMPAPQLLEELGVPPRQ

SEQ ID NO: 9 - protein, 700 aa, synthetic construct
DEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDMWMWPSHTGDFSFYRAYVGKDGKPAPYSKDNVPYRPKHWLKIADTPLGEGDFVMVAGYPGRTDRYALVAEFENTQRWLYPAISKAYKDQIALVEAAAKDNPEIAVKYAAALAGWNPTSKNFDGQLEGFKRNDVLAIKRREEAAVLRWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLRRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKCTGVEPFDCPKALLEAVAAKRYAGLADARLGTVPVNFLADLDITGGNSGSPVLDANGRLVGLAFQVTLESVASNWVFDPVLTRMISVDQRYMRWIMQEVMPAPQLLEELGVPPRQ

SEQ ID NO: 10 - protein, 700 aa, synthetic construct
DEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDMWMWPSHTGDFSFYRAYVGKDGKPAPYSKDNVPYRPKHWLKIADTPLGEGDFVMVAGYPGRTDRYALVAEFENTQRWLYPAISKAYKDQIALVEAAAKDNPEIAVKYAAALAGWNPTSKLFDGQLEGFKRNDVLAIKRREEAAVLRWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLRRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKCTGVEPFDCPKALLEAVAAKRYAGLADARLGTVPVNFLADLDGTGGNSGSPVLDANGRLVGLVFQGTLESVASDWVFDPVLTRCISVDQRYMRWIMQEVMPAPQLLEELGVPPRQ

SEQ ID NO: 11 - protein, 700 aa, synthetic construct
DEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDMWMWPSHTGDFSFYRAYVGKDGKPAPYSKDNVPYRPKHWLKIADTPLGEGDFVMVAGYPGRTDRYALVAEFENTQRWLYPAISKAYKDQIALVEAAAKDNPEIAVKYAAALAGWNPTSKLFDGQLEGFKRNDVLAIKRREEAAVLRWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLRRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKCTGVEPFDCPKALLEAVAAKRYAGLADARLGTVPVNFLADLDTTGGNSGSPVLDANGRLVGLGFQRTLESVASNWVFDPVLTRSISVDQRYMRWIMQEVMPAPQLLEELGVPPRQ

SEQ ID NO: 12 - DNA, 26 bp, synthetic construct
  source 1..26: mol_type = other DNA
tatttatgctactaggaagagtagaa

SEQ ID NO: 13 - DNA, 34 bp, synthetic construct
  source 1..34: mol_type = other DNA
tatataccatatcgtgctactaggaagaggtaaa

SEQ ID NO: 14 - DNA, 34 bp, synthetic construct
  source 1..34: mol_type = other DNA
tatacaaccgagacggctctgaggaagaggtaaa

SEQ ID NO: 15 - DNA, 34 bp, synthetic construct
  source 1..34: mol_type = other DNA
tatgtactgtgtactgctactaggaagaggtaaa

SEQ ID NO: 16 - DNA, 32 bp, synthetic construct
  source 1..32: mol_type = other DNA
tataatatattgtgctactaggaagaggtaaa

SEQ ID NO: 17 - DNA, 34 bp, synthetic construct
  source 1..34: mol_type = other DNA
tattcgaacacaggggctactaggaagaggtaaa

SEQ ID NO: 18 - protein, 8 aa, synthetic construct
  VARIANT 6: note = any one amino acid residue or no residue
LVNDVXDF

SEQ ID NO: 19 - protein, 6 aa, synthetic construct
LVNEVT

SEQ ID NO: 20 - protein, 5 aa, synthetic construct
LVNEV

SEQ ID NO: 21 - protein, 6 aa, synthetic construct
MLEEIM

SEQ ID NO: 22 - protein, 6 aa, synthetic construct
YLYEIA

SEQ ID NO: 23 - protein, 5 aa, synthetic construct
LLDNW

SEQ ID NO: 24 - protein, 5 aa, synthetic construct
IVDLV

SEQ ID NO: 25 - protein, 9 aa, synthetic construct
LVNEVTEFA

SEQ ID NO: 26 - protein, 8 aa, synthetic construct
LVNEVTEF

SEQ ID NO: 27 - protein, 7 aa, synthetic construct
LVNEVTE

SEQ ID NO: 28 - protein, 25 aa, synthetic construct
FLFVVAAATGVQSQVQLVQSGAEVK

SEQ ID NO: 29 - protein, 6 aa, synthetic construct
  VARIANT 3: note = any one amino acid residue or no residue
FFXFVV

SEQ ID NO: 30 - protein, 7 aa, synthetic construct
FLVNLVK

SEQ ID NO: 31 - protein, 12 aa, synthetic construct
FLVGPDGIPIMR

SEQ ID NO: 32 - protein, 10 aa, synthetic construct
LILVDYILFK

SEQ ID NO: 33 - protein, 16 aa, synthetic construct
FIQVGVISWGVVDVCK